(19) United States
(12) Patent Application Publication
(10) Pub. No.: US 2025/0090633 A1
(43) Pub. Date: Mar. 20, 2025

(54) THERAPEUTIC COMPOSITIONS AND METHODS OF USE THEREOF

(71) Applicant: Juvena Therapeutics, Inc., Redwood City, CA (US)

(72) Inventors: Hanadie YOUSEF, Palo Alto, CA (US); Jeremy O’CONNELL, Palo Alto, CA (US); Thach MAI, Palo Alto, CA (US)

(21) Appl. No.: 18/889,225

(22) Filed: Sep. 18, 2024

Related U.S. Application Data
(60) Provisional application No. 63/583,536, filed on Sep. 18, 2023.

(51) Int. Cl. A61K 38/18; A61K 38/17; A61P 11/00

(52) U.S. Cl. CPC A61K 38/1825; A61K 38/179; A61K 38/1808; A61P 11/00

(57) ABSTRACT

Described herein are therapeutic compositions comprising polypeptides (e.g., OLFML3, NDP, AREG, FGF1, EFNB2, and/or CCN1) useful for the treatment of IPF. Also described herein are methods of treatment for IPF comprising administering a therapeutically effective amount of a composition comprising a polypeptide (e.g., OLFML3, NDP, AREG, FGF1, EFNB2, and/or CCN1) to a subject in need thereof.

### CROSS-REFERENCE TO RELATED APPLICATIONS

This application claims the benefit of U.S. Provisional Patent Application No. 63/583,536, filed on Sep. 18, 2023, which is incorporated by reference herein in its entirety.

### INCORPORATION BY REFERENCE OF SEQUENCE LISTING

The present application is being filed along with a Sequence Listing in electronic format. The Sequence Listing is provided as a file entitled 54275-711_201_SL.xml, created on Sep. 18, 2024, which is 102,473 bytes in size. The information in the electronic format of the Sequence Listing is incorporated by reference in its entirety.

### BACKGROUND

As the average life span lengthens, increasing emphasis is placed upon “healthy aging”. Individuals would like to live more active lifestyles as they age, and as a result, many aging disorders can have a significant impact on the quality of life of aging individuals. Treatments directed to regenerative ends have utility for treating aging diseases. Additionally, many treatments for aging disorders can be applicable to younger individuals who have suffered illness, injury, or who possess genetic or developmental defects leading to premature tissue loss, wasting, or weakening. Many aging disorders have a poor prognosis with a low chance of recovery, a high risk of disease-associated mortality, or both. Current treatment options for aging disorders are limited.

Interstitial lung disease (ILD) is an umbrella term used to represent a large group of diseases that cause scarring, or fibrosis, of the lungs. An ILD can result from progression of an aging disorder. Lung damage from ILDs often persists in patients, may be irreversible with current treatments, and tends to worsen over time. Pulmonary scarring, or pulmonary fibrosis, causes increased stiffness in the lungs of affected patient, making it difficult to breath and supply oxygen to the bloodstream. There are at least six broad categories of ILDs. In the first category, ILDs may develop due to environmental exposure or occupational-related hazards, (e.g., asbestosis, silicosis, or hypersensitivity pneumonitis). A second cause of ILDs are brought on as a result of particular treatments (e.g., chemotherapy, radiation therapy, or certain medications). Autoimmune or connective tissue diseases represent a third category of ILD. Examples of these include lupus, scleroderma, poly or dermatomyositis, or rheumatoid arthritis-related ILD. A fourth category is an ILD brought on by a granulomatous disease such as sarcoidosis. A fifth category is an ILD induced following a viral illness (e.g., post-COVID-19 interstitial lung disease). Lastly, the sixth category of ILD are idiopathic ILDs.

Idiopathic pulmonary fibrosis (IPF) is a class of ILD and is the most common type of pulmonary fibrosis. IPF is a disease marked by scarring (fibrosis) of the lungs. Idiopathic means it has no known cause. Scarring from IPF causes stiffness in the lungs and makes it difficult to breathe. Chronic pulmonary inflammation is a characteristic of IPF and is often accompanied by an uncontrolled healing response causing progressive scarring or thickening (fibrosis) of tissues between alveoli in the lung.

### SUMMARY

As individuals age, tissue progenitor cells lose their regenerative potential. Described herein, in certain aspects, are heparin-associated polypeptides that can restore some or all of this regenerative potential, and are thus useful in the treatment of aging disorders that result in tissue loss or underperformance, and rehabilitation from injury. Described herein are therapeutic compositions comprising heparin-associated polypeptides and methods of treating disorders associated with aging, injury, or illness. In certain embodiments, the therapeutic compositions comprise one or more heparin-associated polypeptides that possess mitogenic (i.e., regenerative) promoting activity, fibrosis inhibiting activity, lung cell function-improving activity, or a combination thereof, to a somatic cell, such as a lung tissue progenitor cell. In certain embodiments, the therapeutic compositions have activity towards lung progenitor cells. In certain embodiments, the therapeutic compositions have activity towards AT2 cells. In certain embodiments these compositions possess utility in treating interstitial lung disease (ILD). In certain embodiments these compositions possess utility in treating idiopathic pulmonary fibrosis (IPF).

In certain aspects, disclosed herein is a composition comprising: OLFML3, NDP, AREG, FGF1, EFNB2, and/or CCN1 or any combination thereof. In certain aspects, the composition comprises OLFML3 and/or a sequence comprising at least about 90% identity to one of SEQ ID NO: 1-3, 16, or 24. In certain aspects, the composition comprises NDP and/or a sequence comprising at least about 90% identity to one of SEQ ID NO: 4, 17, or 18. In certain aspects, the composition comprises AREG and/or a sequence comprising at least about 90% identity to one of SEQ ID NO: 15 or 23. In certain aspects, the composition comprises FGF1 and/or a sequence comprising at least about 90% identity to one of SEQ ID NO: 5-8, 19, or 20. In certain aspects, the composition comprises EFNB2 and/or a sequence comprising at least about 90% identity to one of SEQ ID NO: 9-13, 21, or 25. In certain aspects, the composition comprises CCN1 and/or a sequence comprising at least about 90% identity to one of SEQ ID NO: 14, 22, or 26. In certain aspects, the therapeutic polypeptide of the composition has been produced in a mammalian, yeast, insect or bacteria cell. In certain aspects, the therapeutic polypeptide of the composition has been purified from a human biological sample. In certain aspects, the therapeutic polypeptide of the composition has been produced in a mammalian cell and the mammalian cell is a human cell, Chinese Hamster Ovary (CHO) cell or mouse myeloma cell. In certain aspects, the composition comprises a polypeptide having an amino acid sequence comprising at least about 90% identity SEQ ID NO: 1. In certain aspects, the composition comprises a polypeptide having an amino acid sequence comprising at least about 90% identity SEQ ID NO: 2. In certain aspects, the composition comprises a polypeptide having an amino acid sequence comprising at least about 90% identity SEQ ID NO: 3. In certain aspects, the composition comprises a polypeptide having an amino acid sequence comprising at least about 90% identity SEQ ID NO: 4. In certain aspects, the composition comprises a polypeptide having an amino acid sequence comprising at least about 90% identity SEQ ID NO: 5. In certain aspects, the composition comprises a polypeptide having an amino acid sequence comprising at least about 90% identity SEQ ID NO: 6. In certain aspects, the composition comprises a polypeptide having an amino acid sequence comprising at least about 90% identity SEQ ID NO: 7. In certain aspects, the composition comprises a polypeptide having an amino acid sequence comprising at least about 90% identity SEQ ID NO: 8. In certain aspects, the composition comprises a polypeptide having an amino acid sequence comprising at least about 90% identity SEQ ID NO: 9. In certain aspects, the composition comprises a polypeptide having an amino acid sequence comprising at least about 90% identity SEQ ID NO: 10. In certain aspects, the composition comprises a polypeptide having an amino acid sequence comprising at least about 90% identity SEQ ID NO: 11. In certain aspects, the composition comprises a polypeptide having an amino acid sequence comprising at least about 90% identity SEQ ID NO: 12. In certain aspects, the composition comprises a polypeptide having an amino acid sequence comprising at least about 90% identity SEQ ID NO: 13. In certain aspects, the composition comprises a polypeptide having an amino acid sequence comprising at least about 90% identity SEQ ID NO: 14. In certain aspects, the composition comprises a polypeptide having an amino acid sequence comprising at least about 90% identity SEQ ID NO: 15. In certain aspects, the composition comprises a polypeptide having an amino acid sequence comprising at least about 90% identity SEQ ID NO: 16. In certain aspects, the composition comprises a polypeptide having an amino acid sequence comprising at least about 90% identity SEQ ID NO: 17. In certain aspects, the composition comprises a polypeptide having an amino acid sequence comprising at least about 90% identity SEQ ID NO: 18. In certain aspects, the composition comprises a polypeptide having an amino acid sequence comprising at least about 90% identity SEQ ID NO: 19. In certain aspects, the composition comprises a polypeptide having an amino acid sequence comprising at least about 90% identity SEQ ID NO: 20. In certain aspects, the composition comprises a polypeptide having an amino acid sequence comprising at least about 90% identity SEQ ID NO: 21. In certain aspects, the composition comprises a polypeptide having an amino acid sequence comprising at least about 90% identity SEQ ID NO: 22. In certain aspects, the composition comprises a polypeptide having an amino acid sequence comprising at least about 90% identity SEQ ID NO: 23. In certain aspects, the composition comprises a polypeptide having an amino acid sequence comprising at least about 90% identity SEQ ID NO: 24. In certain aspects, the composition comprises a polypeptide having an amino acid sequence comprising at least about 90% identity SEQ ID NO: 25. In certain aspects, the composition comprises a polypeptide having an amino acid sequence comprising at least about 90% identity SEQ ID NO: 26. In certain aspects, the composition comprises a polypeptide having an amino acid sequence comprising at least about 95%, 96%, 97%, 98%, or 99% identity selected from one of SEQ ID Nos: 1-26. In certain aspects, the composition comprises a polypeptide comprising the amino acid sequence of SEQ ID NO: 1. In certain aspects, the composition comprises a polypeptide comprising the amino acid sequence of SEQ ID NO: 2. In certain aspects, the composition comprises a polypeptide comprising the amino acid sequence of SEQ ID NO: 3. In certain aspects the composition comprises a polypeptide comprising the amino acid sequence of SEQ ID NO: 4. In certain aspects, the composition comprises a polypeptide comprising the amino acid sequence of SEQ ID NO: 5. In certain aspects, the composition comprises a polypeptide comprising the amino acid sequence of SEQ ID NO: 6. In certain aspects, the composition comprises a polypeptide comprising the amino acid sequence of SEQ ID NO: 7. In certain aspects, the composition comprises a polypeptide comprising the amino acid sequence of SEQ ID NO: 8. In certain aspects, the composition comprises a polypeptide comprising the amino acid sequence of SEQ ID NO: 9. In certain aspects, the composition comprises a polypeptide comprising the amino acid sequence of SEQ ID NO: 10. In certain aspects, the composition comprises a polypeptide comprising the amino acid sequence of SEQ ID NO: 11. In certain aspects, the composition comprises a polypeptide comprising the amino acid sequence of SEQ ID NO:12. In certain aspects, the composition comprises a polypeptide comprising the amino acid sequence of SEQ ID NO: 13. In certain aspects, the composition comprises a polypeptide comprising the amino acid sequence of SEQ ID NO: 14. In certain aspects, the composition comprises a polypeptide comprising the amino acid sequence of SEQ ID NO: 15. In certain aspects, the composition comprises a polypeptide comprising the amino acid sequence of SEQ ID NO: 16. In certain aspects, the composition comprises a polypeptide comprising the amino acid sequence of SEQ ID NO: 17. In certain aspects, the composition comprises a polypeptide comprising the amino acid sequence of SEQ ID NO: 18. In certain aspects, the composition comprises a polypeptide comprising the amino acid sequence of SEQ ID NO: 19. In certain aspects, the composition comprises a polypeptide comprising the amino acid sequence of SEQ ID NO: 20. In certain aspects, the composition comprises a polypeptide comprising the amino acid sequence of SEQ ID NO: 21. In certain aspects, the composition comprises a polypeptide comprising the amino acid sequence of SEQ ID NO: 22. In certain aspects, the composition comprises a polypeptide comprising the amino acid sequence of SEQ ID NO: 23. In certain aspects, the composition comprises a polypeptide comprising the amino acid sequence of SEQ ID NO: 24. In certain aspects, the composition comprises a polypeptide comprising the amino acid sequence of SEQ ID NO: 25. In certain aspects, the composition comprises a polypeptide comprising the amino acid sequence of SEQ ID NO: 26.

In certain aspects, the composition further comprises a pharmaceutically acceptable excipient. In certain embodiments, the composition is formulated for administration by injection. In certain aspects, the injection is intramuscular injection, subcutaneous injection, or intravenous injection. In certain aspects, disclosed herein is a composition comprising OLFML3, NDP, AREG, FGF1, EFNB2, and/or CCN1 or any combination thereof, and a pharmaceutically acceptable excipient. In certain aspects, the composition comprises OLFML3 and/or a sequence comprising at least about 90% identity to one of SEQ ID NO: 1-3, 16, or 24. In certain aspects, the composition comprises NDP and/or a sequence comprising at least about 90% identity to one of SEQ ID NO: 4, 17, or 18. In certain aspects, the composition comprises AREG and/or a sequence comprising at least about 90% identity to one of SEQ ID NO: 15 or 23. In certain aspects, the composition comprises FGF1 and/or a sequence comprising at least about 90% identity to one of SEQ ID NO: 5-8. 19, or 20. In certain aspects, the composition comprises EFNB2 and/or a sequence comprising at least about 90% identity to one of SEQ ID NO: 9-13, 21, or 25. In certain aspects, the composition comprises CCN1 and/or a sequence comprising at least about 90% identity to one of SEQ ID NO: 14, 22, or 26 . . . . In certain aspects, the polypeptide of the composition has been expressed from a human cell, Chinese Hamster Ovary (CHO) cell, or mouse myeloma (NS0) cell, or a bacterial cell. In certain aspects, the polypeptide is synthetically produced, or has been purified from a human biological sample. In certain aspects, the polypeptide possesses mitogenic activity. In certain aspects, the polypeptide possesses lung regenerative activity. In certain aspects, the polypeptide possesses lung fibrosis inhibiting activity. In certain aspects, the polypeptide possesses lung cell function improvement activity. Also disclosed is a nucleic acid encoding the polypeptide(s) disclosed herein. Also disclosed is a cell line comprising the nucleic acid disclosed herein. Also disclosed is a method of treating a disease or condition (e.g., IPF) in a subject in need thereof, the method comprising administering to the subject the composition disclosed herein. In certain aspects, the disease or condition comprises an ILD. In some aspects, the ILD develops due to an environmental exposure or an occupational-related hazard. In some aspects, the ILD develops due to a result of particular therapeutic treatment. In some embodiments, the therapeutic treatment comprises chemotherapy. In some embodiments, the therapeutic treatment comprises radiation therapy. In some embodiments, the therapeutic treatment comprises ILD-inducing small molecule therapy. In some aspects, the ILD develops due to an autoimmune disorder. In some aspects, the ILD develops due to a connective tissue disease. In some aspects, the ILD develops due to progression of a granulomatous disorder. In some aspects, the ILD develops due to COVID-19 infection and respiratory illness. In some aspects, the ILD develops due to progression of an Idiopathic pulmonary fibrosis (IPF). In certain aspects, the subject has Idiopathic pulmonary fibrosis.

Described herein are methods of treating a subject having an interstitial lung disease (ILD), the methods comprising administering to the subject an effective amount of an OLFML3 protein or functional derivative thereof, wherein the effective amount improves one or more symptoms associated with pulmonary fibrosis in the subject. Described herein are methods of treating a subject having an interstitial lung disease (ILD), the methods comprising administering to the subject an effective amount of an OLFML3 protein or functional derivative thereof, wherein the effective amount increases alveolar regeneration in the subject, promotes repair of pulmonary fibrosis in the subject, restores at least partial lung function in the subject, or any combination thereof. Described herein are methods of treating a subject having an interstitial lung disease (ILD), the methods comprising administering to the subject an effective amount of an NDP protein or functional derivative thereof, wherein the effective amount improves one or more symptoms associated with pulmonary fibrosis in the subject. Described herein are methods of treating a subject having an interstitial lung disease (ILD), the methods comprising administering to the subject an effective amount of an NDP protein or functional derivative thereof, wherein the effective amount increases alveolar regeneration in the subject, promotes repair of pulmonary fibrosis in the subject, restores at least partial lung function in the subject, or any combination thereof. Described herein are methods of treating a subject having an interstitial lung disease (ILD), the methods comprising administering to the subject an effective amount of an FGF1 protein or functional derivative thereof, wherein the effective amount improves one or more symptoms associated with pulmonary fibrosis in the subject. Described herein are methods of treating a subject having an interstitial lung disease (ILD), the methods comprising administering to the subject an effective amount of an FGF1 protein or functional derivative thereof, wherein the effective amount increases alveolar regeneration in the subject, promotes repair of pulmonary fibrosis in the subject, restores at least partial lung function in the subject, or any combination thereof. Described herein are methods of treating a subject having an interstitial lung disease (ILD), the methods comprising administering to the subject an effective amount of an EFNB2 protein or functional derivative thereof, wherein the effective amount improves one or more symptoms associated with pulmonary fibrosis in the subject. Described herein are methods of treating a subject having an interstitial lung disease (ILD), the methods comprising administering to the subject an effective amount of an EFNB2 protein or functional derivative thereof, wherein the effective amount increases alveolar regeneration in the subject, promotes repair of pulmonary fibrosis in the subject, restores at least partial lung function in the subject, or any combination thereof. Described herein are methods of treating a subject having an interstitial lung disease (ILD), the methods comprising administering to the subject an effective amount of an CCN1 protein or functional derivative thereof, wherein the effective amount improves one or more symptoms associated with pulmonary fibrosis in the subject. Described herein are methods of treating a subject having an interstitial lung disease (ILD), the methods comprising administering to the subject an effective amount of an CCN1 protein or functional derivative thereof, wherein the effective amount increases alveolar regeneration in the subject, promotes repair of pulmonary fibrosis in the subject, restores at least partial lung function in the subject, or any combination thereof. Described herein are methods of treating a subject having an interstitial lung disease (ILD), the methods comprising administering to the subject an effective amount of an AREG protein or functional derivative thereof, wherein the effective amount improves one or more symptoms associated with pulmonary fibrosis in the subject. Described herein are methods of treating a subject having an interstitial lung disease (ILD), the methods comprising administering to the subject an effective amount of an AREG protein or functional derivative thereof, wherein the effective amount increases alveolar regeneration in the subject, promotes repair of pulmonary fibrosis in the subject, restores at least partial lung function in the subject, or any combination thereof

Described herein are methods of treating a subject having an idiopathic pulmonary fibrosis (IPF), the methods comprising administering to the subject an effective amount of an OLFML3 protein or functional derivative thereof, wherein the effective amount improves one or more symptoms associated with pulmonary fibrosis in the subject. Described herein are methods of treating a subject having an idiopathic pulmonary fibrosis (IPF), the methods comprising administering to the subject an effective amount of an OLFML3 protein or functional derivative thereof, wherein the effective amount increases alveolar regeneration in the subject, promotes repair of pulmonary fibrosis in the subject, restores at least partial lung function in the subject, or any combination thereof. Described herein are methods of treating a subject having an idiopathic pulmonary fibrosis (IPF), the methods comprising administering to the subject an effective amount of an NDP protein or functional derivative thereof, wherein the effective amount improves one or more symptoms associated with pulmonary fibrosis in the subject. Described herein are methods of treating a subject having an idiopathic pulmonary fibrosis (IPF), the methods comprising administering to the subject an effective amount of an NDP protein or functional derivative thereof, wherein the effective amount increases alveolar regeneration in the subject, promotes repair of pulmonary fibrosis in the subject, restores at least partial lung function in the subject, or any combination thereof. Described herein are methods of treating a subject having an idiopathic pulmonary fibrosis (IPF), the methods comprising administering to the subject an effective amount of an FGF1 protein or functional derivative thereof, wherein the effective amount improves one or more symptoms associated with pulmonary fibrosis in the subject. Described herein are methods of treating a subject having an idiopathic pulmonary fibrosis (IPF), the methods comprising administering to the subject an effective amount of an FGF1 protein or functional derivative thereof, wherein the effective amount increases alveolar regeneration in the subject, promotes repair of pulmonary fibrosis in the subject, restores at least partial lung function in the subject, or any combination thereof. Described herein are methods of treating a subject having an idiopathic pulmonary fibrosis (IPF), the methods comprising administering to the subject an effective amount of an EFNB2 protein or functional derivative thereof, wherein the effective amount improves one or more symptoms associated with pulmonary fibrosis in the subject. Described herein are methods of treating a subject having an idiopathic pulmonary fibrosis (IPF), the methods comprising administering to the subject an effective amount of an EFNB2 protein or functional derivative thereof, wherein the effective amount increases alveolar regeneration in the subject, promotes repair of pulmonary fibrosis in the subject, restores at least partial lung function in the subject, or any combination thereof. Described herein are methods of treating a subject having an idiopathic pulmonary fibrosis (IPF), the methods comprising administering to the subject an effective amount of an CCN1 protein or functional derivative thereof, wherein the effective amount improves one or more symptoms associated with pulmonary fibrosis in the subject. Described herein are methods of treating a subject having an idiopathic pulmonary fibrosis (IPF), the methods comprising administering to the subject an effective amount of an CCN1 protein or functional derivative thereof, wherein the effective amount increases alveolar regeneration in the subject, promotes repair of pulmonary fibrosis in the subject, restores at least partial lung function in the subject, or any combination thereof. Described herein are methods of treating a subject having an idiopathic pulmonary fibrosis (IPF), the methods comprising administering to the subject an effective amount of an AREG protein or functional derivative thereof, wherein the effective amount improves one or more symptoms associated with pulmonary fibrosis in the subject. Described herein are methods of treating a subject having an idiopathic pulmonary fibrosis (IPF), the methods comprising administering to the subject an effective amount of an AREG protein or functional derivative thereof, wherein the effective amount increases alveolar regeneration in the subject, promotes repair of pulmonary fibrosis in the subject, restores at least partial lung function in the subject, or any combination thereof.

In an aspect described herein are methods of treating a subject having an idiopathic pulmonary fibrosis (IPF), the methods comprising administering to the subject an effective amount of (i) an OLFML3 protein or functional derivative thereof, (ii) an NDP protein or functional derivative thereof, (iii) an AREG protein or functional derivative thereof, (iv) an FGF1 protein or functional derivative thereof, (v) an EFNB2 protein or functional derivative thereof or an EFNB2-Fc fusion protein, or (vi) a CCN1 protein or functional derivative thereof or a CCN1-Fc fusion protein, wherein the effective amount increases alveolar regeneration in the subject, promotes repair of pulmonary fibrosis in the subject, restores at least partial lung function in the subject, or any combination thereof. In some embodiments, the effective amount of the EFNB2-Fc fusion protein is administered, and repair of pulmonary fibrosis is promoted in the subject. In some embodiments, the effective amount of the EFNB2-Fc fusion protein is administered, and epithelial-mesenchymal transition (EMT) in AT2 cells is inhibited in the subject. In some embodiments, the effective amount of the EFNB2-Fc fusion protein is administered, and expression of Vimentin per area on the surface of AT2 cells is decreased in the subject. In some embodiments, the effective amount of the FGF1 protein or functional derivative thereof is administered, and alveolar regeneration is increased in the subject. In some embodiments, the effective amount of the FGF1 protein or functional derivative thereof is administered, and AT2 cell viability is increased in the subject. In some embodiments, the effective amount of the FGF1 protein or functional derivative thereof is administered, and lung resistance is decreased in the subject. In some embodiments, the effective amount of the FGF1 protein or functional derivative thereof is administered, and lung elastance is decreased in the subject. In some embodiments, the effective amount of the FGF1 protein or functional derivative thereof is administered, and lung compliance is improved in the subject. In some embodiments, the effective amount of the AREG protein or functional derivative thereof is administered, and alveolar regeneration is increased in the subject. In some embodiments, the effective amount of the AREG protein or functional derivative thereof is administered, and AT2 cell viability is increased in the subject. In some embodiments, the effective amount of the AREG protein or functional derivative thereof is administered, and surfactant SP-C total area of cell surface expression in AT2 cells is increased in the subject. In some embodiments, the effective amount of the CCN1-Fc fusion protein is administered, and at least partial lung function is restored in the subject. In some embodiments, the effective amount of the CCN1-Fc fusion protein is administered, and surfactant SP-C production is increased in AT2 cells in the subject. In some embodiments, the effective amount of the CCN1-Fc fusion protein is administered, and surfactant SP-C total area of cell surface expression in AT2 cells is increased in the subject.

In an aspect described herein are methods of treating a subject having an idiopathic pulmonary fibrosis (IPF), the methods comprising administering to the subject an effective amount of (i) an OLFML3 protein or functional derivative thereof, (ii) an NDP protein or functional derivative thereof, (iii) an AREG protein or functional derivative thereof, (iv) an FGF1 protein or functional derivative thereof, (v) an EFNB2 protein or functional derivative thereof or an EFNB2-Fc fusion protein, or (vi) a CCN1 protein or functional derivative thereof or a CCN1-Fc fusion protein, wherein the effective amount improves one or more symptoms associated with pulmonary fibrosis in the subject.

In an aspect described herein are methods of treating a subject having an interstitial lung disease (ILD), the methods comprising administering to the subject an effective amount of an (i) an OLFML3 protein or functional derivative thereof, (ii) an NDP protein or functional derivative thereof, (iii) an AREG protein or functional derivative thereof, (iv) an FGF1 protein or functional derivative thereof, (v) an EFNB2 protein or functional derivative thereof or an EFNB2-Fc fusion protein, or (vi) a CCN1 protein or functional derivative thereof or a CCN1-Fc fusion protein, wherein the effective amount increases alveolar regeneration in the subject, promotes repair of pulmonary fibrosis in the subject, restores at least partial lung function in the subject, or any combination thereof.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1A-FIG. 1B show schematic diagrams of secretomes from pluripotent stem cells to be isolated for analysis. In FIG. 1A, a secretome from human embryonic stem cells (hESCs) is shown in a diagram capable of eliciting functional effects in lung stem cells and derivatives thereof. In FIG. 1B, a secretome from differentiated stem cells is shown in a diagram capable of eliciting functional effects in lung stem cells and derivatives thereof.

FIG. 2A-FIG. 2B show mapping of disease-modifying effects through in vitro phenotype mapping and quantitative mass spectrometry analysis of individual protein composition and abundance of each secretome. In FIG. 2A, in vitro phenotype mapping of differentiation, immunomodulatory effects, antifibrotic effects, and anti-apoptosis in lung stem cells and derivatives thereof are shown. In FIG. 2B, quantitative mass spectrometry is shown being used to determine the individual protein composition and abundance of each secretome.

FIG. 3 shows proteins with pro-regenerative properties selected and ranked using bioinformatics analysis and machine learning models trained on a proprietary feature set comprised of multi-dimensional map of proteins and their diseases-modifying effects across different organs and indications.

FIG. 4A-FIG. 4E show the elements considered in screening candidate ranking. Disease context, chronological expression, ligand-receptor mapping, safety profile, and stem-cell secretome features are considered in screening candidate ranking. FIG. 4A shows results of disease context via analysis of expression changes identified between various data sets and subsequent pathway analysis. FIG. 4B shows results of chronological expression analysis of various genes up-regulated or down-regulated in various age groups. FIG. 4C shows a diagram of representative forms of ligand-receptor mapping for pathways of interest. FIG. 4D shows graphs of effects considering the safety profile of pathway implicated by gene effect (top graph) and probability of survival as a function of time (bottom graph). FIG. 4E shows diagrams of implicated proteins from analyzed secretomes and analysis via quantitative mass spectrometry analysis of individual protein abundance.

FIG. 5 shows a schematic diagram of a high throughput screening pipeline of pre-ranked secreted proteins identifying several factors that promote AT2 regeneration under fibrotic stress and assays used therein to screen for viability, epithelialization, and surfactant expression.

FIG. 6 shows heatmap clustering of factors that alter AT2 phenotypes in the screen from FIG. 5. Cells: Human AT2 cells. Media: Basal media (BM) (n=3 human donor cells).

FIG. 7A-FIG. 7B show representative dose range curves of factors that improve surfactant C expression and increase AT2 cell counts. FIG. 7A shows a dose range curve for CCN1 increasing surfactant C expression in AT2 cells. FIG. 7B shows a dose range curve for CCN1 increasing AT2 cell counts.

FIG. 8A-FIG. 8D shows effects of various secreted factors promoting AT2 function or inhibiting EMT under fibrotic conditions in AT2 cells from healthy donors. FIG. 8A shows a graph demonstrating effects of EFNB2 inhibiting AT2 cell EMT. FIG. 8B shows a graph demonstrating effects of FGF1 in promoting AT2 cell viability. FIG. 8C shows a graph demonstrating effects of F212 in promoting AT2 cell viability. FIG. 8D shows a graph demonstrating effects of CCN1 in promoting SP-C production in cells derived from AT2 cells from healthy donors.

FIG. 9A-FIG. 9D shows effects of various secreted factors promoting AT2 function or inhibiting EMT under fibrotic conditions in AT2 cells from an IPF donor. FIG. 9A shows a graph demonstrating effects of EFNB2 inhibiting AT2 cell EMT in cells from a subject with IPF.

FIG. 9B shows a graph demonstrating effects of FGF1 in promoting AT2 cell viability in cells from a subject with IPF. FIG. 9C shows a graph demonstrating effects of F212 in promoting AT2 cell viability in cells from a subject with IPF. FIG. 9D shows a graph demonstrating effects of CCN1 in promoting SP-C production in cells derived from AT2 cells from an IPF donor.

FIG. 10 shows a schematic of air-liquid-interface (ALI) model of chronic fibrosis used to assay secreted factors for effects in human alveolar epithelial cells following drug treatment.

FIG. 11A-FIG. 11B show graphs of EMT phenotype and SP-C production of human AT2 cells under fibrotic stress using the model shown in FIG. 10. FIG. 11A shows a graph demonstrating extent of EMT as measured by total vimentin area fold change following treatment. FIG. 11B shows a graph demonstrating extent of change in SP-C production following treatment.

FIG. 12 shows a heatmap of fibrotic and cellular stress pathways upregulated by TGFβ1 treatment that are attenuated by multiple secreted proteins as described in Example 4.

FIG. 13 shows a line plot of weight change in mice across the study (n=10). 12 week old male C57BL/6 mice were injected with 1.3 mg/kg bleomycin. Vehicle or FGF1 was injected I.P. daily starting day 7 post-bleomycin.

FIG. 14A-FIG. 14B show functional effects in the lungs following treatment in the bleomycin model of IPF. FIG. 14A shows a bar graph of lung resistance, a metric of the level of constriction (n=8-10) according to sham, bleomycin+vehicle, and bleomycin+FGF1 treatment groups. FIG. 14B shows a bar graph of lung elastance, a metric of stiffness (n=8-10) according to sham, bleomycin+vehicle, and bleomycin+FGF1 treatment groups.

### DETAILED DESCRIPTION

In one aspect, described herein is a composition comprising a regenerative-promoting polypeptide, a fibrosis-inhibiting polypeptide, or a lung cell function-improving polypeptide, or any combination thereof, wherein the polypeptide is a heparin-associated polypeptide secreted from a stem cell or a transformed cell line, wherein the heparin-associated polypeptide possesses regeneration promoting activity, fibrosis inhibiting activity, or lung cell function improvement activity. In certain aspects, the composition is for use in a method of treating an interstitial lung disease (ILD). In certain aspects, the composition is for use in a method of treating an idiopathic pulmonary fibrosis (IPF). In certain aspects, the composition is for use in a method of increasing proliferation of a lung cell precursor in an individual. In certain aspects, the composition is for use in a method of increasing proliferation of a AT2 lung cell in an individual. In certain embodiments, the individual is afflicted with or suspected of being afflicted with an interstitial lung disease (ILD). In certain aspects, the ILD is due to an environmental exposure or an occupational-related hazard. In certain aspects, the ILD is due to a result of a particular treatment (e.g., a chemotherapy, a radiation therapy, or certain medications). In certain aspects, the ILD is due to an autoimmune or a connective tissue disease. In some embodiments, the autoimmune or connective tissue disease is lupus, scleroderma, poly or dermatomyositis, or rheumatoid arthritis-related ILD. In certain aspects, the ILD is due to sarcoidosis. In certain aspects, the ILD is due to an ILD produced following COVID-19 infection and respiratory illness. In certain aspects, the ILD is due to idiopathic pulmonary fibrosis (IPF). In certain embodiments, the individual is afflicted with or suspected of being afflicted with an idiopathic pulmonary fibrosis (IPF). In certain aspects, the IPF is associated with smoking, certain genetic factors (e.g., risk factors such as certain alleles of MUC5B, TERT, TERC, DKC1, RTEL1, AKAP13, DSP, FAM13A, DPP9, and TOLLIP), viral infections, air pollution, and exposure to certain chemicals. In certain aspects, described herein, is a composition comprising an OLFML3 protein or functional derivative thereof, an NDP protein or functional derivative thereof, an AREG protein or functional derivative thereof, an FGF1 protein or functional derivative thereof, an EFNB2 protein or functional derivative thereof, and/or a CCN1 protein or functional derivative thereof, or any combination thereof. In certain aspects, described herein, is a composition comprising OLFML3, NDP, AREG, FGF1, EFNB2, and/or CCN1 or any combination thereof. In certain aspects, the composition comprises OLFML3 and/or a sequence comprising at least about 90% identity one of SEQ ID NO: 1-3, 16, or 24. In certain aspects, the composition comprises NDP and/or a sequence comprising at least about 90% identity to one of SEQ ID NO: 4, 17, or 18. In certain aspects, the composition comprises AREG and/or a sequence comprising at least about 90% identity to one of SEQ ID NO: 15 or 23. In certain aspects, the composition comprises FGF1 and/or a sequence comprising at least about 90% identity to one of SEQ ID NO: 5-8, 19 or 20. In certain aspects, the composition comprises EFNB2 and/or a sequence comprising at least about 90% identity to one of SEQ ID NO: 9-13, 21 or 25. In certain aspects, the composition comprises CCN1 and/or a sequence comprising at least about 90% identity to one of SEQ ID NO: 14, 22, or 26. In certain aspects, the composition is for use in a method of treating an ILD. In certain aspects, the composition is for use in a method of treating an IPF. In certain aspects, the composition is for use in a method of increasing proliferation a lung cell precursor in an individual. In certain aspects, the composition is for use in a method of increasing proliferation an AT2 cell in an individual. In certain aspects, the individual is afflicted with or suspected of being afflicted with an IPF. In certain aspects, the IPF is associated with smoking, certain genetic factors (e.g., risk factors such as certain alleles of MUC5B, TERT, TERC, DKC1, RTEL1, AKAP13, DSP, FAM13A, DPP9, and TOLLIP), viral infections, air pollution, and exposure to certain chemicals.

### Definitions

In the following description, certain specific details are set forth in order to provide a thorough understanding of various embodiments. However, one skilled in the art will understand that the embodiments provided may be practiced without these details. Unless the context requires otherwise, throughout the specification and claims that follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein a composition that is “consisting essentially” of the recited components is a composition that only has the recited elements as active ingredients, but can comprise other non-active components that do not appreciably modify the function or activity of the recited components. Any list disclosed herein that is recited as “comprising” can be recited as “consisting essentially,” to exclude non-recited polypeptide or protein components.

As used herein “heparin-associated polypeptide” means any polypeptide that directly binds to heparin with a K_{D} of less than 1 micromolar, or any polypeptide that associates with one or more polypeptides that bind directly to heparin with a K_{D} of less than 1 micromolar. This K_{D} can be measured using a method such as surface plasmon resonance. See e.g., Nguyen et al., “Surface plasmon resonance: a versatile technique for biosensor applications.” Sensors (Basel). 2015 May 5; 15 (5): 10481-510. Alternatively, a heparin-associated polypeptide is one that is enriched by a factor of at least 5-fold, 10-fold, 100-fold, or 1,000 from a complex mixture of polypeptides (e.g., a cell supernatant) by the use of heparin bound to a bead or other matrix support, or co-purifies with such a polypeptide.

As used herein “heparin-binding polypeptide” (HBP) means any polypeptide that directly binds to heparin with a K_{D} of less than 1 micromolar. Heparin-binding polypeptides can interact with heparin at steady-state under normal growth conditions, but in other instances heparin-binding polypeptides may interact with heparin transiently under normal growth conditions or only under certain conditions as a result of a signaling or environmental stimulus. Heparin binding-polypeptides may interact with heparin as a result of post-translational modifications such as phosphorylation, dephosphorylation, acetylation, deacetylation, lipidation, delipidation, glycosylation, or deglycosylation, or combinations thereof.

As used herein “pluripotent stem cell” or “pluripotent cell” (PSC) means a cell that has the ability to differentiate into several different cell types that are derivatives of all of the three germinal layers (endoderm, mesoderm, and ectoderm). Pluripotent stem cells are capable of forming teratomas. Examples of pluripotent stem cells are embryonic stem cells (ESCs), embryonic germ stem cells (EGCs), embryonic Carcinoma Cells (ECCs), and induced pluripotent stem cells (iPSCs). PSC may be from any organism of interest, including, primate, human (hPSCs); canine; feline; murine; equine; porcine; avian; camel; bovine; ovine, and so on. Human embryonic stem cells (hESCs) are an example of a PCS.

As used herein “somatic cell” means any cell of an organism that, in the absence of experimental manipulation, does not ordinarily give rise to all types of cells in an organism. In other words, somatic cells are cells that have differentiated sufficiently that they will not naturally generate cells of all three germ layers of the body, i.e., ectoderm, mesoderm and endoderm. For example, somatic cells would include lung cells and lung progenitor cells, the latter of which may be able to self-renew and naturally give rise to all or some cell types of the lung cells but cannot give rise to cells of the ectoderm lineages.

As used herein the term “about” refers to an amount that is near the stated amount by 10% or less.

As used herein the terms “individual” “subject,” and “patient” are interchangeable. The individual can be mammal such as a horse, cow, pig, chicken, goat, rabbit, mouse, rat, dog, or cat. In certain embodiments, the individual is a human person.

The terms “polypeptide” and “protein” are used interchangeably to refer to a polymer of amino acid residues. Polypeptides, including the provided polypeptide chains and other peptides, e.g., linkers and binding peptides, may include amino acid residues including natural and/or non-natural amino acid residues. The terms also include post-translational modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts that produce the proteins, errors due to PCR amplification, or errors in protein translation.

As used herein the terms “functional derivative thereof” refer to a single chain polypeptide in which the primary amino acid sequence encodes for a functional portion of a protein or a functional fragment of a protein. A functional derivative thereof retains sufficient biological function of a full length protein to which it shares a significant percentage identity to the amino acid sequence. A functional derivative of a protein may comprise a fragment of the primary amino acid sequence of a full length version of the protein. A functional derivative of a protein may comprise one or more amino acid substitutions, additions, or deletions in relation to a full length version of the protein. A functional derivative of a protein retains sufficient biological activity compared to a full length version of the protein that the functional derivative thereof is effective at achieving a therapeutic effect on cells of the subject, tissues of the subject, organs of the subject, and/or the subject itself. A significant percentage identity shared between the amino acid sequence of the functional derivative thereof to at least a portion of the amino acid sequence of a full-length version of the protein is generally at least about 90% sequence identity.

In some embodiments, a recombinant protein is a protein expressed in a system other than a human, e.g., the protein is expressed from bacteria, yeast, or mammalian cells in culture. In some cases, the protein is expressed from Chinese Hamster Ovary cells (CHO cells). In some cases, the protein is expressed from mouse myeloma cells, e.g., (NS0) cells. In some cases, the protein is expressed from E. coli.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

“Exogenous” with respect to a nucleic acid or polynucleotide indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid also can be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. The exogenous elements may be added to a construct, for example using genetic recombination. Genetic recombination is the breaking and rejoining of DNA strands to form new molecules of DNA encoding a novel set of genetic information. Often exogenous nucleic acids will include a translatable sequence lacking introns that has been cloned from a cDNA.

As described herein a “mitogenic polypeptide” is one that induces one or more stages of mitosis, including interphase, prophase, metaphase, anaphase, and telophase. In certain embodiments, a mitogenic polypeptide is one that induces mitosis in any one or more of a basal stem cell, a secretory stem cell, a bronchoalveolar stem cell (BASC), or an alveolar type II (AT2) stem cell.

As described herein a “regenerative-promoting polypeptide” is one that may induce one or more stages of mitosis, including interphase, prophase, metaphase, anaphase, and telophase. In certain embodiments, a mitogenic polypeptide is one that induces mitosis in any one or more of a basal stem cell, a secretory stem cell, a bronchoalveolar stem cell (BASC), or an alveolar type II (AT2) stem cell. A regenerative-promoting polypeptide may influence stem cell fate determination to lead to an increased production of differentiated lung cells following a pathological loss of functional differentiated lung cells in a subject. A regenerative-promoting polypeptide may promote AT2 cell renewal. A regenerative-promoting polypeptide may preserve viability in any one or more of a basal stem cell, a secretory stem cell, a bronchoalveolar stem cell (BASC), an alveolar type II (AT2) stem cell, or an alveolar type I (AT1) cell.

As described herein a “fibrosis-inhibiting polypeptide” is one that may inhibit the development of one or more pathological markers of lung fibrosis. A fibrosis-inhibiting polypeptide may serve as prophylactic for further fibrosis or scarring in a lung tissue in a subject. A fibrosis-inhibiting polypeptide may improve one or more pathological features of lung fibrosis in a subject.

As described herein a “lung cell function-improving polypeptide” is one that may improve one or more functional attributes of a healthy lung cell or healthy lung tissue. A lung cell function-improving polypeptide may improve a measurement of respiratory system resistance (Rrs) in a subject. A lung cell function-improving polypeptide may improve a measurement of elastance (Ers) in a subject. A lung cell function-improving polypeptide may improve a measurement of compliance (Crs) in a subject. A lung cell function-improving polypeptide may improve a measurement of airway resistance (Raw) in a subject. A lung cell function-improving polypeptide may improve a measurement of tissue damping (G) in a subject. A lung cell function-improving polypeptide may improve a measurement of tissue elastance (H). Improvements may be determined by comparing various time points and groups. A lung cell function-improving polypeptide may inhibit epithelial-mesenchymal transition (EMT) in an AT2 lung cell. A lung cell function-improving polypeptide may increase expression, production, and/or secretion of a surfactant in a lung cell.

In some embodiments, reference to a conjugate, polypeptide conjugate, or protein conjugate refers to a synthetically and/or recombinantly produced molecule comprising a chemical entity covalently bound to one or more amino acids of an amino acid sequence. In some cases, the conjugation is selective such that the chemical entity is connected to a specific amino acid of the amino acid sequence. In some embodiments, the amino acid sequence comprises a polypeptide described herein. For example, the polypeptide described herein is a polypeptide comprising OLFML3, NDP, AREG, FGF1, EFNB2, and/or CCN1 or any combination thereof.

In some embodiments, a polypeptide described herein is a proteoform of a protein comprising OLFML3, NDP, AREG, FGF1, EFNB2, and/or CCN1 or any combination thereof. In some aspects, as used herein a proteoform describes a molecular form of a protein product arising from a gene encoding a protein, such as a protein including, for example, OLFML3, NDP, AREG, FGF1, EFNB2, and/or CCN1 or any combination thereof. In some cases, a proteoform includes proteins that arise from the same gene as a result of genetic variation, alternatively spliced RNA transcripts, post-translational modifications, or polypeptide cleavage event.

### Polypeptides

In one aspect, polypeptides described herein that are useful for treating an ILD comprise one or more polypeptides secreted from an induced pluripotent stem cell, an embryonic stem cell, a tissue progenitor cell, or a transformed cell line that bind to heparin. In one aspect, polypeptides described herein that are useful for treating an IPF comprise one or more polypeptides secreted from an induced pluripotent stem cell, an embryonic stem cell, a tissue progenitor cell, or a transformed cell line that bind to heparin. In certain embodiments, a plurality of 1, 2, 3, 4, 5, 6, 7, 8, 9, 10 or more HAPs are included in a composition comprising a pharmaceutically acceptable excipient, carrier, or diluent. In some cases, the composition comprises one, two, three, four, five, six, seven, eight, nine, ten or more polypeptides disclosed in U.S. Ser. No. 17/843,676, which is incorporated by reference in its entirety for all purposes. In some embodiments, a plurality of 1, 2, 3, 4, 5, 6, 7, 8, 9, 10 or more HAPs have been identified as therapeutic proteins secreted by one or more types of human stem cells. In some embodiments, a plurality of 1, 2, 3, 4, 5, 6, 7, 8, 9, 10 or more HAPs have been identified as therapeutic proteins secreted by one or more types of human stem cells and the plurality of 1, 2, 3, 4, 5, 6, 7, 8, 9, 10 or more HAPs, or functional derivatives thereof, are produced by in vitro techniques and included in a composition to be administered to a subject having an ILD. In some embodiments, a plurality of 1, 2, 3, 4, 5, 6, 7, 8, 9, 10 or more HAPs have been identified as therapeutic proteins secreted by one or more types of human stem cells and the plurality of 1, 2, 3, 4, 5, 6, 7, 8, 9, 10 or more HAPs, or functional derivatives thereof, are produced by in vitro techniques and included in a composition to be administered to a subject having IPF.

In certain aspects, there are three biochemical features that are common across all potential therapeutic HAPs: 1) they are secreted by human pluripotent stem cells; 2) they can be purified by heparin agarose beads from a complex mixture, and 3) their molecular weight equals or exceeds 3.5 kDa.

In certain aspects, there are certain structure-function relationships that potentially link disparate therapeutic polypeptides into a genus of heparin-associated therapeutic polypeptides. Included among these are the ability to be secreted, which may require: 1) an N-terminal signal sequence (approx. 15-30 amino acids in length); and/or 2) the presence of one or more post translational modifications added in the Endoplasmic Reticulum or the Golgi apparatus to promote stability, such as glycosylation or disulfide bonds. It is estimated that 2,000 to 3,000 genes encoded by the human genome produce a secreted polypeptide in one or more cell types. In addition to being secretory polypeptides the therapeutic polypeptides may comprise a heparin-binding domain, or, alternatively associate with heparin-binding domain comprising polypeptides. Heparin is a linear polymer of saccharides in 1-4 alpha linkages that form a spiraling chain, commonly associated with its role in binding plasma proteins to reduce clotting (See Capila and Lindhart, “Heparin-protein interactions” Angew Chem Int Ed Engl. 2002 Feb. 1; 41 (3): 391-412). Currently, predicting heparin-binding from protein sequence alone is a challenge for the field due to the structural heterogeneity of heparin polymers and the large and variable number of shallow binding pockets thought to be important for stabilizing the interaction. Several hundred HAPs have been empirically tested for heparin binding, using a few heparin chain configurations. Based on these studies many binding motifs have been proposed, but none have been proven necessary and sufficient. One common motif appears to be a sequence of repeating basic residues that orient onto a common surface of the secondary structure for interacting with the matching pattern of sulfate groups on heparin chains. Therefore, many heparin-binding therapeutic polypeptides may contain patterns of basic residues (arginine or lysine) clustered in some part of the protein, though agnostic to the exact sequence.

In certain aspects, the therapeutic polypeptide is an OLFML3 protein or functional derivative thereof, an NDP protein or functional derivative thereof, an AREG protein or functional derivative thereof, an FGF1 protein or functional derivative thereof, an EFNB2 protein or functional derivative thereof, and/or a CCN1 protein or functional derivative thereof. In certain aspects, the therapeutic polypeptide is OLFML3, NDP, AREG, FGF1, EFNB2, and/or CCN1.

In certain aspects, the heparin-associated therapeutic polypeptide is a secreted polypeptide that comprises 1, 2, 3, 4, 5, 6, 7, 8, 9, 10 or more disulfide bonds. In certain aspects, the heparin-associated therapeutic polypeptide is a secreted polypeptide that comprises 1, 2, 3, 4, 5, 6, 7, 8, 9, 10 or N-liked or O-linked glycans. In certain aspects, the heparin-associated therapeutic polypeptide is one that comprises a region exhibiting enrichment for basic amino acids arginine or lysine. The region can be about 5, 10, 15, 20, 25, 30, 35, 40, 45, 50, 55, 60, 65, 70, 75, 80, 85, 90, 95, or 100 amino acids in length, and comprise an amount of basic residues that is greater than would be 10%, 20%, 30%, 40%, 50%, 60%, 70%, 80%, 100%, 150%, or 200% greater than expected given random chance. In certain aspects, the heparin-associated therapeutic polypeptide does not comprise a basic DNA binding motif, such as those found in bZIP transcription factors. In a certain embodiment, the HAP is heparin binding polypeptide.

The therapeutic polypeptides (e.g., OLFML3, NDP, AREG, FGF1, EFNB2, and/or CCN1), described herein, can comprise one or more amino acid modifications that promote stability and/or facilitate production. In certain aspects, the polypeptide (e.g., OLFML3, NDP, AREG, FGF1, EFNB2, and/or CCN1) can comprise one or more covalent modifications that promote stability (e.g., PEGylation). Other modifications of the polypeptide(s) (e.g., OLFML3, NDP, AREG, FGF1, EFNB2, and/or CCN1) are contemplated herein. For example, the polypeptide(s) (e.g., OLFML3, NDP, AREG, FGF1, EFNB2, and/or CCN1) may be linked to one of a variety of non-proteinaceous polymers, e.g., polyethylene glycol, polypropylene glycol, polyoxyalkylenes, or copolymers of polyethylene glycol, polysialic acid, glycolic acid, or polypropylene glycol.

The polypeptides (e.g., OLFML3, NDP, AREG, FGF1, EFNB2, and/or CCN1) described herein can be encapsulated in nanospheres or nanoparticles to increase stability. In certain aspects, the nanospheres or nanoparticles are biodegradable or bioabsorbable. Certain types of nanospheres can be deployed such as polylactic acid (PLA), polyglycolic acid (PGA), or Poly(D,L-lactic-coglycolic-acid) (PLGA) microspheres or nanospheres. In certain aspects, the polypeptide (e.g., OLFML3, NDP, AREG, FGF1, EFNB2, and/or CCN1) is included in a composition comprising a biodegradable or bioabsorbable carrier that promotes polypeptide stability. In certain aspects, the biodegradable or bioabsorbable carrier comprises polylactic acid (PLA), polyglycolic acid (PGA), or Poly(D,L-lactic-coglycolic-acid) (PLGA). In certain aspects, the biodegradable or bioabsorbable carrier comprises polylactic acid (PLA). In certain aspects, the biodegradable or bioabsorbable carrier comprises polyglycolic acid (PGA). In certain aspects, the biodegradable or bioabsorbable carrier comprises Poly(D,L-lactic-coglycolic-acid) (PLGA).

In certain aspects, the polypeptide (e.g., OLFML3, NDP, AREG, FGF1, EFNB2, and/or CCN1) may be concatemerized to increase stability and or bioavailability. In certain aspects, the polypeptide (e.g., OLFML3, NDP, AREG, FGF1, EFNB2, and/or CCN1) comprises concatemers of the same or of different polypeptides. Concatemers can be separated by polypeptide linkers, for example a Gly-Ser linker of any suitable length. In certain embodiments, the Gly-Ser liker comprises a G₄S₁ linker. In certain aspects, the concatemers comprise 1, 2, 3, 4, 5 or more of the same polypeptide (e.g., OLFML3, NDP, AREG, FGF1, EFNB2 OLFML3, NDP, AREG, FGF1, EFNB2, and/or CCN1) separated by a Gly-Ser linker. In certain aspects, the concatemers comprise 1, 2, 3, 4, 5 or more different polypeptides (e.g., OLFML3, NDP, AREG, FGF1, EFNB2, and/or CCN1) as a single polypeptide separated by a Gly-Ser linker. In certain aspects, the concatemers comprise 1, 2, 3, 4, 5 or more of the same polypeptide (e.g., OLFML3, NDP, AREG, FGF1, EFNB2, and/or CCN1) covalently linked through a non-peptide linkage, such as for example a disulfide bridge. In certain aspects, the concatemers comprise 1, 2, 3, 4, 5 or more different polypeptides (e.g., OLFML3, NDP, AREG, FGF1, EFNB2, and/or CCN1) covalently linked through a non-peptide linkage, such as for example a disulfide bridge. In certain aspects, the concatemers comprise 1, 2, 3, 4, 5 or more of the same polypeptide (e.g., OLFML3, NDP, AREG, FGF1, EFNB2, and/or CCN1) non-covalently linked, such as for example, by a streptavidin-biotin interaction or protein-protein interaction. In certain aspects, the concatemers comprise 1, 2, 3, 4, 5 or more different polypeptides (e.g., OLFML3, NDP, AREG, FGF1, EFNB2, and/or CCN1) non-covalently linked such as for example, by a streptavidin-biotin interaction or protein-protein interaction.

Additional modifications to the polypeptides (e.g., OLFML3, NDP, AREG, FGF1, EFNB2, and/or CCN1) comprise deletions of 1, 2, 3, 4, 5, 6, 7, 8, 9, 10, 15, 20, 25, 30, 35, 40, 45, 50, 55, 60, 65, 70, 75, 80, 85, 90, 95, 100, or more amino acids from the N-terminal or C-terminal ends of the HAP. In certain aspects, the polypeptide (e.g., OLFML3, NDP, AREG, FGF1, EFNB2, and/or CCN1) comprises the deletion of known inhibitory domains or deletion of domains not associated with the heparin-associated-polypeptides functions in inducing proliferation of lung cell precursors, in inducing regeneration of lung cell precursors, or in inducing AT2 renewal.

The polypeptides (e.g., OLFML3, NDP, AREG, FGF1, EFNB2, and/or CCN1) herein can comprise cleavage products of a pro-protein. Cleavage of a pro-protein can result in activation or higher activity of said pro-protein. In certain aspects, polypeptides (e.g., OLFML3, NDP, AREG, FGF1, EFNB2, and/or CCN1) are produced that correspond to a cleaved or active form of the pro-protein. In certain aspects, the polypeptides (e.g., OLFML3, NDP, AREG, FGF1, EFNB2, and/or CCN1) comprise only the active domain of a heparin associated pro-protein (e.g., the minimal portion sufficient to create a biological effect).

In certain embodiments, the polypeptide comprises one or more of the polypeptides from U.S. Ser. No. 17/843,676, which is incorporated by reference in its entirety for all purposes. In certain aspects, the polypeptide is at least about 85%, 90%, 95%, 97%, 98%, 99%, or 100% identical to a polypeptide from U.S. Ser. No. 17/843,676.

In certain aspects, the polypeptide is at least about 85%, 90%, 95%, 97%, 98%, 99%, or 100% identical to a polypeptide from U.S. Ser. No. 17/843,676, or a proteoform thereof. In certain aspects, the polypeptide comprises a polypeptide at least about 85%, 90%, 95%, 97%, 98%, 99%, or 100% identical to an OLFML3 protein, an NDP protein, an FGF1 protein, an EFNB2 protein, and/or a CCN1 protein, or any combination thereof. In certain aspects, the polypeptide comprises OLFML3. In certain aspects, the polypeptide comprises NDP. In certain aspects, the polypeptide comprises FGF1. In certain aspects, the polypeptide comprises EFNB2. In certain aspects, the polypeptide comprises CCN1. In certain aspects, described herein, is a composition comprising any 1, 2, 3, 4, 5, or more polypeptides at least about 85%, 90%, 95%, 97%, 98%, 99%, or 100% identical to OLFML3, NDP, AREG, FGF1, EFNB2, CCN1, and/or a polypeptide from U.S. Ser. No. 17/843,676, a proteoform thereof, or a combination thereof; and a pharmaceutically acceptable excipient, carrier, or diluent. In certain aspects, the composition comprises OLFML3. In certain aspects, the composition comprises NDP. In certain aspects, the composition comprises FGF1. In certain aspects, the composition comprises EFNB2. In certain aspects, the composition comprises CCN1.

In certain aspects, the composition consists essentially of an OLFML3 protein or functional derivative thereof, and a pharmaceutically acceptable excipient, carrier, or diluent. In certain aspects, the composition consists essentially of an NDP protein or functional derivative thereof, and a pharmaceutically acceptable excipient, carrier, or diluent. In certain aspects, the composition consists essentially of an AREG protein or functional derivative thereof, and a pharmaceutically acceptable excipient, carrier, or diluent. In certain aspects, the composition consists essentially of an FGF1 protein or functional derivative thereof, and a pharmaceutically acceptable excipient, carrier, or diluent. In certain aspects, the composition consists essentially of an EFNB2 protein or functional derivative thereof, and a pharmaceutically acceptable excipient, carrier, or diluent. In certain aspects, the composition consists essentially of an CCN1 protein or functional derivative thereof, and a pharmaceutically acceptable excipient, carrier, or diluent.

In certain aspects, described herein is a composition comprising a polypeptide comprising at least about 85%, 90%, 95%, 97%, 98%, 99%, or 100% homology or identity to SEQ ID NO: 1, and a pharmaceutically acceptable excipient, carrier, or diluent. In some cases, the polypeptide does not comprise a signal sequence of SEQ ID NO: 1. In certain aspects, described herein is a composition comprising a polypeptide comprising at least about 85%, 90%, 95%, 97%, 98%, 99%, or 100% homology or identity to SEQ ID NO: 2, and a pharmaceutically acceptable excipient, carrier, or diluent. In some cases, the polypeptide does not comprise a signal sequence of SEQ ID NO: 2. In certain aspects, described herein is a composition comprising a polypeptide comprising at least about 85%, 90%, 95%, 97%, 98%, 99%, or 100% homology or identity to SEQ ID NO: 3, and a pharmaceutically acceptable excipient, carrier, or diluent. In some cases, the polypeptide does not comprise a signal sequence of SEQ ID NO: 3. In certain aspects, described herein is a composition comprising a polypeptide comprising at least about 85%, 90%, 95%, 97%, 98%, 99%, or 100% homology or identity to SEQ ID NO: 4, and a pharmaceutically acceptable excipient, carrier, or diluent. In some cases, the polypeptide does not comprise a signal sequence of SEQ ID NO: 4. In certain aspects, described herein is a composition comprising a polypeptide comprising at least about 85%, 90%, 95%, 97%, 98%, 99%, or 100% homology or identity to SEQ ID NO: 5, and a pharmaceutically acceptable excipient, carrier, or diluent. In some cases, the polypeptide does not comprise a signal sequence of SEQ ID NO: 5. In certain aspects, described herein is a composition comprising a polypeptide comprising at least about 85%, 90%, 95%, 97%, 98%, 99%, or 100% homology or identity to SEQ ID NO: 6, and a pharmaceutically acceptable excipient, carrier, or diluent. In some cases, the polypeptide does not comprise a signal sequence of SEQ ID NO: 6. In certain aspects, described herein is a composition comprising a polypeptide comprising at least about 85%, 90%, 95%, 97%, 98%, 99%, or 100% homology or identity to SEQ ID NO: 7, and a pharmaceutically acceptable excipient, carrier, or diluent. In some cases, the polypeptide does not comprise a signal sequence of SEQ ID NO: 7. In certain aspects, described herein is a composition comprising a polypeptide comprising at least about 85%, 90%, 95%, 97%, 98%, 99%, or 100% homology or identity to SEQ ID NO: 8, and a pharmaceutically acceptable excipient, carrier, or diluent. In some cases, the polypeptide does not comprise a signal sequence of SEQ ID NO: 8. In certain aspects, described herein is a composition comprising a polypeptide comprising at least about 85%, 90%, 95%, 97%, 98%, 99%, or 100% homology or identity to SEQ ID NO: 9, and a pharmaceutically acceptable excipient, carrier, or diluent. In some cases, the polypeptide does not comprise a signal sequence of SEQ ID NO: 9. In certain aspects, described herein is a composition comprising a polypeptide comprising at least about 85%, 90%, 95%, 97%, 98%, 99%, or 100% homology or identity to SEQ ID NO: 10, and a pharmaceutically acceptable excipient, carrier, or diluent. In some cases, the polypeptide does not comprise a signal sequence of SEQ ID NO: 10. In certain aspects, described herein is a composition comprising a polypeptide comprising at least about 85%, 90%, 95%, 97%, 98%, 99%, or 100% homology or identity to SEQ ID NO: 11, and a pharmaceutically acceptable excipient, carrier, or diluent. In some cases, the polypeptide does not comprise a signal sequence of SEQ ID NO: 11. In certain aspects, described herein is a composition comprising a polypeptide comprising at least about 85%, 90%, 95%, 97%, 98%, 99%, or 100% homology or identity to SEQ ID NO: 12, and a pharmaceutically acceptable excipient, carrier, or diluent. In some cases, the polypeptide does not comprise a signal sequence of SEQ ID NO: 12. In certain aspects, described herein is a composition comprising a polypeptide comprising at least about 85%, 90%, 95%, 97%, 98%, 99%, or 100% homology or identity to SEQ ID NO: 13, and a pharmaceutically acceptable excipient, carrier, or diluent. In some cases, the polypeptide does not comprise a signal sequence of SEQ ID NO: 13. In certain aspects, described herein is a composition comprising a polypeptide comprising at least about 85%, 90%, 95%, 97%, 98%, 99%, or 100% homology or identity to SEQ ID NO: 14, and a pharmaceutically acceptable excipient, carrier, or diluent. In some cases, the polypeptide does not comprise a signal sequence of SEQ ID NO: 14. In certain aspects, described herein is a composition comprising a polypeptide comprising at least about 85%, 90%, 95%, 97%, 98%, 99%, or 100% homology or identity to SEQ ID NO: 15, and a pharmaceutically acceptable excipient, carrier, or diluent. In some cases, the polypeptide does not comprise a signal sequence of SEQ ID NO: 15. In certain aspects, described herein is a composition comprising a polypeptide comprising at least about 85%, 90%, 95%, 97%, 98%, 99%, or 100% homology or identity to SEQ ID NO: 16, and a pharmaceutically acceptable excipient, carrier, or diluent. In some cases, the polypeptide does not comprise a signal sequence of SEQ ID NO: 16. In certain aspects, described herein is a composition comprising a polypeptide comprising at least about 85%, 90%, 95%, 97%, 98%, 99%, or 100% homology or identity to SEQ ID NO: 17, and a pharmaceutically acceptable excipient, carrier, or diluent. In some cases, the polypeptide does not comprise a signal sequence of SEQ ID NO: 17. In certain aspects, described herein is a composition comprising a polypeptide comprising at least about 85%, 90%, 95%, 97%, 98%, 99%, or 100% homology or identity to SEQ ID NO: 18, and a pharmaceutically acceptable excipient, carrier, or diluent. In some cases, the polypeptide does not comprise a signal sequence of SEQ ID NO: 18. In certain aspects, described herein is a composition comprising a polypeptide comprising at least about 85%, 90%, 95%, 97%, 98%, 99%, or 100% homology or identity to SEQ ID NO: 19, and a pharmaceutically acceptable excipient, carrier, or diluent. In some cases, the polypeptide does not comprise a signal sequence of SEQ ID NO: 19. In certain aspects, described herein is a composition comprising a polypeptide comprising at least about 85%, 90%, 95%, 97%, 98%, 99%, or 100% homology or identity to SEQ ID NO: 20, and a pharmaceutically acceptable excipient, carrier, or diluent. In some cases, the polypeptide does not comprise a signal sequence of SEQ ID NO: 20. In certain aspects, described herein is a composition comprising a polypeptide comprising at least about 85%, 90%, 95%, 97%, 98%, 99%, or 100% homology or identity to SEQ ID NO: 21, and a pharmaceutically acceptable excipient, carrier, or diluent. In some cases, the polypeptide does not comprise a signal sequence of SEQ ID NO: 21. In certain aspects, described herein is a composition comprising a polypeptide comprising at least about 85%, 90%, 95%, 97%, 98%, 99%, or 100% homology or identity to SEQ ID NO: 22, and a pharmaceutically acceptable excipient, carrier, or diluent. In some cases, the polypeptide does not comprise a signal sequence of SEQ ID NO: 22. In certain aspects, described herein is a composition comprising a polypeptide comprising at least about 85%, 90%, 95%, 97%, 98%, 99%, or 100% homology or identity to SEQ ID NO: 23, and a pharmaceutically acceptable excipient, carrier, or diluent. In some cases, the polypeptide does not comprise a signal sequence of SEQ ID NO: 23. In certain aspects, described herein is a composition comprising a polypeptide comprising at least about 85%, 90%, 95%, 97%, 98%, 99%, or 100% homology or identity to SEQ ID NO: 24, and a pharmaceutically acceptable excipient, carrier, or diluent. In some cases, the polypeptide does not comprise a signal sequence of SEQ ID NO: 24. In certain aspects, described herein is a composition comprising a polypeptide comprising at least about 85%, 90%, 95%, 97%, 98%, 99%, or 100% homology or identity to SEQ ID NO: 25, and a pharmaceutically acceptable excipient, carrier, or diluent. In some cases, the polypeptide does not comprise a signal sequence of SEQ ID NO: 25. In certain aspects, described herein is a composition comprising a polypeptide comprising at least about 85%, 90%, 95%, 97%, 98%, 99%, or 100% homology or identity to SEQ ID NO: 26, and a pharmaceutically acceptable excipient, carrier, or diluent. In some cases, the polypeptide does not comprise a signal sequence of SEQ ID NO: 26.

In certain aspects, polypeptides and compositions of polypeptides (e.g., OLFML3, NDP, AREG, FGF1, EFNB2, and/or CCN1) herein comprise polypeptides that increase the proliferation of AT2 cells or AT2 cell precursors, and/or increase their differentiation into AT1 cells. In certain aspects, the polypeptides (e.g., OLFML3, NDP, AREG, FGF1, EFNB2, and/or CCN1) increase proliferation of an AT2 cell or AT2 cell precursor by at least about 20%, 30%, 40%, 50%, or 100% compared to an AT2 cell or AT2 cell precursor not treated with the polypeptide. Proliferation can be measured by BrdU or EdU incorporation, which can be quantified using suitable methods such as, by way of non-limiting embodiment, microscopy, flow cytometry, or ELISA.

In certain aspects, the polypeptides (e.g., OLFML3, NDP, AREG, FGF1, EFNB2, and/or CCN1) increase differentiation of an AT2 by at least about 50%, 75%, 100%, 200%, or 500% compared to an AT2 cell not treated with the polypeptide.

In certain aspects, polypeptides and compositions of polypeptides (e.g., OLFML3, NDP, AREG, FGF1, EFNB2, and/or CCN1) herein comprise polypeptides that preserve viability of AT2 cells or AT2 cell precursors, and/or increase their differentiation into AT1 cells. In certain aspects, the polypeptides (e.g., OLFML3, NDP, AREG, FGF1, EFNB2, and/or CCN1) preserve viability of an AT2 cell or AT2 cell precursor by at least about 20%, 30%, 40%, 50%, or 100% compared to an AT2 cell or AT2 cell precursor not treated with the polypeptide. Viability preservation can be measured by BrdU or EdU incorporation, which can be quantified using suitable methods such as, by way of non-limiting embodiment, microscopy, flow cytometry, or ELISA. Viability preservation can be measured by counting nuclei in a tissue specimen following staining with Hoechst fluorescent dye.

In certain aspects, polypeptides and compositions of polypeptides (e.g., OLFML3, NDP, AREG, FGF1, EFNB2, and/or CCN1) herein comprise polypeptides that inhibit EMT in AT2 cells. In some embodiments, EMT is inhibited by at least about 20%, 30%, 40%, 50%, or 100% in AT2 cells treated with the polypeptide compared AT2 cells not treated with the polypeptide. In some embodiments, an extent of epithelialization is measured by expression pattern or quantification of expression of one or more markers of epithelization. In some embodiments, E-Cadherin, Vimentin, or E-Cadherin and Vimentin are markers of epithelization used to measure an extent of epithelization. In some embodiments, E-Cadherin, Vimentin, or E-Cadherin and Vimentin are assayed in lung cells by immunofluorescent labeling with one or more antibodies that bind specifically to E-Cadherin, Vimentin, or E-Cadherin and Vimentin.

In certain aspects, polypeptides and compositions of polypeptides (e.g., OLFML3, NDP, AREG, FGF1, EFNB2, and/or CCN1) herein comprise polypeptides that increase expression, production, or secretion of one or more surfactants in lung cells. In some embodiments, expression, production, or secretion of one or more surfactants is increased by at least about 20%, 30%, 40%, 50%, or 100% in lung cells treated with the polypeptide compared to lung cells not treated with the polypeptide. In some embodiments, the one or more surfactants comprise SP-A, SP-B, or SP-C, or any combination thereof. In some embodiments, the one or more surfactants comprise SP-C. In some embodiments, expression of the one or more surfactants is assayed in lung cells by immunofluorescent labeling with one or more antibodies that bind specifically to SP-A, SP-B, or SP-C, or any combination thereof.

The polypeptides (e.g., OLFML3, NDP, AREG, FGF1, EFNB2, and/or CCN1) that increase lung tissue cell precursor proliferation and/or differentiation are useful in methods of treating IPF. The IPF can be associated with smoking, certain genetic factors (e.g., risk factors such as certain alleles of MUC5B, TERT, TERC, DKC1, RTEL1, AKAP13, DSP, FAM13A, DPP9, and TOLLIP), viral infections, air pollution, and exposure to certain chemicals.

In certain aspects, a polypeptide composition comprises an OLFML3 protein or functional derivative thereof. In certain aspects, a polypeptide composition comprises an OLFML3 protein at least about 90%, 91%, 92%, 93%, 94%, 95%, 96%, 97%, 98%, or 99% identical to an OLFML3 protein listed in Table 1. In some embodiments, a polypeptide composition comprises an OLFML3 protein at least about 90%, 91%, 92%, 93%, 94%, 95%, 96%, 97%, 98%, or 99% identical to an OLFML3 protein listed in Table 1, wherein the OLFML3 protein has one or more amino acid substitutions at positions that show variations in amino acid sequence when two or more OLFML3 proteins listed in Table 1 are aligned. In certain aspects, a polypeptide composition comprises an OLFML3 protein comprising an amino acid sequence 100% identical to an OLFML3 protein sequence listed in Table 1. In certain aspects, a polypeptide composition comprises OLFML3. OLFML3 protein is produced from the OLFML3 gene locus (official full name: olfactomedin like 3; also known as: OLF44; HNOEL-iso). OLFML3 may be further included in the composition with any one, two, three, or four polypeptides selected from NDP, FGF1, EFNB2, and/or CCN1. Various isoforms of human OLFML3 is disclosed in SEQ ID NO: 1-3, or 16. In certain aspects, the OLFML3 of the composition comprises an amino acid sequence that is at least about 80%, 85%, 90%, 95%, 97%, 98%, 99%, or 100% identical to SEQ ID NO: 1. In certain aspects, the OLFML3 of the composition comprises an amino acid sequence that is at least about 80%, 85%, 90%, 95%, 97%, 98%, 99%, or 100% identical to SEQ ID NO: 2. In certain aspects, the OLFML3 of the composition comprises an amino acid sequence that is at least about 80%, 85%, 90%, 95%, 97%, 98%, 99%, or 100% identical to SEQ ID NO: 3. In certain aspects, the OLFML3 of the composition comprises an amino acid sequence that is at least about 80%, 85%, 90%, 95%, 97%, 98%, 99%, or 100% identical to SEQ ID NO: 16. In certain aspects, the OLFML3 polypeptide lacks a secretory leader sequence. In certain aspects, the OLFML3 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain aspects, the OLFML3 polypeptide comprises one or more additional modifications to increase stability. In certain aspects, the OLFML3 polypeptide is covalently conjugated to polyethylene glycol, polypropylene glycol, polyoxyalkylenes, or copolymers of polyethylene glycol, polysialic acid, glycolic acid, or polypropylene glycol. In certain aspects, the OLFML3 is fused or conjugated to another protein to increase stability and or bioavailability. In certain aspects, the OLFML3 polypeptide is present in a concatemer with one, two, three, four or more distinct polypeptides selected from NDP, AREG, FGF1, EFNB2, and/or CCN1. In certain aspects, the OLFML3 polypeptide is present in a concatemer with one, two, three, four, or more distinct OLFML3 polypeptides. In certain aspects, the OLFML3 polypeptide is included in a composition comprising a biodegradable or bioabsorbable carrier that promotes polypeptide stability. In certain aspects, the biodegradable or bioabsorbable carrier comprises polylactic acid (PLA), polyglycolic acid (PGA), or Poly(D,L-lactic-coglycolic-acid) (PLGA). In certain aspects, the biodegradable or bioabsorbable carrier comprises polylactic acid (PLA). In certain aspects, the biodegradable or bioabsorbable carrier comprises polyglycolic acid (PGA). In certain aspects, the biodegradable or bioabsorbable carrier comprises Poly(D,L-lactic-coglycolic-acid) (PLGA). In some cases, the OLFML3 polypeptide is prepared recombinantly in an expression system (e.g., bacteria, yeast, mammalian, insect). In some cases, the OLFML3 polypeptide is prepared by chemical synthesis. Table 1 lists exemplary OLFML3 proteins identified by NCBI Reference Sequence Entry and Accession Number.

TABLE 1
Exemplary OLFML3 proteins

| Protein | | | | | |
|---|---|---|---|---|---|
| Family | Accession | Accession | Accession | Accession | Accession |
| OLFML3 | XP_059120918.1 | XP_051494848.1 | XP_039697186.1 | XP_017935137.1 | XP_019341557.1 |
| proteins | XP_058994307.1 | XP_051021698.1 | XP_039697185.1 | XP_026516898.1 | XP_019325887.1 |
| | XP_058916977.1 | XP_050810318.1 | XP_039555344.1 | XP_029803233.1 | XP_018421449.1 |
| | XP_058711885.1 | XP_050634868.1 | XP_039421417.1 | XP_029631524.1 | XP_018421448.1 |
| | XP_058675433.1 | XP_050183020.1 | XP_039421416.1 | XP_029538910.1 | XP_005677907.1 |
| | XP_058572131.1 | XP_050013453.1 | XP_027602052.2 | XP_029429701.1 | XP_017725268.1 |
| | XP_046314177.1 | XP_052587630.1 | XP_003933538.1 | XP_008832217.1 | XP_017676775.1 |
| | XP_038417748.1 | NP_001035107.1 | XP_039389254.1 | XP_021051807.1 | XP_017582673.1 |
| | XP_058394838.1 | NP_001273282.1 | XP_039218397.1 | XP_029357579.1 | XP_008642911.2 |
| | XP_058295428.1 | NP_001273281.1 | XP_039105052.1 | XP_021014077.1 | XP_008408224.1 |
| | XP_032010818.1 | NP_064575.1 | XP_038605088.1 | XP_029286773.1 | XP_003220620.1 |
| | XP_004463017.1 | NP_001068665.1 | XP_038547755.1 | XP_020500795.1 | XP_007540789.2 |
| | XP_058034169.1 | NP_598620.2 | XP_540242.1 | XP_015672464.1 | XP_001373047.1 |
| | XP_057895867.1 | NP_001088445.1 | XP_038018687.1 | XP_018614084.1 | XP_016159579.1 |
| | XP_057560829.1 | NP_001070719.1 | XP_027300934.1 | XP_029082696.1 | XP_016100293.1 |
| | XP_057649490.1 | XP_025309271.1 | XP_007975683.1 | XP_028942009.1 | XP_016078874.1 |
| | XP_007169420.1 | XP_048781891.1 | XP_017379733.1 | XP_028853675.1 | XP_007530245.1 |
| | XP_057356507.1 | XP_048682886.1 | XP_037682245.1 | XP_028590377.1 | XP_006763097.1 |
| | XP_057282858.1 | XP_015357692.1 | XP_037266723.1 | XP_028608844.1 | XP_015266920.1 |
| | XP_057240218.1 | XP_048354640.1 | XP_028715176.1 | XP_028430968.1 | XP_015226100.1 |
| | XP_003805700.2 | XP_048298213.1 | XP_036623782.1 | XP_028302391.1 | XP_006628437.1 |
| | XP_006233139.1 | XP_048207780.1 | XP_017504315.1 | XP_028262260.1 | XP_014899889.1 |
| | XP_056700557.1 | XP_048184320.1 | XP_036688148.1 | XP_027859516.1 | XP_014803909.1 |
| | XP_056414326.1 | XP_048184319.1 | XP_036198222.1 | XP_027957878.1 | XP_014728261.1 |
| | XP_056414325.1 | XP_026339489.2 | XP_036128198.1 | XP_027716190.1 | XP_005860036.1 |
| | XP_056367260.1 | XP_013045244.2 | XP_015979870.1 | XP_027753200.1 | XP_005988508.1 |
| | XP_056217949.1 | XP_045657415.1 | XP_036046501.1 | XP_006149692.1 | XP_005887168.1 |
| | XP_055995057.1 | XP_045870050.1 | XP_035944366.1 | XP_027515556.1 | XP_005530195.1 |
| | XP_005447170.1 | NP_001248266.1 | XP_030181114.1 | XP_027551055.1 | XP_013854932.1 |
| | XP_005230360.1 | XP_045672705.1 | XP_030181113.1 | XP_027391877.1 | XP_013909527.1 |
| | XP_055484126.1 | XP_036308765.1 | XP_028358625.1 | XP_010745437.1 | XP_013806604.1 |
| | XP_055440658.1 | XP_045402825.1 | XP_035750758.1 | XP_026946761.1 | XP_005739581.1 |
| | XP_055212265.1 | XP_045402824.1 | XP_027469399.1 | XP_026719919.1 | XP_005389054.1 |
| | XP_004026428.4 | XP_045334672.1 | XP_027249639.1 | XP_005495773.2 | XP_012880115.1 |
| | XP_055257742.1 | XP_010966710.1 | XP_003505860.1 | XP_005357199.1 | XP_012585436.1 |
| | XP_055168470.1 | XP_045221272.1 | XP_035202795.1 | XP_026568811.1 | XP_004689684.1 |
| | XP_004616308.1 | XP_004699217.1 | XP_034979722.1 | XP_026530923.1 | XP_004401350.1 |
| | XP_007108867.1 | XP_006064877.1 | XP_034846135.1 | XP_026249938.1 | XP_011938688.1 |
| | XP_016780764.1 | XP_004667603.1 | XP_034626524.1 | XP_026023989.1 | XP_011856178.1 |
| | XP_514400.2 | XP_044889248.1 | XP_034510028.1 | XP_025967835.1 | XP_011856177.1 |
| | XP_002810446.3 | XP_003990536.2 | XP_034510027.1 | XP_025933130.1 | XP_011856176.1 |
| | XP_054319306.1 | XP_004769911.1 | XP_034510026.1 | XP_025933129.1 | XP_011856175.1 |
| | XP_054836608.1 | XP_044872915.1 | XP_019654159.1 | XP_025899995.1 | XP_011788516.1 |
| | XP_054660224.1 | XP_021545759.1 | XP_002918287.1 | XP_025850399.1 | XP_011788515.1 |
| | XP_008145410.2 | XP_044605072.1 | XP_027796067.1 | XP_025791053.1 | XP_011788514.1 |
| | XP_054504892.1 | XP_014692668.1 | XP_034357098.1 | XP_025791052.1 | XP_011788512.1 |
| | XP_054421038.1 | XP_044531927.1 | XP_034270158.1 | XP_013124647.2 | XP_010829069.1 |
| | XP_053070609.1 | XP_043928834.1 | XP_033774918.1 | XP_025728267.1 | XP_010779893.1 |
| | XP_045752864.1 | XP_044275231.1 | XP_010629543.1 | XP_025243093.1 | XP_009330241.1 |
| | XP_037356194.1 | XP_044139398.1 | XP_004328643.1 | XP_025243082.1 | XP_010576924.1 |
| | XP_036254278.1 | XP_044139397.1 | XP_018864522.1 | XP_025243071.1 | XP_010297707.1 |
| | XP_014921601.2 | XP_044092418.1 | XP_026163853.1 | XP_025243063.1 | XP_010283759.1 |
| | NP_001101178.1 | XP_043860931.1 | XP_033052796.1 | XP_025047731.1 | XP_010215118.1 |
| | XP_054251925.1 | XP_043734973.1 | XP_033008892.1 | XP_025038265.1 | XP_010128075.1 |
| | XP_054193730.1 | XP_007059660.1 | XP_030329477.1 | XP_007429659.1 | XP_010206064.1 |
| | XP_016857337.1 | XP_043431834.1 | XP_030329476.1 | XP_008318340.1 | XP_010190650.1 |
| | XP_002751308.1 | XP_038236019.1 | XP_030329474.1 | XP_024894276.1 | XP_010156300.1 |
| | XP_054146772.1 | XP_043316107.1 | XP_032753489.1 | XP_011735303.1 | XP_010131802.1 |
| | XP_054037262.1 | XP_042854179.1 | XP_032737281.1 | XP_004545061.1 | XP_010000628.1 |
| | XP_054032403.1 | XP_007076410.2 | XP_032622509.1 | XP_024620701.1 | XP_009982208.1 |
| | XP_053943622.1 | XP_042809444.1 | XP_032566566.1 | XP_024620700.1 | XP_010018697.1 |
| | XP_002715799.3 | XP_042809443.1 | XP_032477647.1 | XP_023845077.1 | XP_010018696.1 |
| | XP_053881930.1 | XP_042740045.1 | XP_032406145.1 | XP_004069164.1 | XP_009959055.1 |
| | XP_053854200.1 | XP_032843192.1 | XP_006193212.1 | XP_023797921.1 | XP_009872366.1 |
| | XP_053820242.1 | XP_005294972.1 | XP_015740701.1 | XP_023690664.1 | XP_009937351.1 |
| | XP_053447559.1 | XP_042689632.1 | XP_032271633.1 | XP_006092555.1 | XP_009886754.1 |
| | XP_037008414.2 | XP_007947146.1 | XP_032216483.1 | XP_023557561.1 | XP_009695403.1 |
| | XP_053562543.1 | XP_032936019.1 | XP_032105572.1 | XP_023497899.1 | XP_009805412.1 |
| | XP_053562542.1 | XP_042548248.1 | XP_032073208.1 | XP_001499736.3 | XP_009670576.1 |
| | XP_053313524.1 | XP_042320218.1 | XP_032059056.1 | XP_023391412.1 | XP_009579434.1 |
| | XP_053313523.1 | XP_004002400.1 | XP_031988791.1 | XP_011365089.1 | XP_009502751.1 |
| | XP_047725653.1 | XP_006980862.1 | XP_031988790.1 | XP_003479297.1 | XP_009460710.1 |
| | XP_047640915.1 | NP_001072318.1 | XP_031823191.1 | XP_023403259.1 | XP_009490982.1 |
| | XP_047584212.1 | XP_036894769.1 | XP_023078524.1 | XP_003409645.1 | XP_009068886.1 |
| | XP_047413567.1 | XP_038167314.1 | XP_012820584.1 | XP_003793899.3 | XP_008944414.1 |
| | XP_046940809.1 | XP_041869442.1 | XP_031702629.1 | XP_023279277.1 | XP_008572681.1 |
| | XP_046940808.1 | XP_041869441.1 | XP_031679725.1 | XP_005804356.2 | XP_008540347.1 |
| | XP_046501422.1 | XP_041610874.1 | XP_020343366.1 | XP_022600932.1 | XP_008282927.1 |
| | XP_024426125.2 | XP_041577243.1 | XP_006213734.1 | XP_008056573.1 | XP_007462541.1 |
| | XP_019313452.1 | XP_030147846.1 | XP_003892475.1 | XP_012322053.1 | XP_006865952.1 |
| | XP_004263258.1 | XP_041494058.1 | XP_031412871.1 | XP_021489869.1 | XP_006895758.1 |
| | XP_018777785.2 | XP_041316478.1 | XP_031461577.1 | XP_021233019.1 | |
| | XP_049472489.1 | XP_041278718.1 | XP_021391005.1 | XP_013225131.2 | |
| | XP_049472488.1 | XP_029856886.1 | XP_010974446.1 | XP_004853832.1 | |
| | XP_035417499.1 | XP_040855276.1 | XP_031230902.1 | XP_020945682.1 | |
| | XP_032949987.1 | XP_040855275.1 | XP_031230901.1 | XP_020945681.1 | |
| | XP_050767402.1 | XP_005076603.1 | XP_005428195.1 | XP_005655478.1 | |
| | XP_049735979.1 | XP_008695973.1 | XP_006751201.1 | XP_020857888.1 | |
| | XP_049735978.1 | XP_040472448.1 | XP_030821501.1 | XP_020763890.1 | |
| | XP_049689157.1 | XP_040392216.1 | XP_010370276.1 | XP_020636221.1 | |
| | XP_049621806.1 | XP_032898222.1 | XP_030724965.1 | XP_020448794.1 | |
| | XP_053249515.1 | XP_040327301.1 | XP_003268058.1 | XP_012633882.1 | |
| | XP_053098516.1 | XP_040279853.1 | XP_022439719.1 | XP_020021925.1 | |
| | XP_052671742.1 | XP_040279852.1 | XP_030585499.1 | XP_019812330.1 | |
| | XP_052554086.1 | XP_040193383.1 | XP_030417632.1 | XP_019736568.1 | |
| | XP_052494133.1 | XP_040193382.1 | XP_030321327.1 | XP_019736567.1 | |
| | XP_052494132.1 | XP_005337707.1 | XP_030274488.1 | XP_019585948.1 | |
| | XP_052035570.1 | XP_040086927.1 | XP_030231861.1 | XP_019402344.1 | |
| | XP_051848934.1 | XP_039941438.1 | XP_030077342.1 | XP_019514703.1 | |
| | XP_051664587.1 | XP_028924812.1 | XP_029990699.1 | XP_019369649.1 | |

In certain aspects, a polypeptide composition comprises an NDP protein or functional derivative thereof. In certain aspects, a polypeptide composition comprises an NDP protein at least about 90%, 91%, 92%, 93%, 94%, 95%, 96%, 97%, 98%, or 99% identical to an NDP protein listed in Table 2. In some embodiments, a polypeptide composition comprises an NDP protein at least about 90%, 91%, 92%, 93%, 94%, 95%, 96%, 97%, 98%, or 99% identical to an NDP protein listed in Table 2, wherein the NDP protein has one or more amino acid substitutions at positions that show variations in amino acid sequence when two or more NDP proteins listed in Table 2 are aligned. In certain aspects, a polypeptide composition comprises an NDP protein comprising an amino acid sequence 100% identical to an NDP protein sequence listed in Table 2. In certain aspects, a polypeptide composition comprises NDP. NDP protein is produced from the NDP gene locus (official full name: norrin cystine knot growth factor ndp; also known as: ND; EVR2; FEVR). NDP may be further included in the composition with any one, two, three, or four polypeptides selected from OLFML3, FGF1, EFNB2, and/or CCN1. Human NDP is disclosed in SEQ ID NO: 4. In certain aspects, the NDP of the composition comprises an amino acid sequence that is at least about 80%, 85%, 90%, 95%, 97%, 98%, 99%, or 100% identical to one or SEQ ID NO: 4, 17, or 18. In certain aspects, the NDP polypeptide lacks a secretory leader sequence. In certain aspects, the NDP polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain aspects, the NDP polypeptide comprises one or more additional modifications to increase stability. In certain aspects, the NDP polypeptide is covalently conjugated to polyethylene glycol, polypropylene glycol, polyoxyalkylenes, or copolymers of polyethylene glycol, polysialic acid, glycolic acid, or polypropylene glycol. In certain aspects, the NDP is fused or conjugated to another protein to increase stability and or bioavailability. In certain aspects, the NDP polypeptide is present in a concatemer with one, two, three, four or more distinct polypeptides selected from OLFML3, AREG, FGF1, EFNB2, and/or CCN1. In certain aspects, the NDP polypeptide is present in a concatemer with one, two, three, four, or more distinct NDP polypeptides. In certain aspects, the NDP polypeptide is included in a composition comprising a biodegradable or bioabsorbable carrier that promotes polypeptide stability. In certain aspects, the biodegradable or bioabsorbable carrier comprises polylactic acid (PLA), polyglycolic acid (PGA), or Poly(D,L-lactic-coglycolic-acid) (PLGA). In certain aspects, the biodegradable or bioabsorbable carrier comprises polylactic acid (PLA). In certain aspects, the biodegradable or bioabsorbable carrier comprises polyglycolic acid (PGA). In certain aspects, the biodegradable or bioabsorbable carrier comprises Poly(D,L-lactic-coglycolic-acid) (PLGA). In some cases, the NDP polypeptide is prepared recombinantly in an expression system (e.g., bacteria, yeast, mammalian, insect). In some cases, the NDP polypeptide is prepared by chemical synthesis. Table 2 lists exemplary NDP proteins identified by NCBI Reference Sequence Entry and Accession Number.

TABLE 2
Exemplary NDP proteins

| Protein Family | Accession | Accession | Accession | Accession |
|---|---|---|---|---|
| NDP proteins | NP_001154869.1 | XP_057261058.1 | XP_054022060.1 | XP_051565653.1 |
| | NP_001102284.1 | XP_057261057.1 | XP_053296482.1 | XP_051465061.1 |
| | NP_000257.1 | XP_057261056.1 | XP_053942126.1 | XP_051465060.1 |
| | NP_001039555.1 | XP_057234208.1 | XP_053942120.1 | XP_050998122.1 |
| | NP_001253901.1 | XP_057234207.1 | XP_009559193.2 | XP_050974537.1 |
| | XP_027672755.1 | XP_057234206.1 | XP_017324677.1 | XP_050792703.1 |
| | XP_027638352.1 | XP_026369122.1 | XP_017324676.1 | XP_050631729.1 |
| | XP_005431982.1 | XP_057192809.1 | XP_053861496.1 | XP_050631728.1 |
| | XP_005234548.1 | XP_056889950.1 | XP_053861491.1 | XP_050187202.1 |
| | XP_004064083.1 | XP_056889865.1 | XP_053861483.1 | XP_050187201.1 |
| | XP_003808420.1 | XP_056416696.1 | XP_053861477.1 | XP_050187200.1 |
| | XP_033843224.1 | XP_056416695.1 | XP_053827610.1 | XP_050187199.1 |
| | XP_004612959.1 | XP_056416694.1 | XP_053827609.1 | XP_050016797.1 |
| | XP_020792003.1 | XP_056416693.1 | XP_053827608.1 | XP_049904416.1 |
| | XP_007102091.1 | XP_056357990.1 | XP_053827607.1 | XP_049904415.1 |
| | XP_016799622.1 | XP_056357984.1 | XP_053792236.1 | XP_049904414.1 |
| | XP_037368396.1 | XP_056357975.1 | XP_053792235.1 | XP_049904413.1 |
| | XP_036243958.1 | XP_056357967.1 | XP_053792234.1 | XP_049904412.1 |
| | XP_036243947.1 | XP_056246176.1 | XP_053792233.1 | XP_049904411.1 |
| | XP_036243937.1 | XP_056714646.1 | XP_053436551.1 | XP_049904410.1 |
| | XP_036243928.1 | XP_056610505.1 | XP_053436550.1 | XP_049904409.1 |
| | XP_036243920.1 | XP_056321955.1 | XP_036995031.2 | XP_049904408.1 |
| | XP_035800768.1 | XP_056281957.1 | XP_053562474.1 | XP_049904406.1 |
| | XP_035800766.1 | XP_056281952.1 | XP_053562473.1 | XP_039912735.1 |
| | XP_026909874.1 | XP_056185672.1 | XP_053562472.1 | XP_039912734.1 |
| | XP_023118911.1 | XP_056185671.1 | XP_053562471.1 | XP_039912733.1 |
| | XP_023118910.1 | XP_056144937.1 | XP_053482794.1 | XP_001366493.1 |
| | XP_008151244.1 | XP_056088989.1 | XP_053482793.1 | XP_007227885.1 |
| | XP_002762854.1 | XP_055987213.1 | XP_053482792.1 | XP_025324582.1 |
| | XP_002719919.1 | XP_055726302.1 | XP_053352772.1 | XP_049449341.1 |
| | XP_035629505.1 | XP_055454242.1 | XP_047700301.1 | XP_048812112.1 |
| | XP_047621589.1 | XP_055421176.1 | XP_047571178.1 | XP_048812106.1 |
| | XP_047621588.1 | XP_055294580.1 | XP_046932357.1 | XP_048812097.1 |
| | XP_047392522.1 | XP_055294579.1 | XP_046529546.1 | XP_048812089.1 |
| | XP_004283455.1 | XP_055194476.1 | XP_024435845.2 | XP_048812074.1 |
| | XP_035398013.1 | XP_055194475.1 | XP_053733483.1 | XP_048812065.1 |
| | XP_035398012.1 | XP_055123532.1 | XP_053733482.1 | XP_048720811.1 |
| | XP_035398011.1 | XP_055123531.1 | XP_051259359.1 | XP_048720801.1 |
| | XP_032963276.1 | XP_055071587.1 | XP_051259358.1 | NP_001106528.1 |
| | XP_030090500.1 | XP_054915859.1 | XP_050932324.1 | NP_001265016.1 |
| | XP_028655594.1 | XP_002831603.2 | XP_050932293.1 | NP_001265015.1 |
| | XP_026765437.1 | XP_054327624.1 | XP_050932284.1 | XP_040924617.1 |
| | XP_022057204.1 | XP_054327623.1 | XP_050932246.1 | XP_028991581.1 |
| | XP_022057203.1 | XP_054830177.1 | XP_050932223.1 | XP_028991580.1 |
| | XP_022057202.1 | XP_054830176.1 | XP_049500558.1 | XP_028991579.1 |
| | XP_009087853.1 | XP_054830175.1 | XP_049500557.1 | XP_028991578.1 |
| | XP_059198698.1 | XP_054672554.1 | XP_049500556.1 | XP_015821164.1 |
| | XP_059106966.1 | XP_054672546.1 | XP_018552850.1 | XP_019272883.1 |
| | XP_059014301.1 | XP_054672537.1 | XP_051034883.1 | XP_019272882.1 |
| | XP_059014300.1 | XP_054672527.1 | XP_050767424.1 | XP_019272881.1 |
| | XP_058906288.1 | XP_054672517.1 | XP_049727284.1 | XP_040554545.1 |
| | XP_033866635.1 | XP_054672512.1 | XP_049690966.1 | XP_015155933.1 |
| | XP_033866634.1 | XP_054672502.1 | XP_049690965.1 | XP_015155927.1 |
| | XP_058685536.1 | XP_054672493.1 | XP_049690964.1 | XP_014982712.1 |
| | XP_058685535.1 | XP_054672484.1 | XP_049583168.1 | XP_005228347.1 |
| | XP_058685534.1 | XP_054672474.1 | XP_049623340.1 | XP_005228346.1 |
| | XP_058656221.1 | XP_054672468.1 | XP_053242856.1 | XP_005228345.1 |
| | XP_058656220.1 | XP_054672460.1 | XP_053242855.1 | XP_020935032.1 |
| | XP_058643711.1 | XP_054672451.1 | XP_053185268.1 | XP_058569111.1 |
| | XP_058643710.1 | XP_054642795.1 | XP_053164690.1 | XP_058569110.1 |
| | XP_058479269.1 | XP_054642793.1 | XP_053164689.1 | XP_058569109.1 |
| | XP_004587912.1 | XP_054642792.1 | XP_053164688.1 | XP_058569108.1 |
| | XP_046309817.1 | XP_054642791.1 | XP_052650922.1 | XP_055360839.1 |
| | XP_058391197.1 | XP_054642790.1 | XP_052650921.1 | XP_054603214.1 |
| | XP_032612653.1 | XP_054642789.1 | XP_052530175.1 | XP_053313446.1 |
| | XP_058247102.1 | XP_054642788.1 | XP_052530174.1 | XP_053313445.1 |
| | XP_058147064.1 | XP_054483697.1 | XP_052530173.1 | XP_019272884.1 |
| | XP_058041705.1 | XP_054483696.1 | XP_052530172.1 | XP_052592477.1 |
| | XP_057938897.1 | XP_054483695.1 | XP_052530171.1 | XP_052592406.1 |
| | XP_057874740.1 | XP_054471944.1 | XP_052530170.1 | XP_046763875.1 |
| | XP_057874739.1 | XP_054422369.1 | XP_052530169.1 | XP_046763874.1 |
| | XP_057874738.1 | XP_045746066.1 | XP_052530168.1 | XP_046763873.1 |
| | XP_057707391.1 | XP_054245777.1 | XP_052519770.1 | XP_055360838.1 |
| | XP_057574972.1 | XP_054147278.1 | XP_052420728.1 | |
| | XP_057616203.1 | XP_054147277.1 | XP_052027387.1 | |
| | XP_007180324.2 | XP_054072952.1 | XP_052004946.1 | |
| | XP_007180323.2 | XP_054072941.1 | XP_052004945.1 | |
| | XP_036736791.2 | XP_054072931.1 | XP_051937773.1 | |
| | XP_057261060.1 | XP_054072921.1 | XP_051840952.1 | |

In certain aspects, a polypeptide composition comprises an FGF1 protein or functional derivative thereof. In certain aspects, a polypeptide composition comprises an FGF1 protein at least about 90%, 91%, 92%, 93%, 94%, 95%, 96%, 97%, 98%, or 99% identical to an OLFML3 protein listed in Table 3. In some embodiments, a polypeptide composition comprises an FGF1 protein at least about 90%, 91%, 92%, 93%, 94%, 95%, 96%, 97%, 98%, or 99% identical to an FGF1 protein listed in Table 3, wherein the FGF1 protein has one or more amino acid substitutions at positions that show variations in amino acid sequence when two or more FGF1 proteins listed in Table 3 are aligned. In certain aspects, a polypeptide composition comprises an FGF1 protein comprising an amino acid sequence 100% identical to an FGF1 protein sequence listed in Table 3. In certain aspects, a polypeptide composition comprises FGF1. FGF1 protein is produced from the FGF1 gene locus (official full name: fibroblast growth factor 1; also known as: AFGF; ECGF; FGFA; ECGFA; ECGFB; FGF-1; HBGF1; HBGF-1; GLIO703; ECGF-beta; FGF-alpha). FGF1 may be further included in the composition with any one, two, three, or four polypeptides selected from OLFML3, NDP, EFNB2, and/or CCN1. Various isoforms of human FGF1 are disclosed in SEQ ID NO: 5-8, 19, or 20. In certain aspects, the FGF1 of the composition comprises an amino acid sequence that is at least about 80%, 85%, 90%, 95%, 97%, 98%, 99%, or 100% identical to SEQ ID NO: 5. In certain aspects, the FGF1 of the composition comprises an amino acid sequence that is at least about 80%, 85%, 90%, 95%, 97%, 98%, 99%, or 100% identical to SEQ ID NO: 6. In certain aspects, the FGF1 of the composition comprises an amino acid sequence that is at least about 80%, 85%, 90%, 95%, 97%, 98%, 99%, or 100% identical to SEQ ID NO: 7. In certain aspects, the FGF1 of the composition comprises an amino acid sequence that is at least about 80%, 85%, 90%, 95%, 97%, 98%, 99%, or 100% identical to SEQ ID NO: 8. In certain aspects, the FGF1 of the composition comprises an amino acid sequence that is at least about 80%, 85%, 90%, 95%, 97%, 98%, 99%, or 100% identical to SEQ ID NO: 19. In certain aspects, the FGF1 of the composition comprises an amino acid sequence that is at least about 80%, 85%, 90%, 95%, 97%, 98%, 99%, or 100% identical to SEQ ID NO: 20. In certain aspects, the FGF1 polypeptide lacks a secretory leader sequence. In certain aspects, the FGF1 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain aspects, the FGF1 polypeptide comprises one or more additional modifications to increase stability. In certain aspects, the FGF1 polypeptide is covalently conjugated to polyethylene glycol, polypropylene glycol, polyoxyalkylenes, or copolymers of polyethylene glycol, polysialic acid, glycolic acid, or polypropylene glycol. In certain aspects, the FGF1 is fused or conjugated to another protein to increase stability and or bioavailability. In certain aspects, the FGF1 polypeptide is present in a concatemer with one, two, three, four or more distinct polypeptides selected from OLFML3, NDP, AREG, EFNB2, and/or CCN1. In certain aspects, the FGF1 polypeptide is present in a concatemer with one, two, three, four, or more distinct FGF1 polypeptides. In certain aspects, the FGF1 polypeptide is included in a composition comprising a biodegradable or bioabsorbable carrier that promotes polypeptide stability. In certain aspects, the biodegradable or bioabsorbable carrier comprises polylactic acid (PLA), polyglycolic acid (PGA), or Poly(D,L-lactic-coglycolic-acid) (PLGA). In certain aspects, the biodegradable or bioabsorbable carrier comprises polylactic acid (PLA). In certain aspects, the biodegradable or bioabsorbable carrier comprises polyglycolic acid (PGA). In certain aspects, the biodegradable or bioabsorbable carrier comprises Poly(D,L-lactic-coglycolic-acid) (PLGA). In some cases, the FGF1 polypeptide is prepared recombinantly in an expression system (e.g., bacteria, yeast, mammalian, insect). In some cases, the FGF1 polypeptide is prepared by chemical synthesis. Table 3 lists exemplary FGF1 proteins identified by NCBI Reference Sequence Entry and Accession Number.

TABLE 3
Exemplary FGF1 proteins

| Protein Family | Accession | Accession | Accession | Accession |
|---|---|---|---|---|
| FGF1 proteins | NP_000791.1 | XP_006525710.1 | XP_028705030.1 | XP_038386410.1 |
| | NP_001019381.1 | XP_006525711.1 | XP_028705031.1 | XP_038514694.1 |
| | NP_001019382.1 | XP_006525712.1 | XP_028705032.1 | XP_038514695.1 |
| | NP_001019383.1 | XP_011245143.1 | XP_028705033.1 | XP_038514696.1 |
| | NP_001019384.1 | XP_011245144.1 | XP_028705034.1 | XP_038514697.1 |
| | NP_001138364.1 | XP_011245145.1 | XP_028705035.1 | XP_849274.1 |
| | NP_001138406.1 | XP_017173319.1 | XP_028705036.1 | NP_001180060.1 |
| | NP_001138407.1 | XP_017173320.1 | XP_028705037.1 | NP_001300935.1 |
| | NP_001244134.1 | XP_017173321.1 | XP_028705038.1 | NP_776480.1 |
| | NP_001244135.1 | XP_036016878.1 | XP_028705039.1 | XP_015327761.1 |
| | NP_001244136.1 | XP_036016879.1 | XP_028705040.1 | XP_015327762.1 |
| | NP_001244137.1 | XP_036016880.1 | XP_028705041.1 | XP_015327763.1 |
| | NP_001244138.1 | XP_036016881.1 | NP_001162557.1 | XP_015327764.1 |
| | NP_001244139.1 | NP_990027.1 | XP_021794872.1 | XP_015327765.1 |
| | NP_001244140.1 | NP_990511.1 | XP_021794873.1 | XP_015327766.1 |
| | NP_001244141.1 | XP_015149495.1 | XP_021794874.1 | XP_024849601.1 |
| | NP_001341880.1 | XP_015149496.1 | XP_021794875.1 | XP_024849602.1 |
| | NP_001341881.1 | XP_015149497.1 | XP_021794876.1 | XP_024849603.1 |
| | NP_001341882.1 | XP_025010582.1 | XP_021794877.1 | XP_058944700.1 |
| | NP_001341883.1 | XP_025010583.1 | XP_021794878.1 | XP_058944701.1 |
| | NP_001341884.1 | XP_025010584.1 | XP_021794879.1 | XP_058949163.1 |
| | NP_001341885.1 | XP_040502790.1 | XP_021794880.1 | XP_058949166.1 |
| | NP_001341886.1 | XP_040502791.1 | XP_021794881.1 | XP_058950614.1 |
| | NP_001341887.1 | XP_046756265.1 | XP_021794882.1 | XP_058950615.1 |
| | NP_001341888.1 | XP_046782818.1 | XP_031521911.1 | XP_058950657.1 |
| | NP_001341890.1 | XP_046782819.1 | XP_031521912.1 | XP_058950829.1 |
| | NP_001341891.1 | XP_046782820.1 | XP_031521913.1 | XP_058950846.1 |
| | NP_001341892.1 | XP_046782821.1 | XP_011795124.1 | XP_058959318.1 |
| | NP_001341893.1 | XP_046782822.1 | XP_011795125.1 | XP_058959319.1 |
| | NP_004125.3 | XP_046782823.1 | XP_011795126.1 | XP_058959320.1 |
| | NP_005970.1 | XP_046782824.1 | XP_011795127.1 | NP_001086257.1 |
| | NP_149127.1 | XP_046782825.1 | XP_011795128.1 | NP_001127073.1 |
| | NP_149128.1 | XP_046782826.1 | XP_011795129.1 | NP_001135376.1 |
| | NP_001103610.1 | XP_001153487.1 | XP_011795130.1 | NP_001157358.1 |
| | NP_001239501.1 | XP_001153608.1 | XP_011795131.1 | NP_001279975.1 |
| | NP_001344045.1 | XP_001153676.1 | XP_011795132.1 | NP_957054.1 |
| | NP_031829.2 | XP_009448129.1 | XP_011795133.1 | XP_002165496.3 |
| | NP_032014.3 | XP_009448131.1 | XP_011795134.1 | XP_003970487.1 |
| | NP_032038.1 | XP_009448132.1 | XP_011795135.1 | XP_004541004.1 |
| | NP_033852.1 | XP_009448133.1 | XP_011795136.1 | XP_004541005.1 |
| | NP_034327.1 | XP_009448134.1 | XP_011795137.1 | XP_004550404.1 |
| | NP_034960.1 | XP_016809468.1 | XP_013963577.1 | XP_004550405.1 |
| | NP_038484.1 | XP_016809469.1 | XP_022264627.1 | XP_004586565.1 |
| | XP_006525704.1 | XP_024212534.1 | XP_022264638.1 | XP_004643899.1 |
| | XP_006525705.1 | XP_024212535.1 | XP_038386407.1 | XP_005069251.1 |
| | XP_006525707.1 | XP_024212536.1 | XP_038386408.1 | XP_005440753.1 |
| | XP_006525708.1 | NP_001252958.1 | XP_038386409.1 | XP_005440753.1 |

In certain aspects, a polypeptide composition comprises an EFNB2 protein or functional derivative thereof. In certain aspects, a polypeptide composition comprises an EFNB2 protein at least about 90%, 91%, 92%, 93%, 94%, 95%, 96%, 97%, 98%, or 99% identical to an EFNB2 protein listed in Table 4. In some embodiments, a polypeptide composition comprises an EFNB2 protein at least about 90%, 91%, 92%, 93%, 94%, 95%, 96%, 97%, 98%, or 99% identical to an EFNB2 protein listed in Table 4, wherein the EFNB2 protein has one or more amino acid substitutions at positions that show variations in amino acid sequence when two or more EFNB2 proteins listed in Table 4 are aligned. In certain aspects, a polypeptide composition comprises an EFNB2 protein comprising an amino acid sequence 100% identical to an EFNB2protein sequence listed in Table 4. In certain aspects, a polypeptide composition comprises EFNB2. EFNB2 protein is produced from the EFNB2 gene locus (official full name: ephrin B2; also known as: HTKL; EPLG5; Htk-L; LERK5; ephrin-B2). EFNB2 may be further included in the composition with any one, two, three, or four polypeptides selected from OLFML3, NDP, FGF1, and/or CCN1. Various isoforms of human EFNB2 are disclosed in SEQ ID NO: 9-13, or 21. In certain aspects, the EFNB2 of the composition comprises an amino acid sequence that is at least about 80%, 85%, 90%, 95%, 97%, 98%, 99%, or 100% identical to SEQ ID NO: 9. In certain aspects, the EFNB2 of the composition comprises an amino acid sequence that is at least about 80%, 85%, 90%, 95%, 97%, 98%, 99%, or 100% identical to SEQ ID NO: 10. In certain aspects, the EFNB2 of the composition comprises an amino acid sequence that is at least about 80%, 85%, 90%, 95%, 97%, 98%, 99%, or 100% identical to SEQ ID NO: 11. In certain aspects, the EFNB2 of the composition comprises an amino acid sequence that is at least about 80%, 85%, 90%, 95%, 97%, 98%, 99%, or 100% identical to SEQ ID NO: 12. In certain aspects, the EFNB2 of the composition comprises an amino acid sequence that is at least about 80%, 85%, 90%, 95%, 97%, 98%, 99%, or 100% identical to SEQ ID NO: 13. In certain aspects, the EFNB2 of the composition comprises an amino acid sequence that is at least about 80%, 85%, 90%, 95%, 97%, 98%, 99%, or 100% identical to SEQ ID NO: 21. In certain aspects, the EFNB2 polypeptide lacks a secretory leader sequence. In certain aspects, the EFNB2 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain aspects, the EFNB2 polypeptide comprises one or more additional modifications to increase stability. In certain aspects, the EFNB2 polypeptide is covalently conjugated to polyethylene glycol, polypropylene glycol, polyoxyalkylenes, or copolymers of polyethylene glycol, polysialic acid, glycolic acid, or polypropylene glycol. In certain aspects, the EFNB2 is fused or conjugated to another protein to increase stability and or bioavailability. In some embodiments, the EFNB2 protein may be fused to an fragment crystallizable (Fc) region from a human antibody to create an EFNB2-Fc fusion protein with increased biostability and serum half-life. In some embodiments, the Fc region is fused to an N-terminal portion of EFNB2. In some embodiments, the Fc region is fused to a C-terminal portion of EFNB2. In some embodiments, the EFNB2 polypeptide can comprise an Fc region from a human antibody. In some embodiments, the Fc region from a human antibody is derived from an IgG1 human antibody. In some embodiments, the Fc region from a human antibody is derived from an IgG4 human antibody. In some embodiments, the Fc region may contain one or more amino acid substitutions to produce a fusion protein with having reduced antibody effector function. In some embodiments, the one or more amino acid substitutions in the Fc region comprise the combination of L234A and L235A (LALA). In some embodiments, the one or more amino acid substitutions in the Fc region comprise P329G (PG) substitution. In some embodiments, the one or more amino acid substitutions in the Fc region comprise LALA-PG substitutions. In some embodiments, fusions proteins described herein comprising LALA or LALA-PG substitutions have reduced antibody effector function and maintain a sufficient serum half-life to be used as a therapeutic protein. In certain aspects, the EFNB2 polypeptide is present in a concatemer with one, two, three, four or more distinct polypeptides selected from OLFML3, NDP, AREG, FGF1, and/or CCN1. In certain aspects, the EFNB2 polypeptide is present in a concatemer with one, two, three, four, or more distinct EFNB2 polypeptides. In certain aspects, the EFNB2 polypeptide is included in a composition comprising a biodegradable or bioabsorbable carrier that promotes polypeptide stability. In certain aspects, the biodegradable or bioabsorbable carrier comprises polylactic acid (PLA), polyglycolic acid (PGA), or Poly(D,L-lactic-coglycolic-acid) (PLGA). In certain aspects, the biodegradable or bioabsorbable carrier comprises polylactic acid (PLA). In certain aspects, the biodegradable or bioabsorbable carrier comprises polyglycolic acid (PGA). In certain aspects, the biodegradable or bioabsorbable carrier comprises Poly(D,L-lactic-coglycolic-acid) (PLGA). In some cases, the EFNB2 polypeptide is prepared recombinantly in an expression system (e.g., bacteria, yeast, mammalian, insect). In some cases, the EFNB2 polypeptide is prepared by chemical synthesis. Table 4 lists exemplary EFNB2 proteins identified by NCBI Reference Sequence Entry and Accession Number.

TABLE 4
Exemplary EFNB2 proteins

| Protein | | | | | |
|---|---|---|---|---|---|
| Family | Accession | Accession | Accession | Accession | Accession |
| EFNB2 | NP_001079954.1 | XP_009468628.1 | XP_021551625.1 | XP_032258535.1 | XP_045233341.1 |
| proteins | NP_001093709.1 | XP_009478728.1 | XP_021570905.1 | XP_032258536.1 | XP_045337677.1 |
| | NP_001100798.1 | XP_009512452.1 | XP_022432502.1 | XP_032352572.1 | XP_045337685.1 |
| | NP_001107758.1 | XP_009563412.1 | XP_022609657.1 | XP_032385745.1 | XP_045422840.1 |
| | NP_001107768.1 | XP_009578197.1 | XP_023008132.1 | XP_032467510.1 | XP_045628851.1 |
| | NP_001107816.1 | XP_009637188.1 | XP_023073395.1 | XP_032536778.1 | XP_045677448.1 |
| | NP_001108415.1 | XP_009663988.1 | XP_023273393.1 | XP_032645975.1 | XP_045725647.1 |
| | NP_001116069.1 | XP_009707604.1 | XP_023416364.1 | XP_032736954.1 | XP_045725648.1 |
| | NP_001116361.1 | XP_009872487.1 | XP_023570953.1 | XP_032775428.1 | XP_045833897.1 |
| | NP_001153043.1 | XP_009890447.1 | XP_023803002.1 | XP_032878097.1 | XP_046310731.1 |
| | NP_001155897.1 | XP_009897004.1 | XP_023837341.1 | XP_032878098.1 | XP_046519780.1 |
| | NP_001277099.1 | XP_009926561.1 | XP_023978998.1 | XP_032908355.1 | XP_046941696.1 |
| | NP_001277363.1 | XP_009931790.1 | XP_024065020.1 | XP_032960470.1 | XP_047409437.1 |
| | NP_001277682.1 | XP_009944608.1 | XP_024434660.1 | XP_033003666.1 | XP_047576328.1 |
| | NP_001292125.1 | XP_009984716.1 | XP_024609793.1 | XP_033003668.1 | XP_047611889.1 |
| | NP_001355228.1 | XP_009996231.1 | XP_024855934.1 | XP_033033193.1 | XP_047715548.1 |
| | NP_001358985.1 | XP_010022966.1 | XP_025053636.1 | XP_033804614.1 | XP_047715557.1 |
| | NP_001358986.1 | XP_010075238.1 | XP_025053637.1 | XP_033804615.1 | XP_047936753.1 |
| | NP_001358987.1 | XP_010123911.1 | XP_025220533.1 | XP_033804616.1 | XP_048151897.1 |
| | NP_001407841.1 | XP_010134525.1 | XP_025296797.1 | XP_034274481.1 | XP_048197062.1 |
| | NP_001407842.1 | XP_010149508.1 | XP_025730711.1 | XP_034274489.1 | XP_048312112.1 |
| | NP_001407843.1 | XP_010191023.1 | XP_025779781.1 | XP_034376351.1 | XP_048348991.1 |
| | NP_002196.4 | XP_010193721.1 | XP_025864663.1 | XP_034491845.1 | XP_048348992.1 |
| | NP_004084.1 | XP_010214314.1 | XP_025890361.1 | XP_034491846.1 | XP_048656603.1 |
| | NP_031962.2 | XP_010310350.1 | XP_025922597.1 | XP_034614076.1 | XP_048710749.1 |
| | NP_034237.3 | XP_010351374.1 | XP_025977426.1 | XP_034614077.1 | XP_048815459.1 |
| | NP_034241.2 | XP_010576648.1 | XP_026000310.1 | XP_034792849.1 | XP_049503156.1 |
| | NP_034271.3 | XP_010602952.1 | XP_026151784.1 | XP_034858958.1 | XP_049634105.1 |
| | NP_034272.1 | XP_010786507.1 | XP_026256030.1 | XP_034858959.1 | XP_049671961.1 |
| | NP_034273.1 | XP_010786508.1 | XP_026349366.1 | XP_034970982.1 | XP_049723308.1 |
| | NP_034274.4 | XP_010829769.1 | XP_026524693.1 | XP_034970983.1 | XP_049992825.1 |
| | NP_571098.1 | XP_010950653.1 | XP_026564130.1 | XP_035149696.1 | XP_050188152.1 |
| | NP_990155.1 | XP_010984339.1 | XP_026644610.1 | XP_035174314.1 | XP_050622605.1 |
| | XP_001134800.1 | XP_011240338.1 | XP_026649432.1 | XP_035413010.1 | XP_050622606.1 |
| | XP_001365491.1 | XP_011745920.1 | XP_026701534.1 | XP_035920331.1 | XP_050622607.1 |
| | XP_002198538.1 | XP_011789257.1 | XP_026921004.1 | XP_035920332.1 | XP_050765907.1 |
| | XP_002742574.1 | XP_011824041.1 | XP_026971660.1 | XP_036022671.1 | XP_050765915.1 |
| | XP_002824462.1 | XP_011890705.1 | XP_027136844.1 | XP_036022672.1 | XP_050765923.1 |
| | XP_002915907.1 | XP_011890706.1 | XP_027136847.1 | XP_036105929.1 | XP_050806739.1 |
| | XP_003218789.1 | XP_012296393.1 | XP_027243690.1 | XP_036153717.1 | XP_051025975.1 |
| | XP_003414021.1 | XP_012493412.1 | XP_027413744.1 | XP_036153718.1 | XP_051037919.1 |
| | XP_003451948.1 | XP_012607436.1 | XP_027445768.1 | XP_036242077.1 | XP_051498938.1 |
| | XP_003765793.1 | XP_012878406.1 | XP_027445769.1 | XP_036314121.1 | XP_051665069.1 |
| | XP_003789661.1 | XP_012955430.1 | XP_027499858.1 | XP_036613170.1 | XP_051665071.1 |
| | XP_003832119.1 | XP_013372271.1 | XP_027530529.1 | XP_036687697.1 | XP_051840127.1 |
| | XP_003914107.1 | XP_013809619.1 | XP_027586589.1 | XP_036749566.1 | XP_052018233.1 |
| | XP_003928287.1 | XP_013858500.1 | XP_027674044.2 | XP_036923563.1 | XP_052506010.1 |
| | XP_004012283.1 | XP_013909716.1 | XP_027757886.1 | XP_036998522.1 | XP_052520411.1 |
| | XP_004054766.1 | XP_013909717.1 | XP_027794722.1 | XP_037233997.1 | XP_052607583.1 |
| | XP_004273661.1 | XP_014163194.2 | XP_027967797.1 | XP_037384602.1 | XP_052607584.1 |
| | XP_004313204.1 | XP_014348841.1 | XP_028249517.1 | XP_037656201.1 | XP_052641744.1 |
| | XP_004406011.1 | XP_014387439.1 | XP_028249519.1 | XP_037863805.1 | XP_053167808.1 |
| | XP_004458433.1 | XP_014728827.1 | XP_028382086.1 | XP_037993404.1 | XP_053167809.1 |
| | XP_004577417.1 | XP_014799712.1 | XP_028447441.1 | XP_038029342.1 | XP_053240414.1 |
| | XP_004631231.1 | XP_014799713.1 | XP_028584000.1 | XP_038184291.1 | XP_053311247.1 |
| | XP_004663457.1 | XP_014842599.1 | XP_028584001.1 | XP_038233634.1 | XP_053419795.1 |
| | XP_004680333.1 | XP_014901344.1 | XP_028584002.1 | XP_038615092.1 | XP_053563769.1 |
| | XP_004704353.1 | XP_014976912.1 | XP_028622132.1 | XP_038950533.1 | XP_053792462.1 |
| | XP_004746112.1 | XP_014976913.1 | XP_028728148.1 | XP_039082620.1 | XP_053792463.1 |
| | XP_004858622.1 | XP_015217324.1 | XP_028940637.1 | XP_039182666.1 | XP_053827800.1 |
| | XP_004858623.1 | XP_015217325.1 | XP_029065948.1 | XP_039376269.1 | XP_053827801.1 |
| | XP_005037725.1 | XP_015226844.1 | XP_029065949.1 | XP_039420975.1 | XP_053868990.1 |
| | XP_005077418.1 | XP_015277330.1 | XP_029315732.1 | XP_039581583.1 | XP_054037164.1 |
| | XP_005238428.1 | XP_015339578.1 | XP_029460403.1 | XP_039722244.1 | XP_054127902.1 |
| | XP_005305053.1 | XP_015416563.1 | XP_029483022.1 | XP_039912466.1 | XP_054230125.1 |
| | XP_005317052.1 | XP_015416564.1 | XP_029559006.1 | XP_040123230.1 | XP_054239436.1 |
| | XP_005366367.1 | XP_015495657.1 | XP_029792313.1 | XP_040137646.1 | XP_054239445.1 |
| | XP_005440995.2 | XP_015706945.1 | XP_029892208.1 | XP_040192128.1 | XP_054239453.1 |
| | XP_005507866.1 | XP_015862275.1 | XP_029936959.1 | XP_040281175.1 | XP_054302618.1 |
| | XP_005516541.1 | XP_015975096.1 | XP_029936960.1 | XP_040343033.1 | XP_054302619.1 |
| | XP_005586270.1 | XP_016059347.1 | XP_030057172.1 | XP_040343034.1 | XP_054385186.1 |
| | XP_005744257.1 | XP_016527868.1 | XP_030057174.1 | XP_040393496.1 | XP_054385187.1 |
| | XP_005884537.1 | XP_016875895.1 | XP_030057175.1 | XP_040440436.1 | XP_054430356.1 |
| | XP_005901456.1 | XP_017357096.1 | XP_030090641.1 | XP_040847083.1 | XP_054510845.1 |
| | XP_006004210.1 | XP_017511442.1 | XP_030169730.1 | XP_040847085.1 | XP_054521517.1 |
| | XP_006018665.1 | XP_017672113.1 | XP_030199444.1 | XP_040847086.1 | XP_054557190.1 |
| | XP_006018667.1 | XP_017718479.1 | XP_030284550.1 | XP_041278098.1 | XP_054575933.1 |
| | XP_006072338.1 | XP_017911861.1 | XP_030284551.1 | XP_041324492.1 | XP_054664524.1 |
| | XP_006086978.1 | XP_017926600.1 | XP_030333743.1 | XP_041529619.1 | XP_054830311.1 |
| | XP_006115623.1 | XP_018100276.1 | XP_030424148.1 | XP_041587045.1 | XP_054989728.1 |
| | XP_006159695.1 | XP_018425060.1 | XP_030613672.1 | XP_042315620.1 | XP_055100520.1 |
| | XP_006265936.1 | XP_018598800.1 | XP_030642637.1 | XP_042315621.1 | XP_055100521.1 |
| | XP_006759426.1 | XP_018598801.1 | XP_030642638.1 | XP_042553244.1 | XP_055100522.1 |
| | XP_006846741.1 | XP_019208762.1 | XP_030642640.1 | XP_042662099.1 | XP_055100523.1 |
| | XP_006894675.1 | XP_019299468.1 | XP_030669533.1 | XP_042666970.1 | XP_055191802.1 |
| | XP_006988390.1 | XP_019299469.1 | XP_030669534.1 | XP_042700105.1 | XP_055216344.1 |
| | XP_007196841.1 | XP_019380340.1 | XP_030669535.1 | XP_042700106.1 | XP_055282637.1 |
| | XP_007464450.1 | XP_019385222.1 | XP_030712636.1 | XP_042723705.1 | XP_055398965.1 |
| | XP_007539923.1 | XP_019519273.1 | XP_030778148.1 | XP_042786058.1 | XP_055464454.1 |
| | XP_007642044.1 | XP_019586614.1 | XP_030805668.1 | XP_042835861.1 | XP_055965784.1 |
| | XP_007666176.2 | XP_019586615.1 | XP_030857271.2 | XP_042835870.1 | XP_056185412.1 |
| | XP_007937111.1 | XP_019689948.1 | XP_030885096.1 | XP_043333632.1 | XP_056353360.1 |
| | XP_007959045.1 | XP_019827252.1 | XP_031194838.1 | XP_043438692.1 | XP_056411695.1 |
| | XP_008142173.1 | XP_019955309.1 | XP_031413624.1 | XP_043438700.1 | XP_056718056.1 |
| | XP_008258392.2 | XP_019955310.1 | XP_031469567.1 | XP_043747719.1 | XP_056718057.1 |
| | XP_008278067.1 | XP_020011730.1 | XP_031511516.1 | XP_043853277.1 | XP_057229715.1 |
| | XP_008327174.1 | XP_020317237.1 | XP_031540252.1 | XP_043853278.1 | XP_057256326.1 |
| | XP_008327175.1 | XP_020462280.1 | XP_031725826.1 | XP_043928681.1 | XP_057563654.1 |
| | XP_008402376.1 | XP_020462281.1 | XP_031790789.1 | XP_043928682.1 | XP_057608745.1 |
| | XP_008490857.1 | XP_020491115.1 | XP_031956037.1 | XP_044106016.1 | XP_057901821.1 |
| | XP_008514708.1 | XP_020649980.1 | XP_032007187.1 | XP_044140901.1 | XP_058041454.1 |
| | XP_008569943.1 | XP_020649981.1 | XP_032007189.1 | XP_044284804.1 | XP_058041455.1 |
| | XP_008627543.2 | XP_020744729.1 | XP_032007190.1 | XP_044526472.1 | XP_058404445.1 |
| | XP_008683203.2 | XP_020825731.1 | XP_032054898.1 | XP_044526473.1 | XP_058599079.1 |
| | XP_008819797.1 | XP_021025339.1 | XP_032054906.1 | XP_044526474.1 | XP_058599088.1 |
| | XP_008943878.1 | XP_021074657.1 | XP_032082815.1 | XP_044635661.1 | XP_058680799.1 |
| | XP_009069484.1 | XP_021271316.1 | XP_032082816.1 | XP_044864248.1 | |
| | XP_009284465.1 | XP_021385899.1 | XP_032102086.1 | XP_045001725.1 | |
| | XP_009331628.1 | XP_021500497.1 | XP_032170314.1 | XP_045233340.1 | |
| | XP_058718274.1 | XP_058897565.1 | XP_059000615.1 | XP_059100582.1 | |

In certain aspects, a polypeptide composition comprises a CCN1 protein or functional derivative thereof. In certain aspects, a polypeptide composition comprises a CCN1 protein at least about 90%, 91%, 92%, 93%, 94%, 95%, 96%, 97%, 98%, or 99% identical to a CCN1 protein listed in Table 5. In some embodiments, a polypeptide composition comprises a CCN1 protein at least about 90%, 91%, 92%, 93%, 94%, 95%, 96%, 97%, 98%, or 99% identical to a CCN1 protein listed in Table 5, wherein the CCN1 protein has one or more amino acid substitutions at positions that show variations in amino acid sequence when two or more CCN1 proteins listed in Table 5 are aligned. In certain aspects, a polypeptide composition comprises an CCN1 protein comprising an amino acid sequence 100% identical to an CCN1 protein sequence listed in Table 5. In certain aspects, a polypeptide composition comprises CCN1. CCN1 protein is produced from the CCN1 gene locus (official full name: cellular communication network factor 1; also known as: GIG1; CYR61; IGFBP10). CCN1 may be further included in the composition with any one, two, three, or four polypeptides selected from OLFML3, NDP, FGF1, and/or EFNB2. Human CCN1 is disclosed in SEQ ID NO: 14 or 22. In certain aspects, the CCN1 of the composition comprises an amino acid sequence that is at least about 80%, 85%, 90%, 95%, 97%, 98%, 99%, or 100% identical to SEQ ID NO: 14. In certain aspects, the CCN1 of the composition comprises an amino acid sequence that is at least about 80%, 85%, 90%, 95%, 97%, 98%, 99%, or 100% identical to SEQ ID NO: 22. In certain aspects, the CCN1 polypeptide lacks a secretory leader sequence. In certain aspects, the CCN1 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain aspects, the CCN1 polypeptide comprises one or more additional modifications to increase stability. In certain aspects, the CCN1 polypeptide is covalently conjugated to polyethylene glycol, polypropylene glycol, polyoxyalkylenes, or copolymers of polyethylene glycol, polysialic acid, glycolic acid, or polypropylene glycol. In certain aspects, the CCN1 is fused or conjugated to another protein to increase stability and or bioavailability. In some embodiments, the CCN1 protein may be fused to an fragment crystallizable (Fc) region from a human antibody to create an CCN1-Fc fusion protein with increased biostability and serum half-life. In some embodiments, the Fc region is fused to an N-terminal portion of CCN1. In some embodiments, the Fc region is fused to a C-terminal portion of CCN1. In some embodiments, the CCN1 polypeptide can comprise an Fc region from a human antibody. In some embodiments, the Fc region from a human antibody is derived from an IgG1 human antibody. In some embodiments, the Fc region from a human antibody is derived from an IgG4 human antibody. In some embodiments, the Fc region may contain one or more amino acid substitutions to produce a fusion protein with having reduced antibody effector function. In some embodiments, the one or more amino acid substitutions in the Fc region comprise the combination of L234A and L235A (LALA). In some embodiments, the one or more amino acid substitutions in the Fc region comprise P329G (PG) substitution. In some embodiments, the one or more amino acid substitutions in the Fc region comprise LALA-PG substitutions. In some embodiments, fusions proteins described herein comprising LALA or LALA-PG substitutions have reduced antibody effector function and maintain a sufficient serum half-life to be used as a therapeutic protein. In certain aspects, the CCN1 polypeptide is present in a concatemer with one, two, three, four or more distinct polypeptides selected from OLFML3, NDP, AREG, FGF1, and/or EFNB2. In certain aspects, the CCN1 polypeptide is present in a concatemer with one, two, three, four, or more distinct CCN1 polypeptides. In certain aspects, the CCN1 polypeptide is included in a composition comprising a biodegradable or bioabsorbable carrier that promotes polypeptide stability. In certain aspects, the biodegradable or bioabsorbable carrier comprises polylactic acid (PLA), polyglycolic acid (PGA), or Poly(D,L-lactic-coglycolic-acid) (PLGA). In certain aspects, the biodegradable or bioabsorbable carrier comprises polylactic acid (PLA). In certain aspects, the biodegradable or bioabsorbable carrier comprises polyglycolic acid (PGA). In certain aspects, the biodegradable or bioabsorbable carrier comprises Poly(D,L-lactic-coglycolic-acid) (PLGA). In some cases, the CCN1 polypeptide is prepared recombinantly in an expression system (e.g., bacteria, yeast, mammalian, insect). In some cases, the CCN1 polypeptide is prepared by chemical synthesis. Table 5 lists exemplary CCN1 proteins identified by NCBI Reference Sequence Entry and Accession Number.

TABLE 5
Exemplary CCN1 proteins

| Protein Family | Accession | Accession | Accession | Accession |
|---|---|---|---|---|
| CCN1 proteins | NP_001001826.2 | XP_033947962.1 | XP_048876725.1 | XP_054240055.1 |
| | NP_001017653.1 | XP_033992182.1 | XP_049342102.1 | XP_054334064.1 |
| | NP_001026734.1 | XP_034027206.1 | XP_049434775.1 | XP_054422810.1 |
| | NP_001029512.1 | XP_034152985.1 | XP_049444414.1 | XP_054458545.1 |
| | NP_001074456.2 | XP_034397751.1 | XP_049474436.1 | XP_054466913.1 |
| | NP_001079908.1 | XP_034451720.1 | XP_049585210.1 | XP_054466914.1 |
| | NP_001085010.1 | XP_034547149.1 | XP_049627214.1 | XP_054494428.1 |
| | NP_001092023.1 | XP_034727706.1 | XP_049663446.1 | XP_054645578.1 |
| | NP_001098984.1 | XP_035022778.2 | XP_049735625.1 | XP_054688893.1 |
| | NP_001108294.1 | XP_035246269.1 | XP_049906328.1 | XP_054837439.1 |
| | NP_001545.2 | XP_035392185.1 | XP_049906329.1 | XP_054915027.1 |
| | NP_034347.2 | XP_035393089.1 | XP_049911389.1 | XP_055024828.1 |
| | NP_034646.1 | XP_035393090.1 | XP_050016171.1 | XP_055044850.1 |
| | NP_112617.3 | XP_035496793.1 | XP_050162755.1 | XP_055055511.1 |
| | NP_113787.1 | XP_035514911.1 | XP_050628309.1 | XP_055196315.1 |
| | NP_113820.1 | XP_035514912.1 | XP_050757421.1 | XP_055257962.1 |
| | XP_002715930.1 | XP_035631416.1 | XP_050818167.1 | XP_055441281.1 |
| | XP_002749894.2 | XP_036244183.1 | XP_050931179.1 | XP_055470421.1 |
| | XP_002810701.3 | XP_036384690.1 | XP_050951743.1 | XP_055470422.1 |
| | XP_003805344.2 | XP_036418872.1 | XP_050965961.1 | XP_055470423.1 |
| | XP_004026115.1 | XP_036434190.1 | XP_050974133.1 | XP_055486758.1 |
| | XP_004263026.1 | XP_036746076.1 | XP_051021617.1 | XP_055516336.1 |
| | XP_004480958.1 | XP_036832723.1 | XP_051050756.1 | XP_055580462.1 |
| | XP_004582138.1 | XP_036952780.1 | XP_051233163.1 | XP_055671094.1 |
| | XP_004603367.2 | XP_036952781.1 | XP_051278319.1 | XP_055731282.1 |
| | XP_005949227.1 | XP_036992448.1 | XP_051278320.1 | XP_055775198.1 |
| | XP_005949228.1 | XP_037337198.1 | XP_051481586.1 | XP_055979245.1 |
| | XP_006802606.1 | XP_037337199.1 | XP_051513484.1 | XP_056089248.1 |
| | XP_007120942.1 | XP_037354384.1 | XP_051659987.1 | XP_056102473.1 |
| | XP_007173101.1 | XP_037632519.1 | XP_051737167.1 | XP_056123393.1 |
| | XP_007243420.3 | XP_038560288.1 | XP_051751399.1 | XP_056133929.1 |
| | XP_007480654.1 | XP_038637898.1 | XP_051758264.1 | XP_056141527.1 |
| | XP_008137708.2 | XP_038825988.1 | XP_051806514.1 | XP_056141529.1 |
| | XP_009898668.2 | XP_039515577.1 | XP_051855209.1 | XP_056212462.1 |
| | XP_010874495.2 | XP_039658745.1 | XP_051869053.1 | XP_056231397.1 |
| | XP_010888003.2 | XP_039658746.1 | XP_051893305.1 | XP_056233751.1 |
| | XP_013998005.1 | XP_039658747.1 | XP_051924257.1 | XP_056270204.1 |
| | XP_014185389.1 | XP_039929501.1 | XP_051929432.1 | XP_056305400.1 |
| | XP_015826504.1 | XP_040010230.1 | XP_051929433.1 | XP_056312922.1 |
| | XP_017314928.1 | XP_040052660.1 | XP_051950663.1 | XP_056320562.1 |
| | XP_017323181.1 | XP_040897864.1 | XP_051956175.1 | XP_056353453.1 |
| | XP_017343621.1 | XP_041049445.1 | XP_051966520.1 | XP_056388480.1 |
| | XP_017551781.1 | XP_041049446.1 | XP_052034973.1 | XP_056449015.1 |
| | XP_017552510.1 | XP_041643957.1 | XP_052412831.1 | XP_056449016.1 |
| | XP_018525192.1 | XP_041753485.1 | XP_052419814.1 | XP_056588396.1 |
| | XP_018525193.1 | XP_041793159.1 | XP_052492950.1 | XP_056602258.1 |
| | XP_018549515.1 | XP_041856491.1 | XP_052522300.1 | XP_056602259.1 |
| | XP_019269567.1 | XP_041914960.1 | XP_052587020.1 | XP_056628278.1 |
| | XP_020374246.1 | XP_041957961.1 | XP_052657414.1 | XP_056701628.1 |
| | XP_020774323.1 | XP_042344568.1 | XP_053103766.1 | XP_056894971.1 |
| | XP_020793719.1 | XP_042580116.1 | XP_053178776.1 | XP_057175321.1 |
| | XP_022065535.2 | XP_043094563.1 | XP_053181792.1 | XP_057177776.1 |
| | XP_022070660.1 | XP_043103942.1 | XP_053247845.1 | XP_057212060.1 |
| | XP_022520170.2 | XP_043575827.1 | XP_053277608.1 | XP_057230515.1 |
| | XP_023122889.2 | XP_043881289.1 | XP_053286725.1 | XP_057285005.1 |
| | XP_023141022.2 | XP_044051917.1 | XP_053324927.1 | XP_057570307.1 |
| | XP_024237382.1 | XP_045055559.2 | XP_053339035.1 | XP_057649489.1 |
| | XP_024865806.1 | XP_045753243.1 | XP_053341594.1 | XP_057687272.1 |
| | XP_025273565.1 | XP_045891975.1 | XP_053359472.1 | XP_057697449.1 |
| | XP_025273566.1 | XP_046224270.1 | XP_053447147.1 | XP_057886151.1 |
| | XP_026198051.1 | XP_046241598.1 | XP_053474687.1 | XP_057928873.1 |
| | XP_026353465.1 | XP_046299229.1 | XP_053480087.1 | XP_058034172.1 |
| | XP_026777228.2 | XP_046501440.1 | XP_053480088.1 | XP_058238636.1 |
| | XP_026778075.3 | XP_046694714.1 | XP_053480089.1 | XP_058246122.1 |
| | XP_026801502.3 | XP_046726654.1 | XP_053480090.1 | XP_058266654.1 |
| | XP_026854345.1 | XP_046904367.1 | XP_053480091.1 | XP_058394398.1 |
| | XP_026879982.2 | XP_046941923.1 | XP_053480092.1 | XP_058492388.1 |
| | XP_026914508.1 | XP_047011333.1 | XP_053480094.1 | XP_058494493.1 |
| | XP_027021196.1 | XP_047413342.1 | XP_053480095.1 | XP_058572731.1 |
| | XP_028667354.1 | XP_047446879.1 | XP_053480096.1 | XP_058620411.1 |
| | XP_029003000.1 | XP_047580339.1 | XP_053480097.1 | XP_058632756.1 |
| | XP_030082152.1 | XP_047641370.1 | XP_053480098.1 | XP_058641015.1 |
| | XP_031156727.1 | XP_047664946.1 | XP_053499887.1 | XP_058664463.1 |
| | XP_031156728.1 | XP_047678793.1 | XP_053549866.1 | XP_058699199.1 |
| | XP_031426169.1 | XP_047729383.1 | XP_053710240.1 | XP_058917586.1 |
| | XP_031434304.2 | XP_047729384.1 | XP_053726327.1 | XP_058992284.1 |
| | XP_031605664.1 | XP_048044387.1 | XP_053806324.1 | XP_059121027.1 |
| | XP_032011358.1 | XP_048066945.1 | XP_053841204.1 | XP_059193494.1 |
| | XP_032971885.1 | XP_048098578.1 | XP_053893188.1 | XP_059197147.1 |
| | XP_033474391.1 | XP_048114128.1 | XP_053928423.1 | |
| | XP_033820727.1 | XP_048717975.1 | XP_054069220.1 | |
| | XP_033829281.1 | XP_048802037.1 | XP_054141932.1 | |

In certain aspects, a polypeptide composition comprises an AREG protein or functional derivative thereof. In certain aspects, a polypeptide composition comprises AREG. AREG protein is produced from the AREG gene locus (official full name: amphiregulin; also known as: AR; SDGF; AREGB; or CRDGF). AREG may be further included in the composition with any one, two, three, or four polypeptides selected from OLFML3, NDP, CCN1, FGF1, and/or EFNB2. Human AREG is disclosed in SEQ ID NO: 15 or 23. In certain aspects, the AREG of the composition comprises an amino acid sequence that is at least about 80%, 85%, 90%, 95%, 97%, 98%, 99%, or 100% identical to SEQ ID NO: 15. In certain aspects, the AREG of the composition comprises an amino acid sequence that is at least about 80%, 85%, 90%, 95%, 97%, 98%, 99%, or 100% identical to SEQ ID NO: 23. In certain aspects, the CCN1 polypeptide lacks a secretory leader sequence. In certain aspects, the CCN1 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain aspects, the AREG polypeptide comprises one or more additional modifications to increase stability. In certain aspects, the AREG polypeptide is covalently conjugated to polyethylene glycol, polypropylene glycol, polyoxyalkylenes, or copolymers of polyethylene glycol, polysialic acid, glycolic acid, or polypropylene glycol. In certain aspects, the AREG is fused or conjugated to another protein to increase stability and or bioavailability. In certain aspects, the AREG polypeptide is present in a concatemer with one, two, three, four or more distinct polypeptides selected from OLFML3, NDP, AREG, FGF1, and/or EFNB2. In certain aspects, the AREG polypeptide is present in a concatemer with one, two, three, four, or more distinct AREG polypeptides. In certain aspects, the AREG polypeptide is included in a composition comprising a biodegradable or bioabsorbable carrier that promotes polypeptide stability. In certain aspects, the biodegradable or bioabsorbable carrier comprises polylactic acid (PLA), polyglycolic acid (PGA), or Poly(D,L-lactic-coglycolic-acid) (PLGA). In certain aspects, the biodegradable or bioabsorbable carrier comprises polylactic acid (PLA). In certain aspects, the biodegradable or bioabsorbable carrier comprises polyglycolic acid (PGA). In certain aspects, the biodegradable or bioabsorbable carrier comprises Poly(D,L-lactic-coglycolic-acid) (PLGA). In some cases, the AREG polypeptide is prepared recombinantly in an expression system (e.g., bacteria, yeast, mammalian, insect). In some cases, the AREG polypeptide is prepared by chemical synthesis.

In certain aspects, a polypeptide composition comprises an OLFML3 protein or functional derivative thereof, wherein the OLFML3 protein or functional derivative thereof comprises an amino acid sequence having at least 90%, 91%, 92%, 93%, 94%, 95%, 96%, 97%, 98%, or 99% sequence identity to any one of the sequences represented in Table 1 and one or more conservative amino acid substitutions. In some embodiments, the polypeptide composition comprises an OLFML3 protein or functional derivative thereof, wherein the OLFML3 protein or functional derivative thereof comprises an amino acid sequence having at least 90%, 91%, 92%, 93%, 94%, 95%, 96%, 97%, 98%, or 99% sequence identity to any one of SEQ ID NOs: 1-3, 16, or 24, and one or more conservative amino acid substitutions. In certain aspects, a polypeptide composition comprises an NDP protein or functional derivative thereof, wherein the NDP protein or functional derivative thereof comprises an amino acid sequence having at least 90%, 91%, 92%, 93%, 94%, 95%, 96%, 97%, 98%, or 99% sequence identity to any one of the sequences represented in Table 2 and one or more conservative amino acid substitutions. In some embodiments, the polypeptide composition comprises an NDP protein or functional derivative thereof, wherein the NDP protein or functional derivative thereof comprises an amino acid sequence having at least 90%, 91%, 92%, 93%, 94%, 95%, 96%, 97%, 98%, or 99% sequence identity to any one of SEQ ID NOs: 4, 17, or 18, and one or more conservative amino acid substitutions. In certain aspects, a polypeptide composition comprises an FGF1 protein or functional derivative thereof, wherein the FGF1 protein or functional derivative thereof comprises an amino acid sequence having at least 90%, 91%, 92%, 93%, 94%, 95%, 96%, 97%, 98%, or 99% sequence identity to any one of the sequences represented in Table 3 and one or more conservative amino acid substitutions. In some embodiments, the polypeptide composition comprises an FGF1 protein or functional derivative thereof, wherein the FGF1 protein or functional derivative thereof comprises an amino acid sequence having at least 90%, 91%, 92%, 93%, 94%, 95%, 96%, 97%, 98%, or 99% sequence identity to any one of SEQ ID NOs: 5-8, 19, or 20, and one or more conservative amino acid substitutions. In certain aspects, a polypeptide composition comprises an EFNB2 protein or functional derivative thereof, wherein the EFNB2 protein or functional derivative thereof comprises an amino acid sequence having at least 90%, 91%, 92%, 93%, 94%, 95%, 96%, 97%, 98%, or 99% sequence identity to any one of the sequences represented in Table 4 and one or more conservative amino acid substitutions. In some embodiments, the polypeptide composition comprises an EFNB2 protein or functional derivative thereof, wherein the EFNB2 protein or functional derivative thereof comprises an amino acid sequence having at least 90%, 91%, 92%, 93%, 94%, 95%, 96%, 97%, 98%, or 99% sequence identity to any one of SEQ ID NOs: 9-13, 21, or 25, and one or more conservative amino acid substitutions. In certain aspects, a polypeptide composition comprises an CCN1 protein or functional derivative thereof, wherein the CCN1 protein or functional derivative thereof comprises an amino acid sequence having at least 90%, 91%, 92%, 93%, 94%, 95%, 96%, 97%, 98%, or 99% sequence identity to any one of the sequences represented in Table 5 and one or more conservative amino acid substitutions. In some embodiments, the polypeptide composition comprises an CCN1 protein or functional derivative thereof, wherein the CCN1 protein or functional derivative thereof comprises an amino acid sequence having at least 90%, 91%, 92%, 93%, 94%, 95%, 96%, 97%, 98%, or 99% sequence identity to any one of SEQ ID NOs: 14, 22, or 26, and one or more conservative amino acid substitutions. In certain aspects, a polypeptide composition comprises an AREG protein or functional derivative thereof, wherein the AREG protein or functional derivative thereof comprises an amino acid sequence having at least 90%, 91%, 92%, 93%, 94%, 95%, 96%, 97%, 98%, or 99% sequence identity to any one of SEQ ID NOs: 15 or 23, and one or more conservative amino acid substitutions. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine). Exemplary conservative amino acid substitutions are listed in Table 6.

TABLE 6
Exemplary Amino Acid Modifications

| Side Chain | Members |
|---|---|
| Hydrophobic | Met, Ala, Val, Leu Ile |
| Neutral Hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

In certain aspects, a polypeptide composition comprises a human OLFML3 protein or functional derivative thereof. In certain aspects, a polypeptide composition comprises a human NDP protein or functional derivative thereof. In certain aspects, a polypeptide composition comprises a human FGF1 protein or functional derivative thereof. In certain aspects, a polypeptide composition comprises a human EFNB2 protein or functional derivative thereof. In certain aspects, a polypeptide composition comprises a human CCN1 protein or functional derivative thereof. In certain aspects, a polypeptide composition comprises a human AREG protein or functional derivative thereof. In some embodiments, amino acid sequences for exemplary human polypeptides to be included in the polypeptide composition are described in Table 7A. In some embodiments, amino acid sequences for exemplary functional derivatives of human polypeptides to be included in the polypeptide composition are described in Table 7B. In some embodiments, amino acid sequences for exemplary engineered versions of human polypeptides to be included in the polypeptide composition are described in Table 7C.

TABLE 7A
Exemplary human OLFML3, NDP, AREG, FGF1, EFNB2, and CCN1
polypeptide sequences

| SEQ | | | |
|---|---|---|---|
| ID | Sequence | | |
| NO: | name | Description | Sequence |
| 1 | Human | olfactomedin-like | MGPSTPLLILFLLSWSGPLQGQQHHLVEYMERRLAALEERLAQCQDQSSR |
| | OLFML3- | protein 3 isoform | HAAELRDFKNKMLPLLEVAEKEREALRTEADTISGRVDRLEREVDYLETQN |
| | isoform 1 | 1 precursor | PALPCVEFDEKVTGGPGTKGKGRRNEKYDMVTDCGYTISQVRSMKILKRF |
| | | [Homo sapiens] | GGPAGLWTKDPLGQTEKIYVLDGTQNDTAFVFPRLRDFTLAMAARKASRV |
| | | (NP_064575.1) | RVPFPWVGTGQLVYGGFLYFARRPPGRPGGGGEMENTLQLIKFHLANRTV |
| | | | VDSSVFPAEGLIPPYGLTADTYIDLAADEEGLWAVYATREDDRHLCLAKLDP |
| | | | QTLDTEQQWDTPCPRENAEAAFVICGTLYVVYNTRPASRARIQCSFDASGT |
| | | | LTPERAALPYFPRRYGAHASLRYNPRERQLYAWDDGYQIVYKLEMRKKEE |
| | | | EV |
| 2 | Human | olfactomedin-like | MAQWHCSQRTQAGGHGVGERLAQCQDQSSRHAAELRDFKNKMLPLLEV |
| | OLFML3- | protein 3 isoform | AEKEREALRTEADTISGRVDRLEREVDYLETQNPALPCVEFDEKVTGGPGT |
| | isoform 2 | 2 [Homo sapiens] | KGKGRRNEKYDMVTDCGYTISQVRSMKILKRFGGPAGLWTKDPLGQTEKI |
| | | (NP_001273281.1) | YVLDGTQNDTAFVFPRLRDFTLAMAARKASRVRVPFPWVGTGQLVYGGFL |
| | | | YFARRPPGRPGGGGEMENTLQLIKFHLANRTVVDSSVFPAEGLIPPYGLTA |
| | | | DTYIDLAADEEGLWAVYATREDDRHLCLAKLDPQTLDTEQQWDTPCPREN |
| | | | AEAAFVICGTLYVVYNTRPASRARIQCSFDASGTLTPERAALPYFPRRYGAH |
| | | | ASLRYNPRERQLYAWDDGYQIVYKLEMRKKEEEV |
| 3 | Human | olfactomedin-like | MLPLLEVAEKEREALRTEADTISGRVDRLEREVDYLETQNPALPCVEFDEKV |
| | OLFML3- | protein 3 isoform | TGGPGTKGKGRRNEKYDMVTDCGYTISQVRSMKILKRFGGPAGLWTKDPL |
| | isoform 3 | 3 [Homo sapiens] | GQTEKIYVLDGTQNDTAFVFPRLRDFTLAMAARKASRVRVPFPWVGTGQL |
| | | (NP_001273282.1) | VYGGFLYFARRPPGRPGGGGEMENTLQLIKFHLANRTVVDSSVFPAEGLIP |
| | | | PYGLTADTYIDLAADEEGLWAVYATREDDRHLCLAKLDPQTLDTEQQWDTP |
| | | | CPRENAEAAFVICGTLYVVYNTRPASRARIQCSFDASGTLTPERAALPYFPR |
| | | | RYGAHASLRYNPRERQLYAWDDGYQIVYKLEMRKKEEEV |
| 4 | Human NDP | Norrin precursor | MRKHVLAASFSMLSLLVIMGDTDSKTDSSFIMDSDPRRCMRHHYVDSISHP |
| | | [Homo sapiens] | LYKCSSKMVLLARCEGHCSQASRSEPLVSFSTVLKQPFRSSCHCCRPQTS |
| | | (NP_000257.1) | KLKALRLRCSGGMRLTATYRYILSCHCEECNS |
| 5 | Human FGF1- | fibroblast growth | MAEGEITTFTALTEKFNLPPGNYKKPKLLYCSNGGHFLRILPDGTVDGTRDR |
| | isoform 1 | factor 1 isoform 1 | SDQHIQLQLSAESVGEVY |
| | | precursor | IKSTETGQYLAMDTDGLLYGSQTPNEECLFLERLEENHYNTYISKKHAEKN |
| | | [Homo sapiens] | WFVGLKKNGSCKRGPRTHY |
| | | (NP_000791.1) | GQKAILFLPLPVSSD |
| 6 | Human FGF1- | fibroblast growth | MAEGEITTFTALTEKFNLPPGNYKKPKLLYCSNGGHFLRILPDGTVDGTRDR |
| | isoform 2 | factor 1 isoform 2 | SDQHTDTK |
| | | [Homo sapiens] | |
| | | (NP_001341891.1) | |
| 7 | Human FGF1- | fibroblast growth | MAEGEITTFTALTEKFNLPPGNYKKPKLLYCSNGGHFLRILPDGTVDGTRDR |
| | isoform 3 | factor 1 isoform 3 | SDQHNTK |
| | | [Homo sapiens] | |
| | | (NP_149128.1) | |
| 8 | Human FGF1- | fibroblast growth | MAEGEITTFTALTEKFNLPPGNYKKPKLLYCSNGGHFLRILPDGTVDGTRDR |
| | isoform 4 | factor 1 isoform 4 | SDQHIQLQLSAESVGEVYIKSTETGQYLAMDTDGLLYGSTPNEECLFLERLE |
| | | [Homo sapiens] | ENHYNTYISKKHAEKNWFVGLKKNGSCKRGPRTHYGQKAILFLPLPVSSD |
| | | (NP_001341886.1) | |
| 9 | Human | ephrin-B2 isoform | MAVRRDSVWKYCWGVLMVLCRTAISKSIVLEPIYWNSSNSKFLPGQGLVLY |
| | EFNB2- | 1 precursor | PQIGDKLDIICPKVDSKTVGQYEYYKVYMVDKDQADRCTIKKENTPLLNCAK |
| | isoform 1 | [Homo sapiens] | PDQDIKFTIKFQEFSPNLWGLEFQKNKDYYIISTSNGSLEGLDNQEGGVCQT |
| | | (NP_004084.1) | RAMKILMKVGQDASSAGSTRNKDPTRRPELEAGTNGRSSTTSPFVKPNPG |
| | | | SSTDGNSAGHSGNNILGSEVALFAGIASGCIIFIVIIITLVVLLLKYRRRHRKHS |
| | | | PQHTTTLSLSTLATPKRSGNNNGSEPSDIIIPLRTADSVFCPHYEKVSGDYG |
| | | | HPVYIVQEMPPQSPANIYYKV |
| 10 | Human | ephrin-B2 isoform | MAVRRDSVWKYCWGVLMVLCRTAISKSIVLEPIYWNSSNSKFLPGQGLVLY |
| | EFNB2- | 2 [Homo | PQIGDKLDIICPKVDSKTVGQYEYYKVYMVDKDQADRCTIKKENTPLLNCAK |
| | isoform 2 | sapiens] | PDQDIKFTIKFQEFSPNLWGLEFQKNKDYYIIYASSAGSTRNKDPTRRPELE |
| | | (NP_001358985.1) | AGTNGRSSTTSPFVKPNPGSSTDGNSAGHSGNNILGSEVALFAGIASGCIIFI |
| | | | VIIITLVVLLLKYRRRHRKHSPQHTTTLSLSTLATPKRSGNNNGSEPSDIIIPLR |
| | | | TADSVFCPHYEKVSGDYGHPVYIVQEMPPQSPANIYYKV |
| 11 | Human | ephrin-B2 isoform | MAVRRDSVWKYCWGVLMVLCRTAISKSIVLEPIYWNSSNSKFLPGQGLVLY |
| | EFNB2- | 3 [Homo | PQIGDKLDIICPKVDSKTVGQYEYYKVYMVDKDQADRCTIKKENTPLLNCAK |
| | isoform 3 | sapiens] | PDQDIKFTIKFQEFSPNLWGLEFQKNKDYYIISTSNGSLEGLDNQEGGVCQT |
| | | (NP_001358986.1) | RAMKILMKVGQGSSTDGNSAGHSGNNILGSEVALFAGIASGCIIFIVIIITLVVL |
| | | | LLKYRRRHRKHSPQHTTTLSLSTLATPKRSGNNNGSEPSDIIIPLRTADSVFC |
| | | | PHYEKVSGDYGHPVYIVQEMPPQSPANIYYKV |
| 12 | Human | ephrin-B2 isoform | MYFQRLQQHQVAPQRFGFSSRLIYTLWPTVRKLVRRHQMFVLRFLPGQGL |
| | EFNB2- | X1 [Homo | VLYPQIGDKLDIICPKVDSKTVGQYEYYKVYMVDKDQADRCTIKKENTPLLN |
| | isoform X1 | sapiens] | CAKPDQDIKFTIKFQEFSPNLWGLEFQKNKDYYIISTSNGSLEGLDNQEGGV |
| | | (XP_016875895.1) | CQTRAMKILMKVGQDASSAGSTRNKDPTRRPELEAGTNGRSSTTSPFVKP |
| | | | NPGSSTDGNSAGHSGNNILGSEVALFAGIASGCIIFIVIIITLVVLLLKYRRRHR |
| | | | KHSPQHTTTLSLSTLATPKRSGNNNGSEPSDIIIPLRTADSVFCPHYEKVSG |
| | | | DYGHPVYIVQEMPPQSPANIYYKV |
| 13 | Human | ephrin-B2 isoform | MAVRRDSVWKYCWGVLMVLCRTAISKSIVLEPIYWNSSNSKFLPGQGLVLY |
| | EFNB2- | 4 precursor | PQIGDKLDIICPKVDSKTVGQYEYYKVYMVDKDQADRCTIKKENTPLLNCAK |
| | isoform 4 | [Homo sapiens] | PDQDIKFTIKFQEFSPNLWGLEFQKNKDYYIISVIFVIGYSSESCQRLCIWDL |
| | | (NP_001358987.1) | GGYSTTTTLPLSKRNRSTS |
| 14 | Human CCN1- | CCN family | MSSRIARALALVVTLLHLTRLALSTCPAACHCPLEAPKCAPGVGLVRDGCG |
| | precursor | member 1 | CCKVCAKQLNEDCSKTQPCDHTKGLECNFGASSTALKGICRAQSEGRPCE |
| | | precursor [Homo | YNSRIYQNGESFQPNCKHQCTCIDGAVGCIPLCPQELSLPNLGCPNPRLVK |
| | | sapiens] | VTGQCCEEWVCDEDSIKDPMEDQDGLLGKELGFDASEVELTRNNELIAVG |
| | | (NP_001545.2) | KGSSLKRLPVFGMEPRILYNPLQGQKCIVQTTSWSQCSKTCGTGISTRVTN |
| | | | DNPECRLVKETRICEVRPCGQPVYSSLKKGKKCSKTKKSPEPVRFTYAGCL |
| | | | SVKKYRPKYCGSCVDGRCCTPQLTRTVKMRFRCEDGETFSKNVMMIQSC |
| | | | KCNYNCPHANEAAFPFYRLFNDIHKFRD |
| 15 | Human | Amphiregulin | MRAPLLPPAPVVLSLLILGSGHYAAGLDLNDTYSGKREPFSGDHSADGFEV |
| | Amphiregulin | preproprotein | TSRSEMSSGSEISPVSEMPSSSEPSSGADYDYSEEYDNEPQIPGYIVDDSV |
| | preproprotein | [Homo sapiens] | RVEQVVKPPQNKTESENTSDKPKRKKKGGKNGKNRRNRKKKNPCNAEFQ |
| | | (NP_001648.1) | NFCIHGECKYIEHLEAVTCKCQQEYFGERCGEKSMKTHSMIDSSLSKIALAA |
| | | | IAAFMSAVILTAVAVITVQLRRQYVRKYEGEAEERKKLRQENGNVHAIA |

TABLE 7B
Exemplary human OLFML3, NDP, AREG, FGF1, EFNB2, and CCN1 functional
derivative polypeptide sequences

| SEQ | | | |
|---|---|---|---|
| ID | Sequence | | |
| NO: | name | Description | Sequence |
| 16 | Human | olfactomedin-like | KGKGRRNEKYDMVTDCGYTISQVRSMKILKRFGGPAGLWTKDPLGQTEKIYVL |
| | OLFML3- | protein 3 [Homo | DGTQNDTAFVFPRLRDFTLAMAARKASRVRVPFPWVGTGQLVYGGFLYFARR |
| | functional | sapiens]- | PPGRPGGGGEMENTLQLIKFHLANRTVVDSSVFPAEGLIPPYGLTADTYIDLAA |
| | derivative | functional | DEEGLWAVYATREDDRHLCLAKLDPQTLDTEQQWDTPCPRENAEAAFVICGTL |
| | | derivative | YVVYNTRPASRARIQCSFDASGTLTPERAALPYFPRRYGAHASLRYNPRERQL |
| | | | YAWDDGYQIVYKLEMRKKEEEV |
| 17 | Human | Norrin [Homo | MKTDSSFIMDSDPRRCMRHHYVDSISHPLYKCSSKMVLLARCEGHCSQASRS |
| | NDP- | sapiens]- | EPLVSFSTVLKQPFRSSCHCCRPQTSKLKALRLRCSGGMRLTATYRYILSCHC |
| | functional | functional | EECNS |
| | derivative | derivative v1 | |
| | v1 | | |
| 18 | Human | Norrin [Homo | SKTDSSFIMDSDPRRCMRHHYVDSISHPLYKCSSKMVLLARCEGHCSQASRSE |
| | NDP- | sapiens]- | PLVSFSTVLKQPFRSSCHCCRPQTSKLKALRLRCSGGMRLTATYRYILSCHCE |
| | functional | functional | ECNS |
| | derivative | derivative v2 | |
| | v2 | | |
| 19 | Human | fibroblast growth | MAEGEITTFTALTEKFNLPPGNYKKPKLLYCSNGGHFLRILPDGTVDGTRDRSD |
| | FGF1- | factor 1 | QH |
| | functional | [Homo sapiens] | |
| | derivative | functional | |
| | v1 | derivative v1 | |
| 20 | Human | fibroblast growth | MFNLPPGNYKKPKLLYCSNGGHFLRILPDGTVDGTRDRSDQHIQLQLSAESVG |
| | FGF1- | factor 1 | EVYIKSTETGQYLAMDTDGLLYGSQTPNEECLFLERLEENHYNTYISKKHAEKN |
| | isoform 4 | [Homo sapiens] | WFVGLKKNGSCKRGPRTHYGQKAILFLPLPVSSD |
| | functional | isoform 4 | |
| | derivative | functional | |
| | | derivative | |
| 21 | Human | ephrin-B2 [Homo | SKSIVLEPIYWNSSNSKFLPGQGLVLYPQIGDKLDIICPKVDSKTVGQYEYYKVY |
| | EFNB2- | sapiens]- | MVDKDQADRCTIKKENTPLLNCAKPDQDIKFTIKFQEFSPNLWGLEFQKNKDYY |
| | functional | functional | IISTSNGSLEGLDNQEGGVCQTRAMKILMKVGQDA |
| | derivative | derivative | |
| 22 | Human | CCN family | TCPAACHCPLEAPKCAPGVGLVRDGCGCCKVCAKQLNEDCSKTQPCDHTKGL |
| | CCN1- | member 1 [Homo | ECNFGASSTALKGICRAQSEGRPCEYNSRIYQNGESFQPNCKHQCTCIDGAVG |
| | functional | sapiens]- | CIPLCPQELSLPNLGCPNPRLVKVTGQCCEEWVCDEDSIKDPMEDQDGLLGKE |
| | derivative | functional | LGFDASEVELTRNNELIAVGKGSSLKRLPVFGMEPRILYNPLQGQKCIVQTTSW |
| | | derivative | SQCSKTCGTGISTRVTNDNPECRLVKETRICEVRPCGQPVYSSLKKGKKCSKT |
| | | | KKSPEPVRFTYAGCLSVKKYRPKYCGSCVDGRCCTPQLTRTVKMRFRCEDGE |
| | | | TFSKNVMMIQSCKCNYNCPHANEAAFPFYRLFNDIHKFRD |
| 23 | Human | Amphiregulin | MSVRVEQVVKPPQNKTESENTSDKPKRKKKGGKNGKNRRNRKKKNPCNAEF |
| | Amphi- | [Homo sapiens]- | QNFCIHGECKYIEHLEAVTCKCQQEYFGERCGEKSMKTHSMIDSSLSK |
| | regulin- | functional | |
| | functional | derivative | |
| | derivative | | |

TABLE 7C
Exemplary engineered OLFML3, NDP, AREG, FGF1, EFNB2, and CCN1
functional derivative polypeptide sequences

| SEQ | | | |
|---|---|---|---|
| ID | Sequence | | |
| NO: | name | Description | Sequence |
| 24 | Human | olfactomedin- | MKGKGRRNEKYDMVTDCGYTISQVRSMKILKRFGGPAGLWTKDPLGQTEKIYV |
| | OLFML3- | like protein | LDGTQNDTAFVFPRLRDFTLAMAARKASRVRVPFPWVGTGQLVYGGFLYFAR |
| | 6X His | 3 [Homo | RPPGRPGGGGEMENTLQLIKFHLANRTVVDSSVFPAEGLIPPYGLTADTYIDLA |
| | | sapiens]- | ADEEGLWAVYATREDDRHLCLAKLDPQTLDTEQQWDTPCPRENAEAAFVICG |
| | | 6X His tag | TLYWVYNTRPASRARIQCSFDASGTLTPERAALPYFPRRYGAHASLRYNPRER |
| | | | QLYAWDDGYQIVYKLEMRKKEEEVHHHHHH |
| 25 | Human | ephrin-B2 | MAVRRDSVWKYCWGVLMVLCRTAISKSIVLEPIYWNSSNSKFLPGQGLVLYPQI |
| | EFNB2- | isoform 1 | GDKLDIICPKVDSKTVGQYEYYKVYMVDKDQADRCTIKKENTPLLNCAKPDQDI |
| | isoform | [Homo | KFTIKFQEFSPNLWGLEFQKNKDYYIISTSNGSLEGLDNQEGGVCQTRAMKILM |
| | 1-Fc | sapiens]- | KVGQDASSAGSTRNKDPTRRPELEAGTNGRSSTTSPFVKPNPGSSTDGNSAG |
| | | Fc fusion | HSGNNILGSEVALFAIEGRMDPKSCDKTHTCPPCPAPELLGGPSVFLFPPKPKD |
| | | | TLMISRTPEVTCVVVDVSHEDPEVKFNWYVDGVEVHNAKTKPREEQYNSTYRV |
| | | | VSVLTVLHQDWLNGKEYKCKVSNKALPAPIEKTISKAKGQPREPQVYTLPPSRD |
| | | | ELTKNQVSLTCLVKGFYPSDIAVEWESNGQPENNYKTTPPVLDSDGSFFLYSKL |
| | | | TVDKSRWQQGNVFSCSVMHEALHNHYTQKSLSLSPGK |
| 26 | Human | CCN family | MALSTCPAACHCPLEAPKCAPGVGLVRDGCGCCKVCAKQLNEDCSKTQPCD |
| | CCN1- | member 1 | HTKGLECNFGASSTALKGICRAQSEGRPCEYNSRIYQNGESFQPNCKHQCTCI |
| | Fc | [Homo | DGAVGCIPLCPQELSLPNLGCPNPRLVKVTGQCCEEWVCDEDSIKDPMEDQD |
| | | sapiens]-Fc | GLLGKELGFDASEVELTRNNELIAVGKGSSLKRLPVFGMEPRILYNPLQGQKCI |
| | | fusion | VQTTSWSQCSKTCGTGISTRVTNDNPECRLVKETRICEVRPCGQPVYSSLKKG |
| | | | KKCSKTKKSPEPVRFTYAGCLSVKKYRPKYCGSCVDGRCCTPQLTRTVKMRF |
| | | | RCEDGETFSKNVMMIQSCKCNYNCPHANEAAFPFYRLFNDIHKFRDIEGRMDP |
| | | | KSCDKTHTCPPCPAPELLGGPSVFLFPPKPKDTLMISRTPEVTCVVVDVSHEDP |
| | | | EVKFNWYVDGVEVHNAKTKPREEQYNSTYRVVSVLTVLHQDWLNGKEYKCKV |
| | | | SNKALPAPIEKTISKAKGQPREPQVYTLPPSRDELTKNQVSLTCLVKGFYPSDIA |
| | | | VEWESNGQPENNYKTTPPVLDSDGSFFLYSKLTVDKSRWQQGNVFSCSVMH |
| | | | EALHNHYTQKSLSLSPGK |

In certain aspects, the polypeptide composition comprises any two polypeptides selected from OLFML3, NDP, AREG, FGF1, EFNB2, and/or CCN1. In certain aspects, the composition comprises OLFML3. In certain aspects, the composition comprises NDP. In certain aspects, the composition comprises FGF1. In certain aspects, the composition comprises EFNB2. In certain aspects, the composition comprises CCN1. In certain aspects, the composition comprises AREG.

In certain aspects, the polypeptide composition comprising any three polypeptides selected from OLFML3, NDP, AREG, FGF1, EFNB2, and/or CCN1, comprises one or more additional modifications to increase stability. In certain aspects, one or more of the polypeptides is covalently conjugated to polyethylene glycol, polypropylene glycol, polyoxyalkylenes, or copolymers of polyethylene glycol, polysialic acid, glycolic acid, or polypropylene glycol. In certain aspects, one or more of the polypeptide is fused or conjugated to another protein to increase stability and or bioavailability. In certain aspects, one or more of the polypeptides is present in a concatemer with one, two, three, or four other distinct polypeptides selected from OLFML3, NDP, AREG, FGF1, EFNB2, and/or CCN1. In certain aspects, one or more of the polypeptides is present in a concatemer with one, two, three, four, or more distinct polypeptides. In certain aspects, one or more of the polypeptides is included in a composition comprising a biodegradable or bioabsorbable carrier that promotes polypeptide stability. In certain aspects, the biodegradable or bioabsorbable carrier comprises polylactic acid (PLA), polyglycolic acid (PGA), or Poly(D,L-lactic-coglycolic-acid) (PLGA). In certain aspects, the biodegradable or bioabsorbable carrier comprises polylactic acid (PLA). In certain aspects, the biodegradable or bioabsorbable carrier comprises polyglycolic acid (PGA). In certain aspects, the biodegradable or bioabsorbable carrier comprises Poly(D,L-lactic-coglycolic-acid) (PLGA).

In certain aspects, the heparin-associated binding polypeptide composition comprises any four polypeptides selected from OLFML3, NDP, AREG, FGF1, EFNB2, and/or CCN1.

In certain aspects, the heparin-associated binding polypeptide composition comprising any four polypeptides selected from OLFML3, NDP, AREG, FGF1, EFNB2, and/or CCN1, comprising one or more additional modifications to increase stability. In certain aspects, one or more of the polypeptides is covalently conjugated to polyethylene glycol, polypropylene glycol, polyoxyalkylenes, or copolymers of polyethylene glycol, polysialic acid, glycolic acid, or polypropylene glycol. In certain aspects, one or more of the polypeptides is fused or conjugated to another protein to increase stability and or bioavailability. In certain aspects, one or more of the polypeptides is present in a concatemer with one, two, three, four or more distinct polypeptides selected from OLFML3, NDP, AREG, FGF1, EFNB2, and/or CCN1. In certain aspects, one or more of the polypeptides is present in a concatemer with one, two, three, four, or more distinct polypeptides. In certain aspects, one or more of the polypeptides is included in a composition comprising a biodegradable or bioabsorbable carrier that promotes polypeptide stability. In certain aspects, the biodegradable or bioabsorbable carrier comprises polylactic acid (PLA), polyglycolic acid (PGA), or Poly(D,L-lactic-coglycolic-acid) (PLGA). In certain embodiments, the biodegradable or bioabsorbable carrier comprises polylactic acid (PLA). In certain embodiments, the biodegradable or bioabsorbable carrier comprises polyglycolic acid (PGA). In certain embodiments, the biodegradable or bioabsorbable carrier comprises Poly(D,L-lactic-coglycolic-acid) (PLGA).

### Therapeutic Indications and Methods of Treatment

In some aspects described herein are methods of treating an Interstitial Lung Disease (ILD) comprising administering a therapeutically effective amount of a composition described herein to a subject having an ILD. In some aspects, the methods comprise administering to the subject an effective amount of an OLFML3 protein or functional derivative thereof, wherein the effective amount improves one or more symptoms associated with pulmonary fibrosis in the subject. In some aspects, the methods comprise administering to the subject an effective amount of an OLFML3 protein or functional derivative thereof, wherein the effective amount increases alveolar regeneration in the subject, promotes repair of pulmonary fibrosis in the subject, restores at least partial lung function in the subject, or any combination thereof. In some aspects, the methods comprise administering to the subject an effective amount of an NDP protein or functional derivative thereof, wherein the effective amount improves one or more symptoms associated with pulmonary fibrosis in the subject. In some aspects, the methods comprise administering to the subject an effective amount of an NDP protein or functional derivative thereof, wherein the effective amount increases alveolar regeneration in the subject, promotes repair of pulmonary fibrosis in the subject, restores at least partial lung function in the subject, or any combination thereof. In some aspects, the methods comprise administering to the subject an effective amount of an AREG protein or functional derivative thereof, wherein the effective amount improves one or more symptoms associated with pulmonary fibrosis in the subject. In some aspects, the methods comprise administering to the subject an effective amount of an AREG protein or functional derivative thereof, wherein the effective amount increases alveolar regeneration in the subject, promotes repair of pulmonary fibrosis in the subject, restores at least partial lung function in the subject, or any combination thereof. In some aspects, the methods comprise administering to the subject an effective amount of an FGF1 protein or functional derivative thereof, wherein the effective amount improves one or more symptoms associated with pulmonary fibrosis in the subject. In some aspects, the methods comprise administering to the subject an effective amount of an FGF1 protein or functional derivative thereof, wherein the effective amount increases alveolar regeneration in the subject, promotes repair of pulmonary fibrosis in the subject, restores at least partial lung function in the subject, or any combination thereof. In some aspects, the methods comprise administering to the subject an effective amount of an EFNB2 protein or functional derivative thereof, wherein the effective amount improves one or more symptoms associated with pulmonary fibrosis in the subject. In some aspects, the methods comprise administering to the subject an effective amount of an EFNB2 protein or functional derivative thereof, wherein the effective amount increases alveolar regeneration in the subject, promotes repair of pulmonary fibrosis in the subject, restores at least partial lung function in the subject, or any combination thereof. In some aspects, the methods comprise administering to the subject an effective amount of an CCN1 protein or functional derivative thereof, wherein the effective amount improves one or more symptoms associated with pulmonary fibrosis in the subject. In some aspects, the methods comprise administering to the subject an effective amount of an CCN1 protein or functional derivative thereof, wherein the effective amount increases alveolar regeneration in the subject, promotes repair of pulmonary fibrosis in the subject, restores at least partial lung function in the subject, or any combination thereof. In some embodiments, the ILD in the subject has developed due to an environmental exposure or an occupational-related hazard. In some embodiments, the ILD in the subject has developed due to a result of a particular treatment. In some embodiments, the particular treatment comprises chemotherapy. In some embodiments, the particular treatment comprises radiation therapy. In some embodiments, the particular treatment comprises treatment with a ILD-inducing small molecule drug. In some embodiments, the ILD in the subject has developed due to an autoimmune disorder. In some embodiments, the ILD in the subject has developed due to a connective tissue disease. In some embodiments, the autoimmune disorder comprises lupus. In some embodiments, the autoimmune disorder comprises a rheumatoid arthritis-related ILD. In some embodiments, the autoimmune disorder comprises dermatomyositis. In some embodiments, the autoimmune disorder comprises polymyositis. In some embodiments, the connective tissue disease comprises scleroderma. In some embodiments, the connective tissue disease comprises dermatomyositis. In some embodiments, the connective tissue disease comprises polymyositis. In some embodiments, the ILD in the subject has developed due to progression of sarcoidosis. In some embodiments, the ILD in the subject has developed following COVID-19 infection and respiratory illness. In some embodiments, the ILD in the subject has developed due to IPF. In some embodiments, an effective amount comprises an amount of the composition that when administered to the subject leads to a significant improvement in one or more pathological symptoms associated with ILD. In some embodiments, an effective amount comprises an amount of the composition that when administered to the subject leads to a significant improvement in one or more aspects of lung function.

In some aspects described herein are methods of treating an Idiopathic Pulmonary Fibrosis (IPF) comprising administering a therapeutically effective amount of a composition described herein to a subject having an IPF. In some aspects, the methods comprise administering to the subject an effective amount of an OLFML3 protein or functional derivative thereof, wherein the effective amount improves one or more symptoms associated with pulmonary fibrosis in the subject. In some aspects, the methods comprise administering to the subject an effective amount of an OLFML3 protein or functional derivative thereof, wherein the effective amount increases alveolar regeneration in the subject, promotes repair of pulmonary fibrosis in the subject, restores at least partial lung function in the subject, or any combination thereof. In some aspects, the methods comprise administering to the subject an effective amount of an NDP protein or functional derivative thereof, wherein the effective amount improves one or more symptoms associated with pulmonary fibrosis in the subject. In some aspects, the methods comprise administering to the subject an effective amount of an NDP protein or functional derivative thereof, wherein the effective amount increases alveolar regeneration in the subject, promotes repair of pulmonary fibrosis in the subject, restores at least partial lung function in the subject, or any combination thereof. In some aspects, the methods comprise administering to the subject an effective amount of an AREG protein or functional derivative thereof, wherein the effective amount improves one or more symptoms associated with pulmonary fibrosis in the subject. In some aspects, the methods comprise administering to the subject an effective amount of an AREG protein or functional derivative thereof, wherein the effective amount increases alveolar regeneration in the subject, promotes repair of pulmonary fibrosis in the subject, restores at least partial lung function in the subject, or any combination thereof. In some aspects, the methods comprise administering to the subject an effective amount of an FGF1 protein or functional derivative thereof, wherein the effective amount improves one or more symptoms associated with pulmonary fibrosis in the subject. In some aspects, the methods comprise administering to the subject an effective amount of an FGF1 protein or functional derivative thereof, wherein the effective amount increases alveolar regeneration in the subject, promotes repair of pulmonary fibrosis in the subject, restores at least partial lung function in the subject, or any combination thereof. In some aspects, the methods comprise administering to the subject an effective amount of an EFNB2 protein or functional derivative thereof, wherein the effective amount improves one or more symptoms associated with pulmonary fibrosis in the subject. In some aspects, the methods comprise administering to the subject an effective amount of an EFNB2 protein or functional derivative thereof, wherein the effective amount increases alveolar regeneration in the subject, promotes repair of pulmonary fibrosis in the subject, restores at least partial lung function in the subject, or any combination thereof. In some aspects, the methods comprise administering to the subject an effective amount of an CCN1 protein or functional derivative thereof, wherein the effective amount improves one or more symptoms associated with pulmonary fibrosis in the subject. In some aspects, the methods comprise administering to the subject an effective amount of an CCN1 protein or functional derivative thereof, wherein the effective amount increases alveolar regeneration in the subject, promotes repair of pulmonary fibrosis in the subject, restores at least partial lung function in the subject, or any combination thereof. In some embodiments, an effective amount comprises an amount of the composition that when administered to the subject leads to a significant improvement in one or more pathological symptoms associated with IPF. In some embodiments, an effective amount comprises an amount of the composition that when administered to the subject leads to a significant improvement in one or more aspects of lung function. ILD is a chronic lung disease. Symptoms of ILD and the course of the diseases categorized as ILDs are known to vary from person to person. However, a common link between forms of ILD is the presence of inflammation early in the course of the disease. Inflammation may be in the form of bronchiolitis (inflammation that involves the bronchioles). Inflammation may be in the form of alveolitis (inflammation that involves the alveoli). Inflammation may be in the form of vasculitis (inflammation that involves the small blood vessels of the lung). These are the most common symptoms for interstitial lung diseases:
Shortness of breath
Fatigue and weakness
Loss of appetite
Weight loss
Dry cough that does not produce phlegm
Chest discomfort
Labored breathing
Hemorrhage in the lungs

Pulmonary fibrosis often results in a permanent loss of the ability of lung tissue to carry oxygen. Fibrotic scarring in various ILDs destroyed the alveoli and surrounding capillaries. The progression of an ILD may run gradually or proceed to a rapid course. Subjects having an ILD may notice variation in symptoms; from mild, to moderate, to severe. The subject's symptom or symptoms may remain consistent for extended periods of time or may change rapidly. As an ILD progresses, lung tissue thickens and becomes stiff. As this happens, the work of breathing becomes more difficult and demanding for the subject. In some instances, subjects having an ILD are prescribed medications to treat inflammation, supplementary oxygen therapy, or a combination thereof.

A subject may be diagnosed as having an ILD following a clinical assessment. The clinical assessment may include pulmonary function tests, a chest X-ray, blood tests (e.g., an arterial blood gas to analyze the amount of carbon dioxide and oxygen in the blood), CT scan, high-resolution CT scan, CAT scan, bronchoscopy, bronchoalveolar lavage, or lung biopsy, or any combination thereof.

A subject may be diagnosed as having an IPF following a clinical assessment. The diagnosis of an IPF may involve eliminating as possibilities other categories of ILD in the subject. In some instances, a subject diagnosed as having an IPF has been determined to meet the clinical criteria for an ILD, but also have known causes of ILDs rule out as likely causative disease initiating factors. A subject having an IPF may have one or more symptoms comprising shortness of breath, a persistent dry cough, tiredness or excessive fatigue, loss of appetite, loss of weight, rounded and swollen fingertips (clubbed fingers), or any combination thereof.

In some aspects, the methods of treatment described herein alleviate one or more symptoms of IPF in the subject following administering of a composition described herein. In some embodiments, the method of treatment promotes regeneration of lung precursor cells. In some embodiments, the method of treatment promotes regeneration of lung stem cells. In some embodiments, the method of treatment promotes regeneration of lung cells. In some embodiments, the method of treatment promotes regeneration of AT2 cells. In some embodiments, the method of treatment promotes differentiation of AT1 cells. In some embodiments, the method of treatment promotes mitogenesis of AT2 cells. In some embodiments, the method of treatment promotes renewal of AT2 cells. In some embodiments, the method of treatment preserves viability of lung stem cells. In some embodiments, the method of treatment preserves viability of AT2 cells. In some embodiments, the method of treatment inhibits EMT in AT2 cells. In some embodiments, the method of treatment promotes lung cell activity under fibrotic stress. In some embodiments, the method of treatment promotes lung cell activity under fibrotic stress by reducing EMT. In some embodiments, the method of treatment promotes lung cell activity under fibrotic stress by reducing EMT in a plurality of AT2 cells. In some embodiments, the method of treatment promotes lung cell activity under fibrotic stress by improving production of one or more surfactants. In some embodiments, the method of treatment promotes lung cell activity under fibrotic stress by improving expression of one or more surfactants. In some embodiments, the method of treatment promotes lung cell activity under fibrotic stress by improving secretion of one or more surfactants. In some embodiments, the method of treatment promotes lung cell activity under fibrotic stress by improving expression SP-A, SP-B, SP-C, or any combination thereof. In some embodiments, the method of treatment promotes lung cell activity under fibrotic stress by improving expression of SP-C. In some embodiments, the method of treatment promotes lung cell activity under fibrotic stress by improving expression of SP-C in a dose-responsive manner. In some embodiments, the method of treatment improve an IPF-induced increase in lung resistance. In some embodiments, the method of treatment improve an IPF-induced increase in lung elastance. In some embodiments, the method of treatment inhibits lung fibrosis in the subject. In some embodiments, the method of treatment inhibits progression of lung fibrosis in the subject. In some embodiments, the method of treatment significantly delays a progression of lung fibrosis in the subject. In some embodiments, the method of treatment significantly slows a progression of lung fibrosis in the subject. In some embodiments, the method of treatment reverses an extent of lung fibrosis in the subject. In some embodiments, the method of treatment repairs an extent of lung damage in the subject.

### Schedules Routes of Administration and Amounts

In certain aspects, the polypeptide (e.g., OLFML3, NDP, AREG, FGF1, EFNB2, and/or CCN1) can be administered by any suitable route such as, for example, subcutaneous, intravenous, or intramuscular. In certain aspects, the polypeptide (e.g., OLFML3, NDP, AREG, FGF1, EFNB2, and/or CCN1) are administered on a suitable dosage schedule, for example, weekly, twice weekly, monthly, twice monthly, once every three weeks, or once every four weeks. The polypeptide (e.g., OLFML3, NDP, AREG, FGF1, EFNB2, and/or CCN1) can be administered in any therapeutically effective amount. In certain aspects, the therapeutically acceptable amount is about 0.001 mg/kg to about 1 mg/kg. In certain aspects, the therapeutically acceptable amount is about 0.001 mg/kg to about 0.002 mg/kg, about 0.001 mg/kg to about 0.005 mg/kg, about 0.001 mg/kg to about 0.01 mg/kg, about 0.001 mg/kg to about 0.02 mg/kg, about 0.001 mg/kg to about 0.05 mg/kg, about 0.001 mg/kg to about 0.1 mg/kg, about 0.001 mg/kg to about 0.2 mg/kg, about 0.001 mg/kg to about 0.5 mg/kg, about 0.001 mg/kg to about 1 mg/kg, about 0.002 mg/kg to about 0.005 mg/kg, about 0.002 mg/kg to about 0.01 mg/kg, about 0.002 mg/kg to about 0.02 mg/kg, about 0.002 mg/kg to about 0.05 mg/kg, about 0.002 mg/kg to about 0.1 mg/kg, about 0.002 mg/kg to about 0.2 mg/kg, about 0.002 mg/kg to about 0.5 mg/kg, about 0.002 mg/kg to about 1 mg/kg, about 0.005 mg/kg to about 0.01 mg/kg, about 0.005 mg/kg to about 0.02 mg/kg, about 0.005 mg/kg to about 0.05 mg/kg, about 0.005 mg/kg to about 0.1 mg/kg, about 0.005 mg/kg to about 0.2 mg/kg, about 0.005 mg/kg to about 0.5 mg/kg, about 0.005 mg/kg to about 1 mg/kg, about 0.01 mg/kg to about 0.02 mg/kg, about 0.01 mg/kg to about 0.05 mg/kg, about 0.01 mg/kg to about 0.1 mg/kg, about 0.01 mg/kg to about 0.2 mg/kg, about 0.01 mg/kg to about 0.5 mg/kg, about 0.01 mg/kg to about 1 mg/kg, about 0.02 mg/kg to about 0.05 mg/kg, about 0.02 mg/kg to about 0.1 mg/kg, about 0.02 mg/kg to about 0.2 mg/kg, about 0.02 mg/kg to about 0.5 mg/kg, about 0.02 mg/kg to about 1 mg/kg, about 0.05 mg/kg to about 0.1 mg/kg, about 0.05 mg/kg to about 0.2 mg/kg, about 0.05 mg/kg to about 0.5 mg/kg, about 0.05 mg/kg to about 1 mg/kg, about 0.1 mg/kg to about 0.2 mg/kg, about 0.1 mg/kg to about 0.5 mg/kg, about 0.1 mg/kg to about 1 mg/kg, about 0.2 mg/kg to about 0.5 mg/kg, about 0.2 mg/kg to about 1 mg/kg, or about 0.5 mg/kg to about 1 mg/kg. In certain aspects, the therapeutically acceptable amount is about 0.001 mg/kg, about 0.002 mg/kg, about 0.005 mg/kg, about 0.01 mg/kg, about 0.02 mg/kg, about 0.05 mg/kg, about 0.1 mg/kg, about 0.2 mg/kg, about 0.5 mg/kg, or about 1 mg/kg. In certain aspects, the therapeutically acceptable amount is at least about 0.001 mg/kg, about 0.002 mg/kg, about 0.005 mg/kg, about 0.01 mg/kg, about 0.02 mg/kg, about 0.05 mg/kg, about 0.1 mg/kg, about 0.2 mg/kg, or about 0.5 mg/kg. In certain aspects, the therapeutically acceptable amount is at most about 0.002 mg/kg, about 0.005 mg/kg, about 0.01 mg/kg, about 0.02 mg/kg, about 0.05 mg/kg, about 0.1 mg/kg, about 0.2 mg/kg, about 0.5 mg/kg, or about 1 mg/kg. In certain aspects, the therapeutically acceptable amount is about 0.1 mg/kg to about 50 mg/kg. In certain embodiments, the therapeutically acceptable amount is about 0.1 mg/kg to about 0.2 mg/kg, about 0.1 mg/kg to about 0.5 mg/kg, about 0.1 mg/kg to about 1 mg/kg, about 0.1 mg/kg to about 2 mg/kg, about 0.1 mg/kg to about 5 mg/kg, about 0.1 mg/kg to about 10 mg/kg, about 0.1 mg/kg to about 20 mg/kg, about 0.1 mg/kg to about 50 mg/kg, about 0.2 mg/kg to about 0.5 mg/kg, about 0.2 mg/kg to about 1 mg/kg, about 0.2 mg/kg to about 2 mg/kg, about 0.2 mg/kg to about 5 mg/kg, about 0.2 mg/kg to about 10 mg/kg, about 0.2 mg/kg to about 20 mg/kg, about 0.2 mg/kg to about 50 mg/kg, about 0.5 mg/kg to about 1 mg/kg, about 0.5 mg/kg to about 2 mg/kg, about 0.5 mg/kg to about 5 mg/kg, about 0.5 mg/kg to about 10 mg/kg, about 0.5 mg/kg to about 20 mg/kg, about 0.5 mg/kg to about 50 mg/kg, about 1 mg/kg to about 2 mg/kg, about 1 mg/kg to about 5 mg/kg, about 1 mg/kg to about 10 mg/kg, about 1 mg/kg to about 20 mg/kg, about 1 mg/kg to about 50 mg/kg, about 2 mg/kg to about 5 mg/kg, about 2 mg/kg to about 10 mg/kg, about 2 mg/kg to about 20 mg/kg, about 2 mg/kg to about 50 mg/kg, about 5 mg/kg to about 10 mg/kg, about 5 mg/kg to about 20 mg/kg, about 5 mg/kg to about 50 mg/kg, about 10 mg/kg to about 20 mg/kg, about 10 mg/kg to about 50 mg/kg, or about 20 mg/kg to about 50 mg/kg. In certain aspects, the therapeutically acceptable amount is about 0.1 mg/kg, about 0.2 mg/kg, about 0.5 mg/kg, about 1 mg/kg, about 2 mg/kg, about 5 mg/kg, about 10 mg/kg, about 20 mg/kg, or about 50 mg/kg. In certain aspects, the therapeutically acceptable amount is at least about 0.1 mg/kg, about 0.2 mg/kg, about 0.5 mg/kg, about 1 mg/kg, about 2 mg/kg, about 5 mg/kg, about 10 mg/kg, or about 20 mg/kg. In certain aspects, the therapeutically acceptable amount is at most about 0.2 mg/kg, about 0.5 mg/kg, about 1 mg/kg, about 2 mg/kg, about 5 mg/kg, about 10 mg/kg, about 20 mg/kg, or about 50 mg/kg.

### Nucleic Acids

In certain aspects, described herein, are nucleic acids that encode the polypeptide (e.g., OLFML3, NDP, AREG, FGF1, EFNB2, and/or CCN1) described herein. In certain aspects, the nucleic acids are exogenous. In certain aspects, the nucleic acid is a plasmid. In certain aspects, the nucleic acid is a viral vector. In certain aspects, the viral vector is an adenovirus, lentivirus, retrovirus, adeno-associated virus, or vaccinia virus. In certain aspects, the nucleic acid comprises RNA. In certain aspects, the nucleic acid encodes any of the polypeptides disclosed herein (e.g., OLFML3, NDP, AREG, FGF1, EFNB2, and/or CCN1). In certain aspects, the nucleic acid encodes any one or more polypeptide embodiments described herein. Nucleic acids according to this description can comprise additional nucleic acid sequences sufficient to propagate the vector or express a polypeptide encoded by the vector. In certain aspects, the nucleic acid comprises a universal promoter, such as the CMV promoter, or an inducible promoter system such as a TET_{ON}, TET_{OFF} or GAL4. In certain aspects, the nucleic acid is expressed via a tissue specific promoter or one compatible with a eukaryotic or prokaryotic cellular expression system. The nucleic acid can further comprise a sequence encoding a suitable purification tag (e.g., HIS-tag, V5, FLAG, MYC). In some embodiments, a nucleic acid sequence encoding a human OLFML3 polypeptide, a human NDP polypeptide, a human AREG polypeptide, a human FGF1 polypeptide, a human EFNB2 polypeptide, or a human CCN1 polypeptide is listed in Table 8.

TABLE 8
Nucleic acid sequences encoding polypeptides

| SEQ | | | |
|---|---|---|---|
| ID | Sequence | | |
| NO: | name | Description | Sequence |
| 27 | Human | Homo | gactgtacgttccttctactctggcaccactctccaggctgccatggggcccagcacccctctcctcatcttgttccttttgtca |
| | OLFML3- | sapiens | tggtcgggacccctccaaggacagcagcaccaccttgtggagtacatggaacgccgactagctgctttagaggaacg |
| | isoform | olfactomedin | gctggcccagtgccaggaccagagtagtcggcatgctgctgagctgcgggacttcaagaacaagatgctgccactgct |
| | 1-cDNA | like 3 | ggaggtggcagagaaggagcgggaggcactcagaactgaggccgacaccatctccgggagagtggatcgtctgga |
| | | (OLFML3), | gcgggaggtagactatctggagacccagaacccagctctgccctgtgtagagtttgatgagaaggtgactggaggccc |
| | | transcript | tgggaccaaaggcaagggaagaaggaatgagaagtacgatatggtgacagactgtggctacacaatctctcaagtg |
| | | variant 1, | agatcaatgaagattctgaagcgatttggtggcccagctggtctatggaccaaggatccactggggcaaacagagaag |
| | | mRNA | atctacgtgttagatgggacacagaatgacacagcctttgtcttcccaaggctgcgtgacttcacccttgccatggctgccc |
| | | (NM_020190.5) | ggaaagcttcccgagtccgggtgcccttcccctgggtaggcacagggcagctggtatatggtggctttctttattttgctcgg |
| | | | aggcctcctggaagacctggtggaggtggtgagatggagaacactttgcagctaatcaaattccacctggcaaaccga |
| | | | acagtggtggacagctcagtattcccagcagaggggctgatccccccctacggcttgacagcagacacctacatcgac |
| | | | ctggcagctgatgaggaaggtctttgggctgtctatgccacccgggaggatgacaggcacttgtgtctggccaagttaga |
| | | | tccacagacactggacacagagcagcagtgggacacaccatgtcccagagagaatgctgaggctgcctttgtcatctg |
| | | | tgggaccctctatgtcgtctataacacccgtcctgccagtcgggcccgcatccagtgctcctttgatgccagcggcaccct |
| | | | gacccctgaacgggcagcactcccttattttccccgcagatatggtgcccatgccagcctccgctataacccccgagaa |
| | | | cgccagctctatgcctgggatgatggctaccagattgtctataagctggagatgaggaagaaagaggaggaggtttga |
| | | | ggagctagccttgttttttgcatctttctcactcccatacatttatattatatccccactaaatttcttgttcctcattcttcaaatgtg |
| | | | ggccagttgtggctcaaatcctctatatttttagccaatggcaatcaaattctttcagctcctttgtttcatacggaactccagat |
| | | | cctgagtaatccttttagagcccgaagagtcaaaaccctcaatgttccctcctgctctcctgccccatgtcaacaaatttca |
| | | | ggctaaggatgccccagacccagggctctaaccttgtatgcgggcaggcccagggagcaggcagcagtgttcttcccc |
| | | | tcagagtgacttggggagggagaaataggaggagacgtccagctctgtcctctcttcctcactcctcccttcagtgtcctg |
| | | | aggaacaggactttctccacattgttttgtattgcaacattttgcattaaaaggaaaatccactgct |
| 28 | Human | Homo | gactgtacgttccttctactctggcaccactctccaggctgccatggggcccagcacccctctcctcatcttgttccttttgtca |
| | OLFML3- | sapiens | tggtcgggacccctccaaggacagcagcaccaccttgtggagtacatggaacgccgactagctgctttagaggttgag |
| | isoform | olfactomedin | atccgtagcattttagtgtccgagaggataattgctaagggagcagagacctcttcacaccttcatgcgcttagacctgcct |
| | 2-cDNA | like 3 | agaggctcagatggctcagtggcactgctcacagagaacacaggctggaggacatggagtcggggaacggctggc |
| | | (OLFML3), | ccagtgccaggaccagagtagtcggcatgctgctgagctgcgggacttcaagaacaagatgctgccactgctggaggt |
| | | transcript | ggcagagaaggagcgggaggcactcagaactgaggccgacaccatctccgggagagtggatcgtctggagcggg |
| | | variant 2, | aggtagactatctggagacccagaacccagctctgccctgtgtagagtttgatgagaaggtgactggaggccctggga |
| | | mRNA (NM_ | ccaaaggcaagggaagaaggaatgagaagtacgatatggtgacagactgtggctacacaatctctcaagtgagatc |
| | | 001286352.3) | aatgaagattctgaagcgatttggtggcccagctggtctatggaccaaggatccactggggcaaacagagaagatcta |
| | | | cgtgttagatgggacacagaatgacacagcctttgtcttcccaaggctgcgtgacttcacccttgccatggctgcccggaa |
| | | | agcttcccgagtccgggtgcccttcccctgggtaggcacagggcagctggtatatggtggctttctttattttgctcggaggc |
| | | | ctcctggaagacctggtggaggtggtgagatggagaacactttgcagctaatcaaattccacctggcaaaccgaacag |
| | | | tggtggacagctcagtattcccagcagaggggctgatccccccctacggcttgacagcagacacctacatcgacctgg |
| | | | cagctgatgaggaaggtctttgggctgtctatgccacccgggaggatgacaggcacttgtgtctggccaagttagatcca |
| | | | cagacactggacacagagcagcagtgggacacaccatgtcccagagagaatgctgaggctgcctttgtcatctgtggg |
| | | | accctctatgtcgtctataacacccgtcctgccagtcgggcccgcatccagtgctcctttgatgccagcggcaccctgacc |
| | | | cctgaacgggcagcactcccttattttccccgcagatatggtgcccatgccagcctccgctataacccccgagaacgcc |
| | | | agctctatgcctgggatgatggctaccagattgtctataagctggagatgaggaagaaagaggaggaggtttgaggag |
| | | | ctagccttgttttttgcatctttctcactcccatacatttatattatatccccactaaatttcttgttcctcattcttcaaatgtgggcca |
| | | | gttgtggctcaaatcctctatatttttagccaatggcaatcaaattctttcagctcctttgtttcatacggaactccagatcctga |
| | | | gtaatccttttagagcccgaagagtcaaaaccctcaatgttccctcctgctctcctgccccatgtcaacaaatttcaggcta |
| | | | aggatgccccagacccagggctctaaccttgtatgcgggcaggcccagggagcaggcagcagtgttcttcccctcaga |
| | | | gtgacttggggagggagaaataggaggagacgtccagctctgtcctctcttcctcactcctcccttcagtgtcctgaggaa |
| | | | caggactttctccacattgttttgtattgcaacattttgcattaaaaggaaaatccactgct |
| 29 | Human | Homo | gactgtacgttccttctactctggcaccactctccaggctgccatggggcccagcacccctctcctcatcttgttccttttgtca |
| | OLFML3- | sapiens | tggtcgggacccctccaaggacagcagcaccaccttgtggagtacatggaacgccgactagctgctttagaggagtgg |
| | isoform | olfactomedin | aaggggatattgaagcctggctgagtttgctctttttttcccattcctgactcccgagttgggcctctccctgcctgggagttgt |
| | 3-cDNA | like 3 | aaccctgcagcctccctcctgatcccccatcccttcaggaacggctggcccagtgccaggaccagagtagtcggcatg |
| | | (OLFML3), | ctgctgagctgcgggacttcaagaacaagatgctgccactgctggaggtggcagagaaggagcgggaggcactcag |
| | | transcript | aactgaggccgacaccatctccgggagagtggatcgtctggagcgggaggtagactatctggagacccagaaccca |
| | | variant 3, | gctctgccctgtgtagagtttgatgagaaggtgactggaggccctgggaccaaaggcaagggaagaaggaatgaga |
| | | mRNA (NM_ | agtacgatatggtgacagactgtggctacacaatctctcaagtgagatcaatgaagattctgaagcgatttggtggccca |
| | | 001286353.3) | gctggtctatggaccaaggatccactggggcaaacagagaagatctacgtgttagatgggacacagaatgacacagc |
| | | | ctttgtcttcccaaggctgcgtgacttcacccttgccatggctgcccggaaagcttcccgagtccgggtgcccttcccctgg |
| | | | gtaggcacagggcagctggtatatggtggctttctttattttgctcggaggcctcctggaagacctggtggaggtggtgaga |
| | | | tggagaacactttgcagctaatcaaattccacctggcaaaccgaacagtggtggacagctcagtattcccagcagagg |
| | | | ggctgatccccccctacggcttgacagcagacacctacatcgacctggcagctgatgaggaaggtctttgggctgtctat |
| | | | gccacccgggaggatgacaggcacttgtgtctggccaagttagatccacagacactggacacagagcagcagtggg |
| | | | acacaccatgtcccagagagaatgctgaggctgcctttgtcatctgtgggaccctctatgtcgtctataacacccgtcctgc |
| | | | cagtcgggcccgcatccagtgctcctttgatgccagcggcaccctgacccctgaacgggcagcactcccttattttcccc |
| | | | gcagatatggtgcccatgccagcctccgctataacccccgagaacgccagctctatgcctgggatgatggctaccagat |
| | | | tgtctataagctggagatgaggaagaaagaggaggaggtttgaggagctagccttgttttttgcatctttctcactcccatac |
| | | | atttatattatatccccactaaatttcttgttcctcattcttcaaatgtgggccagttgtggctcaaatcctctatatttttagccaat |
| | | | ggcaatcaaattctttcagctcctttgtttcatacggaactccagatcctgagtaatccttttagagcccgaagagtcaaaac |
| | | | cctcaatgttccctcctgctctcctgccccatgtcaacaaatttcaggctaaggatgccccagacccagggctctaaccttg |
| | | | tatgcgggcaggcccagggagcaggcagcagtgttcttcccctcagagtgacttggggagggagaaataggaggag |
| | | | acgtccagctctgtcctctcttcctcactcctcccttcagtgtcctgaggaacaggactttctccacattgttttgtattgcaacat |
| | | | tttgcattaaaaggaaaatccactgct |
| 30 | Human | Homo | agaagaacaaaagcatttggaagtaacaggacctctttctagctctcagaaaagtctgagaagaaaggagccctgcgt |
| | NDP- | sapiens | tcccctaagctgtgcagcagatactgtgatgatggattgcaagtgcaaagagtaagacaaaactccagcacataaagg |
| | cDNA | norrin | acaatgacaaccagaaagcttcagcccgatcctgccctttccttgaacgggactggatcctaggaggtgaagccatttcc |
| | | cystine knot | aattttttgtcctctgcctccctctgctgttcttctagagaagtttttccttacaacaatgagaaaacatgtactagctgcatccttt |
| | | growth | tctatgctctccctgctggtgataatgggagatacagacagtaaaacggacagctcattcataatggactcggaccctcg |
| | | factor NDP | acgctgcatgaggcaccactatgtggattctatcagtcacccattgtacaagtgtagctcaaagatggtgctcctggccag |
| | | (NDP), mRNA | gtgcgaggggcactgcagccaggcgtcacgctccgagcctttggtgtcgttcagcactgtcctcaagcaacccttccgtt |
| | | (NM_000266.4) | cctcctgtcactgctgccggccccagacttccaagctgaaggcactgcggctgcgatgctcagggggcatgcgactcac |
| | | | tgccacctaccggtacatcctctcctgtcactgcgaggaatgcaattcctgaggcccgctgctgtgtgtggcttctggatgg |
| | | | gacaactgtagaggcagttcgaccagccagggaaagactggcaagaaaagagttaaggcaaaaaaggatgcaac |
| | | | aattctcccgggactctgcatattctagtaataaagactctacatgcttgttgacagagagagatactctgggaacttctttg |
| | | | cagttcccatctcctttctctggtacaatttcttttggttcattttcagattcaggcattttcccccttggctctcaatgctgtttgggttt |
| | | | ccaacaattcagcattagtgggaaaaagtgggccctcatacacaagcgtgtcaggctgtcagtgtttggtgcacgctgg |
| | | | ggaagaatttactttggaaagtagaaaagcccagcttttcctgggacatcttctgttattgttgatgtttttttttaccttgtcatttt |
| | | | ggtctaaggttgccattgctgctaaaggttaccgatttcaaagtccagataccaagcatgtggatatgtttagctacgtttact |
| | | | cacagccagcgaactgacattaaaataactaacaaacagattcttttatgtgatgctggaactcttgacagctataattatt |
| | | | attcagaaatgactttttgaaagtaaaagcagcataaagaatttgtcacaggaaggctgtctcagataaattatggtaaaa |
| | | | ttttgtaagggagcagacttttaaagacttgcacaaatacggatcctgcactgactctggaaaaggcatatatgtactagtg |
| | | | gcatggagaatgcaccatactcatgcatgcaaattagacaaccaagtatgaatctatttgtgggtgtgctatagctttagcc |
| | | | gtgtcacgggcatcattctctaatatccacttgtccatgtgaaacatgttgccaaaatggtggcctggcttgtcttctgaacgtt |
| | | | tggttcaaatgtgttttggtcctggaggctcaaattttgagttattcccacgttttgaaataaaaagagtatattcaaaa |
| 31 | Human | Homo | agagctgcagtagcctggaggttcagagagccgggctactctgagaagaagacaccaagtggattctgcttcccctgg |
| | FGF1- | sapiens | gacagcactgagcgagtgtggagagaggtacagccctcggcctacaagctctttagtcttgaaagcgccacaagcag |
| | isoform | fibroblast | cagctgctgagccatggctgaaggggaaatcaccaccttcacagccctgaccgagaagtttaatctgcctccagggaa |
| | 1-cDNA | growth | ttacaagaagcccaaactcctctactgtagcaacgggggccacttcctgaggatccttccggatggcacagtggatggg |
| | | factor 1 | acaagggacaggagcgaccagcacattcagctgcagctcagtgcggaaagcgtgggggaggtgtatataaagagta |
| | | (FGF1) | ccgagactggccagtacttggccatggacaccgacgggcttttatacggctcacagacaccaaatgaggaatgtttgttc |
| | | transcript | ctggaaaggctggaggagaaccattacaacacctatatatccaagaagcatgcagagaagaattggtttgttggcctca |
| | | variant 1, | agaagaatgggagctgcaaacgcggtcctcggactcactatggccagaaagcaatcttgtttctccccctgccagtctctt |
| | | mRNA | ctgattaaagagatctgttctgggtgttgaccactccagagaagtttcgaggggtcctcacctggttgacccaaaaatgttc |
| | | (NM_000800.5) | ccttgaccattggctgcgctaacccccagcccacagagcctgaatttgtaagcaacttgcttctaaatgcccagttcacttc |
| | | | tttgcagagccttttacccctgcacagtttagaacagagggaccaaattgcttctaggagtcaactggctggccagtctgg |
| | | | gtctgggtttggatctccaattgcctcttgcaggctgagtccctccatgcaaaagtggggctaaatgaagtgtgttaagggg |
| | | | tcggctaagtgggacattagtaactgcacactatttccctctactgagtaaaccctatctgtgattcccccaaacatctggca |
| | | | tggctcccttttgtccttcctgtgccctgcaaatattagcaaagaagcttcatgccaggttaggaaggcagcattccatgac |
| | | | cagaaacagggacaaagaaatccccccttcagaacagaggcatttaaaatggaaaagagagattggattttggtggg |
| | | | taacttagaaggatggcatctccatgtagaataaatgaagaaagggaggcccagccgcaggaaggcagaataaatc |
| | | | cttgggagtcattaccacgccttgaccttcccaaggttactcagcagcagagagccctgggtgacttcaggtggagagc |
| | | | actagaagtggtttcctgataacaagcaaggatatcagagctgggaaattcatgtggatctggggactgagtgtgggagt |
| | | | gcagagaaagaaagggaaactggctgaggggataccataaaaagaggatgatttcagaaggagaaggaaaaag |
| | | | aaagtaatgccacacattgtgcttggcccctggtaagcagaggctttggggtcctagcccagtgcttctccaacactgaa |
| | | | gtgcttgcagatcatctggggacctggtttgaatggagattctgattcagtgggttgggggcagagtttctgcagttccatca |
| | | | ggtcccccccaggtgcaggtgctgacaatactgctgccttacccgccatacattaaggagcagggtcctggtcctaaag |
| | | | agttattcaaatgaaggtggttcgacgccccgaacctcacctgacctcaactaacccttaaaaatgcacacctcatgagt |
| | | | ctacctgagcattcaggcagcactgacaatagttatgcctgtactaaggagcatgattttaagaggctttggcccaatgcct |
| | | | ataaaatgcccatttcgaagatatacaaaaacatacttcaaaaatgttaaacccttaccaacagcttttcccaggagacc |
| | | | atttgtattaccattacttgtataaatacacttcctgcttaaacttgacccaggtggctagcaaattagaaacaccattcatctc |
| | | | taacatatgatactgatgccatgtaaaggcctttaataagtcattgaaatttactgtgagactgtatgttttaattgcatttaaaa |
| | | | atatatagcttgaaagcagttaaactgattagtattcaggcactgagaatgatagtaataggatacaatgtataagctactc |
| | | | acttatctgatacttatttacctataaaatgagatttttgttttccactgtgctattacaaattttcttttgaaagtaggaactcttaa |
| | | | gcaatggtaattgtgaataaaaattgatgagagtgttagctcctgtttcatatgaaattgaagtaattgttaactaaaaacaa |
| | | | ttccttagtaactgaactgtcatatttagaatggaaggaaaatgacagtttgtgaaagttcaaagcaatagtgcaattgaag |
| | | | aattgacctaagtaagctgacattatggttaataatagtattttagatttgtgcagcaaaataatttcataacttttttgtttttgtta |
| | | | cttggataagatcaatctgttttattttagtaaatctttgcaggcaagttagagaaaatgcagtgtggcttaacgtctctttagta |
| | | | tgaagatttggccagaaaaagatacccagagaggaaatctaagataattataatggtccatactttttattgtatgaatcaa |
| | | | actcaagcataacattggccaaggaaaattaaataccattgctaacttgtgaaatggaagtctgtgatttcggagatgca |
| | | | aagcattgtagtaaaaacaccaatgtgacctcgaccatctcagcccagatatcattcatatatctgttcaatgactattaag |
| | | | gtgcctactgtgtgctaggcactgtactggatactggggaccttgtctgtctggtttgctgctgtatcttctcccagggcattata |
| | | | tttatgatgaaagatgctgtggattcaattctttcagtcaagaataaacacagactttgtaggttcctgctgaataaagcaaa |
| | | | tcccagaaacccagattttggaagaatcagcaaccccagcataaaataaacccctatcaaaatgtcagaggacatgg |
| | | | caaggtaaacttagcattttcaactttagaaccgggtcagcttcagggggactgctttcaaatcagccaaagagcctgtca |
| | | | gatcttcttagaaggaagaggttggtagttccctgctctgttttgaacatgctctagtttattaacctggggacattcccattgct |
| | | | gtcttaagtaagtctcatagccagctcctgtcacgtgactctcatatggattcattttcgggccagctctgaacaaagcatca |
| | | | tgaacatatgtgcttttggtcgtttgcaatgtgatggtggtggaggtaggtattggtttccttggaaggcatgataagaaagat |
| | | | tcacaatggccaacagtgtgtatgaacaaaaaactgattggagcatcagctagtactgaaggtccttgctttgtgtcagag |
| | | | gcaaaggaacccaaggcgccaagtcctcagccttgagtgtactgctgacaactaaactcacaggctgcaaagcaga |
| | | | cctctgatgaagatgcctgttatttcacatcactgtctttttgtgtatcatagtctgcaccttacaaatattaataaatgttccaat |
| | | | aataggtga |
| 32 | Human | Homo | gtgggcataccagtgtcagctgcacttgtaggggcccaagtgcctcatgacccactcggcagccttcctctccaggatcc |
| | FGF1- | sapiens | ccaaggctaggaggccaacctactaacagtcttgaaagcgccacaagcagcagctgctgagccatggctgaagggg |
| | isoform | fibroblast | aaatcaccaccttcacagccctgaccgagaagtttaatctgcctccagggaattacaagaagcccaaactcctctactgt |
| | 2-cDNA | growth | agcaacgggggccacttcctgaggatccttccggatggcacagtggatgggacaagggacaggagcgaccagcac |
| | | factor 1 | acagacaccaaatgaggaatgtttgttcctggaaaggctggaggagaaccattacaacacctatatatccaagaagca |
| | | (FGF1), | tgcagagaagaattggtttgttggcctcaagaagaatgggagctgcaaacgcggtcctcggactcactatggccagaa |
| | | transcript | agcaatcttgtttctccccctgccagtctcttctgattaaagagatctgttctgggtgttgaccactccagagaagtttcgagg |
| | | variant 25, | ggtcctcacctggttgacccaaaaatgttcccttgaccattggctgcgctaacccccagcccacagagcctgaatttgtaa |
| | | mRNA (NM_ | gcaacttgcttctaaatgcccagttcacttctttgcagagccttttacccctgcacagtttagaacagagggaccaaattgct |
| | | 001354962.2) | tctaggagtcaactggctggccagtctgggtctgggtttggatctccaattgcctcttgcaggctgagtccctccatgcaaa |
| | | | agtggggctaaatgaagtgtgttaaggggtcggctaagtgggacattagtaactgcacactatttccctctactgagtaaa |
| | | | ccctatctgtgattcccccaaacatctggcatggctcccttttgtccttcctgtgccctgcaaatattagcaaagaagcttcat |
| | | | gccaggttaggaaggcagcattccatgaccagaaacagggacaaagaaatccccccttcagaacagaggcatttaa |
| | | | aatggaaaagagagattggattttggtgggtaacttagaaggatggcatctccatgtagaataaatgaagaaagggag |
| | | | gcccagccgcaggaaggcagaataaatccttgggagtcattaccacgccttgaccttcccaaggttactcagcagcag |
| | | | agagccctgggtgacttcaggtggagagcactagaagtggtttcctgataacaagcaaggatatcagagctgggaaat |
| | | | tcatgtggatctggggactgagtgtgggagtgcagagaaagaaagggaaactggctgaggggataccataaaaaga |
| | | | ggatgatttcagaaggagaaggaaaaagaaagtaatgccacacattgtgcttggcccctggtaagcagaggctttggg |
| | | | gtcctagcccagtgcttctccaacactgaagtgcttgcagatcatctggggacctggtttgaatggagattctgattcagtgg |
| | | | gttgggggcagagtttctgcagttccatcaggtcccccccaggtgcaggtgctgacaatactgctgccttacccgccatac |
| | | | attaaggagcagggtcctggtcctaaagagttattcaaatgaaggtggttcgacgccccgaacctcacctgacctcaact |
| | | | aacccttaaaaatgcacacctcatgagtctacctgagcattcaggcagcactgacaatagttatgcctgtactaaggagc |
| | | | atgattttaagaggctttggcccaatgcctataaaatgcccatttcgaagatatacaaaaacatacttcaaaaatgttaaac |
| | | | ccttaccaacagcttttcccaggagaccatttgtattaccattacttgtataaatacacttcctgcttaaacttgacccaggtg |
| | | | gctagcaaattagaaacaccattcatctctaacatatgatactgatgccatgtaaaggcctttaataagtcattgaaatttac |
| | | | tgtgagactgtatgttttaattgcatttaaaaatatatagcttgaaagcagttaaactgattagtattcaggcactgagaatga |
| | | | tagtaataggatacaatgtataagctactcacttatctgatacttatttacctataaaatgagatttttgttttccactgtgctatta |
| | | | caaattttcttttgaaagtaggaactcttaagcaatggtaattgtgaataaaaattgatgagagtgttagctcctgtttcatatg |
| | | | aaattgaagtaattgttaactaaaaacaattccttagtaactgaactgtcatatttagaatggaaggaaaatgacagtttgt |
| | | | gaaagttcaaagcaatagtgcaattgaagaattgacctaagtaagctgacattatggttaataatagtattttagatttgtgc |
| | | | agcaaaataatttcataacttttttgtttttgttacttggataagatcaatctgttttattttagtaaatctttgcaggcaagttagag |
| | | | aaaatgcagtgtggcttaacgtctctttagtatgaagatttggccagaaaaagatacccagagaggaaatctaagataat |
| | | | tataatggtccatactttttattgtatgaatcaaactcaagcataacattggccaaggaaaattaaataccattgctaacttgt |
| | | | gaaatggaagtctgtgatttcggagatgcaaagcattgtagtaaaaacaccaatgtgacctcgaccatctcagcccaga |
| | | | tatcattcatatatctgttcaatgactattaaggtgcctactgtgtgctaggcactgtactggatactggggaccttgtctgtctg |
| | | | gtttgctgctgtatcttctcccagggcattatatttatgatgaaagatgctgtggattcaattctttcagtcaagaataaacaca |
| | | | gactttgtaggttcctgctgaataaagcaaatcccagaaacccagattttggaagaatcagcaaccccagcataaaata |
| | | | aacccctatcaaaatgtcagaggacatggcaaggtaaacttagcattttcaactttagaaccgggtcagcttcaggggg |
| | | | actgctttcaaatcagccaaagagcctgtcagatcttcttagaaggaagaggttggtagttccctgctctgttttgaacatgc |
| | | | tctagtttattaacctggggacattcccattgctgtcttaagtaagtctcatagccagctcctgtcacgtgactctcatatggatt |
| | | | cattttcgggccagctctgaacaaagcatcatgaacatatgtgcttttggtcgtttgcaatgtgatggtggtggaggtaggta |
| | | | ttggtttccttggaaggcatgataagaaagattcacaatggccaacagtgtgtatgaacaaaaaactgattggagcatca |
| | | | gctagtactgaaggtccttgctttgtgtcagaggcaaaggaacccaaggcgccaagtcctcagccttgagtgtactgctg |
| | | | acaactaaactcacaggctgcaaagcagacctctgatgaagatgcctgttatttcacatcactgtctttttgtgtatcatagtc |
| | | | tgcaccttacaaatattaataaatgttccaataataggtga |
| 33 | Human | Homo | gtgggcataccagtgtcagctgcacttgtaggggcccaagtgcctcatgacccactcggcagccttcctctccaggatcc |
| | FGF1- | sapiens | ccaaggctaggaggccaacctactaacagtcttgaaagcgccacaagcagcagctgctgagccatggctgaagggg |
| | isoform | fibroblast | aaatcaccaccttcacagccctgaccgagaagtttaatctgcctccagggaattacaagaagcccaaactcctctactgt |
| | 3-cDNA | growth | agcaacgggggccacttcctgaggatccttccggatggcacagtggatgggacaagggacaggagcgaccagcac |
| | | factor 1 | aacaccaaatgaggaatgtttgttcctggaaaggctggaggagaaccattacaacacctatatatccaagaagcatgc |
| | | (FGF1), | agagaagaattggtttgttggcctcaagaagaatgggagctgcaaacgcggtcctcggactcactatggccagaaagc |
| | | transcript | aatcttgtttctccccctgccagtctcttctgattaaagagatctgttctgggtgttgaccactccagagaagtttcgaggggtc |
| | | variant 3, | ctcacctggttgacccaaaaatgttcccttgaccattggctgcgctaacccccagcccacagagcctgaatttgtaagca |
| | | mRNA | acttgcttctaaatgcccagttcacttctttgcagagccttttacccctgcacagtttagaacagagggaccaaattgcttcta |
| | | (NM_033137.4) | ggagtcaactggctggccagtctgggtctgggtttggatctccaattgcctcttgcaggctgagtccctccatgcaaaagtg |
| | | | gggctaaatgaagtgtgttaaggggtcggctaagtgggacattagtaactgcacactatttccctctactgagtaaaccct |
| | | | atctgtgattcccccaaacatctggcatggctcccttttgtccttcctgtgccctgcaaatattagcaaagaagcttcatgcca |
| | | | ggttaggaaggcagcattccatgaccagaaacagggacaaagaaatccccccttcagaacagaggcatttaaaatg |
| | | | gaaaagagagattggattttggtgggtaacttagaaggatggcatctccatgtagaataaatgaagaaagggaggccc |
| | | | agccgcaggaaggcagaataaatccttgggagtcattaccacgccttgaccttcccaaggttactcagcagcagagag |
| | | | ccctgggtgacttcaggtggagagcactagaagtggtttcctgataacaagcaaggatatcagagctgggaaattcatgt |
| | | | ggatctggggactgagtgtgggagtgcagagaaagaaagggaaactggctgaggggataccataaaaagaggatg |
| | | | atttcagaaggagaaggaaaaagaaagtaatgccacacattgtgcttggcccctggtaagcagaggctttggggtccta |
| | | | gcccagtgcttctccaacactgaagtgcttgcagatcatctggggacctggtttgaatggagattctgattcagtgggttgg |
| | | | gggcagagtttctgcagttccatcaggtcccccccaggtgcaggtgctgacaatactgctgccttacccgccatacattaa |
| | | | ggagcagggtcctggtcctaaagagttattcaaatgaaggtggttcgacgccccgaacctcacctgacctcaactaacc |
| | | | cttaaaaatgcacacctcatgagtctacctgagcattcaggcagcactgacaatagttatgcctgtactaaggagcatgat |
| | | | tttaagaggctttggcccaatgcctataaaatgcccatttcgaagatatacaaaaacatacttcaaaaatgttaaaccctta |
| | | | ccaacagcttttcccaggagaccatttgtattaccattacttgtataaatacacttcctgcttaaacttgacccaggtggctag |
| | | | caaattagaaacaccattcatctctaacatatgatactgatgccatgtaaaggcctttaataagtcattgaaatttactgtga |
| | | | gactgtatgttttaattgcatttaaaaatatatagcttgaaagcagttaaactgattagtattcaggcactgagaatgatagta |
| | | | ataggatacaatgtataagctactcacttatctgatacttatttacctataaaatgagatttttgttttccactgtgctattacaaat |
| | | | tttcttttgaaagtaggaactcttaagcaatggtaattgtgaataaaaattgatgagagtgttagctcctgtttcatatgaaatt |
| | | | gaagtaattgttaactaaaaacaattccttagtaactgaactgtcatatttagaatggaaggaaaatgacagtttgtgaaa |
| | | | gttcaaagcaatagtgcaattgaagaattgacctaagtaagctgacattatggttaataatagtattttagatttgtgcagca |
| | | | aaataatttcataacttttttgtttttgttacttggataagatcaatctgttttattttagtaaatctttgcaggcaagttagagaaaa |
| | | | tgcagtgtggcttaacgtctctttagtatgaagatttggccagaaaaagatacccagagaggaaatctaagataattataa |
| | | | tggtccatactttttattgtatgaatcaaactcaagcataacattggccaaggaaaattaaataccattgctaacttgtgaaat |
| | | | ggaagtctgtgatttcggagatgcaaagcattgtagtaaaaacaccaatgtgacctcgaccatctcagcccagatatcat |
| | | | tcatatatctgttcaatgactattaaggtgcctactgtgtgctaggcactgtactggatactggggaccttgtctgtctggtttgc |
| | | | tgctgtatcttctcccagggcattatatttatgatgaaagatgctgtggattcaattctttcagtcaagaataaacacagacttt |
| | | | gtaggttcctgctgaataaagcaaatcccagaaacccagattttggaagaatcagcaaccccagcataaaataaaccc |
| | | | ctatcaaaatgtcagaggacatggcaaggtaaacttagcattttcaactttagaaccgggtcagcttcagggggactgctt |
| | | | tcaaatcagccaaagagcctgtcagatcttcttagaaggaagaggttggtagttccctgctctgttttgaacatgctctagttt |
| | | | attaacctggggacattcccattgctgtcttaagtaagtctcatagccagctcctgtcacgtgactctcatatggattcattttc |
| | | | gggccagctctgaacaaagcatcatgaacatatgtgcttttggtcgtttgcaatgtgatggtggtggaggtaggtattggttt |
| | | | ccttggaaggcatgataagaaagattcacaatggccaacagtgtgtatgaacaaaaaactgattggagcatcagctag |
| | | | tactgaaggtccttgctttgtgtcagaggcaaaggaacccaaggcgccaagtcctcagccttgagtgtactgctgacaac |
| | | | taaactcacaggctgcaaagcagacctctgatgaagatgcctgttatttcacatcactgtctttttgtgtatcatagtctgcac |
| | | | cttacaaatattaataaatgttccaataataggtga |
| 34 | Human | Homo | ggagacagaacaatcttgaaagcgccacaagcagcagctgctgagccatggctgaaggggaaatcaccaccttcac |
| | isoform | sapiens | gtgggcataccagtgtcagctgcacttgtaggggcccaagtgcctcatgacccactcggcagccttcctctccaggatcc |
| | 4-FGF1- | fibroblast | ccaaggctaggaggccaacctactaacagcctgcctgcagctgtcctggtagaacagtgtggacattgcagaagctgt |
| | cDNA | growth | agccctgaccgagaagtttaatctgcctccagggaattacaagaagcccaaactcctctactgtagcaacgggggcca |
| | | factor 1 | cttcctgaggatccttccggatggcacagtggatgggacaagggacaggagcgaccagcacattcagctgcagctca |
| | | (FGF1), | gtgcggaaagcgtgggggaggtgtatataaagagtaccgagactggccagtacttggccatggacaccgacgggcttt |
| | | transcript | cactgccccagaaagaaagcaccccagagccaaggcaaagaggtgattccagtgcacactcgagcttgagaacca |
| | | variant 21, | ctggtctcggtactgagtatataacagtgagaagacaaagtcctgctctccttgtggaatttgtattccagtgtatgtggtagg |
| | | mRNA (NM_ | tatacggctcaacaccaaatgaggaatgtttgttcctggaaaggctggaggagaaccattacaacacctatatatccaa |
| | | 001354957.2) | gaagcatgcagagaagaattggtttgttggcctcaagaagaatgggagctgcaaacgcggtcctcggactcactatgg |
| | | | tcgaggggtcctcacctggttgacccaaaaatgttcccttgaccattggctgcgctaacccccagcccacagagcctga |
| | | | ccagaaagcaatcttgtttctccccctgccagtctcttctgattaaagagatctgttctgggtgttgaccactccagagaagtt |
| | | | atttgtaagcaacttgcttctaaatgcccagttcacttctttgcagagccttttacccctgcacagtttagaacagagggacc |
| | | | aaattgcttctaggagtcaactggctggccagtctgggtctgggtttggatctccaattgcctcttgcaggctgagtccctcc |
| | | | atgcaaaagtggggctaaatgaagtgtgttaaggggtcggctaagtgggacattagtaactgcacactatttccctctact |
| | | | gagtaaaccctatctgtgattcccccaaacatctggcatggctcccttttgtccttcctgtgccctgcaaatattagcaaaga |
| | | | agcttcatgccaggttaggaaggcagcattccatgaccagaaacagggacaaagaaatccccccttcagaacagag |
| | | | gcatttaaaatggaaaagagagattggattttggtgggtaacttagaaggatggcatctccatgtagaataaatgaagaa |
| | | | agggaggcccagccgcaggaaggcagaataaatccttgggagtcattaccacgccttgaccttcccaaggttactcag |
| | | | cagcagagagccctgggtgacttcaggtggagagcactagaagtggtttcctgataacaagcaaggatatcagagctg |
| | | | ggaaattcatgtggatctggggactgagtgtgggagtgcagagaaagaaagggaaactggctgaggggataccata |
| | | | aaaagaggatgatttcagaaggagaaggaaaaagaaagtaatgccacacattgtgcttggcccctggtaagcagag |
| | | | gctttggggtcctagcccagtgcttctccaacactgaagtgcttgcagatcatctggggacctggtttgaatggagattctga |
| | | | ttcagtgggttgggggcagagtttctgcagttccatcaggtcccccccaggtgcaggtgctgacaatactgctgccttaccc |
| | | | gccatacattaaggagcagggtcctggtcctaaagagttattcaaatgaaggtggttcgacgccccgaacctcacctga |
| | | | cctcaactaacccttaaaaatgcacacctcatgagtctacctgagcattcaggcagcactgacaatagttatgcctgtact |
| | | | aaggagcatgattttaagaggctttggcccaatgcctataaaatgcccatttcgaagatatacaaaaacatacttcaaaa |
| | | | atgttaaacccttaccaacagcttttcccaggagaccatttgtattaccattacttgtataaatacacttcctgcttaaacttga |
| | | | cccaggtggctagcaaattagaaacaccattcatctctaacatatgatactgatgccatgtaaaggcctttaataagtcatt |
| | | | gaaatttactgtgagactgtatgttttaattgcatttaaaaatatatagcttgaaagcagttaaactgattagtattcaggcact |
| | | | gagaatgatagtaataggatacaatgtataagctactcacttatctgatacttatttacctataaaatgagatttttgttttccac |
| | | | tgtgctattacaaattttcttttgaaagtaggaactcttaagcaatggtaattgtgaataaaaattgatgagagtgttagctcct |
| | | | gtttcatatgaaattgaagtaattgttaactaaaaacaattccttagtaactgaactgtcatatttagaatggaaggaaaatg |
| | | | acagtttgtgaaagttcaaagcaatagtgcaattgaagaattgacctaagtaagctgacattatggttaataatagtatttta |
| | | | gatttgtgcagcaaaataatttcataacttttttgtttttgttacttggataagatcaatctgttttattttagtaaatctttgcaggca |
| | | | agttagagaaaatgcagtgtggcttaacgtctctttagtatgaagatttggccagaaaaagatacccagagaggaaatct |
| | | | aagataattataatggtccatactttttattgtatgaatcaaactcaagcataacattggccaaggaaaattaaataccattg |
| | | | ctaacttgtgaaatggaagtctgtgatttcggagatgcaaagcattgtagtaaaaacaccaatgtgacctcgaccatctca |
| | | | gcccagatatcattcatatatctgttcaatgactattaaggtgcctactgtgtgctaggcactgtactggatactggggacctt |
| | | | gtctgtctggtttgctgctgtatcttctcccagggcattatatttatgatgaaagatgctgtggattcaattctttcagtcaagaat |
| | | | aaacacagactttgtaggttcctgctgaataaagcaaatcccagaaacccagattttggaagaatcagcaaccccagc |
| | | | ataaaataaacccctatcaaaatgtcagaggacatggcaaggtaaacttagcattttcaactttagaaccgggtcagctt |
| | | | cagggggactgctttcaaatcagccaaagagcctgtcagatcttcttagaaggaagaggttggtagttccctgctctgtttt |
| | | | gaacatgctctagtttattaacctggggacattcccattgctgtcttaagtaagtctcatagccagctcctgtcacgtgactct |
| | | | catatggattcattttcgggccagctctgaacaaagcatcatgaacatatgtgcttttggtcgtttgcaatgtgatggtggtgg |
| | | | aggtaggtattggtttccttggaaggcatgataagaaagattcacaatggccaacagtgtgtatgaacaaaaaactgatt |
| | | | ggagcatcagctagtactgaaggtccttgctttgtgtcagaggcaaaggaacccaaggcgccaagtcctcagccttgag |
| | | | tgtactgctgacaactaaactcacaggctgcaaagcagacctctgatgaagatgcctgttatttcacatcactgtctttttgtg |
| | | | tatcatagtctgcaccttacaaatattaataaatgttccaataataggtga |
| 35 | Human | Homo | gctgactctcctcggcggggaccgcggcgccggccggagcgaggagctgcgcacgcagcgggtcccggcgccctc |
| | EFNB2- | sapiens | cccgcacacaaaggcccgcgcgcgtccggagcccgcggggggaccgagctccctctttcctgccctgggggccgg |
| | isoform | ephrin B2 | gagccgcgcggactgagaaggctcctgcgcgcccggaggcgccctactccgctccgtgctccgggacatggaaccg |
| | 1-cDNA | (EFNB2), | cgccgacgcggcgcccgcgcgctcgggccgctgcccgctgcactggatctatagtcacaggcggccccgctcgggg |
| | | transcript | cgcagcgcccgccgcccgcgccggtcgtctccgctccgggtctttgtgtcccagcccgcacccgccccgcgcccaccc |
| | | variant 1, | gccagccgcggccggcccggcaccctggagccgcacgggagtcggccgtccgagctgcgtccggcgcggcgcccc |
| | | mRNA (NM_ | ggaaccccgagtccgccgcgccgccgcgccccgcgcgtgcgctcccgccccgcgccctgagggccccggagagc |
| | | 004093.4) | gagcgcacctggcccggcgaccgctggagctgcgcagcgcgcctcggagctgcctgcgggcgcacgccgtcttccc |
| | | | cgccagtctgccccggaggattgggggtcccagcctgcgtcccgtcagtcccttcttggcccggagtgcgcggagctgg |
| | | | gagtggcttcgccatggctgtgagaagggactccgtgtggaagtactgctggggtgttttgatggttttatgcagaactgcg |
| | | | atttccaaatcgatagttttagagcctatctattggaattcctcgaactccaaatttctacctggacaaggactggtactatac |
| | | | ccacagataggagacaaattggatattatttgccccaaagtggactctaaaactgttggccagtatgaatattataaagttt |
| | | | atatggttgataaagaccaagcagacagatgcactattaagaaggaaaatacccctctcctcaactgtgccaaaccag |
| | | | accaagatatcaaattcaccatcaagtttcaagaattcagccctaacctctggggtctagaatttcagaagaacaaagat |
| | | | tattacattatatctacatcaaatgggtctttggagggcctggataaccaggagggaggggtgtgccagacaagagccat |
| | | | gaagatcctcatgaaagttggacaagatgcaagttctgctggatcaaccaggaataaagatccaacaagacgtccag |
| | | | aactagaagctggtacaaatggaagaagttcgacaacaagtccctttgtaaaaccaaatccaggttctagcacagacg |
| | | | gcaacagcgccggacattcggggaacaacatcctcggttccgaagtggccttatttgcagggattgcttcaggatgcatc |
| | | | atcttcatcgtcatcatcatcacgctggtggtcctcttgctgaagtaccggaggagacacaggaagcactcgccgcagca |
| | | | cacgaccacgctgtcgctcagcacactggccacacccaagcgcagcggcaacaacaacggctcagagcccagtga |
| | | | cattatcatcccgctaaggactgcggacagcgtcttctgccctcactacgagaaggtcagcggggactacgggcaccc |
| | | | ggtgtacatcgtccaggagatgcccccgcagagcccggcgaacatttactacaaggtctgagagggaccctggtggta |
| | | | cctgtgctttcccagaggacacctaatgtcccgatgcctcccttgagggtttgagagcccgcgtgctggagaattgactga |
| | | | agcacagcaccgggggagagggacactcctcctcggaagagcccgtcgcgctggacagcttacctagtcttgtagcat |
| | | | tcggccttggtgaacacacacgctccctggaagctggaagactgtgcagaagacgcccattcggactgctgtgccgcgt |
| | | | cccacgtctcctcctcgaagccatgtgctgcggtcactcaggcctctgcagaagccaagggaagacagtggtttgtgga |
| | | | cgagagggctgtgagcatcctggcaggtgccccaggatgccacgcctggaagggccggcttctgcctggggtgcattt |
| | | | cccccgcagtgcataccggacttgtcacacggacctcgggctagttaaggtgtgcaaagatctctagagtttagtccttac |
| | | | tgtctcactcgttctgttacccagggctctgcagcacctcacctgagacctccactccacatctgcatcactcatggaacac |
| | | | tcatgtctggagtcccctcctccagccgctggcaacaacagcttcagtccatgggtaatccgttcatagaaattgtgtttgct |
| | | | aacaaggtgccctttagccagatgctaggctgtctgcgaagaaggctaggagttcatagaagggagtggggctgggg |
| | | | aaagggctggctgcaattgcagctcactgctgctgcctctgaaacagaaagttggaaaggaaaaaagaaaaaagca |
| | | | attaggtagcacagcactttggttttgctgagatcgaagaggccagtaggagacacgacagcacacacagtggattcc |
| | | | agtgcatggggaggcactcgctgttatcaaatagcgatgtgcaggaagaaaagcccctcttcattccggggaacaaag |
| | | | acgggtattgttgggaaaggaacaggcttggagggaagggagaaagtaggccgctgatgatatattcgggcaggact |
| | | | gttgtggtactggcaataagatacacagctccgagctgtaggagagtcggtctgctttggatgattttttaagcagactcag |
| | | | ctgctatacttatcacattttattaaacacagggaaagcatttaggagaatagcagagagccaaatctgacctaaaagttg |
| | | | aaaagccaaaggtcaaacaggctgtaattccatcatcatcgttgttattaaagaatccttatctataaaaggtaggtcagat |
| | | | ccccctccccccaggttcctccttcccctcccgattgagccttacgacactttggtttatgcggtgctgtccgggtgccaggg |
| | | | ctgcagggtcggtactgatggaggctgcagcgcccggtgctctgtgtcaaggtgaagcacatacggcagacctcttaga |
| | | | gtccttaagacggaagtaaattatgatgtccagggggagaaggaagataggacgtatttataataggtatatagaacac |
| | | | aagggatataaaatgaaagatttttactaatatatattttaaggttgcacacagtacacaccagaagatgtgaaattcatttg |
| | | | tggcaattaagtggtcccaatgctcagcgcttaaaaaaacaaattggacagctacttctgggaaaaacaacatcattcc |
| | | | aaaaagaacaataatgagagcaaatgcaaaaataaccaagtcctccgaaggcatctcacggaaccgtagactagg |
| | | | aagtacgagccccacagagcaggaagccgatgtgactgcatcatatatttaacaatgacaagatgttccggcgtttattt |
| | | | ctgcgttgggttttcccttgccttatgggctgaagtgttctctagaatccagcaggtcacactgggggcttcaggtgacgattt |
| | | | agctgtggctccctcctcctgtcctcccccgcaccccctcccttctgggaaacaagaagagtaaacaggaaacctactttt |
| | | | tatgtgctatgcaaaatagacatctttaacatagtcctgttactatggtaacactttgctttctgaattggaagggaaaaaaa |
| | | | atgtagcgacagcattttaaggttctcagacctccagtgagtacctgcaaaaatgagttgtcacagaaattatgatcctcta |
| | | | tttcctgaacctggaaatgatgttggtccaaagtgcgtgtgtgtatgtgtgagtgggtgcgtggtatacatgtgtacatatatgt |
| | | | ataatatatatctacaatatatattatatatatctatatcatatttctgtggagggttgccatggtaaccagccacagtacatat |
| | | | gtaattctttccatcaccccaacctctcctttctgtgcattcatgcaagagtttcttgtaagccatcagaagttacttttaggatg |
| | | | ggggagaggggcgagaaggggaaaaatgggaaatagtctgattttaatgaaatcaaatgtatgtatcatcagttggcta |
| | | | cgttttggttctatgctaaactgtgaaaaatcagatgaattgataaaagagttccctgcaaccaattgaaaagtgttctgtgc |
| | | | gtctgttttgtgtctggtgcagaatatgacaatctaccaactgtccctttgtttgaagttggtttagctttggaaagttactgtaaa |
| | | | tgccttgcttgtatgatcgtccctggtcacccgactttggaatttgcaccatcatgtttcagtgaagatgctgtaaataggttca |
| | | | gattttactgtctatggatttggggtgttacagtagccttattcacctttttaataaaaatacacatgaaaacaagaaagaaat |
| | | | ggcttttcttacccagattgtgtacatagagcaatgttggttttttataaagtctaagcaagatgttttgtataaaatctgaattttg |
| | | | caatgtatttagctacagcttgtttaacggcagtgtcattcccctttgcactgtaatgaggaaaaaatggtataaaaggttgc |
| | | | caaattgctgcatatttgtgccgtaattatgtaccatgaatatttatttaaaatttcgttgtccaatttgtaagtaacacagtattat |
| | | | gcctgagttataaatatttttttctttctttgttttattttaatagcctgtcataggttttaaatctgctttagtttcacattgcagttagcc |
| | | | ccagaaaatgaaatccgtgaagtcacattccacatctgtttcaaactgaatttgttcttaaaaaaataaaatatttttttcctat |
| | | | ggaaaaa |
| 36 | Human | Homo | gctgactctcctcggcggggaccgcggcgccggccggagcgaggagctgcgcacgcagcgggtcccggcgccctc |
| | EFNB2- | sapiens | cccgcacacaaaggcccgcgcgcgtccggagcccgcggggggaccgagctccctctttcctgccctgggggccgg |
| | isoform | ephrin B2 | gagccgcgcggactgagaaggctcctgcgcgcccggaggcgccctactccgctccgtgctccgggacatggaaccg |
| | 2-cDNA | (EFNB2), | cgccgacgcggcgcccgcgcgctcgggccgctgcccgctgcactggatctatagtcacaggcggccccgctcgggg |
| | | transcript | cgcagcgcccgccgcccgcgccggtcgtctccgctccgggtctttgtgtcccagcccgcacccgccccgcgcccaccc |
| | | variant 2, | gccagccgcggccggcccggcaccctggagccgcacgggagtcggccgtccgagctgcgtccggcgcggcgcccc |
| | | mRNA (NM_ | ggaaccccgagtccgccgcgccgccgcgccccgcgcgtgcgctcccgccccgcgccctgagggccccggagagc |
| | | 001372056.1) | gagcgcacctggcccggcgaccgctggagctgcgcagcgcgcctcggagctgcctgcgggcgcacgccgtcttccc |
| | | | cgccagtctgccccggaggattgggggtcccagcctgcgtcccgtcagtcccttcttggcccggagtgcgcggagctgg |
| | | | gagtggcttcgccatggctgtgagaagggactccgtgtggaagtactgctggggtgttttgatggttttatgcagaactgcg |
| | | | atttccaaatcgatagttttagagcctatctattggaattcctcgaactccaaatttctacctggacaaggactggtactatac |
| | | | ccacagataggagacaaattggatattatttgccccaaagtggactctaaaactgttggccagtatgaatattataaagttt |
| | | | atatggttgataaagaccaagcagacagatgcactattaagaaggaaaatacccctctcctcaactgtgccaaaccag |
| | | | accaagatatcaaattcaccatcaagtttcaagaattcagccctaacctctggggtctagaatttcagaagaacaaagat |
| | | | tattacattatatatgcaagttctgctggatcaaccaggaataaagatccaacaagacgtccagaactagaagctggtac |
| | | | aaatggaagaagttcgacaacaagtccctttgtaaaaccaaatccaggttctagcacagacggcaacagcgccggac |
| | | | attcggggaacaacatcctcggttccgaagtggccttatttgcagggattgcttcaggatgcatcatcttcatcgtcatcatc |
| | | | atcacgctggtggtcctcttgctgaagtaccggaggagacacaggaagcactcgccgcagcacacgaccacgctgtc |
| | | | gctcagcacactggccacacccaagcgcagcggcaacaacaacggctcagagcccagtgacattatcatcccgcta |
| | | | aggactgcggacagcgtcttctgccctcactacgagaaggtcagcggggactacgggcacccggtgtacatcgtccag |
| | | | gagatgcccccgcagagcccggcgaacatttactacaaggtctgagagggaccctggtggtacctgtgctttcccaga |
| | | | ggacacctaatgtcccgatgcctcccttgagggtttgagagcccgcgtgctggagaattgactgaagcacagcaccgg |
| | | | gggagagggacactcctcctcggaagagcccgtcgcgctggacagcttacctagtcttgtagcattcggccttggtgaa |
| | | | cacacacgctccctggaagctggaagactgtgcagaagacgcccattcggactgctgtgccgcgtcccacgtctcctcc |
| | | | tcgaagccatgtgctgcggtcactcaggcctctgcagaagccaagggaagacagtggtttgtggacgagagggctgtg |
| | | | agcatcctggcaggtgccccaggatgccacgcctggaagggccggcttctgcctggggtgcatttcccccgcagtgcat |
| | | | accggacttgtcacacggacctcgggctagttaaggtgtgcaaagatctctagagtttagtccttactgtctcactcgttctgt |
| | | | tacccagggctctgcagcacctcacctgagacctccactccacatctgcatcactcatggaacactcatgtctggagtcc |
| | | | cctcctccagccgctggcaacaacagcttcagtccatgggtaatccgttcatagaaattgtgtttgctaacaaggtgcccttt |
| | | | agccagatgctaggctgtctgcgaagaaggctaggagttcatagaagggagtggggctggggaaagggctggctgc |
| | | | aattgcagctcactgctgctgcctctgaaacagaaagttggaaaggaaaaaagaaaaaagcaattaggtagcacagc |
| | | | actttggttttgctgagatcgaagaggccagtaggagacacgacagcacacacagtggattccagtgcatggggaggc |
| | | | actcgctgttatcaaatagcgatgtgcaggaagaaaagcccctcttcattccggggaacaaagacgggtattgttggga |
| | | | aaggaacaggcttggagggaagggagaaagtaggccgctgatgatatattcgggcaggactgttgtggtactggcaat |
| | | | aagatacacagctccgagctgtaggagagtcggtctgctttggatgattttttaagcagactcagctgctatacttatcacat |
| | | | tttattaaacacagggaaagcatttaggagaatagcagagagccaaatctgacctaaaagttgaaaagccaaaggtc |
| | | | aaacaggctgtaattccatcatcatcgttgttattaaagaatccttatctataaaaggtaggtcagatccccctccccccag |
| | | | gttcctccttcccctcccgattgagccttacgacactttggtttatgcggtgctgtccgggtgccagggctgcagggtcggta |
| | | | ctgatggaggctgcagcgcccggtgctctgtgtcaaggtgaagcacatacggcagacctcttagagtccttaagacgga |
| | | | agtaaattatgatgtccagggggagaaggaagataggacgtatttataataggtatatagaacacaagggatataaaat |
| | | | gaaagatttttactaatatatattttaaggttgcacacagtacacaccagaagatgtgaaattcatttgtggcaattaagtggt |
| | | | cccaatgctcagcgcttaaaaaaacaaattggacagctacttctgggaaaaacaacatcattccaaaaagaacaata |
| | | | atgagagcaaatgcaaaaataaccaagtcctccgaaggcatctcacggaaccgtagactaggaagtacgagcccca |
| | | | cagagcaggaagccgatgtgactgcatcatatatttaacaatgacaagatgttccggcgtttatttctgcgttgggttttccct |
| | | | tgccttatgggctgaagtgttctctagaatccagcaggtcacactgggggcttcaggtgacgatttagctgtggctccctcct |
| | | | cctgtcctcccccgcaccccctcccttctgggaaacaagaagagtaaacaggaaacctactttttatgtgctatgcaaaat |
| | | | agacatctttaacatagtcctgttactatggtaacactttgctttctgaattggaagggaaaaaaaatgtagcgacagcattt |
| | | | taaggttctcagacctccagtgagtacctgcaaaaatgagttgtcacagaaattatgatcctctatttcctgaacctggaaa |
| | | | tgatgttggtccaaagtgcgtgtgtgtatgtgtgagtgggtgcgtggtatacatgtgtacatatatgtataatatatatctacaa |
| | | | tatatattatatatatctatatcatatttctgtggagggttgccatggtaaccagccacagtacatatgtaattctttccatcacc |
| | | | ccaacctctcctttctgtgcattcatgcaagagtttcttgtaagccatcagaagttacttttaggatgggggagaggggcga |
| | | | gaaggggaaaaatgggaaatagtctgattttaatgaaatcaaatgtatgtatcatcagttggctacgttttggttctatgcta |
| | | | aactgtgaaaaatcagatgaattgataaaagagttccctgcaaccaattgaaaagtgttctgtgcgtctgttttgtgtctggt |
| | | | gcagaatatgacaatctaccaactgtccctttgtttgaagttggtttagctttggaaagttactgtaaatgccttgcttgtatgat |
| | | | cgtccctggtcacccgactttggaatttgcaccatcatgtttcagtgaagatgctgtaaataggttcagattttactgtctatgg |
| | | | atttggggtgttacagtagccttattcacctttttaataaaaatacacatgaaaacaagaaagaaatggcttttcttacccag |
| | | | attgtgtacatagagcaatgttggttttttataaagtctaagcaagatgttttgtataaaatctgaattttgcaatgtatttagcta |
| | | | cagcttgtttaacggcagtgtcattcccctttgcactgtaatgaggaaaaaatggtataaaaggttgccaaattgctgcatat |
| | | | ttgtgccgtaattatgtaccatgaatatttatttaaaatttcgttgtccaatttgtaagtaacacagtattatgcctgagttataaat |
| | | | atttttttctttctttgttttattttaatagcctgtcataggttttaaatctgctttagtttcacattgcagttagccccagaaaatgaaat |
| | | | ccgtgaagtcacattccacatctgtttcaaactgaatttgttcttaaaaaaataaaatatttttttcctatggaaaaa |
| 37 | Human | Homo | gctgactctcctcggcggggaccgcggcgccggccggagcgaggagctgcgcacgcagcgggtcccggcgccctc |
| | EFNB2- | sapiens | cccgcacacaaaggcccgcgcgcgtccggagcccgcggggggaccgagctccctctttcctgccctgggggccgg |
| | isoform | ephrin B2 | gagccgcgcggactgagaaggctcctgcgcgcccggaggcgccctactccgctccgtgctccgggacatggaaccg |
| | 3-cDNA | (EFNB2), | cgccgacgcggcgcccgcgcgctcgggccgctgcccgctgcactggatctatagtcacaggcggccccgctogggg |
| | | transcript | cgcagcgcccgccgcccgcgccggtcgtctccgctccgggtctttgtgtcccagcccgcacccgccccgcgcccaccc |
| | | variant 3, | gccagccgcggccggcccggcaccctggagccgcacgggagtcggccgtccgagctgcgtccggcgcggcgcccc |
| | | mRNA (NM_ | ggaaccccgagtccgccgcgccgccgcgccccgcgcgtgcgctcccgccccgcgccctgagggccccggagagc |
| | | 001372057.1) | gagcgcacctggcccggcgaccgctggagctgcgcagcgcgcctcggagctgcctgcgggcgcacgccgtcttccc |
| | | | cgccagtctgccccggaggattgggggtcccagcctgcgtcccgtcagtcccttcttggcccggagtgcgcggagctgg |
| | | | gagtggcttcgccatggctgtgagaagggactccgtgtggaagtactgctggggtgttttgatggttttatgcagaactgcg |
| | | | atttccaaatcgatagttttagagcctatctattggaattcctcgaactccaaatttctacctggacaaggactggtactatac |
| | | | ccacagataggagacaaattggatattatttgccccaaagtggactctaaaactgttggccagtatgaatattataaagttt |
| | | | atatggttgataaagaccaagcagacagatgcactattaagaaggaaaatacccctctcctcaactgtgccaaaccag |
| | | | accaagatatcaaattcaccatcaagtttcaagaattcagccctaacctctggggtctagaatttcagaagaacaaagat |
| | | | tattacattatatctacatcaaatgggtctttggagggcctggataaccaggagggaggggtgtgccagacaagagccat |
| | | | gaagatcctcatgaaagttggacaaggttctagcacagacggcaacagcgccggacattcggggaacaacatcctcg |
| | | | gttccgaagtggccttatttgcagggattgcttcaggatgcatcatcttcatcgtcatcatcatcacgctggtggtcctcttgct |
| | | | gaagtaccggaggagacacaggaagcactcgccgcagcacacgaccacgctgtcgctcagcacactggccacac |
| | | | ccaagcgcagcggcaacaacaacggctcagagcccagtgacattatcatcccgctaaggactgcggacagcgtcttc |
| | | | tgccctcactacgagaaggtcagcggggactacgggcacccggtgtacatcgtccaggagatgcccccgcagagcc |
| | | | cggcgaacatttactacaaggtctgagagggaccctggtggtacctgtgctttcccagaggacacctaatgtcccgatgc |
| | | | ctcccttgagggtttgagagcccgcgtgctggagaattgactgaagcacagcaccgggggagagggacactcctcctc |
| | | | ggaagagcccgtcgcgctggacagcttacctagtcttgtagcattcggccttggtgaacacacacgctccctggaagctg |
| | | | gaagactgtgcagaagacgcccattcggactgctgtgccgcgtcccacgtctcctcctcgaagccatgtgctgcggtca |
| | | | ctcaggcctctgcagaagccaagggaagacagtggtttgtggacgagagggctgtgagcatcctggcaggtgcccca |
| | | | ggatgccacgcctggaagggccggcttctgcctggggtgcatttcccccgcagtgcataccggacttgtcacacggacc |
| | | | tcgggctagttaaggtgtgcaaagatctctagagtttagtccttactgtctcactcgttctgttacccagggctctgcagcacc |
| | | | tcacctgagacctccactccacatctgcatcactcatggaacactcatgtctggagtcccctcctccagccgctggcaac |
| | | | aacagcttcagtccatgggtaatccgttcatagaaattgtgtttgctaacaaggtgccctttagccagatgctaggctgtctg |
| | | | cgaagaaggctaggagttcatagaagggagtggggctggggaaagggctggctgcaattgcagctcactgctgctgc |
| | | | ctctgaaacagaaagttggaaaggaaaaaagaaaaaagcaattaggtagcacagcactttggttttgctgagatcgaa |
| | | | gaggccagtaggagacacgacagcacacacagtggattccagtgcatggggaggcactcgctgttatcaaatagcg |
| | | | atgtgcaggaagaaaagcccctcttcattccggggaacaaagacgggtattgttgggaaaggaacaggcttggaggg |
| | | | aagggagaaagtaggccgctgatgatatattcgggcaggactgttgtggtactggcaataagatacacagctccgagct |
| | | | gtaggagagtcggtctgctttggatgattttttaagcagactcagctgctatacttatcacattttattaaacacagggaaagc |
| | | | atttaggagaatagcagagagccaaatctgacctaaaagttgaaaagccaaaggtcaaacaggctgtaattccatcat |
| | | | catcgttgttattaaagaatccttatctataaaaggtaggtcagatccccctccccccaggttcctccttcccctcccgattga |
| | | | gccttacgacactttggtttatgcggtgctgtccgggtgccagggctgcagggtcggtactgatggaggctgcagcgccc |
| | | | ggtgctctgtgtcaaggtgaagcacatacggcagacctcttagagtccttaagacggaagtaaattatgatgtccagggg |
| | | | gagaaggaagataggacgtatttataataggtatatagaacacaagggatataaaatgaaagatttttactaatatatattt |
| | | | taaggttgcacacagtacacaccagaagatgtgaaattcatttgtggcaattaagtggtcccaatgctcagcgcttaaaa |
| | | | aaacaaattggacagctacttctgggaaaaacaacatcattccaaaaagaacaataatgagagcaaatgcaaaaat |
| | | | aaccaagtcctccgaaggcatctcacggaaccgtagactaggaagtacgagccccacagagcaggaagccgatgt |
| | | | gactgcatcatatatttaacaatgacaagatgttccggcgtttatttctgcgttgggttttcccttgccttatgggctgaagtgttc |
| | | | tctagaatccagcaggtcacactgggggcttcaggtgacgatttagctgtggctccctcctcctgtcctcccccgcacccc |
| | | | ctcccttctgggaaacaagaagagtaaacaggaaacctactttttatgtgctatgcaaaatagacatctttaacatagtcct |
| | | | gttactatggtaacactttgctttctgaattggaagggaaaaaaaatgtagcgacagcattttaaggttctcagacctccagt |
| | | | gagtacctgcaaaaatgagttgtcacagaaattatgatcctctatttcctgaacctggaaatgatgttggtccaaagtgcgt |
| | | | gtgtgtatgtgtgagtgggtgcgtggtatacatgtgtacatatatgtataatatatatctacaatatatattatatatatctatatc |
| | | | atatttctgtggagggttgccatggtaaccagccacagtacatatgtaattctttccatcaccccaacctctcctttctgtgcatt |
| | | | catgcaagagtttcttgtaagccatcagaagttacttttaggatgggggagaggggcgagaaggggaaaaatgggaa |
| | | | atagtctgattttaatgaaatcaaatgtatgtatcatcagttggctacgttttggttctatgctaaactgtgaaaaatcagatga |
| | | | attgataaaagagttccctgcaaccaattgaaaagtgttctgtgcgtctgttttgtgtctggtgcagaatatgacaatctacca |
| | | | actgtccctttgtttgaagttggtttagctttggaaagttactgtaaatgccttgcttgtatgatcgtccctggtcacccgactttg |
| | | | gaatttgcaccatcatgtttcagtgaagatgctgtaaataggttcagattttactgtctatggatttggggtgttacagtagcctt |
| | | | attcacctttttaataaaaatacacatgaaaacaagaaagaaatggcttttcttacccagattgtgtacatagagcaatgtt |
| | | | ggttttttataaagtctaagcaagatgttttgtataaaatctgaattttgcaatgtatttagctacagcttgtttaacggcagtgtc |
| | | | attcccctttgcactgtaatgaggaaaaaatggtataaaaggttgccaaattgctgcatatttgtgccgtaattatgtaccatg |
| | | | aatatttatttaaaatttcgttgtccaatttgtaagtaacacagtattatgcctgagttataaatatttttttctttctttgttttattttaat |
| | | | agcctgtcataggttttaaatctgctttagtttcacattgcagttagccccagaaaatgaaatccgtgaagtcacattccacat |
| | | | ctgtttcaaactgaatttgttcttaaaaaaataaaatatttttttcctatggaaaaa |
| 38 | Human | Homo | attcagcagagaacagtaatgcctagcttccgtttttaacttaacacttcagtagaacattttcttccaagagggagattttgg |
| | EFNB2- | sapiens | cctaagtaaagtagtgggctcttttttaaaaaaaaattaattttactttaatgtgagcaaatctgtattggtatggtgttctgcaat |
| | isoform | ephrin B2 | gcattacactgactttgaaaatttcgagtactaatgccttatgtctggggttaccattccctgtgcatcacatactagttagtta |
| | X1-cDNA | (EFNB2), | acatagcattttgcttttcccatgtaattttttccctatataatactggattcctgatactaattgacttgatacaaaagaatggct |
| | | transcript | ggatgatatccagataacgtataatacatgggcttcaccacaatcaggctctgaataaatacagacctgtcagagattga |
| | | variant X1, | taaaataaactacaatggatagtgctgtttaaacagtccattcaataacatatataagccagcctgccttccattgtgtctga |
| | | mRNA (XM_ | aattcttatttttgtaggtaaacaaatgcacattcagcactgattgaatagccccttgaactatgctccacagtttgcgtttggg |
| | | 017020406.3) | ttaatcttgtcggttttaatatagagagaaaaaagctcaaagcaccaggggtggaattgttagtgctttcacatccacattcc |
| | | | tcacattttgtcaggatgataaactgtaggtaatggactgtcgttgttctgcaggacaactgagccaggcagagcacaaa |
| | | | gactaagctaaagcgatacctcacaacatgcttggtagccttcttttcagatgagaatttatttgagaatcatgtgtctaggg |
| | | | actgcacatcttaacctcaacagttacagcttcaagccccagaaacaggagctggaggttaagatgatttgctaagcac |
| | | | ctggttctaaatcttttacaaagcataagctgttgacgctggttctgccgacgcaaagacatgcagatgactccaacatttc |
| | | | cagaggcttctgacttaagctaaagtgtgtggacaggtgaattcgccatgggcctggagaccagcttgctaaaaactatg |
| | | | tgtttgaatggttcctccagacagagtcagctgaagaacaattggtggatttatattaaaacctcttgtctgtaaacttactga |
| | | | ggtgcatccttcggttggtggatcagtgagataattgccttcagatggacattgcaactggagcaactaaatccttgctgtct |
| | | | ttccttcctctgaaatcttccaggtagctcccgagagcttcagtatgacaccaaacttcgggcgacgttttagagtgogttca |
| | | | cctaatgggaaactattcgagatcccagcgtgactgcagtaatgcgtcataggaatgggagtggcaggggaaaagga |
| | | | aatacagattgtagaccctaataaaaaaatttttaggaaagatatttctttaacgttttatgagaacttcattcttaaaatactta |
| | | | attgcaaattagacaaatagaagtgctcttctaaggaaggtgattaaactggtcctcctatcagcctaatctctgcctgccttt |
| | | | gctgctgacataaagaacctgtttttcaggtcacttaatatacatctacatagatttgcttatgagctcaccctttgtgtagcgg |
| | | | agtagagccttaaagaggagtgctcaactgtttaaaatattttgattaaaatatgcagaacccatagaactataagcttcta |
| | | | gtcaggaattagctctttcagggaacagctccccccttctttttaaggggggaattagaaggaggctgggggaggaatat |
| | | | aagaacagcaaagaaggaaggatagcaaatgggacatgttccgaacagcttggaaaaactcctgtggcttcattgtct |
| | | | ctataaagccaaagaatacaaagacataagcaattcagcccttctcccatgatggaagatgtaaaccgttgacatgcct |
| | | | cccctgtttaacttgtttaattctcattttaaattcagcacgatactagccgtgtgaactctgaagatttctttagtaatccattttgt |
| | | | agttccgaatcaaaaacaaagtgaaagggtctgacacaatttgcttttatttttaggcaaatcaaccctggtcatagttaata |
| | | | aggggattacaactcagactaggtctttacagatgtgatgtaaatcaagggcagagtataaagaaactgatcccttttgat |
| | | | tgaagtatagtaaaaaggcatagagaaactagcagcagtaatctgattgtatggcaataaaaccaccattttctgtctttc |
| | | | agataaaaataatgtggtaaatccatgcagttcataagatgtaaaggcagataaagggtgaagccatggcaacatata |
| | | | gattagcttgatgttagaaatgacacgtctctgaaaagggcgcgggacgaaggcccttgcctccaggctgttgggcatta |
| | | | tgtgagaaccacacagacttggaaactgggattaggaagtatgaaagctctacttgtggtctgggatggctgaggcagt |
| | | | aaagaaaagctgctcagttcttgctcattggtggtggataatatggcaaaggtagatttcattgactgccttttttatagattga |
| | | | gattggggctgattaaaacttcagatcactgcagttgttagggcctgggagattttcctttttaactcctggcctaacagcag |
| | | | cagccgttctgtaggattaactgcacttcgcggtcgttgccttaatctatttgggcttcaggcagggacatgctgggaagga |
| | | | acagagaccagaggggataggtagggctggggttatctgaaaagaaaacagagaccttttgatttcagccatcttttca |
| | | | gacccagctccctctcccgctgcatgggagaagcaaaggtaaacaggacacattgtccctctccctcagccacagagc |
| | | | tcttctgtgagttttgtctttcccaccctggaaaaaaagataaaatacaatttttaaaaggggagggaggaatttagttttaatt |
| | | | caaatgagtagtaatccaatatgccaaaagcagtgggctctacctagatgtaattttactcgtaaatgtgagtcttaaacttt |
| | | | gagttgaatggggcaggctgttagaggtggtgtaaattacaggattataaaaatgttagtgctgcccagccttaaagtcaa |
| | | | aaacagaaaaatctctgtgctgttgagtcttcccgccctctctcctgaacaaccttgtaagtaagctagacttttgtttttgcctt |
| | | | ccatactttccatttcagccattaaacaaaataagccattgaaaccacgattgggttccatgcagagtgacatccgcaatc |
| | | | gggtcaagccagaaggaaatacttgctcgattgccccctatttggcattacaggaaagtctccacactttggaagagtctg |
| | | | aactctcaagacattgaaaatgccaaaggctgcaaacaccctgtgtctttcttgatggagtgcatcttggtgtgttttacaaa |
| | | | ggggaattcagtgctgtttttttgttgttgttgttgttttttttttttaaagagcagcatagggcccttctagactcttggattctgtgtct |
| | | | gacaaaaatggtcattaaatgagcaatattataatttagacccatttcactgattttgttccaaattctcaactgacttgagcat |
| | | | ctgtttggggctgtagatacattgcccttgttgactgtttttctcgtttctatgggaattactgtagccattactatgtagctttcata |
| | | | gactcaaaacatttttaaagtattgcatataggctggccatatccagtgcctgttactttaccttctttttctaacttaatgcagca |
| | | | gtctgtattaacagatccatttcatttgtctagcttcatcagagagaggctaccccctgatttacaggctgctcacatccaag |
| | | | caccttgcattctacacttgacagtgattgctaatggcccattcaactaaagtatttgcttgttaacagggaacagaacatg |
| | | | ataaatgtccagcaagcttgctgcctccttcagcttttcaaacgcagactggtgcatatttatggcaggcaaatgacaaaa |
| | | | gaaaaagctgaattgccctggcctccagctttctatcagaaacagggttaaagtgattaaagcaatcattcaagaaagc |
| | | | cctgccgtttgtttactaaccttcatccaacatttagctttgtagtctacctgtgagaagatatttcagaagtattagagataag |
| | | | gaaggaggatctagcaaaccagtgaaaagagtaggtgaccagttataaaatgctttccatgcacattgaatgccaggc |
| | | | gaacctatttctgttattccagcagacaatcagcagtggctctagattattaacatattttcctttcatgtataaattcaaatatgt |
| | | | aattctagtccaaagcattctgtggctggtaagcacatacttgctgatttcaaataagaaaacatagcaagggaaagctc |
| | | | cattaaacaagttgtttctgcccttagtaattctctaaacaagataggaagaaaaagtggacagtagtggagtattaatagt |
| | | | gtgctcttttcattctctaaagcacgagtaagtaagcgttcaaactactctgtggtgggcatacatttagagcgctgtgaatg |
| | | | aaccactgctgttctgccatacttaatttatttatattattatttttattttattgttgtttttatgtattattataattatttatttatattactaa |
| | | | tttattttctcaatttaaatcctgttgcatccaattttaattacagtttttgtatctgccttcccatacttgctacccacgtccccattgc |
| | | | cactgcggccttatccatgttttctgtgtacaccactctcgtatcaccccagaataattatgagtgctacccagacttttgaaa |
| | | | ccactagagtcaacatgtttgtctttgaggaaagccaatgatgctttagcatttttggcaggggggatgtgtgtttaagtggg |
| | | | gtgggtgcagctccttattgtctgcctattctactgttgttcccaatccacattccctgcggggcacctaacctgtgtgcatagc |
| | | | aaagaatttccgaccttcagagccagaagtgtttctcaattgatctcttccagcctagggttatagctgatgaattataatcct |
| | | | tgctctttccacacctttacctgggcttaccatggccctaaaacatttgcccagaatcagaattgtctcatgagtgagtgggg |
| | | | caaggcaaatcctgttccagaccagctgagaatgtacctagctgcagaagaagttagaaagtgtcatcttttacttatcta |
| | | | ccagaactatattcgaggtacattttagatttaaaaaaaaagcaagttctcgtaggccttgaatcccccccttgctatggga |
| | | | aaatggatcattattataatggactgtccagtaaagttcatgatttctcctagacatgttctctctctttatgacctagatcaaga |
| | | | gtgatctctttaagtcttttcttcataatcccacagcactttgtacttagatgtacttagaaagaaccatatacacggtacgtcat |
| | | | gattgatatgcaagccttcaccactctacctgtcctaaaagtcagggacacaccttcttcatttcatcagtccctacttctatc |
| | | | cagcattggcatccagtaagtattagtggaatggacagacaacccgaatttgtgctgatggcagtttaccctgttttaactgt |
| | | | catccttctgctactagacatggatgagacctgagacgatgggactgctcagaggtccctggctcttgaactttagggcac |
| | | | cagaatcccctgcagggcttgagaaaacaggggtttctgggccccacccccagagttcctgattcctgaggtctggggt |
| | | | ggggcttgaagatggacatgtttaacaagctcccaggtgacgctggcaactgctgcctcagggccatgctgagaaccct |
| | | | cgccctacacaaacctttctgggaaaacaactcaacattaaagctgtttggggatctctgaagaaatctgtagtccttgcct |
| | | | tgttgggggagcatcagggatctaaccattgatggtggagtatttgttgttaattcagcaagcaactattaagtgttaggcct |
| | | | gttactcggctctaacaatacaaggcagagtgacctgtaccctcgagatttaaagtctaagtcctgtagagagaagccca |
| | | | ggtgggagcaagcacatttagagttaggtgcttggtgcaaggtggggacacagaagaagggaatggcatttgcctctg |
| | | | gaggggtccggaaacagcctagggaggaggagcttgagtcttgaaatactgtgggcatctctaagcaaagtcacagta |
| | | | gacagctgaaataaagaaaatagtaagcaagccaaagaaacagtatttcagccaagggcagcgtgtgtctatcacgt |
| | | | ccacctgtgaacacgtcccaggattctctgcatccggccattgctcaagacagatccctcacaggaacagctaagccac |
| | | | tgatttcagctacctgttcacgtgagaattatcagtacctactgcttttcaaaatgagtatgatcatggataggtgaggcaatt |
| | | | cagtttcgcagagacagtagggcaagtgccactgtagtttagttaagggcacatgctttagagtttggctatgtgagtccaa |
| | | | tcccagtttagccatttattagctgggtagctttaggagcagtagccttagtgtctctcagttgtcccatctctataatagggac |
| | | | aataacataatagtgctgaataaaagagtaacaaaattttggtcaacatttaatgtatttaaagagctaagctccgtgattg |
| | | | gcacaatgaaccaatcaatcaaacaccagttgttattaataaaagtcagttgaatatgtactgtgtgcctggccgtggttca |
| | | | atttgcctttgcatacaaggaaaaaattaaaatactctgttaataaagactatagcataatactttcaccttaaacttcttgatg |
| | | | ttaatttattttgtttacctgccaaacttctactcattccttatgactttctgctacatgaaacaccctttgtaattcttttgtcctattaa |
| | | | attaagttctctctcctctgctttcctgcttttggtgctttctaataacacttttaaccctggactttctcattcagctgtgcaactgtg |
| | | | gactgagaggaggctctttgaattcattttgtatattctagtagagagtactgtgagcagttgggttgttgaatgaatacatta |
| | | | attcaacctggagggatgggcagtattgcattttttacattgatattacatgatatttagaaaactgcttaactggtggacgttg |
| | | | ttttattaacagcattttgtgtatagcactcactatgtgccagctgctattctaactgcctgacaaatactcctgaaaccttcatg |
| | | | gtaaccatatgagggaagcacttttaatatatccataataccaacggggagactgtggccaaattggttaattaacttagc |
| | | | caaagtcatattgaactaataagtggatttaaacccagctagtctggggccagggtccctcttttaatcttctgcctcctgctt |
| | | | atgctgttgcatggagtagtctttatcatataactaaattaagcatgcatttgcttaaagcagtgcatacatgatggatcaaa |
| | | | aagtttgtggtataattggtttaattctgtcattatccattttgatttatagtcactttcttatgatggtcgtgtagttttaaatggaacc |
| | | | tttgaatctttgatataataaggttatgtcaaatcttgggtataataaggttatacccaatggaaacagaataatgatcagcc |
| | | | catttaaaggatgactggagagttattacaatacataatagtcatgcatatattgagtagtattcctttggtaacattttcctttta |
| | | | aaaattgtaacatttgattgttccttgttgggagaaaaggaggtcagatttttgaggggagatccatttggtgagatgctgag |
| | | | tgtgtgtcaagctaaggagatagtatgacatcttttttagagtctagtcacaattaaatgccattttattttggattttgggatccg |
| | | | tgccagcttccagcttgtcagagctgagaagactcaaatcaagtccaggcttatttctacagcaaactgggattctggcttc |
| | | | ttgccggtggattcattcagtacagcccatctggcttttgatgttctgcaagtttggagccatttgttgaaggaagccaggcg |
| | | | gtgaatattggtggtcctggggttctcttgactccaagtggtgccccttggtttgcattttcaccatgcttagcatctgcttacctg |
| | | | gagaccatgcagccgccggccagaggtctccaacaaccaaatcttcatgccttttagaactcagagtccccagcacat |
| | | | cctccttcctcctccttgtccaattactttcatgcagttctcagtagctgcttgtttgaatcacttatagtatttaacttctagggtgtt |
| | | | tttgggttttggtcaaggtaattccaggctgaatgtggtgactaagcaggaaataaatgggtcgtcctcaaagttacagtgg |
| | | | agcgctgtttctattttcctaaggtacacagttgtgggggcgatccgtatggaagtcaggaacccagtctgattttgcttccttt |
| | | | tgatggtagcagtacagacctggctgttttgtagcctgctttgtttttcttccttttcttccctaacttcacgggctgtggcaaagc |
| | | | cctgagacgtgcaggaaaatgtctcctgtcatacgcccacagcagacctagccctgaccctcctctgaagcccaggaa |
| | | | ggaggtatctgtgaagcagcctgcttgtaaagcaattgcacacagccttgtaaactgtgttactgggctgattatacttgatt |
| | | | ggcaaggtgaatctcttatagcaaaagagaacttggagagttttatctcatcttatgccttattaatttgttcattctttaattaca |
| | | | cagccacctattgagcaccctatttatgcaaggtacctggtcgggggtcagagggagggtcccatggtaaacgagaca |
| | | | gactcaatcctggaggagcaggaatggcagcccctcgctgggctgttggccccaccaaaagggaaaggtttcattttaa |
| | | | taatacatgggtgaatcatttttgtcaataggcaaaattctttgtagttaaaaaaaaatatgatggtaggaaggaaagggat |
| | | | gggcagagggttaaaacaaaagatatgctctccctaactctagattgtagtattgttatgcttgtcactgtagctgaattccat |
| | | | ttctttgagttttttcaatgccaaggcattccctgtatgacttacgtgagcctttcatctccgcgatttttcccattcaggtaaatga |
| | | | gcaaatggatttgaacactcatatctaaaacaagagagaaccagctggaaatgccctttgaatttctttctctatgtaaacc |
| | | | atttttctttctggtgcctcacctataaataacaggagttccaccttcctttatagactcttgctgaaagcatggtttggaacaag |
| | | | accgtacaggtgcacacaaattacagttgggaaagaagcctgcagtgcatcttgtctctgaaggttatgaaatcctcctttt |
| | | | agtaatggagctggcgtgatcaagccagcaggatgaaatttggcatttgtgagatcaccccccttctcacttgcccactgt |
| | | | acatagcatcccagccttactcttcaaatctccacattttttcttatctagctacaaaattcataggctgatttttttggggtgcgt |
| | | | gtgtggttttttttttgtttttttggtaaataaagacctgcatttttattttgatataggtggttgagttttgtctttaatttcatgacagag |
| | | | atttaactagtctcaacttttgaaaagacaacaatgatatttggggatcacacacttaaagttagatttctagatgattaatac |
| | | | caaagtagatgattttttagcctcagccatttataggtatgcccttctgtgaattttttatgacagtgaaaatcatggcacagat |
| | | | aaaaattaaataaatacttctgttattttcctgaagaaaaaaaaaaaaagcttaaactatgagaatactgtctttgagcactt |
| | | | taaaataaaattgacttcagccagcaggattttgagcattacatcacaaataaaaaacaagattaacatcaaaaggagt |
| | | | cagttttcattcaattgtgcagcactgtgggctgtgaaatttaatattattttgactcatatgctaattgtagactgacagagga |
| | | | aaatggattgtgtttaaataaaaggatacacagcatcacacgcagctgtatcaaatacaagttgaggtctttgggccagg |
| | | | aactgggggccctctagctctgttattgcagattcaagtttgacaaataaaactttcctttagactgtagtttaattactttttttca |
| | | | aaggtatgcgtgatgaagaggcacaaatacacctcaccttgaagagttgctaaactggtttgtgtgccgatcagttcacc |
| | | | gtgtgtttgaatttctgtgcttctcatctttccttttcttgaaaagattttgcttgtcattggtgtgaattgtaccccccacccccaccc |
| | | | atctagtctttgctctcagatttataacactttaatggttccaaattgtatagcctgctcttagaccccttttcttttccttgaataaat |
| | | | caggttcatgttgcagacgatatttgttttaggaaagtgtgaaagaaggggcacctgtgaaaacacgcaattgttccaac |
| | | | acacatatacatccaaattaaagcagaaaatgtcaaagcctccaatcactaccttatttcttggaggtttaaagccgctga |
| | | | gaagatagtggtgccctcgctggaagttttaaggtaattactttttactctaagcagtagtatctggtaacctaattccgtataa |
| | | | acctgacaccctatcgctacaccccagtatttctctgatttcagaataagtctgcgtagaaacttgttctgatgttaaagtgca |
| | | | aaagggggcagtaaagtgctatccacaaaaaaggaaaaacattttccaagtatttcttattactgcctgtgtctttcgtagg |
| | | | ccctgcctttatttattcattttataacaaaactcttatgtttggggcattcagagaataccttattaagctgttgcagcaatctag |
| | | | cattaaatggaagacatgcaagactgaagatcctgcctgtttatgaagtgtgccatcaaattcacatgctcatgatgcaga |
| | | | gtccttctttgggagtattcgtattcccaagtgcacagagcacttcggaaaggagccttggtctttggtgttaatgctctcctag |
| | | | ctccgtatagatgtggcaggcccaaagtacatggtggggtgaagggtcaagggtttgggcttatccagagcagcgtgca |
| | | | tcctttgtcaggaggtgactggaaacaccagccaattacagcagaactgcagactgctcatctgcattcggaattgcaga |
| | | | tgaaccagtttgtactcgacttctcttcttcactgtaggctttgacatttaattaaaaattaaagccttttatggaaaaagtacat |
| | | | gttttccaaaatggggtaaattcgaagtatacttgatacagaacactggcttgggaataaacctgtgatattacatgactttt |
| | | | ggtttgcaactgctaggctgagcctctttgtaaagctgggatttagaatctttgaaatgtttgtacagttcaatgattaagcata |
| | | | aattgtatatattccctttttttcacttatttgagtaaacaagtttgttactacagcttctgtggactcagagatttatgtattaaata |
| | | | ggccacaacttcaactaggataattttatttatctgcttgttagggaattgcatcaaaagtttaagtctgtaggcattaaatattt |
| | | | taaatgcttatttttaaagtcaattatgaaagatagcacaaagtttttctgaaactacattaaaaaaataatgttttaatcttatc |
| | | | acaaaagcattgactatttattgcaaagaaaacacagaaagctaaaaatcattctaagtccaccattcagtagcccaaa |
| | | | gtggtctcaggtaaaggcggtgtgtgtgaccatttgtttatggttgtctccgtgcagtcagcaaaataaacagaacaacatg |
| | | | ccatatattattgatgtgtatattttcaactgaaattagccatctgcttacaatgatcatatacactaatggtataattttgaaatg |
| | | | aaaagaaaaataaaataattctttgtggagagtaatgcgaattgacttatgaatctcgccctgcttggcagtttgctctaga |
| | | | ggtagaagagctttatgtgtgggcctcctccccccccacacatttattctgctcacacttgcaccagcatccatgtcaggac |
| | | | tcaccttgtcctgttacatgagtaacatggccctgattctcaagtgcatgataactgccataattacacataaatattaaatat |
| | | | ttaaatagatctttacgtgtgtaatattaggtagaagtggctctggatcgaatctgatgctttttaaatagaagctttcccacaa |
| | | | catttccaagcactgtcatcgtgtctgtctcgatttggggtttacctggcctagttatctgtctgggtgtagaaactggtagttcct |
| | | | gtttgtatcttttttgttctgatctctttattctgtgtcagctaaatattcttgcagtcagttactaacatattaactcatccttgtttgga |
| | | | aactttggcatatccttccatggtttccttccgtggacctgtcgcgtctctcaggagagccaccaggtatattgtcacacatttc |
| | | | gcatgtattttcagagactacagcagcatcaagtggccccccagcgatttgggttttcttctcggttaatctacactctttggc |
| | | | caaccgtgagaaaacttgtaagaaggcatcagatgtttgtgctaagatttctacctggacaaggactggtactataccca |
| | | | cagataggagacaaattggatattatttgccccaaagtggactctaaaactgttggccagtatgaatattataaagtttatat |
| | | | ggttgataaagaccaagcagacagatgcactattaagaaggaaaatacccctctcctcaactgtgccaaaccagacc |
| | | | aagatatcaaattcaccatcaagtttcaagaattcagccctaacctctggggtctagaatttcagaagaacaaagattatt |
| | | | acattatatctacatcaaatgggtctttggagggcctggataaccaggagggaggggtgtgccagacaagagccatga |
| | | | agatcctcatgaaagttggacaagatgcaagttctgctggatcaaccaggaataaagatccaacaagacgtccagaa |
| | | | ctagaagctggtacaaatggaagaagttcgacaacaagtccctttgtaaaaccaaatccaggttctagcacagacggc |
| | | | aacagcgccggacattcggggaacaacatcctcggttccgaagtggccttatttgcagggattgcttcaggatgcatcat |
| | | | cttcatcgtcatcatcatcacgctggtggtcctcttgctgaagtaccggaggagacacaggaagcactcgccgcagcac |
| | | | acgaccacgctgtcgctcagcacactggccacacccaagcgcagcggcaacaacaacggctcagagcccagtgac |
| | | | attatcatcccgctaaggactgcggacagcgtcttctgccctcactacgagaaggtcagcggggactacgggcacccg |
| | | | gtgtacatcgtccaggagatgcccccgcagagcccggcgaacatttactacaaggtctgagagggaccctggtggtac |
| | | | ctgtgctttcccagaggacacctaatgtcccgatgcctcccttgagggtttgagagcccgcgtgctggagaattgactgaa |
| | | | gcacagcaccgggggagagggacactcctcctcggaagagcccgtcgcgctggacagcttacctagtcttgtagcatt |
| | | | cggccttggtgaacacacacgctccctggaagctggaagactgtgcagaagacgcccattcggactgctgtgccgcgt |
| | | | cccacgtctcctcctcgaagccatgtgctgcggtcactcaggcctctgcagaagccaagggaagacagtggtttgtgga |
| | | | cgagagggctgtgagcatcctggcaggtgccccaggatgccacgcctggaagggccggcttctgcctggggtgcattt |
| | | | cccccgcagtgcataccggacttgtcacacggacctcgggctagttaaggtgtgcaaagatctctagagtttagtccttac |
| | | | tgtctcactcgttctgttacccagggctctgcagcacctcacctgagacctccactccacatctgcatcactcatggaacac |
| | | | tcatgtctggagtcccctcctccagccgctggcaacaacagcttcagtccatgggtaatccgttcatagaaattgtgtttgct |
| | | | aacaaggtgccctttagccagatgctaggctgtctgcgaagaaggctaggagttcatagaagggagtggggctgggg |
| | | | aaagggctggctgcaattgcagctcactgctgctgcctctgaaacagaaagttggaaaggaaaaaagaaaaaagca |
| | | | attaggtagcacagcactttggttttgctgagatcgaagaggccagtaggagacacgacagcacacacagtggattcc |
| | | | agtgcatggggaggcactcgctgttatcaaatagcgatgtgcaggaagaaaagcccctcttcattccggggaacaaag |
| | | | acgggtattgttgggaaaggaacaggcttggagggaagggagaaagtaggccgctgatgatatattcgggcaggact |
| | | | gttgtggtactggcaataagatacacagctccgagctgtaggagagtcggtctgctttggatgattttttaagcagactcag |
| | | | ctgctatacttatcacattttattaaacacagggaaagcatttaggagaatagcagagagccaaatctgacctaaaagttg |
| | | | aaaagccaaaggtcaaacaggctgtaattccatcatcatcgttgttattaaagaatccttatctataaaaggtaggtcagat |
| | | | ccccctccccccaggttcctccttcccctcccgattgagccttacgacactttggtttatgcggtgctgtccgggtgccaggg |
| | | | ctgcagggtcggtactgatggaggctgcagcgcccggtgctctgtgtcaaggtgaagcacatacggcagacctcttaga |
| | | | gtccttaagacggaagtaaattatgatgtccagggggagaaggaagataggacgtatttataataggtatatagaacac |
| | | | aagggatataaaatgaaagatttttactaatatatattttaaggttgcacacagtacacaccagaagatgtgaaattcatttg |
| | | | tggcaattaagtggtcccaatgctcagcgcttaaaaaaacaaattggacagctacttctgggaaaaacaacatcattcc |
| | | | aaaaagaacaataatgagagcaaatgcaaaaataaccaagtcctccgaaggcatctcacggaaccgtagactagg |
| | | | aagtacgagccccacagagcaggaagccgatgtgactgcatcatatatttaacaatgacaagatgttccggcgtttattt |
| | | | ctgcgttgggttttcccttgccttatgggctgaagtgttctctagaatccagcaggtcacactgggggcttcaggtgacgattt |
| | | | agctgtggctccctcctcctgtcctcccccgcaccccctcccttctgggaaacaagaagagtaaacaggaaacctactttt |
| | | | tatgtgctatgcaaaatagacatctttaacatagtcctgttactatggtaacactttgctttctgaattggaagggaaaaaaa |
| | | | atgtagcgacagcattttaaggttctcagacctccagtgagtacctgcaaaaatgagttgtcacagaaattatgatcctcta |
| | | | tttcctgaacctggaaatgatgttggtccaaagtgcgtgtgtgtatgtgtgagtgggtgcgtggtatacatgtgtacatatatgt |
| | | | ataatatatatctacaatatatattatatatatctatatcatatttctgtggagggttgccatggtaaccagccacagtacatat |
| | | | gtaattctttccatcaccccaacctctcctttctgtgcattcatgcaagagtttcttgtaagccatcagaagttacttttaggatg |
| | | | ggggagaggggcgagaaggggaaaaatgggaaatagtctgattttaatgaaatcaaatgtatgtatcatcagttggcta |
| | | | cgttttggttctatgctaaactgtgaaaaatcagatgaattgataaaagagttccctgcaaccaattgaaaagtgttctgtgc |
| | | | gtctgttttgtgtctggtgcagaatatgacaatctaccaactgtccctttgtttgaagttggtttagctttggaaagttactgtaaa |
| | | | tgccttgcttgtatgatcgtccctggtcacccgactttggaatttgcaccatcatgtttcagtgaagatgctgtaaataggttca |
| | | | gattttactgtctatggatttggggtgttacagtagccttattcacctttttaataaaaatacacatgaaaacaagaaagaaat |
| | | | ggcttttcttacccagattgtgtacatagagcaatgttggttttttataaagtctaagcaagatgttttgtataaaatctgaattttg |
| | | | caatgtatttagctacagcttgtttaacggcagtgtcattcccctttgcactgtaatgaggaaaaaatggtataaaaggttgc |
| | | | caaattgctgcatatttgtgccgtaattatgtaccatgaatatttatttaaaatttcgttgtccaatttgtaagtaacacagtattat |
| | | | gcctgagttataaatatttttttctttctttgttttattttaatagcctgtcataggttttaaatctgctttagtttcacattgcagttagcc |
| | | | ccagaaaatgaaatccgtgaagtcacattccacatctgtttcaaactgaatttgttcttaaaaaaaaaaatatttttttcctat |
| | | | ggaaaaa |
| 39 | Human | Homo | gctgactctcctcggcggggaccgcggcgccggccggagcgaggagctgcgcacgcagcgggtcccggcgccctc |
| | EFNB2- | sapiens | cccgcacacaaaggcccgcgcgcgtccggagcccgcggggggaccgagctccctctttcctgccctgggggccgg |
| | isoform | ephrin B2 | gagccgcgcggactgagaaggctcctgcgcgcccggaggcgccctactccgctccgtgctccgggacatggaaccg |
| | 4-cDNA | (EFNB2), | cgccgacgcggcgcccgcgcgctcgggccgctgcccgctgcactggatctatagtcacaggcggccccgctcgggg |
| | | transcript | cgcagcgcccgccgcccgcgccggtcgtctccgctccgggtctttgtgtcccagcccgcacccgccccgcgcccaccc |
| | | variant 4, | gccagccgcggccggcccggcaccctggagccgcacgggagtcggccgtccgagctgcgtccggcgcggcgcccc |
| | | mRNA (NM_ | ggaaccccgagtccgccgcgccgccgcgccccgcgcgtgcgctcccgccccgcgccctgagggccccggagagc |
| | | 001372058.1) | gagcgcacctggcccggcgaccgctggagctgcgcagcgcgcctcggagctgcctgcgggcgcacgccgtcttccc |
| | | | cgccagtctgccccggaggattgggggtcccagcctgcgtcccgtcagtcccttcttggcccggagtgcgcggagctgg |
| | | | gagtggcttcgccatggctgtgagaagggactccgtgtggaagtactgctggggtgttttgatggttttatgcagaactgcg |
| | | | atttccaaatcgatagttttagagcctatctattggaattcctcgaactccaaatttctacctggacaaggactggtactatac |
| | | | ccacagataggagacaaattggatattatttgccccaaagtggactctaaaactgttggccagtatgaatattataaagttt |
| | | | atatggttgataaagaccaagcagacagatgcactattaagaaggaaaatacccctctcctcaactgtgccaaaccag |
| | | | accaagatatcaaattcaccatcaagtttcaagaattcagccctaacctctggggtctagaatttcagaagaacaaagat |
| | | | tattacattatatctgtcatctttgtaataggatattcatcagaatcttgccagagactgtgcatttgggatottgggggataca |
| | | | gcaccaccaccaccctccccctgtccaagagaaacagatcaacatcttaggttgagagtctggggtctggaagacccg |
| | | | agttcctgagtgccctttgacaagtaacttaacccctgtctgcctcagtctcttcatctgtaaagtggggataatgacagcac |
| | | | ctgcttcacagggttgatgggaatccagatgtggtgggatatagaaaatgcttattacttccacctttgacaccaaatacat |
| | | | ataactaagagttaactttggagcaggggaggaagtgtgaggctccaggctggaggcagacctgtgttcggctgcaag |
| | | | ctggagaggatggaccccaaaagcttggctgatttgaagtccatccataaaatggaactccagagagtttacacgtttca |
| | | | gtaatgctgcataacttaattataagatcttctctctttgtcttctttcagtgttataaaagctcttttgtccttgagcttcctttaccaa |
| | | | gaaacatgcatttatgtatctttttgttcatggaattgcccaagcttgttagcagatcctttgtaagacccaaaagagacaga |
| | | | caggggaggagtcttcagatacatataatcatttttcccaatttccatgttaccagccttgccaggactttttctcagttccctgt |
| | | | tacacaatgaaaatagtgtctctttattgataattttagtagcatcctaatgtggtataaatcgtcttccagagaagaaaatgt |
| | | | gtcagggttgcgttatcactgaggctagctgggaaagtagatcagcccattagtctgataattcgaagcgttgtttctgttattt |
| | | | ctgaacatcatgtgaactccttttctgggtgtattaaaggttttcccagtgtgtgtcagtgagactcctgattgaatttaatatga |
| | | | ataaagataaattctttacatttaagga |
| 40 | Human | Homo | agaccgcgagcgagagcgcccccgagcagcgcccgcgccctccgcgccttctccgccgggacctcgagcgaaag |
| | CCN1- | sapiens | acgcccgcccgccgcccagccctcgcctccctgcccaccgggcccaccgcgccgccaccccgaccccgctgcgca |
| | precursor- | cellular | cggcctgtccgctgcacaccagcttgttggcgtcttcgtcgccgcgctcgccccgggctactcctgcgcgccacaatgag |
| | cDNA | communication | ctcccgcatcgccagggcgctcgccttagtcgtcacccttctccacttgaccaggctggcgctctccacctgccccgctgc |
| | | network | ctgccactgccccctggaggcgcccaagtgcgcgccgggagtcgggctggtccgggacggctgcggctgctgtaagg |
| | | factor 1 | tctgcgccaagcagctcaacgaggactgcagcaaaacgcagccctgcgaccacaccaaggggctggaatgcaactt |
| | | (CCN1), | cggcgccagctccaccgctctgaaggggatctgcagagctcagtcagagggcagaccctgtgaatataactccagaa |
| | | mRNA | tctaccaaaacggggaaagtttccagcccaactgtaaacatcagtgcacatgtattgatggcgccgtgggctgcattcct |
| | | (NM_001554.5) | ctgtgtccccaagaactatctctccccaacttgggctgtcccaaccctcggctggtcaaagttaccgggcagtgctgcga |
| | | | ggagtgggtctgtgacgaggatagtatcaaggaccccatggaggaccaggacggcctccttggcaaggagctgggat |
| | | | tcgatgcctccgaggtggagttgacgagaaacaatgaattgattgcagttggaaaaggcagctcactgaagcggctcc |
| | | | ctgtttttggaatggagcctcgcatcctatacaaccctttacaaggccagaaatgtattgttcaaacaacttcatggtcccag |
| | | | tgctcaaagacctgtggaactggtatctccacacgagttaccaatgacaaccctgagtgccgccttgtgaaagaaaccc |
| | | | ggatttgtgaggtgcggccttgtggacagccagtgtacagcagcctgaaaaagggcaagaaatgcagcaagaccaa |
| | | | gaaatcccccgaaccagtcaggtttacttacgctggatgtttgagtgtgaagaaataccggcccaagtactgcggttcctg |
| | | | cgtggacggccgatgctgcacgccccagctgaccaggactgtgaagatgcggttccgctgcgaagatggggagacat |
| | | | tttccaagaacgtcatgatgatccagtcctgcaaatgcaactacaactgcccgcatgccaatgaagcagcgtttcccttct |
| | | | acaggctgttcaatgacattcacaaatttagggactaaatgctacctgggtttccagggcacacctagacaaacaaggg |
| | | | agaagagtgtcagaatcagaatcatggagaaaatggggggggtggtgtgggtgatgggactcattgtagaaaggaa |
| | | | gccttgctcattcttgaggagcattaaggtatttcgaaactgccaagggtgctggtgcggatggacactaatgcagccac |
| | | | gattggagaatactttgcttcatagtattggagcacatgttactgcttcattttggagcttgtggagttgatgactttctgttttctgt |
| | | | ttgtaaattatttgctaagcatattttctctaggcttttttccttttggggttctacagtcgtaaaagagataataagattagttgga |
| | | | cagtttaaagcttttattcgtcctttgacaaaagtaaatgggagggcattccatcccttcctgaagggggacactccatgag |
| | | | tgtctgtgagaggcagctatctgcactctaaactgcaaacagaaatcaggtgttttaagactgaatgttttatttatcaaaat |
| | | | gtagcttttggggagggaggggaaatgtaatactggaataatttgtaaatgattttaattttatattcagtgaaaagattttattt |
| | | | atggaattaaccatttaataaagaaatatttacctaatatctgagtgtatgccattcggtatttttagaggtgctccaaagtcat |
| | | | taggaacaacctagctcacgtactcaattattcaaacaggacttattgggatacagcagtgaattaagctattaaaataag |
| | | | ataatgattgcttttataccttcagtagagaaaagtctttgcatataaagtaatgtttaaaaaacatgtattgaacacgacatt |
| | | | gtatgaagcacaataaagattctgaagctaaa |
| 41 | Human | Homo | agacgttcgcacacctgggtgccagcgccccagaggtcccgggacagcccgaggcgccgcgcccgccgccccga |
| | AREG- | sapiens | gctccccaagccttcgagagcggcgcacactcccggtctccactcgctcttccaacacccgctcgttttggcggcagctc |
| | cDNA | amphiregulin | gtgtcccagagaccgagttgccccagagaccgagacgccgccgctgcgaaggaccaatgagagccccgctgctac |
| | | (AREG), | cgccggcgccggtggtgctgtcgctcttgatactcggctcaggccattatgctgctggattggacctcaatgacacctactc |
| | | mRNA | tgggaagcgtgaaccattttctggggaccacagtgctgatggatttgaggttacctcaagaagtgagatgtcttcaggga |
| | | (NM_001657.4) | gtgagatttcccctgtgagtgaaatgccttctagtagtgaaccgtcctcgggagccgactatgactactcagaagagtatg |
| | | | ataacgaaccacaaatacctggctatattgtcgatgattcagtcagagttgaacaggtagttaagcccccccaaaacaa |
| | | | gacggaaagtgaaaatacttcagataaacccaaaagaaagaaaaagggaggcaaaaatggaaaaaatagaaga |
| | | | aacagaaagaagaaaaatccatgtaatgcagaatttcaaaatttctgcattcacggagaatgcaaatatatagagcac |
| | | | ctggaagcagtaacatgcaaatgtcagcaagaatatttcggtgaacggtgtggggaaaagtccatgaaaactcacagc |
| | | | atgattgacagtagtttatcaaaaattgcattagcagccatagctgcctttatgtctgctgtgatcctcacagctgttgctgttat |
| | | | tacagtccagcttagaagacaatacgtcaggaaatatgaaggagaagctgaggaacgaaagaaacttcgacaaga |
| | | | gaatggaaatgtacatgctatagcataactgaagataaaattacaggatatcacattggagtcactgccaagtcatagcc |
| | | | ataaatgatgagtcggtcctctttccagtggatcataagacaatggaccctttttgttatgatggttttaaactttcaattgtcact |
| | | | ttttatgctatttctgtatataaaggtgcacgaaggtaaaaagtattttttcaagttgtaaataatttatttaatatttaatggaagt |
| | | | gtatttattttacagctcattaaacttttttaaccaaa |

### Production of Polypeptides

Once a polypeptide or functional derivative thereof (e.g., OLFML3, NDP, AREG, FGF1, EFNB2, and/or CCN1) is selected it can be purified or synthesized in any suitable manner. A nucleic acid encoding the polypeptide can be cloned into a suitable vector and expressed in a suitable cellular system. In certain aspects, the cellular system is a prokaryotic cell system. In certain aspects, the cellular system is a eukaryotic cell system. In certain aspects, the cellular system is a mammalian cell system. The supernatants from such an expression system can be subjected to one or more purification steps involving centrifugation, ultracentrifugation, filtration, diafiltration, tangential-flow filtration, dialysis, chromatography (e.g., cation exchange, ion exchange, hydrophobic interaction, reverse phase, affinity, or size exclusion). The polypeptides can be purified to an extent suitable for human administration. Additionally, polypeptides can be synthesized for inclusion in a formulation to be administered to a human subject. In certain aspects, the polypeptides can be produced by a suitable peptide synthesis method, such as solid-phase synthesis.

### Master Cell Bank and Transgenic Cells

In a certain aspect, described herein is a master cell bank comprising a cell that comprises a nucleic acid encoding one or more polypeptides (e.g., OLFML3, NDP, AREG, FGF1, EFNB2, and/or CCN1) integrated into its genome creating a transgenic cell-line. In some aspects, the master cell bank comprises a plurality of cells that each comprise a nucleic acid encoding a polypeptide (e.g., OLFML3, NDP, AREG, FGF1, EFNB2, and/or CCN1). In certain aspects, the nucleic acid is maintained extrachromosomally on a plasmid or yeast artificial chromosome. In certain aspects, the nucleic acid is integrated into a chromosomal location. In certain aspects, the cell is a yeast cell. In certain aspects, the yeast is Pichia pastoris or Saccharomyces cerevisiae. In certain aspects, the cell is a mammalian cell. In certain aspects, the mammalian cell is a 293T cell or derivative thereof (e.g., 293T-Rex). In certain aspects, the cell is a bacterial cell. In certain aspects, the transgenic mammalian, yeast, or bacterial cell is a master cell bank that comprises a cryopreservative suitable for freezing to at least about −80° or below. In certain aspects, the master cell bank comprises glycerol at between about 10 and about 30%, and is suitable for long-term storage at about −80° or below. In certain aspects, the master cell bank can preserve a transgenic mammalian, yeast, or bacterial strain for at least 1, 2, 3, 4, 5, 6, 7, 8, 9, 10 or more years.

In a certain aspect, described herein is a master cell bank comprising a cell that comprises a nucleic acid encoding one or more polypeptides (e.g., OLFML3, NDP, AREG, FGF1, EFNB2, and/or CCN1) integrated into its genome creating a transgenic cell-line. In some aspects, the master cell bank comprises a plurality of cells that each comprise a nucleic acid encoding a polypeptide (e.g., OLFML3, NDP, AREG, FGF1, EFNB2, and/or CCN1). In certain aspects, the nucleic acid is maintained extrachromosomally on a plasmid or yeast artificial chromosome. In certain aspects, the nucleic acid is integrated into a chromosomal location. In certain aspects, the cell is a yeast cell. In certain aspects, the yeast is Pichia pastoris or Saccharomyces cerevisiae. In certain aspects, the cell is a mammalian cell. In certain aspects, the mammalian cell is a 293T cell or derivative thereof (e.g., 293T-Rex). In certain aspects, the cell is a bacterial cell. In certain aspects, the transgenic mammalian, yeast, or bacterial cell is a master cell bank that comprises a cryopreservative suitable for freezing to at least about −80° or below. In certain aspects, the master cell bank comprises glycerol at between about 10 and about 30%, and is suitable for long-term storage at about −80° or below. In certain aspects, the master cell bank can preserve a transgenic mammalian, yeast, or bacterial strain for at least 1, 2, 3, 4, 5, 6, 7, 8, 9, 10 or more years.

### Pharmaceutically Acceptable Excipients, Carriers, and Diluents

The polypeptide (e.g., OLFML3, NDP, AREG, FGF1, EFNB2, and/or CCN1) described herein can be administered in a pharmaceutical composition that comprises one or more pharmaceutically acceptable excipients, carriers, or diluents. The exact components can differ based upon the preferred route of administration. The excipients used in a pharmaceutical composition can provide additional function to the polypeptide by making the polypeptide suitable for a particular route of administration (e.g., intravenous, topical, subcutaneous, or intramuscular), increasing polypeptide stability, increasing penetration of a desired tissue (e.g., lung or vasculature), increasing residence time at particular site, increasing solubility, enhancing the efficacy of the polypeptide, and/or reducing inflammatory reactions coincident with administration.

In certain aspects, the polypeptides (e.g., OLFML3, NDP, AREG, FGF1, EFNB2, and/or CCN1) described herein are included in a pharmaceutical composition with a solubilizing emulsifying, or dispersing agent. In certain aspects, the solubilizing agent can allow high-concentration solutions of HAPs that exceed at least about 2 mg/mL, 5 mg/mL, 10 mg/mL, 15 mg/mL, or 20 mg/mL. Carbomers in an aqueous pharmaceutical composition serve as emulsifying agents and viscosity modifying agents. In certain aspects, the pharmaceutically acceptable excipient comprises or consists of a carbomer. In certain aspects, the carbomer comprises or consists of carbomer 910, carbomer 934, carbomer 934P, carbomer 940, carbomer 941, carbomer 1342, or combinations thereof. Cyclodextrins in an aqueous pharmaceutical composition serve as solubilizing and stabilizing agents. In certain aspects, the pharmaceutically acceptable excipient comprises or consists of a cyclodextrin. In certain aspects, the cyclodextrin comprises or consists of alpha cyclodextrin, beta cyclodextrin, gamma cyclodextrin, or combinations thereof. Lecithin in a pharmaceutical composition may serve as a solubilizing agent. In certain aspects, the solubilizing agent comprises or consists of lecithin. Poloxamers in a pharmaceutical composition serve as emulsifying agents, solubilizing agents, and dispersing agents. In certain aspects, the pharmaceutically acceptable excipient comprises or consists of a poloxamer. In certain aspects, the poloxamer comprises or consists of poloxamer 124, poloxamer 188, poloxamer 237, poloxamer 338, poloxamer 407, or combinations thereof. Polyoxyethylene sorbitan fatty acid esters in a pharmaceutical composition serve as emulsifying agents, solubilizing agents, surfactants, and dispersing agents. In certain aspects, the pharmaceutically acceptable excipient comprises or consists of a polyoxyethylene sorbitan fatty acid ester. In certain aspects, the polyoxyethylene sorbitan fatty acid ester comprises or consists of polysorbate 20, polysorbate 21, polysorbate 40, polysorbate 60, polysorbate 61, polysorbate 65, polysorbate 80, polysorbate 81, polysorbate 85, polysorbate 120, or combinations thereof. Polyoxyethylene stearates in a pharmaceutical composition serve as emulsifying agents, solubilizing agents, surfactants, and dispersing agents. In certain aspects, the pharmaceutically acceptable excipient comprises or consists of a polyoxyethylene stearate. In certain aspects, the polyoxyethylene stearate comprises or consists of polyoxyl 2 stearate, polyoxyl 4 stearate, polyoxyl 6 stearate, polyoxyl 8 stearate, polyoxyl 12 stearate, polyoxyl 20 stearate, polyoxyl 30 stearate, polyoxyl 40 stearate, polyoxyl 50 stearate, polyoxyl 100 stearate, polyoxyl 150 stearate, polyoxyl 4 distearate, polyoxyl 8 distearate, polyoxyl 12 distearate, polyoxyl 32 distearate, polyoxyl 150 distearate, or combinations thereof. Sorbitan esters in a pharmaceutical composition serve as emulsifying agents, solubilizing agents, and non-ionic surfactants, and dispersing agents. In certain aspects, the pharmaceutically acceptable excipient comprises or consists of a sorbitan ester. In certain aspects, the sorbitan ester comprises or consists of sorbitan laurate, sorbitan oleate, sorbitan palmitate, sorbitan stearate, sorbitan trioleate, sorbitan sesquioleate, or combinations thereof. In certain aspects, solubility can be achieved with a protein carrier. In certain embodiments the protein carrier comprises recombinant human albumin.

In certain aspects, the polypeptide (e.g., OLFML3, NDP, AREG, FGF1, EFNB2, and/or CCN1) of the current disclosure are formulated to increase stability. Polypeptides in aqueous formulations may require stabilization to prevent degradation. In certain aspects, the stabilizer comprises pH buffers, salts, amino acids, polyols/disaccharides/polysaccharides, liposomes, surfactants, antioxidants, reducing agents, or chelating agents. In certain aspects, the stabilizer comprises or consists of a polyol/non-reducing sugar. In certain embodiments, the non-reducing sugar comprises or consists of sucrose, mannitol, trehalose, raffinose, stachyose, xylitol, starch, verbascose, or combinations thereof. Polypeptides can be encapsulated in liposomes to increase stability. In certain aspects, the stabilizer comprises or consists of liposomes. In certain aspects, the liposomes comprise or consists of ipalmitoylphosphatidylcholine (DPPC) liposomes, phosphatidylcholine:cholesterol (PC:Chol) (70:30) liposomes, or dipalmitoylphosphatidylcholine:dipalmitoylphosphatidylserine (DPPC:DPPS) liposomes (70:30). Non-ionic surfactants can increase the stability of a polypeptide. In certain aspects, the stabilizer comprises or consists of a non-ionic surfactant. In certain aspects, the non-ionic surfactant comprises or consists of polysorbates (e.g., poly sorbate 80, poly sorbate 20), alkylsaccharides alkyl ethers and alkyl glyceryl ethers, polyoxyethelene (4) lauryl ether; polyoxyethylene cetyl ethers, polyoxyethylene stearyl ethers, sorbitan fatty acid esters, polyoxyethylene fatty acid esters, or combinations thereof. In certain aspects, the polypeptide is formulated with a protein surfactant, such as recombinant human serum albumin as a stabilizer. Antioxidants or reducing agents can increase the stability of a polypeptide. In certain aspects, the stabilizer comprises or consists of an antioxidant or reducing agent. In certain aspects, the reducing agent comprises or consists of dithiothreitol, ethylenediaminetetraacetic acid, 2-Mercaptoethanol, Tris(2-carboxyethyl) phosphine hydrochloride, Tris(hydroxypropyl)phosphine, or combinations thereof. In certain aspects, the antioxidant comprises or consists of methionine, ascorbic acid, citric acid, alpha tocopherol, sodium bisulfite, ascorbyl palmitate, erythorbic acid, or combinations thereof. Chelating agents can stabilize polypeptides by reducing the activity of proteases. In certain aspects, the stabilizer comprises or consists of a chelating agent. In certain aspects, the chelating agent comprises or consists of ethylenediaminetetraacetic acid (EDTA), ethylene glycol-bis(β-aminoethyl ether)-N,N,N′,N′-tetraacetic acid (EGTA), metal complexes (e.g. Zn-protein complexes), or combinations thereof. Buffer agents can stabilize polypeptides by reducing the acid hydrolysis of polypeptides. In certain aspects, the stabilizer comprises or consists of a buffer agent. In certain aspects, the buffer agent comprises or consists of sucrose octa-sulfate, ammonium carbonate, ammonium phosphate, boric acid, sodium citrate, potassium citrate, lactic acid, 3-(N-morpholino) propanesulfonic acid (MOPS), 2-(N-morpholino) ethanesulfonic acid (MES), hydroxymethylaminomethane (Tris), calcium carbonate, calcium phosphate or combinations thereof. The polypeptide (e.g., OLFML3, NDP, AREG, FGF1, EFNB2, and/or CCN1) also may be entrapped in or associated with microcapsules prepared, for example, by coacervation techniques or by interfacial polymerization (for example, hydroxymethylcellulose or gelatin-microcapsules and poly-(methylmethacylate) microcapsules, respectively), in colloidal delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The polypeptide (e.g., OLFML3, NDP, AREG, FGF1, EFNB2, and/or CCN1) of the current disclosure may be formulated or delivered with an anti-inflammatory agent. In certain aspects, the anti-inflammatory agent comprises or consists of a corticosteroid. In certain aspects, the corticosteroid comprises or consists of hydrocortisone, cortisone, ethamethasoneb (Celestone), prednisone (Prednisone Intensol), prednisolone (Orapred, Prelone), triamcinolone (Aristospan Intra-Articular, Aristospan Intralesional, Kenalog), methylprednisolone (Medrol, Depo-Medrol, Solu-Medrol), or dexamethasone (Dexamethasone Intensol). In certain aspects, the anti-inflammatory comprises or consists of a non-steroidal anti-inflammatory (NSAID). In certain embodiments, the NSAID comprises or consists of aspirin, celecoxib, diclofenac, diflunisal, etodolac, ibuprofen, indomethacin, ketoprofen, ketorolac, nabumetone, naproxen, oxaprozin, piroxicam, salsalate, sulindac, or tolmetin.

In certain aspects, the polypeptide (e.g., OLFML3, NDP, AREG, FGF1, EFNB2, and/or CCN1) of the current disclosure are included in a pharmaceutical composition suitable for intravenous administration comprising one or more pharmaceutically acceptable excipients, carriers, and diluents. In certain aspects, the polypeptides of the current disclosure are administered suspended in a sterile solution. In certain aspects, the solution is one commonly used for administration of biological formulations, and comprises, for example, about 0.9% NaCl or about 5% dextrose. In certain aspects, the solution further comprises one or more of: buffers, for example, acetate, citrate, histidine, succinate, phosphate, potassium phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine, histidine, leucine, or arginine; antioxidants, for example, ascorbic acid, methionine; or chelating agents, for example, EDTA, or EGTA. In certain aspects, the polypeptide (e.g., OLFML3, NDP, AREG, FGF1, EFNB2, and/or CCN1) of the current disclosure are included in a pharmaceutical composition suitable for intramuscular or subcutaneous administration comprising one or more pharmaceutically acceptable excipients, carriers, and diluents. Formulations suitable for intramuscular or subcutaneous injection can include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include ethanol, polyols (inositol, propyleneglycol, polyethylene-glycol, glycerol, cremophor and the like) and suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents. In certain aspects, the polypeptide (e.g., OLFML3, NDP, AREG, FGF1, EFNB2, and/or CCN1) of the current disclosure are formulated for topical administration as a cream, gel, paste, ointment, or emulsion. Excipients in a cream, gel, paste, ointment, or emulsion can comprise gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecyl sulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars, and starches. The excipient used with the polypeptide (e.g., OLFML3, NDP, AREG, FGF1, EFNB2, and/or CCN1) described herein will allow for storage, formulation, or administration of highly concentrated formulations. In certain aspects, a highly concentrated polypeptide (e.g., OLFML3, NDP, AREG, FGF1, EFNB2, and/or CCN1) comprises at least about 1, 2, 3, 4, 5, 6, 7, 8, 9, 10, 15, 20, 25, 20, 25, 40, 45, 50 or more milligrams per milliliter. In certain aspects, the polypeptides of the current disclosure are shipped/stored lyophilized and reconstituted before administration. In certain embodiments, lyophilized HAP formulations comprise a bulking agent such as, mannitol, sorbitol, sucrose, trehalose, and dextran 40. The lyophilized formulation can be contained in a vial comprised of glass. The HAPs when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

### EXAMPLES

The following illustrative examples are representative of embodiments of the compositions and methods described herein and are not meant to be limiting in any way.

### Example 1—Regenerative Protein Library Pipeline for Identifying and Testing Function of Candidate Proteins Modifying AT2 Cells Undergoing Fibrotic Stress

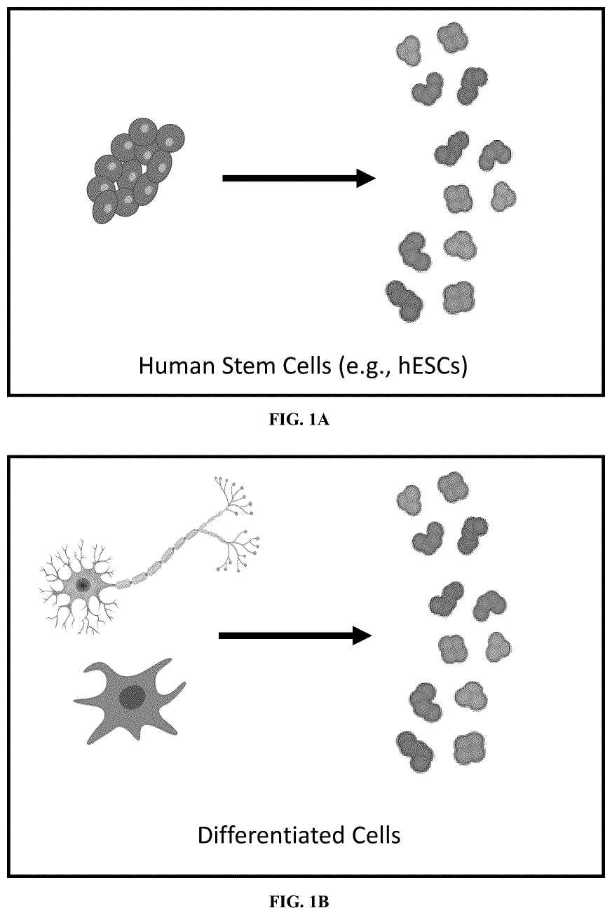

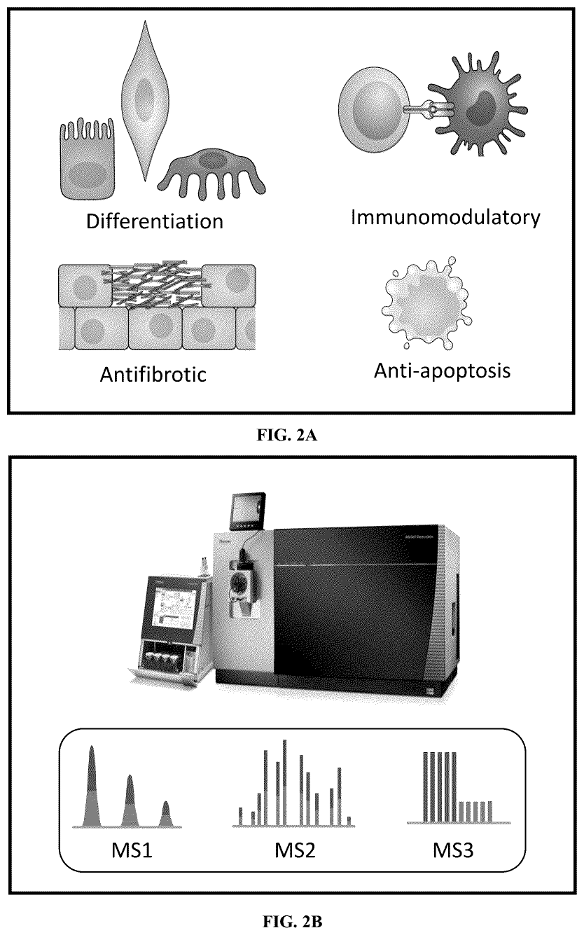

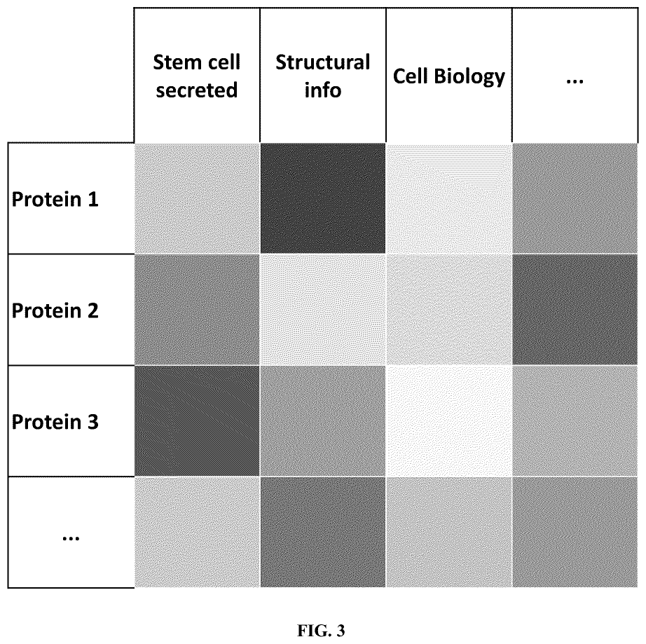

A regenerative protein library generation pipeline was developed and tested to identify secreted protein from pluripotent stem cells that improve the function of AT2 cells undergoing fibrotic stress. The schematics and operation of the pipeline are illustrated in FIG. 1A-FIG. 3. Secretomes from pluripotent stems cells (hESCs in FIG. 1A and other differentiated stem cells in FIG. 1B) were isolated and mapped for disease-modifying effects through in vitro phenotype mapping, examining such cellular processes as differentiation, immunomodulation, fibrosis, and apoptosis (FIG. 2A). Quantitative mass spectrometry was used to determine the individual protein composition and abundance of each secretome via differential analysis (FIG. 2B). Proteins with pro-regenerative properties were selected and ranked using bioinformatics analysis and machine learning models trained on a proprietary feature set comprised of multi-dimensional map of proteins and their diseases-modifying effects across different organs and indications. An example format for a library of disease-modifying candidate proteins is shown in FIG. 3. Features include chemistry properties, sequence information, evolutionary parameters, cell biology and protein-to-protein interaction information. Additional multipotent and totipotent stem cell secretomes are screened using the above pipeline. Secreted proteins from a pluripotent stem cell library were pre-ranked using a suite of artificial intelligence tools for in vitro IPF phenotype screening.

### Example 2—Candidate Ranking Pipeline for Proteins Modifying AT2 Cell Function Undergoing Fibrotic Stress

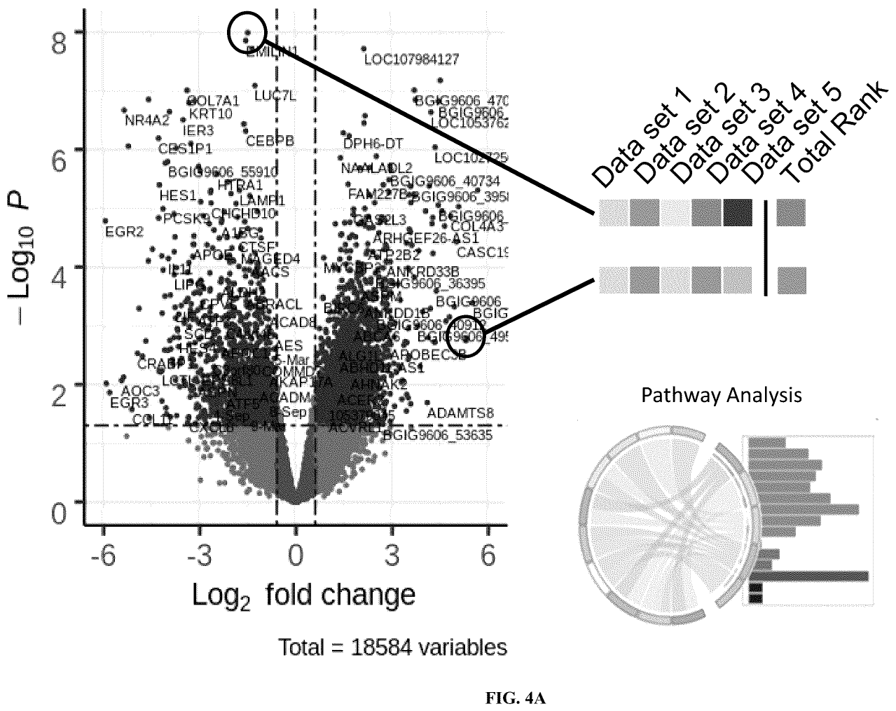

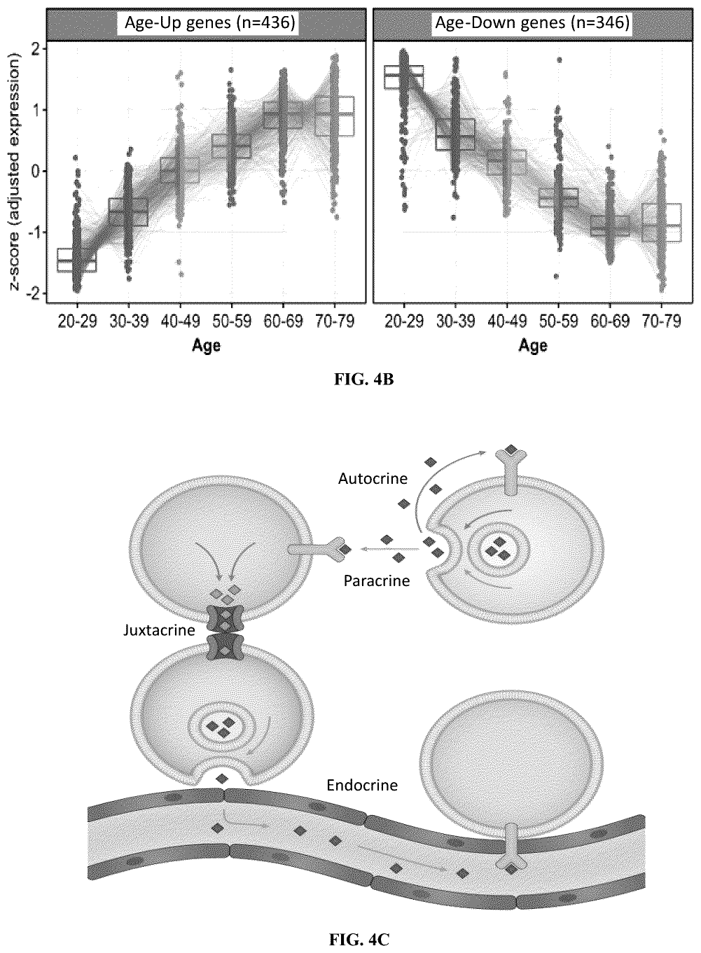

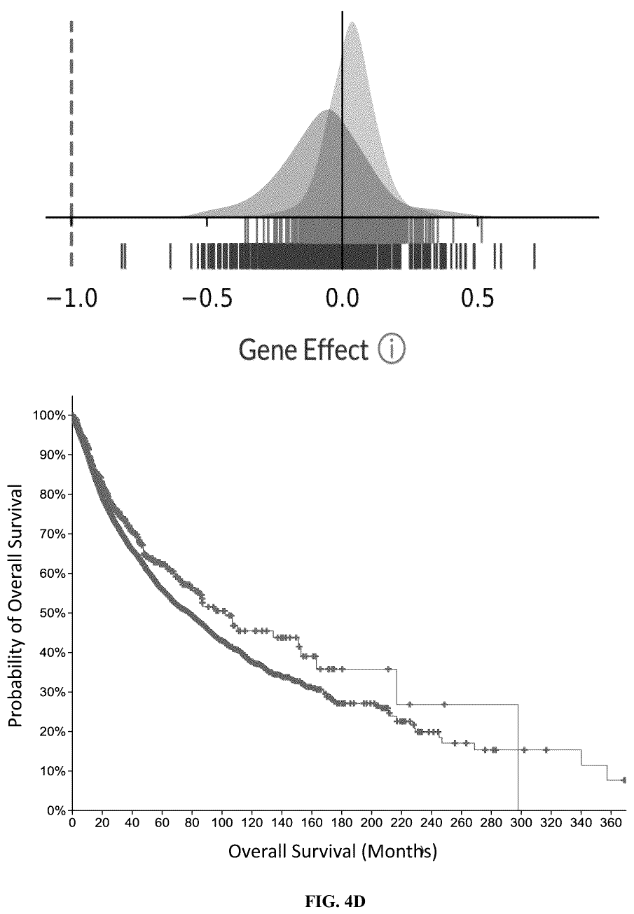

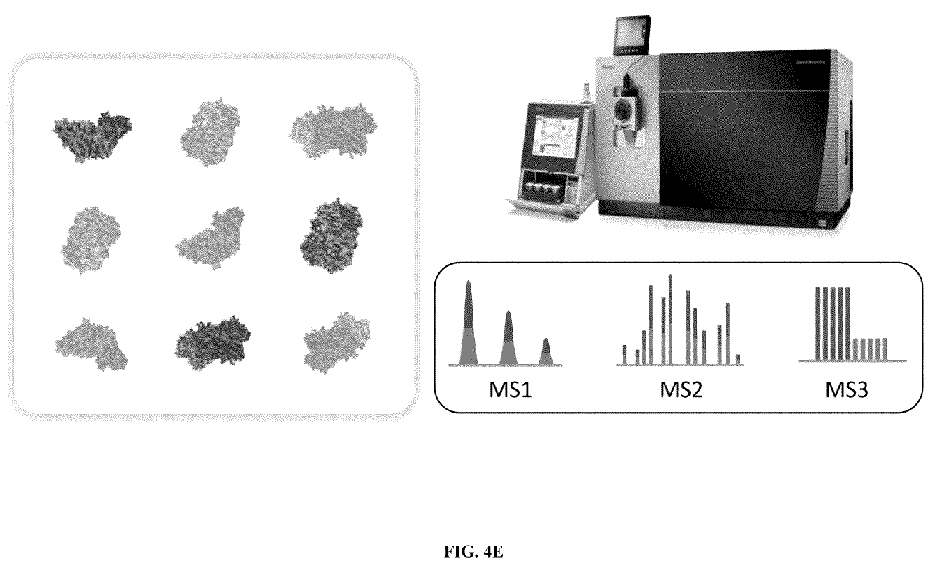

A schematic of the candidate ranking pipeline using the materials and methods described in Example 1 is shown in FIG. 4A-FIG. 4E. An artificial intelligence-enabled platform predicted and rank ordered therapeutic signaling proteins from a disease-modifying secreted protein library to screen and generate hits, followed by target validation, lead selection, and lead optimization to generate a growing pipeline of regenerative biologics for chronic and age-related diseases. The applications of quantitative proteomics, transcriptomics, and computer vision with machine learning fed high dimensional, multimodal data into a compounding database enabled by a growing human therapeutic protein library to hasten the process of systematic biologics drug discovery and development. Disease context (FIG. 4A), chronological expression (FIG. 4B), ligand-receptor mapping (FIG. 4C), safety profile (FIG. 4D), and stem-cell secretome analysis (FIG. 4E) were utilized for the screening candidate ranking for therapeutic modulators of pathological features of IPF.

### Example 3—High Throughput Screening of Pre-Ranked Secreted Proteins Identified Several Factors that Promote AT2 Regeneration

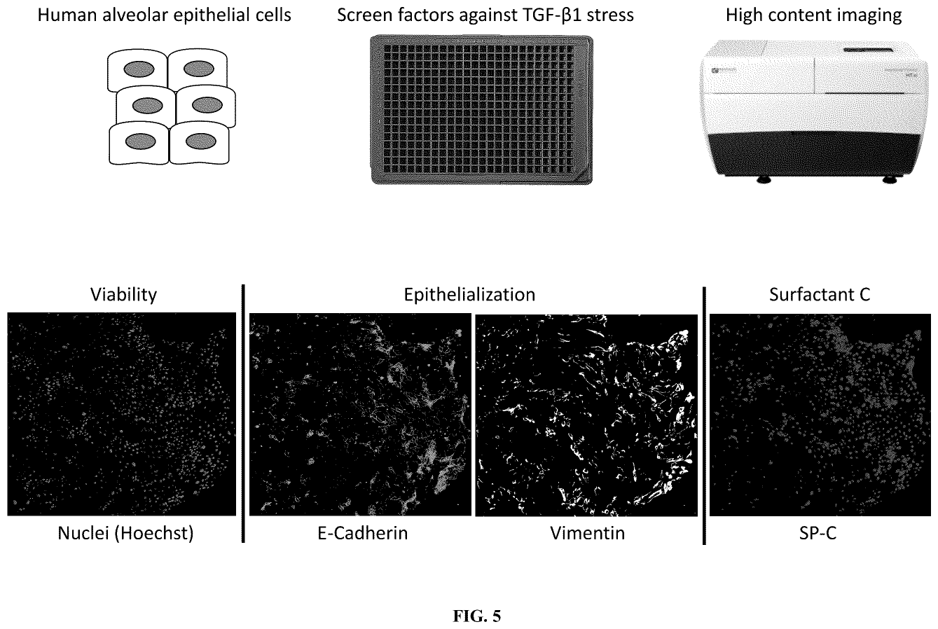

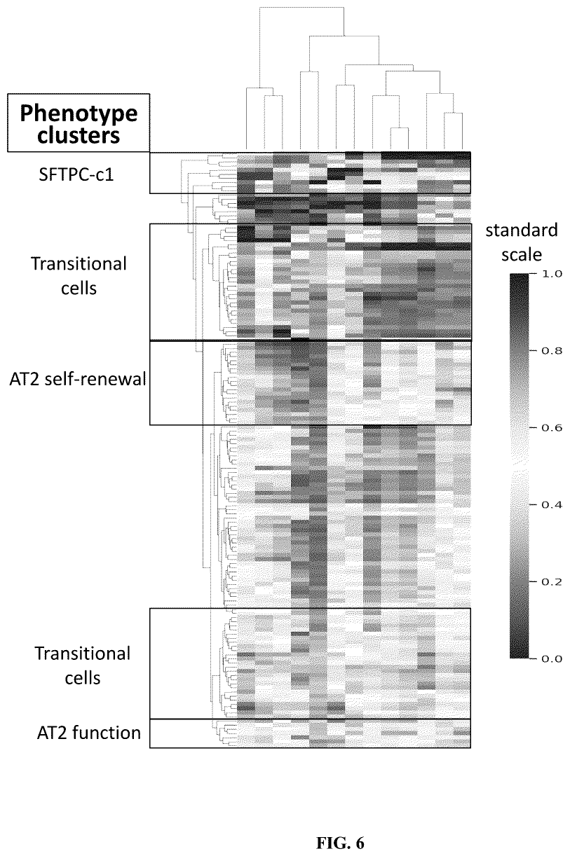

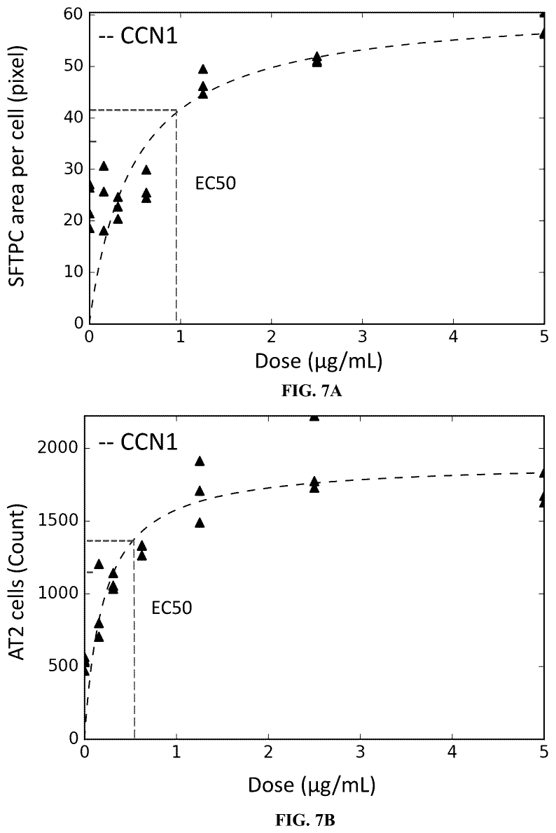

An in vitro assay was utilized to screen for secreted stem cell-derived factors that promote AT2 regeneration in an model of induced fibrosis. AT2 cells derived from healthy donors were screened in this example. FIG. 5 shows a schematic of the screening pipeline to identify factors that promote AT2 function under fibrotic stress. In FIG. 6, heatmap clustering of factors that alter AT2 phenotypes is shown. Cells: Human AT2 cells. Media: Basal media (BM) (n=3 human donor cells). In FIG. 7A-FIG. 7B, representative dose range curves of factors that improve surfactant C (SP-C) expression and increase AT2 cell counts are shown for CCN1. Cells shown in FIG. 7A and in FIG. 7B were treated with CCN1 polypeptide of SEQ ID NO: 26.

### Example 4—Testing of Selected Secreted Proteins for Promotion of AT2 Function or Inhibition of Epithelial-Mesenchymal Transition (EMT) Under TGFβ1-Induced Fibrotic Conditions in Healthy and IPF AT2 Cells

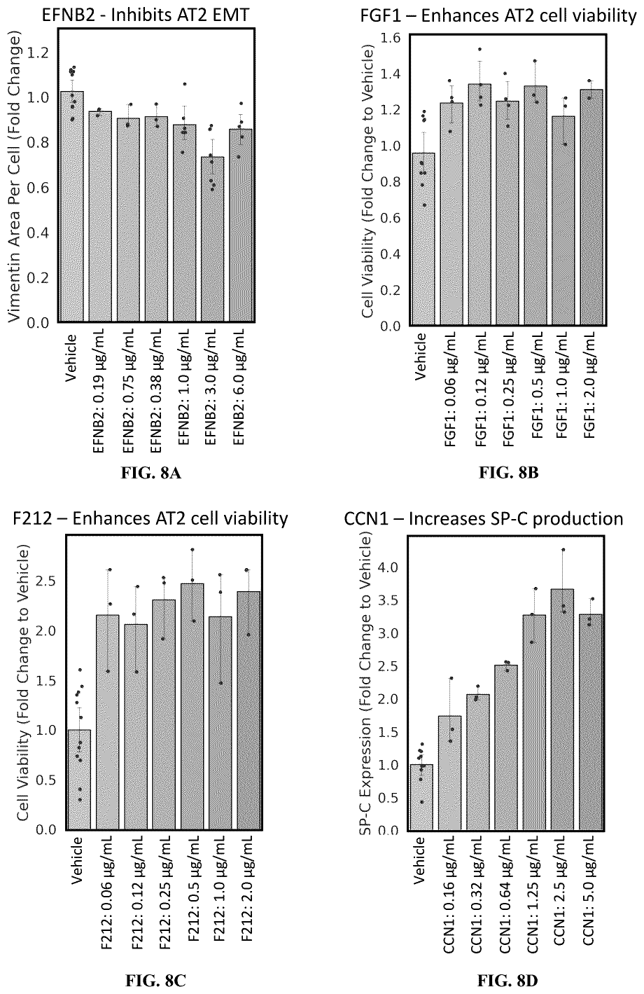

Human AT2 cells derived from healthy donors (31-58 y.o. females) were obtained and cultured in vitro. Vehicle treatment included culturing AT2 cells with basal media. All test conditions were treated with TGFβ1: 1 ng/mL to induce a fibrotic phenotype. Extent of EMT (as measured by Vimentin expression area per cell (fold change)), AT2 cell viability (as measured by fold change compared to vehicle treatment assaying quantifying nuclei by Hoechst staining), and surfactant production (as measured by SP-C expression fold change relative to vehicle treatment) were assayed. Results were graphed in FIG. 8A-FIG. 8D. In FIG. 8A, EFNB2-Fc fusion protein (SEQ ID NO: 25) at various doses is shown to inhibit EMT in AT2 cells. In FIG. 8B, FGF1 (SEQ ID NO: 20) at various doses is shown to enhances AT2 cell viability. In FIG. 8C, F212 (an AREG polypeptide of SEQ ID NO: 23) at various doses is shown to enhances AT2 cell viability. In FIG. 8C, CCN1-Fc fusion protein (SEQ ID NO: 26) at various doses is shown to increase SP-C production.

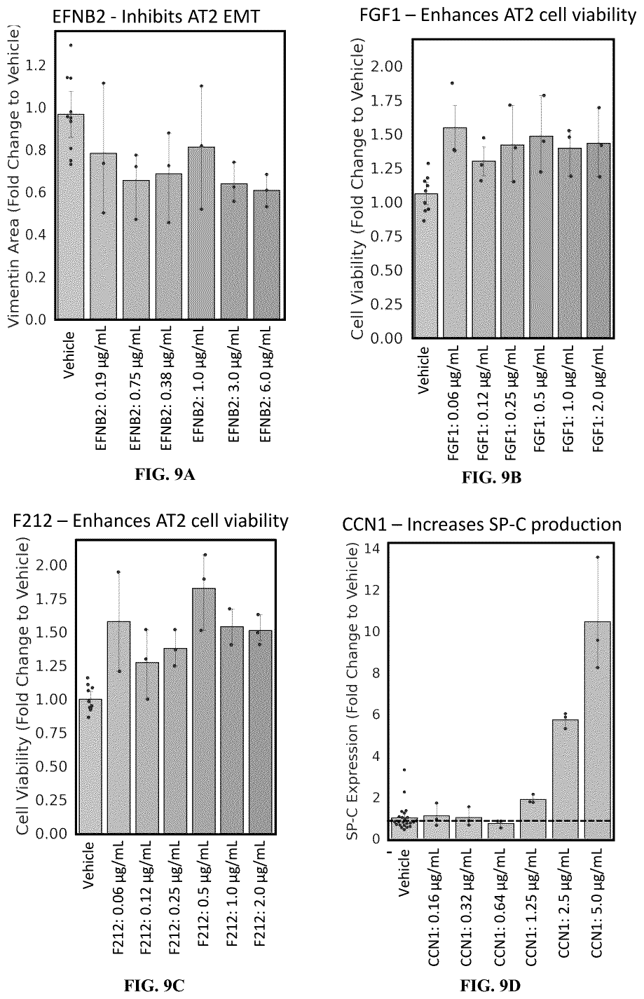

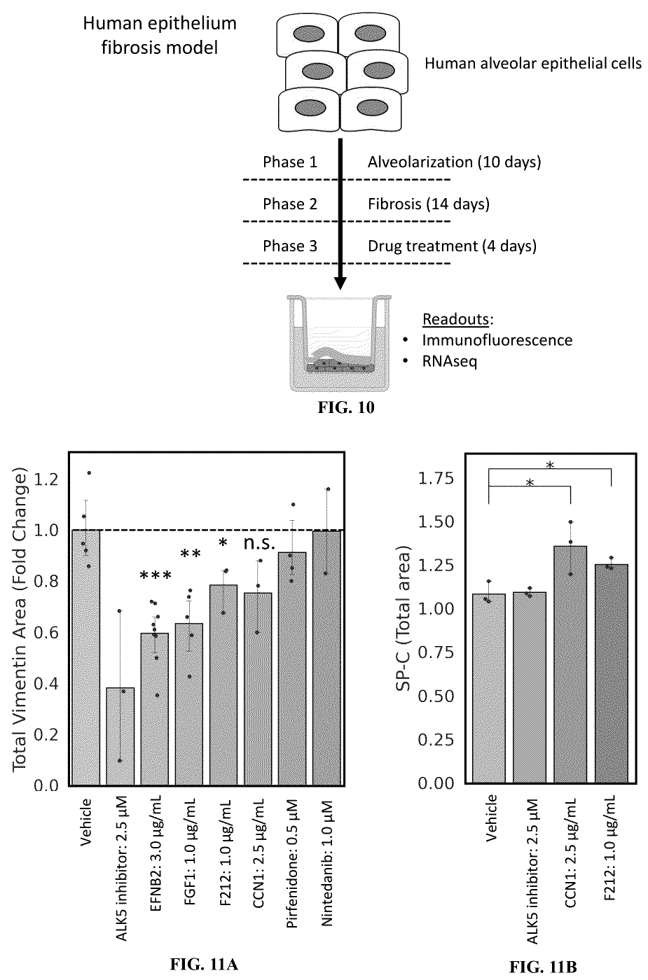

Conclusions: Multiple factors promoted human AT2 cell activity under fibrotic stress by multiple mechanisms including reducing EMT and improving SP-C production. Treatment with EFNB2 inhibits AT2 cell EMT. Treatment with FGF1 enhances AT2 cell viability. Treatment with F212 enhances AT2 cell viability. Treatment with CCN1 increases SP-C production. Treatment with CCN1 increases SP-C production in a dose-responsive manner. Human AT2 cells derived from an IPF donor (56 y.o. male) were obtained and cultured in vitro. Vehicle treatment included culturing AT2 cells with basal media. All test conditions were treated with TGFβ1: 1 ng/ml to induce a fibrotic phenotype. Extent of EMT (as measured by Vimentin expression area per cell (fold change)), AT2 cell viability (as measured by fold change compared to vehicle treatment assaying quantifying nuclei by Hoechst staining), and surfactant production (as measured by SP-C expression fold change relative to vehicle treatment) were assayed. Results were graphed in FIG. 9A-FIG. 9D. Conclusions: Treatment with EFNB2-Fc fusion protein (SEQ ID NO: 25) inhibits AT2 cell EMT in lung cells derived from a subject having IPF in FIG. 9A. Treatment with FGF1 (SEQ ID NO: 20) enhances AT2 cell viability in lung cells derived from a subject having IPF in FIG. 9B. Treatment with F212 (an AREG polypeptide of SEQ ID NO: 23) enhances AT2 cell viability in lung cells derived from a subject having IPF in FIG. 9C. Treatment with CCN1-Fc fusion protein (SEQ ID NO: 26) increases SP-C production in lung cells derived from a subject having IPF in FIG. 9D. Treatment with CCN1-Fc fusion protein (SEQ ID NO: 26) increases SP-C production in a dose-responsive manner in lung cells derived from a subject having IPF in FIG. 9D.

### Example 5—Testing of Selected Secreted Proteins for Promotion of AT2 Function or Inhibition of Epithelial-Mesenchymal Transition (EMT) in an Air-Liquid Interface (ALI) Model of Chronic Fibrosis in Healthy AT2 Cells

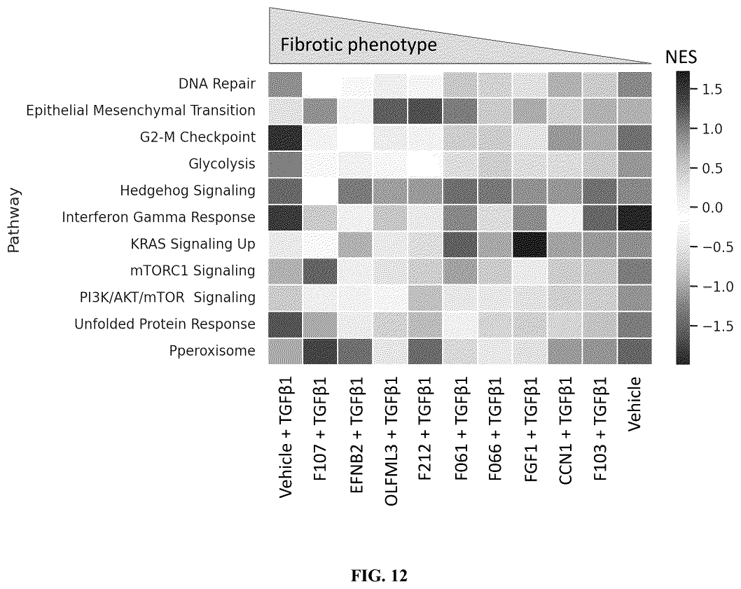

Human AT2 cells derived from healthy donors (3 Caucasian females, 31-78 y.o. females) were obtained and cultured in vitro. Vehicle treatment included culturing AT2 cells with basal media. All test conditions were treated according to the schematic in FIG. 10 to induce a fibrotic phenotype and test for drug treatment effectiveness in an alveolar epithelium fibrosis model. All test conditions treated with TGFβ1: 1 ng/mL to induce fibrosis. Human alveolar epithelial cells derived from healthy lung tissue samples were cultured in vitro. Phase 1 involved alveolization of cultured cells over a period of 10 days in culture. Phase 2 involved induction of fibrosis (ALI model) over a period of 14 days in culture. Phase 3 involved drug treatment for 4 days to test drug effectiveness on induced fibrotic epithelial cells. Extent of EMT (as measured by Vimentin expression area per cell (fold change)), and surfactant production (as measured by SP-C expression fold change relative to vehicle treatment) were assayed. Secreted factors promoted AT2 function and inhibited EMT under fibrotic conditions in this ALI model of chronic fibrosis. Results were graphed in FIG. 11A-FIG. 11B. In FIG. 11A, treatment with EFNB2-Fc fusion protein (SEQ ID NO: 25), FGF1 (SEQ ID NO: 20), and F212 (an AREG polypeptide of SEQ ID NO: 23) significantly inhibited EMT in AT2 cells. Treatment with CCN1-Fc fusion protein (SEQ ID NO: 26) was not shown to be significant for an inhibition of EMT in AT2 cells. In FIG. 11B, treatment with CCN1-Fc fusion protein (SEQ ID NO: 26) and F212 (an AREG polypeptide of SEQ ID NO: 23) significantly increased production of SP-C. RNAseq analysis was undertaken in AT2 cells derived from a Caucasian 57 y.o. male (n=2 technical replicates) having IPF. Control cells were vehicle treated. All test cells were treated with TGFβ1: 1 ng/mL to induce a fibrotic phenotype. Vehicle+TGFβ1 was used to compare treatment of various selected secreted stem cell factors in modifying cellular pathways in TGFβ1-induced fibrotic AT2 cells. Heatmap of fibrotic and cellular stress pathways upregulated by TGFβ1 treatment were attenuated by multiple secreted proteins (FIG. 12). *Pperoxisome is indicative of lipid metabolism and is associated with surfactant production. Pathway analysis of RNAseq data elucidated the mechanism of action (MOA) for different factors in ameliorating fibrosis and promoting alveolar regeneration in AT2 cells from IPF lung.

### Example 6—Secreted Factors Improve Lung Function in a Bleomycin Model of IPF

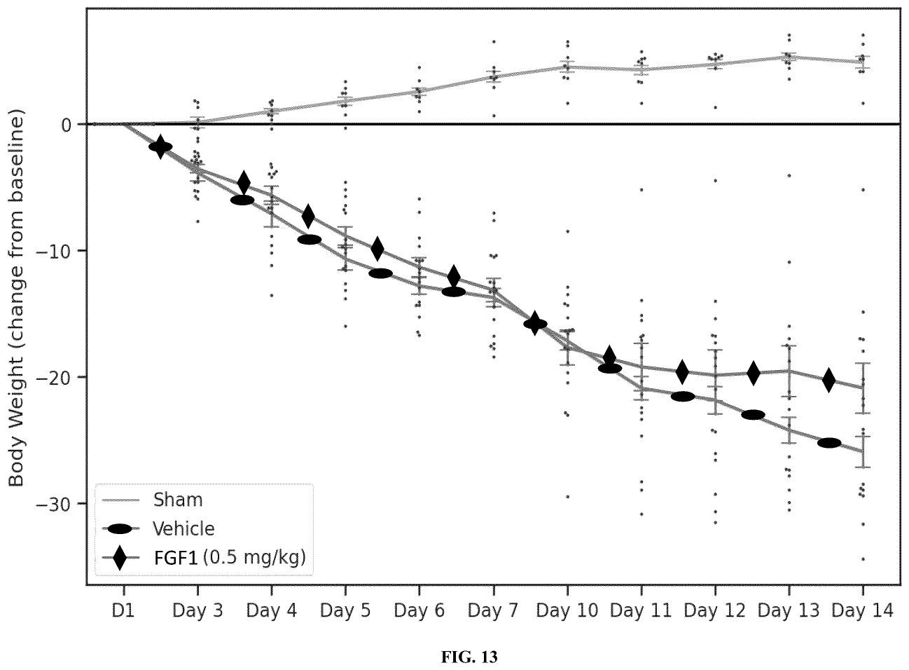

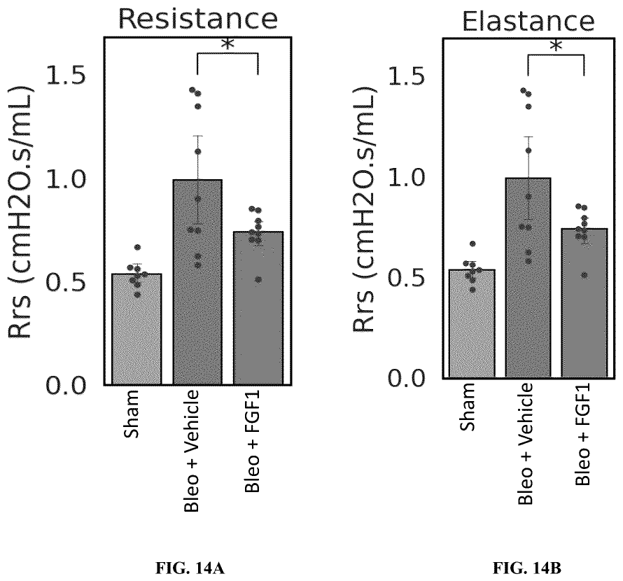

To study the effects of secreted stem cell-derived factors in vivo, 12 week old male C57BL/6 mice were injected with 1.3 mg/kg bleomycin to induce a mouse model of IPF. Vehicle or FGF1 (polypeptide of SEQ ID NO: 20) was injected I.P. daily (at a dose of 0.5 mg/kg body weight) starting day 7 post-bleomycin. As shown in FIG. 13, FGF1 treatment attenuated an extent of weight loss brought on by the bleomycin-induced IPF phenotype after approximately 11 days of treatment. Promising lung function improvement in this animal study was seen by body weight and lung function parameters. As shown in FIG. 14A, FGF1 (polypeptide of SEQ ID NO: 20) treatment significantly improved a bleomycin-induced increase in lung resistance (a metric of the level of constriction). As shown in FIG. 14B, FGF1 (polypeptide of SEQ ID NO: 20) treatment significantly improved a bleomycin-induced increase in lung elastance (a metric of lung stiffness). These results demonstrate that FGF1 treatment in a mouse model of IPF significantly improves several parameters of lung function.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### Aspects

Aspect 1: A method of treating a subject having an interstitial lung disease (ILD), the method comprising administering to the subject an effective amount of an OLFML3 protein or functional derivative thereof, wherein the effective amount improves one or more symptoms associated with pulmonary fibrosis in the subject.

Aspect 2: A method of treating a subject having an interstitial lung disease (ILD), the method comprising administering to the subject an effective amount of an OLFML3 protein or functional derivative thereof, wherein the effective amount increases alveolar regeneration in the subject, promotes repair of pulmonary fibrosis in the subject, restores at least partial lung function in the subject, or any combination thereof.

Aspect 3: A method of treating a subject having an idiopathic pulmonary fibrosis (IPF), the method comprising administering to the subject an effective amount of an OLFML3 protein or functional derivative thereof, wherein the effective amount improves one or more symptoms associated with pulmonary fibrosis in the subject.

Aspect 4: A method of treating a subject having an idiopathic pulmonary fibrosis (IPF), the method comprising administering to the subject an effective amount of an OLFML3 protein or functional derivative thereof, wherein the effective amount increases alveolar regeneration in the subject, promotes repair of pulmonary fibrosis in the subject, restores at least partial lung function in the subject, or any combination thereof.

Aspect 5: A method of treating a subject having an interstitial lung disease (ILD), the method comprising administering to the subject an effective amount of an NDP protein or functional derivative thereof, wherein the effective amount improves one or more symptoms associated with pulmonary fibrosis in the subject.

Aspect 6: A method of treating a subject having an interstitial lung disease (ILD), the method comprising administering to the subject an effective amount of an NDP protein or functional derivative thereof, wherein the effective amount increases alveolar regeneration in the subject, promotes repair of pulmonary fibrosis in the subject, restores at least partial lung function in the subject, or any combination thereof.

Aspect 7: A method of treating a subject having an idiopathic pulmonary fibrosis (IPF), the method comprising administering to the subject an effective amount of an NDP protein or functional derivative thereof, wherein the effective amount improves one or more symptoms associated with pulmonary fibrosis in the subject.

Aspect 8: A method of treating a subject having an idiopathic pulmonary fibrosis (IPF), the method comprising administering to the subject an effective amount of an NDP protein or functional derivative thereof, wherein the effective amount increases alveolar regeneration in the subject, promotes repair of pulmonary fibrosis in the subject, restores at least partial lung function in the subject, or any combination thereof.

Aspect 9: A method of treating a subject having an interstitial lung disease (ILD), the method comprising administering to the subject an effective amount of an FGF1 protein or functional derivative thereof, wherein the effective amount improves one or more symptoms associated with pulmonary fibrosis in the subject.

Aspect 10: A method of treating a subject having an interstitial lung disease (ILD), the method comprising administering to the subject an effective amount of an FGF1 protein or functional derivative thereof, wherein the effective amount increases alveolar regeneration in the subject, promotes repair of pulmonary fibrosis in the subject, restores at least partial lung function in the subject, or any combination thereof.

Aspect 11: A method of treating a subject having an idiopathic pulmonary fibrosis (IPF), the method comprising administering to the subject an effective amount of an FGF1 protein or functional derivative thereof, wherein the effective amount improves one or more symptoms associated with pulmonary fibrosis in the subject.

Aspect 12: A method of treating a subject having an idiopathic pulmonary fibrosis (IPF), the method comprising administering to the subject an effective amount of an FGF1 protein or functional derivative thereof, wherein the effective amount increases alveolar regeneration in the subject, promotes repair of pulmonary fibrosis in the subject, restores at least partial lung function in the subject, or any combination thereof.

Aspect 13: A method of treating a subject having an interstitial lung disease (ILD), the method comprising administering to the subject an effective amount of an EFNB2 protein or functional derivative thereof, wherein the effective amount improves one or more symptoms associated with pulmonary fibrosis in the subject.

Aspect 14: A method of treating a subject having an interstitial lung disease (ILD), the method comprising administering to the subject an effective amount of an EFNB2 protein or functional derivative thereof, wherein the effective amount increases alveolar regeneration in the subject, promotes repair of pulmonary fibrosis in the subject, restores at least partial lung function in the subject, or any combination thereof.

Aspect 15: A method of treating a subject having an idiopathic pulmonary fibrosis (IPF), the method comprising administering to the subject an effective amount of an EFNB2 protein or functional derivative thereof, wherein the effective amount improves one or more symptoms associated with pulmonary fibrosis in the subject.

Aspect 16: A method of treating a subject having an idiopathic pulmonary fibrosis (IPF), the method comprising administering to the subject an effective amount of an EFNB2 protein or functional derivative thereof, wherein the effective amount increases alveolar regeneration in the subject, promotes repair of pulmonary fibrosis in the subject, restores at least partial lung function in the subject, or any combination thereof.

Aspect 17: A method of treating a subject having an interstitial lung disease (ILD), the method comprising administering to the subject an effective amount of an CCN1 protein or functional derivative thereof, wherein the effective amount improves one or more symptoms associated with pulmonary fibrosis in the subject.

Aspect 18: A method of treating a subject having an interstitial lung disease (ILD), the method comprising administering to the subject an effective amount of an CCN1 protein or functional derivative thereof, wherein the effective amount increases alveolar regeneration in the subject, promotes repair of pulmonary fibrosis in the subject, restores at least partial lung function in the subject, or any combination thereof.

Aspect 19: A method of treating a subject having an idiopathic pulmonary fibrosis (IPF), the method comprising administering to the subject an effective amount of an CCN1 protein or functional derivative thereof, wherein the effective amount improves one or more symptoms associated with pulmonary fibrosis in the subject.

Aspect 20: A method of treating a subject having an idiopathic pulmonary fibrosis (IPF), the method comprising administering to the subject an effective amount of an CCN1 protein or functional derivative thereof, wherein the effective amount increases alveolar regeneration in the subject, promotes repair of pulmonary fibrosis in the subject, restores at least partial lung function in the subject, or any combination thereof.

Aspect 21: A method of treating a subject having an interstitial lung disease (ILD), the method comprising administering to the subject an effective amount of an AREG protein or functional derivative thereof, wherein the effective amount improves one or more symptoms associated with pulmonary fibrosis in the subject.

Aspect 22: A method of treating a subject having an interstitial lung disease (ILD), the method comprising administering to the subject an effective amount of an AREG1 protein or functional derivative thereof, wherein the effective amount increases alveolar regeneration in the subject, promotes repair of pulmonary fibrosis in the subject, restores at least partial lung function in the subject, or any combination thereof.

Aspect 23: A method of treating a subject having an idiopathic pulmonary fibrosis (IPF), the method comprising administering to the subject an effective amount of an AREG protein or functional derivative thereof, wherein the effective amount improves one or more symptoms associated with pulmonary fibrosis in the subject.

Aspect 24: A method of treating a subject having an idiopathic pulmonary fibrosis (IPF), the method comprising administering to the subject an effective amount of an AREG protein or functional derivative thereof, wherein the effective amount increases alveolar regeneration in the subject, promotes repair of pulmonary fibrosis in the subject, restores at least partial lung function in the subject, or any combination thereof.

What is claimed is:

1. A method of treating a subject having an idiopathic pulmonary fibrosis (IPF), the method comprising administering to the subject an effective amount of (i) an OLFML3 protein or functional derivative thereof, (ii) an NDP protein or functional derivative thereof, (iii) an AREG protein or functional derivative thereof, (iv) an FGF1 protein or functional derivative thereof, (v) an EFNB2 protein or functional derivative thereof or an EFNB2-Fc fusion protein, or (vi) a CCN1 protein or functional derivative thereof or a CCN1-Fc fusion protein, wherein the effective amount increases alveolar regeneration in the subject, promotes repair of pulmonary fibrosis in the subject, restores at least partial lung function in the subject, or any combination thereof.

2. The method of claim 1, wherein the effective amount of the EFNB2-Fc fusion protein is administered, and wherein repair of pulmonary fibrosis is promoted in the subject.

3. The method of claim 1, wherein the effective amount of the EFNB2-Fc fusion protein is administered, and wherein epithelial-mesenchymal transition (EMT) in AT2 cells is inhibited in the subject.

4. The method of claim 1, wherein the effective amount of the EFNB2-Fc fusion protein is administered, and wherein expression of Vimentin per area on the surface of AT2 cells is decreased in the subject.

5. The method of claim 1, wherein the effective amount of the FGF1 protein or functional derivative thereof is administered, and wherein alveolar regeneration is increased in the subject.

6. The method of claim 1, wherein the effective amount of the FGF1 protein or functional derivative thereof is administered, and wherein AT2 cell viability is increased in the subject.

7. The method of claim 1, wherein the effective amount of the FGF1 protein or functional derivative thereof is administered, and wherein lung resistance is decreased in the subject.

8. The method of claim 1, wherein the effective amount of the FGF1 protein or functional derivative thereof is administered, and wherein lung elastance is decreased in the subject.

9. The method of claim 1, wherein the effective amount of the FGF1 protein or functional derivative thereof is administered, and wherein lung compliance is improved in the subject.

10. The method of claim 1, wherein the effective amount of the AREG protein or functional derivative thereof is administered, and wherein alveolar regeneration is increased in the subject.

11. The method of claim 1, wherein the effective amount of the AREG protein or functional derivative thereof is administered, and wherein AT2 cell viability is increased in the subject.

12. The method of claim 1, wherein the effective amount of the AREG protein or functional derivative thereof is administered, and wherein surfactant SP-C total area of cell surface expression in AT2 cells is increased in the subject.

13. The method of claim 1, wherein the effective amount of the CCN1-Fc fusion protein is administered, and wherein at least partial lung function is restored in the subject.

14. The method of claim 1, wherein the effective amount of the CCN1-Fc fusion protein is administered, and wherein surfactant SP-C production is increased in AT2 cells in the subject.

15. The method of claim 1, wherein the effective amount of the CCN1-Fc fusion protein is administered, and wherein surfactant SP-C total area of cell surface expression in AT2 cells is increased in the subject.

16. A method of treating a subject having an idiopathic pulmonary fibrosis (IPF), the method comprising administering to the subject an effective amount of (i) an OLFML3 protein or functional derivative thereof, (ii) an NDP protein or functional derivative thereof, (iii) an AREG protein or functional derivative thereof, (iv) an FGF1 protein or functional derivative thereof, (v) an EFNB2 protein or functional derivative thereof or an EFNB2-Fc fusion protein, or (vi) a CCN1 protein or functional derivative thereof or a CCN1-Fc fusion protein, wherein the effective amount improves one or more symptoms associated with pulmonary fibrosis in the subject.

17. A method of treating a subject having an interstitial lung disease (ILD), the method comprising administering to the subject an effective amount of an (i) an OLFML3 protein or functional derivative thereof, (ii) an NDP protein or functional derivative thereof, (iii) an AREG protein or functional derivative thereof, (iv) an FGF1 protein or functional derivative thereof, (v) an EFNB2 protein or functional derivative thereof or an EFNB2-Fc fusion protein, or (vi) a CCN1 protein or functional derivative thereof or a CCN1-Fc fusion protein, wherein the effective amount increases alveolar regeneration in the subject, promotes repair of pulmonary fibrosis in the subject, restores at least partial lung function in the subject, or any combination thereof.


SEQUENCE LISTING (41 sequences)

SEQ ID NO: 1 - protein, 406 aa, Homo sapiens
MGPSTPLLILFLLSWSGPLQGQQHHLVEYMERRLAALEERLAQCQDQSSRHAAELRDFKNKMLPLLEVAEKEREALRTEADTISGRVDRLEREVDYLETQNPALPCVEFDEKVTGGPGTKGKGRRNEKYDMVTDCGYTISQVRSMKILKRFGGPAGLWTKDPLGQTEKIYVLDGTQNDTAFVFPRLRDFTLAMAARKASRVRVPFPWVGTGQLVYGGFLYFARRPPGRPGGGGEMENTLQLIKFHLANRTVVDSSVFPAEGLIPPYGLTADTYIDLAADEEGLWAVYATREDDRHLCLAKLDPQTLDTEQQWDTPCPRENAEAAFVICGTLYVVYNTRPASRARIQCSFDASGTLTPERAALPYFPRRYGAHASLRYNPRERQLYAWDDGYQIVYKLEMRKKEEEV

SEQ ID NO: 2 - protein, 386 aa, Homo sapiens
MAQWHCSQRTQAGGHGVGERLAQCQDQSSRHAAELRDFKNKMLPLLEVAEKEREALRTEADTISGRVDRLEREVDYLETQNPALPCVEFDEKVTGGPGTKGKGRRNEKYDMVTDCGYTISQVRSMKILKRFGGPAGLWTKDPLGQTEKIYVLDGTQNDTAFVFPRLRDFTLAMAARKASRVRVPFPWVGTGQLVYGGFLYFARRPPGRPGGGGEMENTLQLIKFHLANRTVVDSSVFPAEGLIPPYGLTADTYIDLAADEEGLWAVYATREDDRHLCLAKLDPQTLDTEQQWDTPCPRENAEAAFVICGTLYVVYNTRPASRARIQCSFDASGTLTPERAALPYFPRRYGAHASLRYNPRERQLYAWDDGYQIVYKLEMRKKEEEV

SEQ ID NO: 3 - protein, 345 aa, Homo sapiens
MLPLLEVAEKEREALRTEADTISGRVDRLEREVDYLETQNPALPCVEFDEKVTGGPGTKGKGRRNEKYDMVTDCGYTISQVRSMKILKRFGGPAGLWTKDPLGQTEKIYVLDGTQNDTAFVFPRLRDFTLAMAARKASRVRVPFPWVGTGQLVYGGFLYFARRPPGRPGGGGEMENTLQLIKFHLANRTVVDSSVFPAEGLIPPYGLTADTYIDLAADEEGLWAVYATREDDRHLCLAKLDPQTLDTEQQWDTPCPRENAEAAFVICGTLYVVYNTRPASRARIQCSFDASGTLTPERAALPYFPRRYGAHASLRYNPRERQLYAWDDGYQIVYKLEMRKKEEEV

SEQ ID NO: 4 - protein, 133 aa, Homo sapiens
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 5 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 6 - protein, 60 aa, Homo sapiens
MAEGEITTFTALTEKFNLPPGNYKKPKLLYCSNGGHFLRILPDGTVDGTRDRSDQHTDTK

SEQ ID NO: 7 - protein, 59 aa, Homo sapiens
MAEGEITTFTALTEKFNLPPGNYKKPKLLYCSNGGHFLRILPDGTVDGTRDRSDQHNTK

SEQ ID NO: 8 - protein, 154 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSTPNEECLFLERLEENHYNTYISKKHAEKNWFVGLKKNGSCKRGPRTHYGQKAILFLPLPVSSD

SEQ ID NO: 9 - protein, 333 aa, Homo sapiens
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 10 - protein, 302 aa, Homo sapiens
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIIYASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 11 - protein, 295 aa, Homo sapiens
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 12 - protein, 335 aa, Homo sapiens
MYFQRLQQHQVAPQRFGFSSRLIYTLWPTVRKLVRRHQMFVLRFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 13 - protein, 175 aa, Homo sapiens
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISVIFVIGYSSESCQRLCIWDLGGYSTTTTLPLSKRNRSTS

SEQ ID NO: 14 - protein, 381 aa, Homo sapiens [sequence omitted]

SEQ ID NO: 15 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 16 - protein, 287 aa, synthetic construct
KGKGRRNEKYDMVTDCGYTISQVRSMKILKRFGGPAGLWTKDPLGQTEKIYVLDGTQNDTAFVFPRLRDFTLAMAARKASRVRVPFPWVGTGQLVYGGFLYFARRPPGRPGGGGEMENTLQLIKFHLANRTVVDSSVFPAEGLIPPYGLTADTYIDLAADEEGLWAVYATREDDRHLCLAKLDPQTLDTEQQWDTPCPRENAEAAFVICGTLYVVYNTRPASRARIQCSFDASGTLTPERAALPYFPRRYGAHASLRYNPRERQLYAWDDGYQIVYKLEMRKKEEEV

SEQ ID NO: 17 - protein, 110 aa, synthetic construct
MKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 18 - protein, 110 aa, synthetic construct
SKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 19 - protein, 56 aa, synthetic construct
MAEGEITTFTALTEKFNLPPGNYKKPKLLYCSNGGHFLRILPDGTVDGTRDRSDQH

SEQ ID NO: 20 - protein, 141 aa, synthetic construct
MFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 21 - protein, 144 aa, synthetic construct
SKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDA

SEQ ID NO: 22 - protein, 357 aa, synthetic construct
TCPAACHCPLEAPKCAPGVGLVRDGCGCCKVCAKQLNEDCSKTQPCDHTKGLECNFGASSTALKGICRAQSEGRPCEYNSRIYQNGESFQPNCKHQCTCIDGAVGCIPLCPQELSLPNLGCPNPRLVKVTGQCCEEWVCDEDSIKDPMEDQDGLLGKELGFDASEVELTRNNELIAVGKGSSLKRLPVFGMEPRILYNPLQGQKCIVQTTSWSQCSKTCGTGISTRVTNDNPECRLVKETRICEVRPCGQPVYSSLKKGKKCSKTKKSPEPVRFTYAGCLSVKKYRPKYCGSCVDGRCCTPQLTRTVKMRFRCEDGETFSKNVMMIQSCKCNYNCPHANEAAFPFYRLFNDIHKFRD

SEQ ID NO: 23 - protein, 99 aa, synthetic construct
MSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSK

SEQ ID NO: 24 - protein, 294 aa, synthetic construct
MKGKGRRNEKYDMVTDCGYTISQVRSMKILKRFGGPAGLWTKDPLGQTEKIYVLDGTQNDTAFVFPRLRDFTLAMAARKASRVRVPFPWVGTGQLVYGGFLYFARRPPGRPGGGGEMENTLQLIKFHLANRTVVDSSVFPAEGLIPPYGLTADTYIDLAADEEGLWAVYATREDDRHLCLAKLDPQTLDTEQQWDTPCPRENAEAAFVICGTLYVVYNTRPASRARIQCSFDASGTLTPERAALPYFPRRYGAHASLRYNPRERQLYAWDDGYQIVYKLEMRKKEEEVHHHHHH

SEQ ID NO: 25 - protein, 466 aa, synthetic construct
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 598 aa, synthetic construct
MALSTCPAACHCPLEAPKCAPGVGLVRDGCGCCKVCAKQLNEDCSKTQPCDHTKGLECNFGASSTALKGICRAQSEGRPCEYNSRIYQNGESFQPNCKHQCTCIDGAVGCIPLCPQELSLPNLGCPNPRLVKVTGQCCEEWVCDEDSIKDPMEDQDGLLGKELGFDASEVELTRNNELIAVGKGSSLKRLPVFGMEPRILYNPLQGQKCIVQTTSWSQCSKTCGTGISTRVTNDNPECRLVKETRICEVRPCGQPVYSSLKKGKKCSKTKKSPEPVRFTYAGCLSVKKYRPKYCGSCVDGRCCTPQLTRTVKMRFRCEDGETFSKNVMMIQSCKCNYNCPHANEAAFPFYRLFNDIHKFRDIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1748 bp, synthetic construct
  source 1..1748: mol_type = other DNA
gactgtacgttccttctactctggcaccactctccaggctgccatggggcccagcacccctctcctcatcttgttccttttgtcatggtcgggacccctccaaggacagcagcaccaccttgtggagtacatggaacgccgactagctgctttagaggaacggctggcccagtgccaggaccagagtagtcggcatgctgctgagctgcgggacttcaagaacaagatgctgccactgctggaggtggcagagaaggagcgggaggcactcagaactgaggccgacaccatctccgggagagtggatcgtctggagcgggaggtagactatctggagacccagaacccagctctgccctgtgtagagtttgatgagaaggtgactggaggccctgggaccaaaggcaagggaagaaggaatgagaagtacgatatggtgacagactgtggctacacaatctctcaagtgagatcaatgaagattctgaagcgatttggtggcccagctggtctatggaccaaggatccactggggcaaacagagaagatctacgtgttagatgggacacagaatgacacagcctttgtcttcccaaggctgcgtgacttcacccttgccatggctgcccggaaagcttcccgagtccgggtgcccttcccctgggtaggcacagggcagctggtatatggtggctttctttattttgctcggaggcctcctggaagacctggtggaggtggtgagatggagaacactttgcagctaatcaaattccacctggcaaaccgaacagtggtggacagctcagtattcccagcagaggggctgatccccccctacggcttgacagcagacacctacatcgacctggcagctgatgaggaaggtctttgggctgtctatgccacccgggaggatgacaggcacttgtgtctggccaagttagatccacagacactggacacagagcagcagtgggacacaccatgtcccagagagaatgctgaggctgcctttgtcatctgtgggaccctctatgtcgtctataacacccgtcctgccagtcgggcccgcatccagtgctcctttgatgccagcggcaccctgacccctgaacgggcagcactcccttattttccccgcagatatggtgcccatgccagcctccgctataacccccgagaacgccagctctatgcctgggatgatggctaccagattgtctataagctggagatgaggaagaaagaggaggaggtttgaggagctagccttgttttttgcatctttctcactcccatacatttatattatatccccactaaatttcttgttcctcattcttcaaatgtgggccagttgtggctcaaatcctctatatttttagccaatggcaatcaaattctttcagctcctttgtttcatacggaactccagatcctgagtaatccttttagagcccgaagagtcaaaaccctcaatgttccctcctgctctcctgccccatgtcaacaaatttcaggctaaggatgccccagacccagggctctaaccttgtatgcgggcaggcccagggagcaggcagcagtgttcttcccctcagagtgacttggggagggagaaataggaggagacgtccagctctgtcctctcttcctcactcctcccttcagtgtcctgaggaacaggactttctccacattgttttgtattgcaacattttgcattaaaaggaaaatccactgct

SEQ ID NO: 28 - DNA, 1900 bp, synthetic construct
  source 1..1900: mol_type = other DNA
gactgtacgttccttctactctggcaccactctccaggctgccatggggcccagcacccctctcctcatcttgttccttttgtcatggtcgggacccctccaaggacagcagcaccaccttgtggagtacatggaacgccgactagctgctttagaggttgagatccgtagcattttagtgtccgagaggataattgctaagggagcagagacctcttcacaccttcatgcgcttagacctgcctagaggctcagatggctcagtggcactgctcacagagaacacaggctggaggacatggagtcggggaacggctggcccagtgccaggaccagagtagtcggcatgctgctgagctgcgggacttcaagaacaagatgctgccactgctggaggtggcagagaaggagcgggaggcactcagaactgaggccgacaccatctccgggagagtggatcgtctggagcgggaggtagactatctggagacccagaacccagctctgccctgtgtagagtttgatgagaaggtgactggaggccctgggaccaaaggcaagggaagaaggaatgagaagtacgatatggtgacagactgtggctacacaatctctcaagtgagatcaatgaagattctgaagcgatttggtggcccagctggtctatggaccaaggatccactggggcaaacagagaagatctacgtgttagatgggacacagaatgacacagcctttgtcttcccaaggctgcgtgacttcacccttgccatggctgcccggaaagcttcccgagtccgggtgcccttcccctgggtaggcacagggcagctggtatatggtggctttctttattttgctcggaggcctcctggaagacctggtggaggtggtgagatggagaacactttgcagctaatcaaattccacctggcaaaccgaacagtggtggacagctcagtattcccagcagaggggctgatccccccctacggcttgacagcagacacctacatcgacctggcagctgatgaggaaggtctttgggctgtctatgccacccgggaggatgacaggcacttgtgtctggccaagttagatccacagacactggacacagagcagcagtgggacacaccatgtcccagagagaatgctgaggctgcctttgtcatctgtgggaccctctatgtcgtctataacacccgtcctgccagtcgggcccgcatccagtgctcctttgatgccagcggcaccctgacccctgaacgggcagcactcccttattttccccgcagatatggtgcccatgccagcctccgctataacccccgagaacgccagctctatgcctgggatgatggctaccagattgtctataagctggagatgaggaagaaagaggaggaggtttgaggagctagccttgttttttgcatctttctcactcccatacatttatattatatccccactaaatttcttgttcctcattcttcaaatgtgggccagttgtggctcaaatcctctatatttttagccaatggcaatcaaattctttcagctcctttgtttcatacggaactccagatcctgagtaatccttttagagcccgaagagtcaaaaccctcaatgttccctcctgctctcctgccccatgtcaacaaatttcaggctaaggatgccccagacccagggctctaaccttgtatgcgggcaggcccagggagcaggcagcagtgttcttcccctcagagtgacttggggagggagaaataggaggagacgtccagctctgtcctctcttcctcactcctcccttcagtgtcctgaggaacaggactttctccacattgttttgtattgcaacattttgcattaaaaggaaaatccactgct

SEQ ID NO: 29 - DNA, 1877 bp, synthetic construct
  source 1..1877: mol_type = other DNA
gactgtacgttccttctactctggcaccactctccaggctgccatggggcccagcacccctctcctcatcttgttccttttgtcatggtcgggacccctccaaggacagcagcaccaccttgtggagtacatggaacgccgactagctgctttagaggagtggaaggggatattgaagcctggctgagtttgctctttttttcccattcctgactcccgagttgggcctctccctgcctgggagttgtaaccctgcagcctccctcctgatcccccatcccttcaggaacggctggcccagtgccaggaccagagtagtcggcatgctgctgagctgcgggacttcaagaacaagatgctgccactgctggaggtggcagagaaggagcgggaggcactcagaactgaggccgacaccatctccgggagagtggatcgtctggagcgggaggtagactatctggagacccagaacccagctctgccctgtgtagagtttgatgagaaggtgactggaggccctgggaccaaaggcaagggaagaaggaatgagaagtacgatatggtgacagactgtggctacacaatctctcaagtgagatcaatgaagattctgaagcgatttggtggcccagctggtctatggaccaaggatccactggggcaaacagagaagatctacgtgttagatgggacacagaatgacacagcctttgtcttcccaaggctgcgtgacttcacccttgccatggctgcccggaaagcttcccgagtccgggtgcccttcccctgggtaggcacagggcagctggtatatggtggctttctttattttgctcggaggcctcctggaagacctggtggaggtggtgagatggagaacactttgcagctaatcaaattccacctggcaaaccgaacagtggtggacagctcagtattcccagcagaggggctgatccccccctacggcttgacagcagacacctacatcgacctggcagctgatgaggaaggtctttgggctgtctatgccacccgggaggatgacaggcacttgtgtctggccaagttagatccacagacactggacacagagcagcagtgggacacaccatgtcccagagagaatgctgaggctgcctttgtcatctgtgggaccctctatgtcgtctataacacccgtcctgccagtcgggcccgcatccagtgctcctttgatgccagcggcaccctgacccctgaacgggcagcactcccttattttccccgcagatatggtgcccatgccagcctccgctataacccccgagaacgccagctctatgcctgggatgatggctaccagattgtctataagctggagatgaggaagaaagaggaggaggtttgaggagctagccttgttttttgcatctttctcactcccatacatttatattatatccccactaaatttcttgttcctcattcttcaaatgtgggccagttgtggctcaaatcctctatatttttagccaatggcaatcaaattctttcagctcctttgtttcatacggaactccagatcctgagtaatccttttagagcccgaagagtcaaaaccctcaatgttccctcctgctctcctgccccatgtcaacaaatttcaggctaaggatgccccagacccagggctctaaccttgtatgcgggcaggcccagggagcaggcagcagtgttcttcccctcagagtgacttggggagggagaaataggaggagacgtccagctctgtcctctcttcctcactcctcccttcagtgtcctgaggaacaggactttctccacattgttttgtattgcaacattttgcattaaaaggaaaatccactgct

SEQ ID NO: 30 - DNA, 1719 bp, synthetic construct
  source 1..1719: mol_type = other DNA
agaagaacaaaagcatttggaagtaacaggacctctttctagctctcagaaaagtctgagaagaaaggagccctgcgttcccctaagctgtgcagcagatactgtgatgatggattgcaagtgcaaagagtaagacaaaactccagcacataaaggacaatgacaaccagaaagcttcagcccgatcctgccctttccttgaacgggactggatcctaggaggtgaagccatttccaattttttgtcctctgcctccctctgctgttcttctagagaagtttttccttacaacaatgagaaaacatgtactagctgcatccttttctatgctctccctgctggtgataatgggagatacagacagtaaaacggacagctcattcataatggactcggaccctcgacgctgcatgaggcaccactatgtggattctatcagtcacccattgtacaagtgtagctcaaagatggtgctcctggccaggtgcgaggggcactgcagccaggcgtcacgctccgagcctttggtgtcgttcagcactgtcctcaagcaacccttccgttcctcctgtcactgctgccggccccagacttccaagctgaaggcactgcggctgcgatgctcagggggcatgcgactcactgccacctaccggtacatcctctcctgtcactgcgaggaatgcaattcctgaggcccgctgctgtgtgtggcttctggatgggacaactgtagaggcagttcgaccagccagggaaagactggcaagaaaagagttaaggcaaaaaaggatgcaacaattctcccgggactctgcatattctagtaataaagactctacatgcttgttgacagagagagatactctgggaacttctttgcagttcccatctcctttctctggtacaatttcttttggttcattttcagattcaggcattttcccccttggctctcaatgctgtttgggtttccaacaattcagcattagtgggaaaaagtgggccctcatacacaagcgtgtcaggctgtcagtgtttggtgcacgctggggaagaatttactttggaaagtagaaaagcccagcttttcctgggacatcttctgttattgttgatgtttttttttaccttgtcattttggtctaaggttgccattgctgctaaaggttaccgatttcaaagtccagataccaagcatgtggatatgtttagctacgtttactcacagccagcgaactgacattaaaataactaacaaacagattcttttatgtgatgctggaactcttgacagctataattattattcagaaatgactttttgaaagtaaaagcagcataaagaatttgtcacaggaaggctgtctcagataaattatggtaaaattttgtaagggagcagacttttaaagacttgcacaaatacggatcctgcactgactctggaaaaggcatatatgtactagtggcatggagaatgcaccatactcatgcatgcaaattagacaaccaagtatgaatctatttgtgggtgtgctatagctttagccgtgtcacgggcatcattctctaatatccacttgtccatgtgaaacatgttgccaaaatggtggcctggcttgtcttctgaacgtttggttcaaatgtgttttggtcctggaggctcaaattttgagttattcccacgttttgaaataaaaagagtatattcaaaa

SEQ ID NO: 31 - DNA, 3748 bp, synthetic construct
  source 1..3748: mol_type = other DNA
agagctgcagtagcctggaggttcagagagccgggctactctgagaagaagacaccaagtggattctgcttcccctgggacagcactgagcgagtgtggagagaggtacagccctcggcctacaagctctttagtcttgaaagcgccacaagcagcagctgctgagccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaaagagatctgttctgggtgttgaccactccagagaagtttcgaggggtcctcacctggttgacccaaaaatgttcccttgaccattggctgcgctaacccccagcccacagagcctgaatttgtaagcaacttgcttctaaatgcccagttcacttctttgcagagccttttacccctgcacagtttagaacagagggaccaaattgcttctaggagtcaactggctggccagtctgggtctgggtttggatctccaattgcctcttgcaggctgagtccctccatgcaaaagtggggctaaatgaagtgtgttaaggggtcggctaagtgggacattagtaactgcacactatttccctctactgagtaaaccctatctgtgattcccccaaacatctggcatggctcccttttgtccttcctgtgccctgcaaatattagcaaagaagcttcatgccaggttaggaaggcagcattccatgaccagaaacagggacaaagaaatccccccttcagaacagaggcatttaaaatggaaaagagagattggattttggtgggtaacttagaaggatggcatctccatgtagaataaatgaagaaagggaggcccagccgcaggaaggcagaataaatccttgggagtcattaccacgccttgaccttcccaaggttactcagcagcagagagccctgggtgacttcaggtggagagcactagaagtggtttcctgataacaagcaaggatatcagagctgggaaattcatgtggatctggggactgagtgtgggagtgcagagaaagaaagggaaactggctgaggggataccataaaaagaggatgatttcagaaggagaaggaaaaagaaagtaatgccacacattgtgcttggcccctggtaagcagaggctttggggtcctagcccagtgcttctccaacactgaagtgcttgcagatcatctggggacctggtttgaatggagattctgattcagtgggttgggggcagagtttctgcagttccatcaggtcccccccaggtgcaggtgctgacaatactgctgccttacccgccatacattaaggagcagggtcctggtcctaaagagttattcaaatgaaggtggttcgacgccccgaacctcacctgacctcaactaacccttaaaaatgcacacctcatgagtctacctgagcattcaggcagcactgacaatagttatgcctgtactaaggagcatgattttaagaggctttggcccaatgcctataaaatgcccatttcgaagatatacaaaaacatacttcaaaaatgttaaacccttaccaacagcttttcccaggagaccatttgtattaccattacttgtataaatacacttcctgcttaaacttgacccaggtggctagcaaattagaaacaccattcatctctaacatatgatactgatgccatgtaaaggcctttaataagtcattgaaatttactgtgagactgtatgttttaattgcatttaaaaatatatagcttgaaagcagttaaactgattagtattcaggcactgagaatgatagtaataggatacaatgtataagctactcacttatctgatacttatttacctataaaatgagatttttgttttccactgtgctattacaaattttcttttgaaagtaggaactcttaagcaatggtaattgtgaataaaaattgatgagagtgttagctcctgtttcatatgaaattgaagtaattgttaactaaaaacaattccttagtaactgaactgtcatatttagaatggaaggaaaatgacagtttgtgaaagttcaaagcaatagtgcaattgaagaattgacctaagtaagctgacattatggttaataatagtattttagatttgtgcagcaaaataatttcataacttttttgtttttgttacttggataagatcaatctgttttattttagtaaatctttgcaggcaagttagagaaaatgcagtgtggcttaacgtctctttagtatgaagatttggccagaaaaagatacccagagaggaaatctaagataattataatggtccatactttttattgtatgaatcaaactcaagcataacattggccaaggaaaattaaataccattgctaacttgtgaaatggaagtctgtgatttcggagatgcaaagcattgtagtaaaaacaccaatgtgacctcgaccatctcagcccagatatcattcatatatctgttcaatgactattaaggtgcctactgtgtgctaggcactgtactggatactggggaccttgtctgtctggtttgctgctgtatcttctcccagggcattatatttatgatgaaagatgctgtggattcaattctttcagtcaagaataaacacagactttgtaggttcctgctgaataaagcaaatcccagaaacccagattttggaagaatcagcaaccccagcataaaataaacccctatcaaaatgtcagaggacatggcaaggtaaacttagcattttcaactttagaaccgggtcagcttcagggggactgctttcaaatcagccaaagagcctgtcagatcttcttagaaggaagaggttggtagttccctgctctgttttgaacatgctctagtttattaacctggggacattcccattgctgtcttaagtaagtctcatagccagctcctgtcacgtgactctcatatggattcattttcgggccagctctgaacaaagcatcatgaacatatgtgcttttggtcgtttgcaatgtgatggtggtggaggtaggtattggtttccttggaaggcatgataagaaagattcacaatggccaacagtgtgtatgaacaaaaaactgattggagcatcagctagtactgaaggtccttgctttgtgtcagaggcaaaggaacccaaggcgccaagtcctcagccttgagtgtactgctgacaactaaactcacaggctgcaaagcagacctctgatgaagatgcctgttatttcacatcactgtctttttgtgtatcatagtctgcaccttacaaatattaataaatgttccaataataggtga

SEQ ID NO: 32 - DNA, 3619 bp, synthetic construct
  source 1..3619: mol_type = other DNA
gtgggcataccagtgtcagctgcacttgtaggggcccaagtgcctcatgacccactcggcagccttcctctccaggatccccaaggctaggaggccaacctactaacagtcttgaaagcgccacaagcagcagctgctgagccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaaagagatctgttctgggtgttgaccactccagagaagtttcgaggggtcctcacctggttgacccaaaaatgttcccttgaccattggctgcgctaacccccagcccacagagcctgaatttgtaagcaacttgcttctaaatgcccagttcacttctttgcagagccttttacccctgcacagtttagaacagagggaccaaattgcttctaggagtcaactggctggccagtctgggtctgggtttggatctccaattgcctcttgcaggctgagtccctccatgcaaaagtggggctaaatgaagtgtgttaaggggtcggctaagtgggacattagtaactgcacactatttccctctactgagtaaaccctatctgtgattcccccaaacatctggcatggctcccttttgtccttcctgtgccctgcaaatattagcaaagaagcttcatgccaggttaggaaggcagcattccatgaccagaaacagggacaaagaaatccccccttcagaacagaggcatttaaaatggaaaagagagattggattttggtgggtaacttagaaggatggcatctccatgtagaataaatgaagaaagggaggcccagccgcaggaaggcagaataaatccttgggagtcattaccacgccttgaccttcccaaggttactcagcagcagagagccctgggtgacttcaggtggagagcactagaagtggtttcctgataacaagcaaggatatcagagctgggaaattcatgtggatctggggactgagtgtgggagtgcagagaaagaaagggaaactggctgaggggataccataaaaagaggatgatttcagaaggagaaggaaaaagaaagtaatgccacacattgtgcttggcccctggtaagcagaggctttggggtcctagcccagtgcttctccaacactgaagtgcttgcagatcatctggggacctggtttgaatggagattctgattcagtgggttgggggcagagtttctgcagttccatcaggtcccccccaggtgcaggtgctgacaatactgctgccttacccgccatacattaaggagcagggtcctggtcctaaagagttattcaaatgaaggtggttcgacgccccgaacctcacctgacctcaactaacccttaaaaatgcacacctcatgagtctacctgagcattcaggcagcactgacaatagttatgcctgtactaaggagcatgattttaagaggctttggcccaatgcctataaaatgcccatttcgaagatatacaaaaacatacttcaaaaatgttaaacccttaccaacagcttttcccaggagaccatttgtattaccattacttgtataaatacacttcctgcttaaacttgacccaggtggctagcaaattagaaacaccattcatctctaacatatgatactgatgccatgtaaaggcctttaataagtcattgaaatttactgtgagactgtatgttttaattgcatttaaaaatatatagcttgaaagcagttaaactgattagtattcaggcactgagaatgatagtaataggatacaatgtataagctactcacttatctgatacttatttacctataaaatgagatttttgttttccactgtgctattacaaattttcttttgaaagtaggaactcttaagcaatggtaattgtgaataaaaattgatgagagtgttagctcctgtttcatatgaaattgaagtaattgttaactaaaaacaattccttagtaactgaactgtcatatttagaatggaaggaaaatgacagtttgtgaaagttcaaagcaatagtgcaattgaagaattgacctaagtaagctgacattatggttaataatagtattttagatttgtgcagcaaaataatttcataacttttttgtttttgttacttggataagatcaatctgttttattttagtaaatctttgcaggcaagttagagaaaatgcagtgtggcttaacgtctctttagtatgaagatttggccagaaaaagatacccagagaggaaatctaagataattataatggtccatactttttattgtatgaatcaaactcaagcataacattggccaaggaaaattaaataccattgctaacttgtgaaatggaagtctgtgatttcggagatgcaaagcattgtagtaaaaacaccaatgtgacctcgaccatctcagcccagatatcattcatatatctgttcaatgactattaaggtgcctactgtgtgctaggcactgtactggatactggggaccttgtctgtctggtttgctgctgtatcttctcccagggcattatatttatgatgaaagatgctgtggattcaattctttcagtcaagaataaacacagactttgtaggttcctgctgaataaagcaaatcccagaaacccagattttggaagaatcagcaaccccagcataaaataaacccctatcaaaatgtcagaggacatggcaaggtaaacttagcattttcaactttagaaccgggtcagcttcagggggactgctttcaaatcagccaaagagcctgtcagatcttcttagaaggaagaggttggtagttccctgctctgttttgaacatgctctagtttattaacctggggacattcccattgctgtcttaagtaagtctcatagccagctcctgtcacgtgactctcatatggattcattttcgggccagctctgaacaaagcatcatgaacatatgtgcttttggtcgtttgcaatgtgatggtggtggaggtaggtattggtttccttggaaggcatgataagaaagattcacaatggccaacagtgtgtatgaacaaaaaactgattggagcatcagctagtactgaaggtccttgctttgtgtcagaggcaaaggaacccaaggcgccaagtcctcagccttgagtgtactgctgacaactaaactcacaggctgcaaagcagacctctgatgaagatgcctgttatttcacatcactgtctttttgtgtatcatagtctgcaccttacaaatattaataaatgttccaataataggtga

SEQ ID NO: 33 - DNA, 3616 bp, synthetic construct
  source 1..3616: mol_type = other DNA
gtgggcataccagtgtcagctgcacttgtaggggcccaagtgcctcatgacccactcggcagccttcctctccaggatccccaaggctaggaggccaacctactaacagtcttgaaagcgccacaagcagcagctgctgagccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacaacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaaagagatctgttctgggtgttgaccactccagagaagtttcgaggggtcctcacctggttgacccaaaaatgttcccttgaccattggctgcgctaacccccagcccacagagcctgaatttgtaagcaacttgcttctaaatgcccagttcacttctttgcagagccttttacccctgcacagtttagaacagagggaccaaattgcttctaggagtcaactggctggccagtctgggtctgggtttggatctccaattgcctcttgcaggctgagtccctccatgcaaaagtggggctaaatgaagtgtgttaaggggtcggctaagtgggacattagtaactgcacactatttccctctactgagtaaaccctatctgtgattcccccaaacatctggcatggctcccttttgtccttcctgtgccctgcaaatattagcaaagaagcttcatgccaggttaggaaggcagcattccatgaccagaaacagggacaaagaaatccccccttcagaacagaggcatttaaaatggaaaagagagattggattttggtgggtaacttagaaggatggcatctccatgtagaataaatgaagaaagggaggcccagccgcaggaaggcagaataaatccttgggagtcattaccacgccttgaccttcccaaggttactcagcagcagagagccctgggtgacttcaggtggagagcactagaagtggtttcctgataacaagcaaggatatcagagctgggaaattcatgtggatctggggactgagtgtgggagtgcagagaaagaaagggaaactggctgaggggataccataaaaagaggatgatttcagaaggagaaggaaaaagaaagtaatgccacacattgtgcttggcccctggtaagcagaggctttggggtcctagcccagtgcttctccaacactgaagtgcttgcagatcatctggggacctggtttgaatggagattctgattcagtgggttgggggcagagtttctgcagttccatcaggtcccccccaggtgcaggtgctgacaatactgctgccttacccgccatacattaaggagcagggtcctggtcctaaagagttattcaaatgaaggtggttcgacgccccgaacctcacctgacctcaactaacccttaaaaatgcacacctcatgagtctacctgagcattcaggcagcactgacaatagttatgcctgtactaaggagcatgattttaagaggctttggcccaatgcctataaaatgcccatttcgaagatatacaaaaacatacttcaaaaatgttaaacccttaccaacagcttttcccaggagaccatttgtattaccattacttgtataaatacacttcctgcttaaacttgacccaggtggctagcaaattagaaacaccattcatctctaacatatgatactgatgccatgtaaaggcctttaataagtcattgaaatttactgtgagactgtatgttttaattgcatttaaaaatatatagcttgaaagcagttaaactgattagtattcaggcactgagaatgatagtaataggatacaatgtataagctactcacttatctgatacttatttacctataaaatgagatttttgttttccactgtgctattacaaattttcttttgaaagtaggaactcttaagcaatggtaattgtgaataaaaattgatgagagtgttagctcctgtttcatatgaaattgaagtaattgttaactaaaaacaattccttagtaactgaactgtcatatttagaatggaaggaaaatgacagtttgtgaaagttcaaagcaatagtgcaattgaagaattgacctaagtaagctgacattatggttaataatagtattttagatttgtgcagcaaaataatttcataacttttttgtttttgttacttggataagatcaatctgttttattttagtaaatctttgcaggcaagttagagaaaatgcagtgtggcttaacgtctctttagtatgaagatttggccagaaaaagatacccagagaggaaatctaagataattataatggtccatactttttattgtatgaatcaaactcaagcataacattggccaaggaaaattaaataccattgctaacttgtgaaatggaagtctgtgatttcggagatgcaaagcattgtagtaaaaacaccaatgtgacctcgaccatctcagcccagatatcattcatatatctgttcaatgactattaaggtgcctactgtgtgctaggcactgtactggatactggggaccttgtctgtctggtttgctgctgtatcttctcccagggcattatatttatgatgaaagatgctgtggattcaattctttcagtcaagaataaacacagactttgtaggttcctgctgaataaagcaaatcccagaaacccagattttggaagaatcagcaaccccagcataaaataaacccctatcaaaatgtcagaggacatggcaaggtaaacttagcattttcaactttagaaccgggtcagcttcagggggactgctttcaaatcagccaaagagcctgtcagatcttcttagaaggaagaggttggtagttccctgctctgttttgaacatgctctagtttattaacctggggacattcccattgctgtcttaagtaagtctcatagccagctcctgtcacgtgactctcatatggattcattttcgggccagctctgaacaaagcatcatgaacatatgtgcttttggtcgtttgcaatgtgatggtggtggaggtaggtattggtttccttggaaggcatgataagaaagattcacaatggccaacagtgtgtatgaacaaaaaactgattggagcatcagctagtactgaaggtccttgctttgtgtcagaggcaaaggaacccaaggcgccaagtcctcagccttgagtgtactgctgacaactaaactcacaggctgcaaagcagacctctgatgaagatgcctgttatttcacatcactgtctttttgtgtatcatagtctgcaccttacaaatattaataaatgttccaataataggtga

SEQ ID NO: 34 - DNA, 3941 bp, synthetic construct
  source 1..3941: mol_type = other DNA
gtgggcataccagtgtcagctgcacttgtaggggcccaagtgcctcatgacccactcggcagccttcctctccaggatccccaaggctaggaggccaacctactaacagcctgcctgcagctgtcctggtagaacagtgtggacattgcagaagctgtcactgccccagaaagaaagcaccccagagccaaggcaaagaggtgattccagtgcacactcgagcttgagaaccactggtctcggtactgagtatataacagtgagaagacaaagtcctgctctccttgtggaatttgtattccagtgtatgtggtaggggagacagaacaatcttgaaagcgccacaagcagcagctgctgagccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcaacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaaagagatctgttctgggtgttgaccactccagagaagtttcgaggggtcctcacctggttgacccaaaaatgttcccttgaccattggctgcgctaacccccagcccacagagcctgaatttgtaagcaacttgcttctaaatgcccagttcacttctttgcagagccttttacccctgcacagtttagaacagagggaccaaattgcttctaggagtcaactggctggccagtctgggtctgggtttggatctccaattgcctcttgcaggctgagtccctccatgcaaaagtggggctaaatgaagtgtgttaaggggtcggctaagtgggacattagtaactgcacactatttccctctactgagtaaaccctatctgtgattcccccaaacatctggcatggctcccttttgtccttcctgtgccctgcaaatattagcaaagaagcttcatgccaggttaggaaggcagcattccatgaccagaaacagggacaaagaaatccccccttcagaacagaggcatttaaaatggaaaagagagattggattttggtgggtaacttagaaggatggcatctccatgtagaataaatgaagaaagggaggcccagccgcaggaaggcagaataaatccttgggagtcattaccacgccttgaccttcccaaggttactcagcagcagagagccctgggtgacttcaggtggagagcactagaagtggtttcctgataacaagcaaggatatcagagctgggaaattcatgtggatctggggactgagtgtgggagtgcagagaaagaaagggaaactggctgaggggataccataaaaagaggatgatttcagaaggagaaggaaaaagaaagtaatgccacacattgtgcttggcccctggtaagcagaggctttggggtcctagcccagtgcttctccaacactgaagtgcttgcagatcatctggggacctggtttgaatggagattctgattcagtgggttgggggcagagtttctgcagttccatcaggtcccccccaggtgcaggtgctgacaatactgctgccttacccgccatacattaaggagcagggtcctggtcctaaagagttattcaaatgaaggtggttcgacgccccgaacctcacctgacctcaactaacccttaaaaatgcacacctcatgagtctacctgagcattcaggcagcactgacaatagttatgcctgtactaaggagcatgattttaagaggctttggcccaatgcctataaaatgcccatttcgaagatatacaaaaacatacttcaaaaatgttaaacccttaccaacagcttttcccaggagaccatttgtattaccattacttgtataaatacacttcctgcttaaacttgacccaggtggctagcaaattagaaacaccattcatctctaacatatgatactgatgccatgtaaaggcctttaataagtcattgaaatttactgtgagactgtatgttttaattgcatttaaaaatatatagcttgaaagcagttaaactgattagtattcaggcactgagaatgatagtaataggatacaatgtataagctactcacttatctgatacttatttacctataaaatgagatttttgttttccactgtgctattacaaattttcttttgaaagtaggaactcttaagcaatggtaattgtgaataaaaattgatgagagtgttagctcctgtttcatatgaaattgaagtaattgttaactaaaaacaattccttagtaactgaactgtcatatttagaatggaaggaaaatgacagtttgtgaaagttcaaagcaatagtgcaattgaagaattgacctaagtaagctgacattatggttaataatagtattttagatttgtgcagcaaaataatttcataacttttttgtttttgttacttggataagatcaatctgttttattttagtaaatctttgcaggcaagttagagaaaatgcagtgtggcttaacgtctctttagtatgaagatttggccagaaaaagatacccagagaggaaatctaagataattataatggtccatactttttattgtatgaatcaaactcaagcataacattggccaaggaaaattaaataccattgctaacttgtgaaatggaagtctgtgatttcggagatgcaaagcattgtagtaaaaacaccaatgtgacctcgaccatctcagcccagatatcattcatatatctgttcaatgactattaaggtgcctactgtgtgctaggcactgtactggatactggggaccttgtctgtctggtttgctgctgtatcttctcccagggcattatatttatgatgaaagatgctgtggattcaattctttcagtcaagaataaacacagactttgtaggttcctgctgaataaagcaaatcccagaaacccagattttggaagaatcagcaaccccagcataaaataaacccctatcaaaatgtcagaggacatggcaaggtaaacttagcattttcaactttagaaccgggtcagcttcagggggactgctttcaaatcagccaaagagcctgtcagatcttcttagaaggaagaggttggtagttccctgctctgttttgaacatgctctagtttattaacctggggacattcccattgctgtcttaagtaagtctcatagccagctcctgtcacgtgactctcatatggattcattttcgggccagctctgaacaaagcatcatgaacatatgtgcttttggtcgtttgcaatgtgatggtggtggaggtaggtattggtttccttggaaggcatgataagaaagattcacaatggccaacagtgtgtatgaacaaaaaactgattggagcatcagctagtactgaaggtccttgctttgtgtcagaggcaaaggaacccaaggcgccaagtcctcagccttgagtgtactgctgacaactaaactcacaggctgcaaagcagacctctgatgaagatgcctgttatttcacatcactgtctttttgtgtatcatagtctgcaccttacaaatattaataaatgttccaataataggtga

SEQ ID NO: 35 - DNA, 4995 bp, synthetic construct
  source 1..4995: mol_type = other DNA
gctgactctcctcggcggggaccgcggcgccggccggagcgaggagctgcgcacgcagcgggtcccggcgccctccccgcacacaaaggcccgcgcgcgtccggagcccgcggcggggaccgagctccctctttcctgccctgggggccgggagccgcgcggactgagaaggctcctgcgcgcccggaggcgccctactccgctccgtgctccgggacatggaaccgcgccgacgcggcgcccgcgcgctcgggccgctgcccgctgcactggatctatagtcacaggcggccccgctcggggcgcagcgcccgccgcccgcgccggtcgtctccgctccgggtctttgtgtcccagcccgcacccgccccgcgcccacccgccagccgcggccggcccggcaccctggagccgcacgggagtcggccgtccgagctgcgtccggcgcggcgccccggaaccccgagtccgccgcgccgccgcgccccgcgcgtgcgctcccgccccgcgccctgagggccccggagagcgagcgcacctggcccggcgaccgctggagctgcgcagcgcgcctcggagctgcctgcgggcgcacgccgtcttccccgccagtctgccccggaggattgggggtcccagcctgcgtcccgtcagtcccttcttggcccggagtgcgcggagctgggagtggcttcgccatggctgtgagaagggactccgtgtggaagtactgctggggtgttttgatggttttatgcagaactgcgatttccaaatcgatagttttagagcctatctattggaattcctcgaactccaaatttctacctggacaaggactggtactatacccacagataggagacaaattggatattatttgccccaaagtggactctaaaactgttggccagtatgaatattataaagtttatatggttgataaagaccaagcagacagatgcactattaagaaggaaaatacccctctcctcaactgtgccaaaccagaccaagatatcaaattcaccatcaagtttcaagaattcagccctaacctctggggtctagaatttcagaagaacaaagattattacattatatctacatcaaatgggtctttggagggcctggataaccaggagggaggggtgtgccagacaagagccatgaagatcctcatgaaagttggacaagatgcaagttctgctggatcaaccaggaataaagatccaacaagacgtccagaactagaagctggtacaaatggaagaagttcgacaacaagtccctttgtaaaaccaaatccaggttctagcacagacggcaacagcgccggacattcggggaacaacatcctcggttccgaagtggccttatttgcagggattgcttcaggatgcatcatcttcatcgtcatcatcatcacgctggtggtcctcttgctgaagtaccggaggagacacaggaagcactcgccgcagcacacgaccacgctgtcgctcagcacactggccacacccaagcgcagcggcaacaacaacggctcagagcccagtgacattatcatcccgctaaggactgcggacagcgtcttctgccctcactacgagaaggtcagcggggactacgggcacccggtgtacatcgtccaggagatgcccccgcagagcccggcgaacatttactacaaggtctgagagggaccctggtggtacctgtgctttcccagaggacacctaatgtcccgatgcctcccttgagggtttgagagcccgcgtgctggagaattgactgaagcacagcaccgggggagagggacactcctcctcggaagagcccgtcgcgctggacagcttacctagtcttgtagcattcggccttggtgaacacacacgctccctggaagctggaagactgtgcagaagacgcccattcggactgctgtgccgcgtcccacgtctcctcctcgaagccatgtgctgcggtcactcaggcctctgcagaagccaagggaagacagtggtttgtggacgagagggctgtgagcatcctggcaggtgccccaggatgccacgcctggaagggccggcttctgcctggggtgcatttcccccgcagtgcataccggacttgtcacacggacctcgggctagttaaggtgtgcaaagatctctagagtttagtccttactgtctcactcgttctgttacccagggctctgcagcacctcacctgagacctccactccacatctgcatcactcatggaacactcatgtctggagtcccctcctccagccgctggcaacaacagcttcagtccatgggtaatccgttcatagaaattgtgtttgctaacaaggtgccctttagccagatgctaggctgtctgcgaagaaggctaggagttcatagaagggagtggggctggggaaagggctggctgcaattgcagctcactgctgctgcctctgaaacagaaagttggaaaggaaaaaagaaaaaagcaattaggtagcacagcactttggttttgctgagatcgaagaggccagtaggagacacgacagcacacacagtggattccagtgcatggggaggcactcgctgttatcaaatagcgatgtgcaggaagaaaagcccctcttcattccggggaacaaagacgggtattgttgggaaaggaacaggcttggagggaagggagaaagtaggccgctgatgatatattcgggcaggactgttgtggtactggcaataagatacacagctccgagctgtaggagagtcggtctgctttggatgattttttaagcagactcagctgctatacttatcacattttattaaacacagggaaagcatttaggagaatagcagagagccaaatctgacctaaaagttgaaaagccaaaggtcaaacaggctgtaattccatcatcatcgttgttattaaagaatccttatctataaaaggtaggtcagatccccctccccccaggttcctccttcccctcccgattgagccttacgacactttggtttatgcggtgctgtccgggtgccagggctgcagggtcggtactgatggaggctgcagcgcccggtgctctgtgtcaaggtgaagcacatacggcagacctcttagagtccttaagacggaagtaaattatgatgtccagggggagaaggaagataggacgtatttataataggtatatagaacacaagggatataaaatgaaagatttttactaatatatattttaaggttgcacacagtacacaccagaagatgtgaaattcatttgtggcaattaagtggtcccaatgctcagcgcttaaaaaaacaaattggacagctacttctgggaaaaacaacatcattccaaaaagaacaataatgagagcaaatgcaaaaataaccaagtcctccgaaggcatctcacggaaccgtagactaggaagtacgagccccacagagcaggaagccgatgtgactgcatcatatatttaacaatgacaagatgttccggcgtttatttctgcgttgggttttcccttgccttatgggctgaagtgttctctagaatccagcaggtcacactgggggcttcaggtgacgatttagctgtggctccctcctcctgtcctcccccgcaccccctcccttctgggaaacaagaagagtaaacaggaaacctactttttatgtgctatgcaaaatagacatctttaacatagtcctgttactatggtaacactttgctttctgaattggaagggaaaaaaaatgtagcgacagcattttaaggttctcagacctccagtgagtacctgcaaaaatgagttgtcacagaaattatgatcctctatttcctgaacctggaaatgatgttggtccaaagtgcgtgtgtgtatgtgtgagtgggtgcgtggtatacatgtgtacatatatgtataatatatatctacaatatatattatatatatctatatcatatttctgtggagggttgccatggtaaccagccacagtacatatgtaattctttccatcaccccaacctctcctttctgtgcattcatgcaagagtttcttgtaagccatcagaagttacttttaggatgggggagaggggcgagaaggggaaaaatgggaaatagtctgattttaatgaaatcaaatgtatgtatcatcagttggctacgttttggttctatgctaaactgtgaaaaatcagatgaattgataaaagagttccctgcaaccaattgaaaagtgttctgtgcgtctgttttgtgtctggtgcagaatatgacaatctaccaactgtccctttgtttgaagttggtttagctttggaaagttactgtaaatgccttgcttgtatgatcgtccctggtcacccgactttggaatttgcaccatcatgtttcagtgaagatgctgtaaataggttcagattttactgtctatggatttggggtgttacagtagccttattcacctttttaataaaaatacacatgaaaacaagaaagaaatggcttttcttacccagattgtgtacatagagcaatgttggttttttataaagtctaagcaagatgttttgtataaaatctgaattttgcaatgtatttagctacagcttgtttaacggcagtgtcattcccctttgcactgtaatgaggaaaaaatggtataaaaggttgccaaattgctgcatatttgtgccgtaattatgtaccatgaatatttatttaaaatttcgttgtccaatttgtaagtaacacagtattatgcctgagttataaatatttttttctttctttgttttattttaatagcctgtcataggttttaaatctgctttagtttcacattgcagttagccccagaaaatgaaatccgtgaagtcacattccacatctgtttcaaactgaatttgttcttaaaaaaataaaatatttttttcctatggaaaaa

SEQ ID NO: 36 - DNA, 4902 bp, synthetic construct
  source 1..4902: mol_type = other DNA
gctgactctcctcggcggggaccgcggcgccggccggagcgaggagctgcgcacgcagcgggtcccggcgccctccccgcacacaaaggcccgcgcgcgtccggagcccgcggcggggaccgagctccctctttcctgccctgggggccgggagccgcgcggactgagaaggctcctgcgcgcccggaggcgccctactccgctccgtgctccgggacatggaaccgcgccgacgcggcgcccgcgcgctcgggccgctgcccgctgcactggatctatagtcacaggcggccccgctcggggcgcagcgcccgccgcccgcgccggtcgtctccgctccgggtctttgtgtcccagcccgcacccgccccgcgcccacccgccagccgcggccggcccggcaccctggagccgcacgggagtcggccgtccgagctgcgtccggcgcggcgccccggaaccccgagtccgccgcgccgccgcgccccgcgcgtgcgctcccgccccgcgccctgagggccccggagagcgagcgcacctggcccggcgaccgctggagctgcgcagcgcgcctcggagctgcctgcgggcgcacgccgtcttccccgccagtctgccccggaggattgggggtcccagcctgcgtcccgtcagtcccttcttggcccggagtgcgcggagctgggagtggcttcgccatggctgtgagaagggactccgtgtggaagtactgctggggtgttttgatggttttatgcagaactgcgatttccaaatcgatagttttagagcctatctattggaattcctcgaactccaaatttctacctggacaaggactggtactatacccacagataggagacaaattggatattatttgccccaaagtggactctaaaactgttggccagtatgaatattataaagtttatatggttgataaagaccaagcagacagatgcactattaagaaggaaaatacccctctcctcaactgtgccaaaccagaccaagatatcaaattcaccatcaagtttcaagaattcagccctaacctctggggtctagaatttcagaagaacaaagattattacattatatatgcaagttctgctggatcaaccaggaataaagatccaacaagacgtccagaactagaagctggtacaaatggaagaagttcgacaacaagtccctttgtaaaaccaaatccaggttctagcacagacggcaacagcgccggacattcggggaacaacatcctcggttccgaagtggccttatttgcagggattgcttcaggatgcatcatcttcatcgtcatcatcatcacgctggtggtcctcttgctgaagtaccggaggagacacaggaagcactcgccgcagcacacgaccacgctgtcgctcagcacactggccacacccaagcgcagcggcaacaacaacggctcagagcccagtgacattatcatcccgctaaggactgcggacagcgtcttctgccctcactacgagaaggtcagcggggactacgggcacccggtgtacatcgtccaggagatgcccccgcagagcccggcgaacatttactacaaggtctgagagggaccctggtggtacctgtgctttcccagaggacacctaatgtcccgatgcctcccttgagggtttgagagcccgcgtgctggagaattgactgaagcacagcaccgggggagagggacactcctcctcggaagagcccgtcgcgctggacagcttacctagtcttgtagcattcggccttggtgaacacacacgctccctggaagctggaagactgtgcagaagacgcccattcggactgctgtgccgcgtcccacgtctcctcctcgaagccatgtgctgcggtcactcaggcctctgcagaagccaagggaagacagtggtttgtggacgagagggctgtgagcatcctggcaggtgccccaggatgccacgcctggaagggccggcttctgcctggggtgcatttcccccgcagtgcataccggacttgtcacacggacctcgggctagttaaggtgtgcaaagatctctagagtttagtccttactgtctcactcgttctgttacccagggctctgcagcacctcacctgagacctccactccacatctgcatcactcatggaacactcatgtctggagtcccctcctccagccgctggcaacaacagcttcagtccatgggtaatccgttcatagaaattgtgtttgctaacaaggtgccctttagccagatgctaggctgtctgcgaagaaggctaggagttcatagaagggagtggggctggggaaagggctggctgcaattgcagctcactgctgctgcctctgaaacagaaagttggaaaggaaaaaagaaaaaagcaattaggtagcacagcactttggttttgctgagatcgaagaggccagtaggagacacgacagcacacacagtggattccagtgcatggggaggcactcgctgttatcaaatagcgatgtgcaggaagaaaagcccctcttcattccggggaacaaagacgggtattgttgggaaaggaacaggcttggagggaagggagaaagtaggccgctgatgatatattcgggcaggactgttgtggtactggcaataagatacacagctccgagctgtaggagagtcggtctgctttggatgattttttaagcagactcagctgctatacttatcacattttattaaacacagggaaagcatttaggagaatagcagagagccaaatctgacctaaaagttgaaaagccaaaggtcaaacaggctgtaattccatcatcatcgttgttattaaagaatccttatctataaaaggtaggtcagatccccctccccccaggttcctccttcccctcccgattgagccttacgacactttggtttatgcggtgctgtccgggtgccagggctgcagggtcggtactgatggaggctgcagcgcccggtgctctgtgtcaaggtgaagcacatacggcagacctcttagagtccttaagacggaagtaaattatgatgtccagggggagaaggaagataggacgtatttataataggtatatagaacacaagggatataaaatgaaagatttttactaatatatattttaaggttgcacacagtacacaccagaagatgtgaaattcatttgtggcaattaagtggtcccaatgctcagcgcttaaaaaaacaaattggacagctacttctgggaaaaacaacatcattccaaaaagaacaataatgagagcaaatgcaaaaataaccaagtcctccgaaggcatctcacggaaccgtagactaggaagtacgagccccacagagcaggaagccgatgtgactgcatcatatatttaacaatgacaagatgttccggcgtttatttctgcgttgggttttcccttgccttatgggctgaagtgttctctagaatccagcaggtcacactgggggcttcaggtgacgatttagctgtggctccctcctcctgtcctcccccgcaccccctcccttctgggaaacaagaagagtaaacaggaaacctactttttatgtgctatgcaaaatagacatctttaacatagtcctgttactatggtaacactttgctttctgaattggaagggaaaaaaaatgtagcgacagcattttaaggttctcagacctccagtgagtacctgcaaaaatgagttgtcacagaaattatgatcctctatttcctgaacctggaaatgatgttggtccaaagtgcgtgtgtgtatgtgtgagtgggtgcgtggtatacatgtgtacatatatgtataatatatatctacaatatatattatatatatctatatcatatttctgtggagggttgccatggtaaccagccacagtacatatgtaattctttccatcaccccaacctctcctttctgtgcattcatgcaagagtttcttgtaagccatcagaagttacttttaggatgggggagaggggcgagaaggggaaaaatgggaaatagtctgattttaatgaaatcaaatgtatgtatcatcagttggctacgttttggttctatgctaaactgtgaaaaatcagatgaattgataaaagagttccctgcaaccaattgaaaagtgttctgtgcgtctgttttgtgtctggtgcagaatatgacaatctaccaactgtccctttgtttgaagttggtttagctttggaaagttactgtaaatgccttgcttgtatgatcgtccctggtcacccgactttggaatttgcaccatcatgtttcagtgaagatgctgtaaataggttcagattttactgtctatggatttggggtgttacagtagccttattcacctttttaataaaaatacacatgaaaacaagaaagaaatggcttttcttacccagattgtgtacatagagcaatgttggttttttataaagtctaagcaagatgttttgtataaaatctgaattttgcaatgtatttagctacagcttgtttaacggcagtgtcattcccctttgcactgtaatgaggaaaaaatggtataaaaggttgccaaattgctgcatatttgtgccgtaattatgtaccatgaatatttatttaaaatttcgttgtccaatttgtaagtaacacagtattatgcctgagttataaatatttttttctttctttgttttattttaatagcctgtcataggttttaaatctgctttagtttcacattgcagttagccccagaaaatgaaatccgtgaagtcacattccacatctgtttcaaactgaatttgttcttaaaaaaataaaatatttttttcctatggaaaaa

SEQ ID NO: 37 - DNA, 4881 bp, synthetic construct
  source 1..4881: mol_type = other DNA
gctgactctcctcggcggggaccgcggcgccggccggagcgaggagctgcgcacgcagcgggtcccggcgccctccccgcacacaaaggcccgcgcgcgtccggagcccgcggcggggaccgagctccctctttcctgccctgggggccgggagccgcgcggactgagaaggctcctgcgcgcccggaggcgccctactccgctccgtgctccgggacatggaaccgcgccgacgcggcgcccgcgcgctcgggccgctgcccgctgcactggatctatagtcacaggcggccccgctcggggcgcagcgcccgccgcccgcgccggtcgtctccgctccgggtctttgtgtcccagcccgcacccgccccgcgcccacccgccagccgcggccggcccggcaccctggagccgcacgggagtcggccgtccgagctgcgtccggcgcggcgccccggaaccccgagtccgccgcgccgccgcgccccgcgcgtgcgctcccgccccgcgccctgagggccccggagagcgagcgcacctggcccggcgaccgctggagctgcgcagcgcgcctcggagctgcctgcgggcgcacgccgtcttccccgccagtctgccccggaggattgggggtcccagcctgcgtcccgtcagtcccttcttggcccggagtgcgcggagctgggagtggcttcgccatggctgtgagaagggactccgtgtggaagtactgctggggtgttttgatggttttatgcagaactgcgatttccaaatcgatagttttagagcctatctattggaattcctcgaactccaaatttctacctggacaaggactggtactatacccacagataggagacaaattggatattatttgccccaaagtggactctaaaactgttggccagtatgaatattataaagtttatatggttgataaagaccaagcagacagatgcactattaagaaggaaaatacccctctcctcaactgtgccaaaccagaccaagatatcaaattcaccatcaagtttcaagaattcagccctaacctctggggtctagaatttcagaagaacaaagattattacattatatctacatcaaatgggtctttggagggcctggataaccaggagggaggggtgtgccagacaagagccatgaagatcctcatgaaagttggacaaggttctagcacagacggcaacagcgccggacattcggggaacaacatcctcggttccgaagtggccttatttgcagggattgcttcaggatgcatcatcttcatcgtcatcatcatcacgctggtggtcctcttgctgaagtaccggaggagacacaggaagcactcgccgcagcacacgaccacgctgtcgctcagcacactggccacacccaagcgcagcggcaacaacaacggctcagagcccagtgacattatcatcccgctaaggactgcggacagcgtcttctgccctcactacgagaaggtcagcggggactacgggcacccggtgtacatcgtccaggagatgcccccgcagagcccggcgaacatttactacaaggtctgagagggaccctggtggtacctgtgctttcccagaggacacctaatgtcccgatgcctcccttgagggtttgagagcccgcgtgctggagaattgactgaagcacagcaccgggggagagggacactcctcctcggaagagcccgtcgcgctggacagcttacctagtcttgtagcattcggccttggtgaacacacacgctccctggaagctggaagactgtgcagaagacgcccattcggactgctgtgccgcgtcccacgtctcctcctcgaagccatgtgctgcggtcactcaggcctctgcagaagccaagggaagacagtggtttgtggacgagagggctgtgagcatcctggcaggtgccccaggatgccacgcctggaagggccggcttctgcctggggtgcatttcccccgcagtgcataccggacttgtcacacggacctcgggctagttaaggtgtgcaaagatctctagagtttagtccttactgtctcactcgttctgttacccagggctctgcagcacctcacctgagacctccactccacatctgcatcactcatggaacactcatgtctggagtcccctcctccagccgctggcaacaacagcttcagtccatgggtaatccgttcatagaaattgtgtttgctaacaaggtgccctttagccagatgctaggctgtctgcgaagaaggctaggagttcatagaagggagtggggctggggaaagggctggctgcaattgcagctcactgctgctgcctctgaaacagaaagttggaaaggaaaaaagaaaaaagcaattaggtagcacagcactttggttttgctgagatcgaagaggccagtaggagacacgacagcacacacagtggattccagtgcatggggaggcactcgctgttatcaaatagcgatgtgcaggaagaaaagcccctcttcattccggggaacaaagacgggtattgttgggaaaggaacaggcttggagggaagggagaaagtaggccgctgatgatatattcgggcaggactgttgtggtactggcaataagatacacagctccgagctgtaggagagtcggtctgctttggatgattttttaagcagactcagctgctatacttatcacattttattaaacacagggaaagcatttaggagaatagcagagagccaaatctgacctaaaagttgaaaagccaaaggtcaaacaggctgtaattccatcatcatcgttgttattaaagaatccttatctataaaaggtaggtcagatccccctccccccaggttcctccttcccctcccgattgagccttacgacactttggtttatgcggtgctgtccgggtgccagggctgcagggtcggtactgatggaggctgcagcgcccggtgctctgtgtcaaggtgaagcacatacggcagacctcttagagtccttaagacggaagtaaattatgatgtccagggggagaaggaagataggacgtatttataataggtatatagaacacaagggatataaaatgaaagatttttactaatatatattttaaggttgcacacagtacacaccagaagatgtgaaattcatttgtggcaattaagtggtcccaatgctcagcgcttaaaaaaacaaattggacagctacttctgggaaaaacaacatcattccaaaaagaacaataatgagagcaaatgcaaaaataaccaagtcctccgaaggcatctcacggaaccgtagactaggaagtacgagccccacagagcaggaagccgatgtgactgcatcatatatttaacaatgacaagatgttccggcgtttatttctgcgttgggttttcccttgccttatgggctgaagtgttctctagaatccagcaggtcacactgggggcttcaggtgacgatttagctgtggctccctcctcctgtcctcccccgcaccccctcccttctgggaaacaagaagagtaaacaggaaacctactttttatgtgctatgcaaaatagacatctttaacatagtcctgttactatggtaacactttgctttctgaattggaagggaaaaaaaatgtagcgacagcattttaaggttctcagacctccagtgagtacctgcaaaaatgagttgtcacagaaattatgatcctctatttcctgaacctggaaatgatgttggtccaaagtgcgtgtgtgtatgtgtgagtgggtgcgtggtatacatgtgtacatatatgtataatatatatctacaatatatattatatatatctatatcatatttctgtggagggttgccatggtaaccagccacagtacatatgtaattctttccatcaccccaacctctcctttctgtgcattcatgcaagagtttcttgtaagccatcagaagttacttttaggatgggggagaggggcgagaaggggaaaaatgggaaatagtctgattttaatgaaatcaaatgtatgtatcatcagttggctacgttttggttctatgctaaactgtgaaaaatcagatgaattgataaaagagttccctgcaaccaattgaaaagtgttctgtgcgtctgttttgtgtctggtgcagaatatgacaatctaccaactgtccctttgtttgaagttggtttagctttggaaagttactgtaaatgccttgcttgtatgatcgtccctggtcacccgactttggaatttgcaccatcatgtttcagtgaagatgctgtaaataggttcagattttactgtctatggatttggggtgttacagtagccttattcacctttttaataaaaatacacatgaaaacaagaaagaaatggcttttcttacccagattgtgtacatagagcaatgttggttttttataaagtctaagcaagatgttttgtataaaatctgaattttgcaatgtatttagctacagcttgtttaacggcagtgtcattcccctttgcactgtaatgaggaaaaaatggtataaaaggttgccaaattgctgcatatttgtgccgtaattatgtaccatgaatatttatttaaaatttcgttgtccaatttgtaagtaacacagtattatgcctgagttataaatatttttttctttctttgttttattttaatagcctgtcataggttttaaatctgctttagtttcacattgcagttagccccagaaaatgaaatccgtgaagtcacattccacatctgtttcaaactgaatttgttcttaaaaaaataaaatatttttttcctatggaaaaa

SEQ ID NO: 38 - DNA, 17410 bp, synthetic construct
  source 1..17410: mol_type = other DNA
attcagcagagaacagtaatgcctagcttccgtttttaacttaacacttcagtagaacattttcttccaagagggagattttggcctaagtaaagtagtgggctcttttttaaaaaaaaattaattttactttaatgtgagcaaatctgtattggtatggtgttctgcaatgcattacactgactttgaaaatttcgagtactaatgccttatgtctggggttaccattccctgtgcatcacatactagttagttaacatagcattttgcttttcccatgtaattttttccctatataatactggattcctgatactaattgacttgatacaaaagaatggctggatgatatccagataacgtataatacatgggcttcaccacaatcaggctctgaataaatacagacctgtcagagattgataaaataaactacaatggatagtgctgtttaaacagtccattcaataacatatataagccagcctgccttccattgtgtctgaaattcttatttttgtaggtaaacaaatgcacattcagcactgattgaatagccccttgaactatgctccacagtttgcgtttgggttaatcttgtcggttttaatatagagagaaaaaagctcaaagcaccaggggtggaattgttagtgctttcacatccacattcctcacattttgtcaggatgataaactgtaggtaatggactgtcgttgttctgcaggacaactgagccaggcagagcacaaagactaagctaaagcgatacctcacaacatgcttggtagccttcttttcagatgagaatttatttgagaatcatgtgtctagggactgcacatcttaacctcaacagttacagcttcaagccccagaaacaggagctggaggttaagatgatttgctaagcacctggttctaaatcttttacaaagcataagctgttgacgctggttctgccgacgcaaagacatgcagatgactccaacatttccagaggcttctgacttaagctaaagtgtgtggacaggtgaattcgccatgggcctggagaccagcttgctaaaaactatgtgtttgaatggttcctccagacagagtcagctgaagaacaattggtggatttatattaaaacctcttgtctgtaaacttactgaggtgcatccttcggttggtggatcagtgagataattgccttcagatggacattgcaactggagcaactaaatccttgctgtctttccttcctctgaaatcttccaggtagctcccgagagcttcagtatgacaccaaacttcgggcgacgttttagagtgcgttcacctaatgggaaactattcgagatcccagcgtgactgcagtaatgcgtcataggaatgggagtggcaggggaaaaggaaatacagattgtagaccctaataaaaaaatttttaggaaagatatttctttaacgttttatgagaacttcattcttaaaatacttaattgcaaattagacaaatagaagtgctcttctaaggaaggtgattaaactggtcctcctatcagcctaatctctgcctgcctttgctgctgacataaagaacctgtttttcaggtcacttaatatacatctacatagatttgcttatgagctcaccctttgtgtagcggagtagagccttaaagaggagtgctcaactgtttaaaatattttgattaaaatatgcagaacccatagaactataagcttctagtcaggaattagctctttcagggaacagctccccccttctttttaaggggggaattagaaggaggctgggggaggaatataagaacagcaaagaaggaaggatagcaaatgggacatgttccgaacagcttggaaaaactcctgtggcttcattgtctctataaagccaaagaatacaaagacataagcaattcagcccttctcccatgatggaagatgtaaaccgttgacatgcctcccctgtttaacttgtttaattctcattttaaattcagcacgatactagccgtgtgaactctgaagatttctttagtaatccattttgtagttccgaatcaaaaacaaagtgaaagggtctgacacaatttgcttttatttttaggcaaatcaaccctggtcatagttaataaggggattacaactcagactaggtctttacagatgtgatgtaaatcaagggcagagtataaagaaactgatcccttttgattgaagtatagtaaaaaggcatagagaaactagcagcagtaatctgattgtatggcaataaaaccaccattttctgtctttcagataaaaataatgtggtaaatccatgcagttcataagatgtaaaggcagataaagggtgaagccatggcaacatatagattagcttgatgttagaaatgacacgtctctgaaaagggcgcgggacgaaggcccttgcctccaggctgttgggcattatgtgagaaccacacagacttggaaactgggattaggaagtatgaaagctctacttgtggtctgggatggctgaggcagtaaagaaaagctgctcagttcttgctcattggtggtggataatatggcaaaggtagatttcattgactgccttttttatagattgagattggggctgattaaaacttcagatcactgcagttgttagggcctgggagattttcctttttaactcctggcctaacagcagcagccgttctgtaggattaactgcacttcgcggtcgttgccttaatctatttgggcttcaggcagggacatgctgggaaggaacagagaccagaggggataggtagggctggggttatctgaaaagaaaacagagaccttttgatttcagccatcttttcagacccagctccctctcccgctgcatgggagaagcaaaggtaaacaggacacattgtccctctccctcagccacagagctcttctgtgagttttgtctttcccaccctggaaaaaaagataaaatacaatttttaaaaggggagggaggaatttagttttaattcaaatgagtagtaatccaatatgccaaaagcagtgggctctacctagatgtaattttactcgtaaatgtgagtcttaaactttgagttgaatggggcaggctgttagaggtggtgtaaattacaggattataaaaatgttagtgctgcccagccttaaagtcaaaaacagaaaaatctctgtgctgttgagtcttcccgccctctctcctgaacaaccttgtaagtaagctagacttttgtttttgccttccatactttccatttcagccattaaacaaaataagccattgaaaccacgattgggttccatgcagagtgacatccgcaatcgggtcaagccagaaggaaatacttgctcgattgccccctatttggcattacaggaaagtctccacactttggaagagtctgaactctcaagacattgaaaatgccaaaggctgcaaacaccctgtgtctttcttgatggagtgcatcttggtgtgttttacaaaggggaattcagtgctgtttttttgttgttgttgttgttttttttttttaaagagcagcatagggcccttctagactcttggattctgtgtctgacaaaaatggtcattaaatgagcaatattataatttagacccatttcactgattttgttccaaattctcaactgacttgagcatctgtttggggctgtagatacattgcccttgttgactgtttttctcgtttctatgggaattactgtagccattactatgtagctttcatagactcaaaacatttttaaagtattgcatataggctggccatatccagtgcctgttactttaccttctttttctaacttaatgcagcagtctgtattaacagatccatttcatttgtctagcttcatcagagagaggctaccccctgatttacaggctgctcacatccaagcaccttgcattctacacttgacagtgattgctaatggcccattcaactaaagtatttgcttgttaacagggaacagaacatgataaatgtccagcaagcttgctgcctccttcagcttttcaaacgcagactggtgcatatttatggcaggcaaatgacaaaagaaaaagctgaattgccctggcctccagctttctatcagaaacagggttaaagtgattaaagcaatcattcaagaaagccctgccgtttgtttactaaccttcatccaacatttagctttgtagtctacctgtgagaagatatttcagaagtattagagataaggaaggaggatctagcaaaccagtgaaaagagtaggtgaccagttataaaatgctttccatgcacattgaatgccaggcgaacctatttctgttattccagcagacaatcagcagtggctctagattattaacatattttcctttcatgtataaattcaaatatgtaattctagtccaaagcattctgtggctggtaagcacatacttgctgatttcaaataagaaaacatagcaagggaaagctccattaaacaagttgtttctgcccttagtaattctctaaacaagataggaagaaaaagtggacagtagtggagtattaatagtgtgctcttttcattctctaaagcacgagtaagtaagcgttcaaactactctgtggtgggcatacatttagagcgctgtgaatgaaccactgctgttctgccatacttaatttatttatattattatttttattttattgttgtttttatgtattattataattatttatttatattactaatttattttctcaatttaaatcctgttgcatccaattttaattacagtttttgtatctgccttcccatacttgctacccacgtccccattgccactgcggccttatccatgttttctgtgtacaccactctcgtatcaccccagaataattatgagtgctacccagacttttgaaaccactagagtcaacatgtttgtctttgaggaaagccaatgatgctttagcatttttggcaggggtggatgtgtgtttaagtggggtgggtgcagctccttattgtctgcctattctactgttgttcccaatccacattccctgcggggcacctaacctgtgtgcatagcaaagaatttccgaccttcagagccagaagtgtttctcaattgatctcttccagcctagggttatagctgatgaattataatccttgctctttccacacctttacctgggcttaccatggccctaaaacatttgcccagaatcagaattgtctcatgagtgagtggggcaaggcaaatcctgttccagaccagctgagaatgtacctagctgcagaagaagttagaaagtgtcatcttttacttatctaccagaactatattcgaggtacattttagatttaaaaaaaaagcaagttctcgtaggccttgaatcccccccttgctatgggaaaatggatcattattataatggactgtccagtaaagttcatgatttctcctagacatgttctctctctttatgacctagatcaagagtgatctctttaagtcttttcttcataatcccacagcactttgtacttagatgtacttagaaagaaccatatacacggtacgtcatgattgatatgcaagccttcaccactctacctgtcctaaaagtcagggacacaccttcttcatttcatcagtccctacttctatccagcattggcatccagtaagtattagtggaatggacagacaacccgaatttgtgctgatggcagtttaccctgttttaactgtcatccttctgctactagacatggatgagacctgagacgatgggactgctcagaggtccctggctcttgaactttagggcaccagaatcccctgcagggcttgagaaaacaggggtttctgggccccacccccagagttcctgattcctgaggtctggggtggggcttgaagatggacatgtttaacaagctcccaggtgacgctggcaactgctgcctcagggccatgctgagaaccctcgccctacacaaacctttctgggaaaacaactcaacattaaagctgtttggggatctctgaagaaatctgtagtccttgccttgttgggggagcatcagggatctaaccattgatggtggagtatttgttgttaattcagcaagcaactattaagtgttaggcctgttactcggctctaacaatacaaggcagagtgacctgtaccctcgagatttaaagtctaagtcctgtagagagaagcccaggtgggagcaagcacatttagagttaggtgcttggtgcaaggtggggacacagaagaagggaatggcatttgcctctggaggggtccggaaacagcctagggaggaggagcttgagtcttgaaatactgtgggcatctctaagcaaagtcacagtagacagctgaaataaagaaaatagtaagcaagccaaagaaacagtatttcagccaagggcagcgtgtgtctatcacgtccacctgtgaacacgtcccaggattctctgcatccggccattgctcaagacagatccctcacaggaacagctaagccactgatttcagctacctgttcacgtgagaattatcagtacctactgcttttcaaaatgagtatgatcatggataggtgaggcaattcagtttcgcagagacagtagggcaagtgccactgtagtttagttaagggcacatgctttagagtttggctatgtgagtccaatcccagtttagccatttattagctgggtagctttaggagcagtagccttagtgtctctcagttgtcccatctctataatagggacaataacataatagtgctgaataaaagagtaacaaaattttggtcaacatttaatgtatttaaagagctaagctccgtgattggcacaatgaaccaatcaatcaaacaccagttgttattaataaaagtcagttgaatatgtactgtgtgcctggccgtggttcaatttgcctttgcatacaaggaaaaaattaaaatactctgttaataaagactatagcataatactttcaccttaaacttcttgatgttaatttattttgtttacctgccaaacttctactcattccttatgactttctgctacatgaaacaccctttgtaattcttttgtcctattaaattaagttctctctcctctgctttcctgcttttggtgctttctaataacacttttaaccctggactttctcattcagctgtgcaactgtggactgagaggaggctctttgaattcattttgtatattctagtagagagtactgtgagcagttgggttgttgaatgaatacattaattcaacctggagggatgggcagtattgcattttttacattgatattacatgatatttagaaaactgcttaactggtggacgttgttttattaacagcattttgtgtatagcactcactatgtgccagctgctattctaactgcctgacaaatactcctgaaaccttcatggtaaccatatgagggaagcacttttaatatatccataataccaacggggagactgtggccaaattggttaattaacttagccaaagtcatattgaactaataagtggatttaaacccagctagtctggggccagggtccctcttttaatcttctgcctcctgcttatgctgttgcatggagtagtctttatcatataactaaattaagcatgcatttgcttaaagcagtgcatacatgatggatcaaaaagtttgtggtataattggtttaattctgtcattatccattttgatttatagtcactttcttatgatggtcgtgtagttttaaatggaacctttgaatctttgatataataaggttatgtcaaatcttgggtataataaggttatacccaatggaaacagaataatgatcagcccatttaaaggatgactggagagttattacaatacataatagtcatgcatatattgagtagtattcctttggtaacattttccttttaaaaattgtaacatttgattgttccttgttgggagaaaaggaggtcagatttttgaggggagatccatttggtgagatgctgagtgtgtgtcaagctaaggagatagtatgacatcttttttagagtctagtcacaattaaatgccattttattttggattttgggatccgtgccagcttccagcttgtcagagctgagaagactcaaatcaagtccaggcttatttctacagcaaactgggattctggcttcttgccggtggattcattcagtacagcccatctggcttttgatgttctgcaagtttggagccatttgttgaaggaagccaggcggtgaatattggtggtcctggggttctcttgactccaagtggtgccccttggtttgcattttcaccatgcttagcatctgcttacctggagaccatgcagccgccggccagaggtctccaacaaccaaatcttcatgccttttagaactcagagtccccagcacatcctccttcctcctccttgtccaattactttcatgcagttctcagtagctgcttgtttgaatcacttatagtatttaacttctagggtgtttttgggttttggtcaaggtaattccaggctgaatgtggtgactaagcaggaaataaatgggtcgtcctcaaagttacagtggagcgctgtttctattttcctaaggtacacagttgtgggggcgatccgtatggaagtcaggaacccagtctgattttgcttccttttgatggtagcagtacagacctggctgttttgtagcctgctttgtttttcttccttttcttccctaacttcacgggctgtggcaaagccctgagacgtgcaggaaaatgtctcctgtcatacgcccacagcagacctagccctgaccctcctctgaagcccaggaaggaggtatctgtgaagcagcctgcttgtaaagcaattgcacacagccttgtaaactgtgttactgggctgattatacttgattggcaaggtgaatctcttatagcaaaagagaacttggagagttttatctcatcttatgccttattaatttgttcattctttaattacacagccacctattgagcaccctatttatgcaaggtacctggtcgggggtcagagggagggtcccatggtaaacgagacagactcaatcctggaggagcaggaatggcagcccctcgctgggctgttggccccaccaaaagggaaaggtttcattttaataatacatgggtgaatcatttttgtcaataggcaaaattctttgtagttaaaaaaaaatatgatggtaggaaggaaagggatgggcagagggttaaaacaaaagatatgctctccctaactctagattgtagtattgttatgcttgtcactgtagctgaattccatttctttgagttttttcaatgccaaggcattccctgtatgacttacgtgagcctttcatctccgcgatttttcccattcaggtaaatgagcaaatggatttgaacactcatatctaaaacaagagagaaccagctggaaatgccctttgaatttctttctctatgtaaaccatttttctttctggtgcctcacctataaataacaggagttccaccttcctttatagactcttgctgaaagcatggtttggaacaagaccgtacaggtgcacacaaattacagttgggaaagaagcctgcagtgcatcttgtctctgaaggttatgaaatcctccttttagtaatggagctggcgtgatcaagccagcaggatgaaatttggcatttgtgagatcaccccccttctcacttgcccactgtacatagcatcccagccttactcttcaaatctccacattttttcttatctagctacaaaattcataggctgatttttttggggtgcgtgtgtggttttttttttgtttttttggtaaataaagacctgcatttttattttgatataggtggttgagttttgtctttaatttcatgacagagatttaactagtctcaacttttgaaaagacaacaatgatatttggggatcacacacttaaagttagatttctagatgattaataccaaagtagatgattttttagcctcagccatttataggtatgcccttctgtgaattttttatgacagtgaaaatcatggcacagataaaaattaaataaatacttctgttattttcctgaagaaaaaaaaaaaaagcttaaactatgagaatactgtctttgagcactttaaaataaaattgacttcagccagcaggattttgagcattacatcacaaataaaaaacaagattaacatcaaaaggagtcagttttcattcaattgtgcagcactgtgggctgtgaaatttaatattattttgactcatatgctaattgtagactgacagaggaaaatggattgtgtttaaataaaaggatacacagcatcacacgcagctgtatcaaatacaagttgaggtctttgggccaggaactgggggccctctagctctgttattgcagattcaagtttgacaaataaaactttcctttagactgtagtttaattactttttttcaaaggtatgcgtgatgaagaggcacaaatacacctcaccttgaagagttgctaaactggtttgtgtgccgatcagttcaccgtgtgtttgaatttctgtgcttctcatctttccttttcttgaaaagattttgcttgtcattggtgtgaattgtaccccccacccccacccatctagtctttgctctcagatttataacactttaatggttccaaattgtatagcctgctcttagaccccttttcttttccttgaataaatcaggttcatgttgcagacgatatttgttttaggaaagtgtgaaagaaggggcacctgtgaaaacacgcaattgttccaacacacatatacatccaaattaaagcagaaaatgtcaaagcctccaatcactaccttatttcttggaggtttaaagccgctgagaagatagtggtgccctcgctggaagttttaaggtaattactttttactctaagcagtagtatctggtaacctaattccgtataaacctgacaccctatcgctacaccccagtatttctctgatttcagaataagtctgcgtagaaacttgttctgatgttaaagtgcaaaagggggcagtaaagtgctatccacaaaaaaggaaaaacattttccaagtatttcttattactgcctgtgtctttcgtaggccctgcctttatttattcattttataacaaaactcttatgtttggggcattcagagaataccttattaagctgttgcagcaatctagcattaaatggaagacatgcaagactgaagatcctgcctgtttatgaagtgtgccatcaaattcacatgctcatgatgcagagtccttctttgggagtattcgtattcccaagtgcacagagcacttcggaaaggagccttggtctttggtgttaatgctctcctagctccgtatagatgtggcaggcccaaagtacatggtggggtgaagggtcaagggtttgggcttatccagagcagcgtgcatcctttgtcaggaggtgactggaaacaccagccaattacagcagaactgcagactgctcatctgcattcggaattgcagatgaaccagtttgtactcgacttctcttcttcactgtaggctttgacatttaattaaaaattaaagccttttatggaaaaagtacatgttttccaaaatggggtaaattcgaagtatacttgatacagaacactggcttgggaataaacctgtgatattacatgacttttggtttgcaactgctaggctgagcctctttgtaaagctgggatttagaatctttgaaatgtttgtacagttcaatgattaagcataaattgtatatattccctttttttcacttatttgagtaaacaagtttgttactacagcttctgtggactcagagatttatgtattaaataggccacaacttcaactaggataattttatttatctgcttgttagggaattgcatcaaaagtttaagtctgtaggcattaaatattttaaatgcttatttttaaagtcaattatgaaagatagcacaaagtttttctgaaactacattaaaaaaataatgttttaatcttatcacaaaagcattgactatttattgcaaagaaaacacagaaagctaaaaatcattctaagtccaccattcagtagcccaaagtggtctcaggtaaaggcggtgtgtgtgaccatttgtttatggttgtctccgtgcagtcagcaaaataaacagaacaacatgccatatattattgatgtgtatattttcaactgaaattagccatctgcttacaatgatcatatacactaatggtataattttgaaatgaaaagaaaaataaaataattctttgtggagagtaatgcgaattgacttatgaatctcgccctgcttggcagtttgctctagaggtagaagagctttatgtgtgggcctcctccccccccacacatttattctgctcacacttgcaccagcatccatgtcaggactcaccttgtcctgttacatgagtaacatggccctgattctcaagtgcatgataactgccataattacacataaatattaaatatttaaatagatctttacgtgtgtaatattaggtagaagtggctctggatcgaatctgatgctttttaaatagaagctttcccacaacatttccaagcactgtcatcgtgtctgtctcgatttggggtttacctggcctagttatctgtctgggtgtagaaactggtagttcctgtttgtatcttttttgttctgatctctttattctgtgtcagctaaatattcttgcagtcagttactaacatattaactcatccttgtttggaaactttggcatatccttccatggtttccttccgtggacctgtcgcgtctctcaggagagccaccaggtatattgtcacacatttcgcatgtattttcagagactacagcagcatcaagtggccccccagcgatttgggttttcttctcggttaatctacactctttggccaaccgtgagaaaacttgtaagaaggcatcagatgtttgtgctaagatttctacctggacaaggactggtactatacccacagataggagacaaattggatattatttgccccaaagtggactctaaaactgttggccagtatgaatattataaagtttatatggttgataaagaccaagcagacagatgcactattaagaaggaaaatacccctctcctcaactgtgccaaaccagaccaagatatcaaattcaccatcaagtttcaagaattcagccctaacctctggggtctagaatttcagaagaacaaagattattacattatatctacatcaaatgggtctttggagggcctggataaccaggagggaggggtgtgccagacaagagccatgaagatcctcatgaaagttggacaagatgcaagttctgctggatcaaccaggaataaagatccaacaagacgtccagaactagaagctggtacaaatggaagaagttcgacaacaagtccctttgtaaaaccaaatccaggttctagcacagacggcaacagcgccggacattcggggaacaacatcctcggttccgaagtggccttatttgcagggattgcttcaggatgcatcatcttcatcgtcatcatcatcacgctggtggtcctcttgctgaagtaccggaggagacacaggaagcactcgccgcagcacacgaccacgctgtcgctcagcacactggccacacccaagcgcagcggcaacaacaacggctcagagcccagtgacattatcatcccgctaaggactgcggacagcgtcttctgccctcactacgagaaggtcagcggggactacgggcacccggtgtacatcgtccaggagatgcccccgcagagcccggcgaacatttactacaaggtctgagagggaccctggtggtacctgtgctttcccagaggacacctaatgtcccgatgcctcccttgagggtttgagagcccgcgtgctggagaattgactgaagcacagcaccgggggagagggacactcctcctcggaagagcccgtcgcgctggacagcttacctagtcttgtagcattcggccttggtgaacacacacgctccctggaagctggaagactgtgcagaagacgcccattcggactgctgtgccgcgtcccacgtctcctcctcgaagccatgtgctgcggtcactcaggcctctgcagaagccaagggaagacagtggtttgtggacgagagggctgtgagcatcctggcaggtgccccaggatgccacgcctggaagggccggcttctgcctggggtgcatttcccccgcagtgcataccggacttgtcacacggacctcgggctagttaaggtgtgcaaagatctctagagtttagtccttactgtctcactcgttctgttacccagggctctgcagcacctcacctgagacctccactccacatctgcatcactcatggaacactcatgtctggagtcccctcctccagccgctggcaacaacagcttcagtccatgggtaatccgttcatagaaattgtgtttgctaacaaggtgccctttagccagatgctaggctgtctgcgaagaaggctaggagttcatagaagggagtggggctggggaaagggctggctgcaattgcagctcactgctgctgcctctgaaacagaaagttggaaaggaaaaaagaaaaaagcaattaggtagcacagcactttggttttgctgagatcgaagaggccagtaggagacacgacagcacacacagtggattccagtgcatggggaggcactcgctgttatcaaatagcgatgtgcaggaagaaaagcccctcttcattccggggaacaaagacgggtattgttgggaaaggaacaggcttggagggaagggagaaagtaggccgctgatgatatattcgggcaggactgttgtggtactggcaataagatacacagctccgagctgtaggagagtcggtctgctttggatgattttttaagcagactcagctgctatacttatcacattttattaaacacagggaaagcatttaggagaatagcagagagccaaatctgacctaaaagttgaaaagccaaaggtcaaacaggctgtaattccatcatcatcgttgttattaaagaatccttatctataaaaggtaggtcagatccccctccccccaggttcctccttcccctcccgattgagccttacgacactttggtttatgcggtgctgtccgggtgccagggctgcagggtcggtactgatggaggctgcagcgcccggtgctctgtgtcaaggtgaagcacatacggcagacctcttagagtccttaagacggaagtaaattatgatgtccagggggagaaggaagataggacgtatttataataggtatatagaacacaagggatataaaatgaaagatttttactaatatatattttaaggttgcacacagtacacaccagaagatgtgaaattcatttgtggcaattaagtggtcccaatgctcagcgcttaaaaaaacaaattggacagctacttctgggaaaaacaacatcattccaaaaagaacaataatgagagcaaatgcaaaaataaccaagtcctccgaaggcatctcacggaaccgtagactaggaagtacgagccccacagagcaggaagccgatgtgactgcatcatatatttaacaatgacaagatgttccggcgtttatttctgcgttgggttttcccttgccttatgggctgaagtgttctctagaatccagcaggtcacactgggggcttcaggtgacgatttagctgtggctccctcctcctgtcctcccccgcaccccctcccttctgggaaacaagaagagtaaacaggaaacctactttttatgtgctatgcaaaatagacatctttaacatagtcctgttactatggtaacactttgctttctgaattggaagggaaaaaaaatgtagcgacagcattttaaggttctcagacctccagtgagtacctgcaaaaatgagttgtcacagaaattatgatcctctatttcctgaacctggaaatgatgttggtccaaagtgcgtgtgtgtatgtgtgagtgggtgcgtggtatacatgtgtacatatatgtataatatatatctacaatatatattatatatatctatatcatatttctgtggagggttgccatggtaaccagccacagtacatatgtaattctttccatcaccccaacctctcctttctgtgcattcatgcaagagtttcttgtaagccatcagaagttacttttaggatgggggagaggggcgagaaggggaaaaatgggaaatagtctgattttaatgaaatcaaatgtatgtatcatcagttggctacgttttggttctatgctaaactgtgaaaaatcagatgaattgataaaagagttccctgcaaccaattgaaaagtgttctgtgcgtctgttttgtgtctggtgcagaatatgacaatctaccaactgtccctttgtttgaagttggtttagctttggaaagttactgtaaatgccttgcttgtatgatcgtccctggtcacccgactttggaatttgcaccatcatgtttcagtgaagatgctgtaaataggttcagattttactgtctatggatttggggtgttacagtagccttattcacctttttaataaaaatacacatgaaaacaagaaagaaatggcttttcttacccagattgtgtacatagagcaatgttggttttttataaagtctaagcaagatgttttgtataaaatctgaattttgcaatgtatttagctacagcttgtttaacggcagtgtcattcccctttgcactgtaatgaggaaaaaatggtataaaaggttgccaaattgctgcatatttgtgccgtaattatgtaccatgaatatttatttaaaatttcgttgtccaatttgtaagtaacacagtattatgcctgagttataaatatttttttctttctttgttttattttaatagcctgtcataggttttaaatctgctttagtttcacattgcagttagccccagaaaatgaaatccgtgaagtcacattccacatctgtttcaaactgaatttgttcttaaaaaaataaaatatttttttcctatggaaaaa

SEQ ID NO: 39 - DNA, 2120 bp, synthetic construct
  source 1..2120: mol_type = other DNA
gctgactctcctcggcggggaccgcggcgccggccggagcgaggagctgcgcacgcagcgggtcccggcgccctccccgcacacaaaggcccgcgcgcgtccggagcccgcggcggggaccgagctccctctttcctgccctgggggccgggagccgcgcggactgagaaggctcctgcgcgcccggaggcgccctactccgctccgtgctccgggacatggaaccgcgccgacgcggcgcccgcgcgctcgggccgctgcccgctgcactggatctatagtcacaggcggccccgctcggggcgcagcgcccgccgcccgcgccggtcgtctccgctccgggtctttgtgtcccagcccgcacccgccccgcgcccacccgccagccgcggccggcccggcaccctggagccgcacgggagtcggccgtccgagctgcgtccggcgcggcgccccggaaccccgagtccgccgcgccgccgcgccccgcgcgtgcgctcccgccccgcgccctgagggccccggagagcgagcgcacctggcccggcgaccgctggagctgcgcagcgcgcctcggagctgcctgcgggcgcacgccgtcttccccgccagtctgccccggaggattgggggtcccagcctgcgtcccgtcagtcccttcttggcccggagtgcgcggagctgggagtggcttcgccatggctgtgagaagggactccgtgtggaagtactgctggggtgttttgatggttttatgcagaactgcgatttccaaatcgatagttttagagcctatctattggaattcctcgaactccaaatttctacctggacaaggactggtactatacccacagataggagacaaattggatattatttgccccaaagtggactctaaaactgttggccagtatgaatattataaagtttatatggttgataaagaccaagcagacagatgcactattaagaaggaaaatacccctctcctcaactgtgccaaaccagaccaagatatcaaattcaccatcaagtttcaagaattcagccctaacctctggggtctagaatttcagaagaacaaagattattacattatatctgtcatctttgtaataggatattcatcagaatcttgccagagactgtgcatttgggatcttgggggatacagcaccaccaccaccctccccctgtccaagagaaacagatcaacatcttaggttgagagtctggggtctggaagacccgagttcctgagtgccctttgacaagtaacttaacccctgtctgcctcagtctcttcatctgtaaagtggggataatgacagcacctgcttcacagggttgatgggaatccagatgtggtgggatatagaaaatgcttattacttccacctttgacaccaaatacatataactaagagttaactttggagcaggggaggaagtgtgaggctccaggctggaggcagacctgtgttcggctgcaagctggagaggatggaccccaaaagcttggctgatttgaagtccatccataaaatggaactccagagagtttacacgtttcagtaatgctgcataacttaattataagatcttctctctttgtcttctttcagtgttataaaagctcttttgtccttgagcttcctttaccaagaaacatgcatttatgtatctttttgttcatggaattgcccaagcttgttagcagatcctttgtaagacccaaaagagacagacaggggaggagtcttcagatacatataatcatttttcccaatttccatgttaccagccttgccaggactttttctcagttccctgttacacaatgaaaatagtgtctctttattgataattttagtagcatcctaatgtggtataaatcgtcttccagagaagaaaatgtgtcagggttgcgttatcactgaggctagctgggaaagtagatcagcccattagtctgataattcgaagcgttgtttctgttatttctgaacatcatgtgaactccttttctgggtgtattaaaggttttcccagtgtgtgtcagtgagactcctgattgaatttaatatgaataaagataaattctttacatttaagga

SEQ ID NO: 40 - DNA, 2278 bp, synthetic construct
  source 1..2278: mol_type = other DNA
agaccgcgagcgagagcgcccccgagcagcgcccgcgccctccgcgccttctccgccgggacctcgagcgaaagacgcccgcccgccgcccagccctcgcctccctgcccaccgggcccaccgcgccgccaccccgaccccgctgcgcacggcctgtccgctgcacaccagcttgttggcgtcttcgtcgccgcgctcgccccgggctactcctgcgcgccacaatgagctcccgcatcgccagggcgctcgccttagtcgtcacccttctccacttgaccaggctggcgctctccacctgccccgctgcctgccactgccccctggaggcgcccaagtgcgcgccgggagtcgggctggtccgggacggctgcggctgctgtaaggtctgcgccaagcagctcaacgaggactgcagcaaaacgcagccctgcgaccacaccaaggggctggaatgcaacttcggcgccagctccaccgctctgaaggggatctgcagagctcagtcagagggcagaccctgtgaatataactccagaatctaccaaaacggggaaagtttccagcccaactgtaaacatcagtgcacatgtattgatggcgccgtgggctgcattcctctgtgtccccaagaactatctctccccaacttgggctgtcccaaccctcggctggtcaaagttaccgggcagtgctgcgaggagtgggtctgtgacgaggatagtatcaaggaccccatggaggaccaggacggcctccttggcaaggagctgggattcgatgcctccgaggtggagttgacgagaaacaatgaattgattgcagttggaaaaggcagctcactgaagcggctccctgtttttggaatggagcctcgcatcctatacaaccctttacaaggccagaaatgtattgttcaaacaacttcatggtcccagtgctcaaagacctgtggaactggtatctccacacgagttaccaatgacaaccctgagtgccgccttgtgaaagaaacccggatttgtgaggtgcggccttgtggacagccagtgtacagcagcctgaaaaagggcaagaaatgcagcaagaccaagaaatcccccgaaccagtcaggtttacttacgctggatgtttgagtgtgaagaaataccggcccaagtactgcggttcctgcgtggacggccgatgctgcacgccccagctgaccaggactgtgaagatgcggttccgctgcgaagatggggagacattttccaagaacgtcatgatgatccagtcctgcaaatgcaactacaactgcccgcatgccaatgaagcagcgtttcccttctacaggctgttcaatgacattcacaaatttagggactaaatgctacctgggtttccagggcacacctagacaaacaagggagaagagtgtcagaatcagaatcatggagaaaatgggcgggggtggtgtgggtgatgggactcattgtagaaaggaagccttgctcattcttgaggagcattaaggtatttcgaaactgccaagggtgctggtgcggatggacactaatgcagccacgattggagaatactttgcttcatagtattggagcacatgttactgcttcattttggagcttgtggagttgatgactttctgttttctgtttgtaaattatttgctaagcatattttctctaggcttttttccttttggggttctacagtcgtaaaagagataataagattagttggacagtttaaagcttttattcgtcctttgacaaaagtaaatgggagggcattccatcccttcctgaagggggacactccatgagtgtctgtgagaggcagctatctgcactctaaactgcaaacagaaatcaggtgttttaagactgaatgttttatttatcaaaatgtagcttttggggagggaggggaaatgtaatactggaataatttgtaaatgattttaattttatattcagtgaaaagattttatttatggaattaaccatttaataaagaaatatttacctaatatctgagtgtatgccattcggtatttttagaggtgctccaaagtcattaggaacaacctagctcacgtactcaattattcaaacaggacttattgggatacagcagtgaattaagctattaaaataagataatgattgcttttataccttcagtagagaaaagtctttgcatataaagtaatgtttaaaaaacatgtattgaacacgacattgtatgaagcacaataaagattctgaagctaaa

SEQ ID NO: 41 - DNA, 1234 bp, synthetic construct
  source 1..1234: mol_type = other DNA
agacgttcgcacacctgggtgccagcgccccagaggtcccgggacagcccgaggcgccgcgcccgccgccccgagctccccaagccttcgagagcggcgcacactcccggtctccactcgctcttccaacacccgctcgttttggcggcagctcgtgtcccagagaccgagttgccccagagaccgagacgccgccgctgcgaaggaccaatgagagccccgctgctaccgccggcgccggtggtgctgtcgctcttgatactcggctcaggccattatgctgctggattggacctcaatgacacctactctgggaagcgtgaaccattttctggggaccacagtgctgatggatttgaggttacctcaagaagtgagatgtcttcagggagtgagatttcccctgtgagtgaaatgccttctagtagtgaaccgtcctcgggagccgactatgactactcagaagagtatgataacgaaccacaaatacctggctatattgtcgatgattcagtcagagttgaacaggtagttaagcccccccaaaacaagacggaaagtgaaaatacttcagataaacccaaaagaaagaaaaagggaggcaaaaatggaaaaaatagaagaaacagaaagaagaaaaatccatgtaatgcagaatttcaaaatttctgcattcacggagaatgcaaatatatagagcacctggaagcagtaacatgcaaatgtcagcaagaatatttcggtgaacggtgtggggaaaagtccatgaaaactcacagcatgattgacagtagtttatcaaaaattgcattagcagccatagctgcctttatgtctgctgtgatcctcacagctgttgctgttattacagtccagcttagaagacaatacgtcaggaaatatgaaggagaagctgaggaacgaaagaaacttcgacaagagaatggaaatgtacatgctatagcataactgaagataaaattacaggatatcacattggagtcactgccaagtcatagccataaatgatgagtcggtcctctttccagtggatcataagacaatggaccctttttgttatgatggttttaaactttcaattgtcactttttatgctatttctgtatataaaggtgcacgaaggtaaaaagtattttttcaagttgtaaataatttatttaatatttaatggaagtgtatttattttacagctcattaaacttttttaaccaaa